(19) United States
(12) Patent Application Publication
(10) Pub. No.: US 2022/0112489 A1
(43) Pub. Date: Apr. 14, 2022

(54) TREM COMPOSITIONS AND USES THEREOF

(71) Applicant: FLAGSHIP PIONEERING, INC., Cambridge, MA (US)

(72) Inventors: David Arthur Berry, Newton, MA (US); Theonie Anastassiadis, Boston, MA (US); Noubar Boghos Afeyan, Lexington, MA (US); Christine Elizabeth Hajdin, Boston, MA (US)

(21) Appl. No.: 17/423,700

(22) PCT Filed: Jan. 17, 2020
(86) PCT No.: PCT/US2020/014092

§ 371 Date: Jul. 16, 2021

Related U.S. Application Data
(60) Provisional application No. 62/794,342, filed on Jan. 18, 2019.
(60) Provisional application No. 62/855,547, filed on May 31, 2019.

(51) Int. Cl. C12N 15/11; A61K 31/7105; C12N 5/09

(52) U.S. Cl. CPC C12N 15/11; C12N 2330/50; C12N 5/0693; A61K 31/7105

(57) ABSTRACT

The invention relates generally to tRNA-based effector molecules and methods relating thereto.

### CROSS-REFERENCE TO RELATED APPLICATIONS

This application claims priority to U.S. Provisional Application 62/794,342 filed on Jan. 18, 2019, and U.S. Provisional Application 62/855,547 filed on May 31, 2019, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Jan. 8, 2020, is named F2099-7000WO_SL.txt and is 228,808 bytes in size.

### BACKGROUND

tRNAs are complex RNA molecules that possess a number of functions including the initiation and elongation of proteins.

### SUMMARY

In an aspect, the disclosure provides a method of making a purified tRNA effector molecule (TREM) pharmaceutical composition, comprising:

providing a mammalian host cell comprising an exogenous nucleic acid, e.g., a DNA or RNA, encoding the TREM;

maintaining the mammalian cell under conditions sufficient to express the TREM;

purifying the TREM from the mammalian host cell, e.g., according to a method described herein; and

formulating the purified TREM as a pharmaceutical composition, e.g., by combining the TREM with a pharmaceutical excipient,

thereby making the TREM pharmaceutical composition.

In an embodiment, the nucleic acid comprises an RNA, which upon reverse transcription, results in a DNA which can be transcribed into the TREM.

In an embodiment, the nucleic acid comprises an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof.

In an embodiment, the nucleic acid comprises an RNA sequence comprising a consensus sequence, e.g., as provided herein, e.g., a consensus sequence of Formula I_{ZZZ}, Formula II_{ZZZ}, or Formula III_{ZZZ}, wherein _{ZZZ} indicates any of the twenty amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an embodiment, the mammalian host cell is chosen from: a non-human cell or cell line, or a human cell or cell line, e.g., a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.

In an embodiment, the purification step comprises one, two or all of the following steps, e.g., in the order recited:

(i) separating nucleic acids from cellular debris to provide an RNA preparation;

(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; or/and

(iii) separating a TREM from other RNA species in the small RNA preparation by affinity-based separation, e.g., sequence affinity-based separation.

In one aspect, the invention features a method of making a tRNA effector molecule (TREM) composition, comprising:

(a) providing a host cell, comprising exogenous nucleic acid, e.g., a DNA or RNA, encoding a TREM under conditions sufficient to express the TREM, and

(b) purifying the expressed TREM from the host cell culture to produce a TREM composition,

thereby making a TREM composition.

In an embodiment, the TREM composition is a pharmaceutically acceptable composition.

In another aspect, the invention features a method of making a pharmaceutical TREM composition, comprising:

a) providing a purified TREM composition, e.g., a purified TREM composition made by culturing a mammalian host cell comprising DNA or RNA encoding a TREM under conditions sufficient to express the TREM, and purifying the expressed TREM from the host cell culture to produce a purified TREM composition,

b) providing a value, e.g., by evaluating or testing, for a characteristic described herein (e.g., a characteristic related to identity (e.g., sequence), purity (e.g., process impurity such as TREM fragments, host cell protein or host cell DNA), activity (e.g., adaptor activity)),

c) optionally, formulating the purified TREM composition as a pharmaceutical drug product (e.g., combining the TREM composition with a pharmaceutical excipient) if it meets a reference criterion for the one or more characteristic,

thereby making the pharmaceutical TREM composition.

In another aspect, the invention features a method of making a pharmaceutical TREM composition comprising:

combining

a) a TREM, e.g., a purified TREM composition, e.g., a TREM composition made by a method described herein; and

b) a pharmaceutically acceptable component, e.g., an excipient,

thereby making a pharmaceutical TREM composition.

In another aspect, the present disclosure provides a composition comprising a purified tRNA effector molecule (TREM) (e.g., a purified TREM composition made according to a method described herein), comprising an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof.

In an aspect, the present disclosure provides a composition comprising a purified tRNA effector molecule (TREM) (e.g., a purified TREM composition made according to a method described herein), comprising an RNA sequence comprising a consensus sequence provided herein, e.g., a consensus sequence of Formula I_{ZZZ}, Formula II_{ZZZ}, or Formula III_{ZZZ}, wherein _{ZZZ} indicates any of the twenty amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In another aspect, the invention features a GMP-grade, recombinant TREM composition (e.g., a TREM composition made in compliance with cGMP, and/or in accordance with similar requirements) comprising an RNA sequence at least 80% (e.g., at least 85%, at least 90%, at least 95%, at least 97%, at least 98%, at least 99%) identical to an RNA encoded by a DNA sequence listed in Table 1, or a fragment or functional fragment thereof.

In another aspect, the invention features a GMP-grade, recombinant TREM composition (e.g., a TREM composition made in compliance with cGMP, and/or in accordance with similar requirements) comprising an RNA sequence comprising a consensus sequence provided herein.

In an aspect, the invention features a TREM comprising a consensus sequence provided herein.

In an aspect, the invention features a TREM comprising a consensus sequence of Formula I_{ZZZ}, wherein _{ZZZ} indicates any of the twenty amino acids and Formula I corresponds to all species.

In an aspect, the invention features a TREM comprising a consensus sequence of Formula II_{ZZZ}, wherein _{ZZZ} indicates any of the twenty amino acids and Formula II corresponds to mammals.

In an aspect, the invention features a TREM comprising a consensus sequence of Formula III_{ZZZ}, wherein _{ZZZ} indicates any of the twenty amino acids and Formula III corresponds to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an aspect, the invention features a GMP-grade, recombinant TREM composition comprising an RNA sequence comprising a consensus sequence provided herein.

In an embodiment of any of the TREM compositions or pharmaceutical TREM compositions provided herein, the composition comprises one or more, e.g., a plurality, of TREMs.

In an embodiment of any of the TREM compositions or pharmaceutical TREM compositions provided herein, the composition comprises at least 1, 2, 3, 4, 5, 6, 7, 8, 9 or 10 species of TREMs.

In an embodiment of any of the TREM compositions or pharmaceutical TREM compositions provided herein, the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) Fragments of less than 0.1%, 1%, 2%, 3%, 4%, 5%, 6%, 7%, 8%, 9% or 10%;
(vi) low levels or absence of endotoxins, e.g., a negative result as measured by the Limulus amebocyte lysate (LAL) test;
(vii) in-vitro translation activity, e.g., as measured by an assay described in Example 15; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of, or an undetectable level of viral contamination.

In another aspect, the invention features, a cell comprising an exogenous nucleic acid comprising:

a nucleic acid sequence, e.g., DNA or RNA, that encodes a TREM, wherein the nucleic acid sequence comprises:
(i) a control region sequence;
(ii) a sequence encoding a modified TREM;
(iii) a sequence encoding more than one TREM;
(iv) a sequence other than a tRNA^{Met} sequence; or
(v) a promoter sequence that comprises a Pol III recognition site, e.g., a U6 promoter, a 7SK promoter or a H1 promoter, or a fragment thereof.

In an aspect, the invention features a method of modulating a tRNA pool in a cell comprising:

providing a purified TREM composition, and contacting the cell with the TREM composition,

thereby modulating the tRNA pool in the cell.

In another aspect, the invention features a method of delivering a TREM to a cell, tissue, or subject, comprising:

providing a cell, tissue, or subject, and contacting the cell, tissue, or subject, with a TREM composition comprising the TREM, e.g., a pharmaceutical TREM composition comprising the TREM.

In another aspect, the invention features a method of treating a subject, e.g., modulating the metabolism, e.g., the translational capacity of a cell, in a subject, comprising:

providing, e.g., administering to the subject, an exogenous nucleic acid, e.g., a DNA or RNA, which encodes a TREM, thereby treating the subject.

In an embodiment of any of the methods disclosed herein, the TREM composition is made by:

providing a mammalian host cell comprising an exogenous nucleic acid, e.g., a DNA or RNA, encoding the TREM;

maintaining the mammalian cell under conditions sufficient to express the TREM; and/or purifying the TREM from the mammalian host cell, e.g., according to a method described herein.

In an embodiment of any of the methods disclosed herein, the mammalian host cell is a non-human cell or cell line, or a human cell or cell line chosen from: a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.

In an embodiment of any of the methods disclosed herein, the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species by affinity-based separation, e.g., sequence affinity-based separation.

In an embodiment of any of the methods disclosed herein, the TREM comprises:
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein.

In an aspect, the disclosure provides a method of making a purified tRNA effector molecule (TREM) pharmaceutical composition, comprising:

providing an insect host cell comprising an exogenous nucleic acid, e.g., a DNA or RNA, encoding the TREM;

maintaining the insect host cell under conditions sufficient to express the TREM;

purifying the TREM from the insect host cell, e.g., according to a method described herein; and

formulating the purified TREM as a pharmaceutical composition, e.g., by combining the TREM with a pharmaceutical excipient,

thereby making the TREM pharmaceutical composition.

In an embodiment, the insect host cell is chosen from: an insect cell or cell line, e.g., a Sf9 cell or cell line.

In an embodiment, the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

In an aspect, the disclosure provides a method of making a purified tRNA effector molecule (TREM) pharmaceutical composition, comprising:

providing a yeast host cell comprising an exogenous nucleic acid, e.g., a DNA or RNA, encoding the TREM;

maintaining the yeast host cell under conditions sufficient to express the TREM;

purifying the TREM from the yeast host cell, e.g., according to a method described herein; and

formulating the purified TREM as a pharmaceutical composition, e.g., by combining the TREM with a pharmaceutical excipient,

thereby making the TREM pharmaceutical composition.

In an embodiment, the yeast host cell is chosen from: a yeast cell or cell line, e.g., a S. cerevisiae or S. pombe cell or cell line.

In an embodiment, the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

As disclosed herein tRNA-based effector molecules (TREMs) are complex molecules which can mediate a variety of cellular processes. Pharmaceutical TREM compositions can be administered to cells, tissues or subjects to modulate these functions, e.g., in vitro or in vivo. Disclosed herein are TREM compositions, preparations, methods of making TREM compositions and preparations, and methods of using TREM compositions and preparations.

Additional features of any of the aforesaid TREM compositions, preparations, methods of making TREM compositions and preparations, and methods of using TREM compositions and preparations include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A method of making a purified tRNA effector molecule (TREM) pharmaceutical composition, comprising:

providing a mammalian host cell comprising an exogenous nucleic acid, e.g., a DNA or RNA, encoding the TREM;

maintaining the mammalian host cell under conditions sufficient to express the TREM; purifying the TREM from the mammalian host cell, e.g., according to a method described herein; and

formulating the purified TREM as a pharmaceutical composition, e.g., by combining the TREM with a pharmaceutical excipient,

thereby making the TREM pharmaceutical composition.

2. A method of making a tRNA effector molecule (TREM) composition, comprising:

(a) providing a mammalian host cell comprising exogenous nucleic acid, e.g., a DNA or RNA, encoding a TREM under conditions sufficient to express the TREM, and

(b) purifying the expressed TREM from the mammalian host cell to produce a TREM composition,

thereby making the TREM composition.

3. The method of embodiment 2, the TREM composition is formulated as a pharmaceutical composition, e.g., by combining the TREM with a pharmaceutical excipient,
4. A method of making a pharmaceutical TREM composition comprising:

combining

a) a TREM, e.g., a purified TREM composition, e.g., a TREM composition made by a method described herein; and

b) a pharmaceutically acceptable component, e.g., an excipient,

thereby making a pharmaceutical TREM composition.

5. The method of claim 4, wherein the TREM is purified from a mammalian host cell, e.g., according to a method described herein.
6. A method of making a purified tRNA effector molecule (TREM) pharmaceutical composition, comprising:

purifying the TREM from a mammalian host cell;

formulating the purified TREM as a pharmaceutical composition, e.g., by combining the TREM with a pharmaceutical excipient,

thereby making the TREM pharmaceutical composition.

7. The method of claim 5 or 6, wherein the mammalian host cell comprises an exogenous nucleic acid, e.g., a DNA or RNA, encoding the TREM.
8. The method of any one of embodiments 1-7, wherein the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation;
(iii) separating a TREM from other RNA species in the small RNA preparation by affinity-based separation, e.g., sequence affinity.
   9. The method of embodiment 8, comprising step (i).
   10. The method of embodiment 8 or 9, comprising step (ii).
   11. The method of any one of embodiments 8 to 10, comprising step (iii).
   12. The method of any one of embodiments 8, or 10-11, comprising performing:

step (i) before step (ii).

13. The method of any one of embodiments 8, or 11-12 comprising performing step (ii) before step (iii).
14. The method of any one of embodiments 8-13, wherein (i) comprises extracting the nucleic acids from protein.
15. The method of any one of embodiments 8-14, wherein (i) comprises a phenol/chloroform extraction.
16. The method of any one of embodiments 8-10 or 12-15, wherein (ii) comprises separating RNA of less than a first size class from RNA of a second, larger, size class.
17. The method of embodiment 16, wherein the first size class is less than 200 nt.
18. The method of any one of embodiments 8, or 9-16, wherein (ii) comprises performing a salt precipitation to enrich for RNA of less than 200 nt.
19. The method of embodiment 18, wherein the salt comprises LiCl.
20. The method of any one of embodiments 8-10 or 12-19, wherein (ii) further comprises performing a desalting or buffer exchange step.
21. The method of any one of embodiments 8, or 11-20, wherein (iii) comprises performing an affinity-based separation to enrich for a TREM.
22. The method of embodiment 21, wherein the affinity-based separation comprises a sequence based separation, e.g., using a probe comprising a sequence that binds to a TREM.
23. The method of any one of the preceding embodiments, wherein the TREM composition is a pharmaceutically acceptable composition.
24. The method of any one of embodiments 1-3 or 7-23, comprising introducing the exogenous DNA or RNA into the mammalian host cell.
25. The method of any one of embodiments 1-3 or 7-24, wherein the nucleic acid comprises a DNA, which upon transcription, expresses a TREM.
26. The method of any one of embodiments 1-3 or 7-25, wherein the nucleic acid comprises an RNA, which upon reverse transcription, results in a DNA which can be transcribed to provide the TREM.
27. The method of any one of the preceding embodiments, wherein the TREM recognizes a stop codon.
28. The method of claim 27, wherein the TREM mediates acceptance and incorporation of an amino acid.
29. The method of any one of embodiments 1 to 27, wherein the TREM does not recognize a stop codon.
30. The method of any one of embodiments 1 to 29, wherein the TREM comprises:
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or

(ii) an RNA sequence comprising a consensus sequence provided herein.

31. The method of any one of the preceding embodiments, wherein the TREM composition comprises a TREM fragment, e.g., as described herein, optionally wherein the TREM fragment is produced in vivo, in the host cell.
32. The method of embodiment 31, wherein the TREM fragment is produced by fragmenting an expressed TREM after production of the TREM by the cell, e.g., a TREM produced by the host cell is fragmented after release or purification from the host cell, e.g., the TREM is fragmented ex vivo.
33. The method of any one of the preceding embodiments, wherein the method results in an increase, e.g., at least a 2.2, 2.5, 3, 4, 5, 6, 7, 8, 9, 10, or 20-fold increase in the production of total endogenous tRNA and TREM in the host cell (e.g., as measured by an assay described in any of Examples 7-11), e.g., as compared with a reference cell, e.g., a similar cell but not engineered or modified to express a TREM.
34. The method of embodiment 33, wherein the method results in an increase in TREM production and/or tRNA production between 2.2 to 20-fold, between 2.2 to 15-fold, between 2.2 to 10-fold, between 2.2 to 9-fold, between 2.2 to 8-fold, between 2.2 to 7-fold, between 2.2 to 6-fold, between 2.2 to 5-fold, between 2.2 to 4-fold, between 2.2 to 3-fold, between 2.2 to 2.5-fold, between 2.5 to 20-fold, between 3 to 20-fold, between 4 to 20-fold, between 5 to 20-fold, between 6 to 20-fold, between 7 to 20-fold, between 8 to 20-fold, between 9 to 20-fold, between 10 to 20-fold, or between 15 to 20-fold.
35. The method of any one of the preceding embodiments, wherein the method results in a detectable level of TREM in the host cell, e.g., as measured by an assay described in any of Examples 7-11.
36. The method of any one of the preceding embodiments, wherein the host cell is capable of a post-transcriptional modification, of the TREM.
37. The method of any one of the preceding embodiments, wherein the host cell is capable of a post-transcriptional modification, of the TREM, e.g., a post-transcriptional modification selected from Table 2.
38. The method of any one of the preceding embodiments, wherein the host cell has been modified to modulate, e.g., increase, its ability to provide a post-transcriptional modification, of the TREM, e.g., a post-transcriptional modification selected from Table 2, e.g., the host cell has been modified to provide for, an increase, or decrease in, the expression of a gene, e.g., a gene encoding an enzyme from Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC.
39. The method of any one of the preceding embodiments, wherein the host cell is a mammalian cell capable of a post-transcriptional modification, of the TREM, e.g., a post-transcriptional modification selected from Table 2.
40. The method of any one of the preceding embodiments, wherein the host cell comprises a cell selected from a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.
41. The method of any one of the preceding embodiments, wherein the host cell comprises a HeLa cell, a HEK293 cell, a HT-1080 cell, a PER.C6 cell, a HKB-11 cell, a CAP cell or a HuH-7 cell.
42. The method of any one of the preceding embodiments, wherein the host cell has increased expression of an oncogene, e.g., Ras, c-myc or c-jun.
43. The method of any one of the preceding embodiments, wherein the host cell has decreased expression of a tumor suppressor, e.g., p53 or Rb.
44. The method of any one of the preceding embodiments, wherein the host cell has increased expression of RNA Polymerase III (RNA Pol III).
45. The method of any one of the preceding embodiments, wherein the host cell has increased expression of a tRNA^{Met}, e.g., tRNA^{iMet} or. tRNA^{eMet}.
46. The method of any one of the preceding embodiments, comprising culturing the host cell in a medium that promotes cell hyperproliferation (e.g., which promotes a signaling pathway amplified in cancer cells).
47. The method of any one of the preceding embodiments, comprising culturing the host cell in a medium that promotes growth, e.g., medium comprising or supplemented with one or a combination of growth factors, cytokines or hormones, e.g., one or a combination of serum (e.g., fetal bovine serum (FBS)), fibroblast growth factor (FGF), epidermal growth factors (EGF), insulin-like growth factors (IGF), transforming growth factor beta (TGFb), platelet derived growth factor (PDGF), hepatocyte growth factor (HGF), or tumor necrosis factor (TNF).
48. The method of any one of the preceding embodiments, comprising culturing the host cell in a medium that promotes post-transcriptional processing, e.g., of the TREM.
49. The method of any one of the preceding embodiments, comprising culturing the host cell under conditions, e.g., a medium that promotes overexpression or hyperactivation of enzymes involved in post-transcriptional processing, e.g., under conditions that promote:

a) removal of a 5′ leader sequence e.g., by RNase P;

b) 3′ trailer sequence exonuclease activity, e.g., RNase II, PNPase, RNase PH or RNase T activity;

c) CCA addition at a 3′ end, e.g., by a nucleotidyltransferase;

d) intron splicing, e.g., by one or more (e.g., all) of: a splicing endonuclease, a cyclic phosphodiesterase, an adenylyltransferase, a ligase, or a 2′ phosphotransferase;

e) a modification, e.g., by a modification enzyme, e.g., an enzyme that has one or more of the following enzymatic activities:
(i) adenosine A₃₄ to inosine I₃₄ deamination;
(ii) methylation of adenosine m¹A₅₈;
(iii) making a ncm⁵Um₃₄ or ncm⁵s²U₃₄ modification;
(iv) making a ct⁶A modification; isopentylation i⁶A₃₇ modification; A₃₇ to i⁶A₃₇ modification; or
(v) making a modification listed in Table 2; or

f) a synthetase involved in amino acid charging.

50. The method of any one of the preceding embodiments, comprising culturing the host cell in a medium that has an excess of nutrients, e.g., is not nutrient limiting.
51. The method of any one of the preceding embodiments, comprising culturing the host cell in a medium that promotes expression, e.g., increases expression and/or activity, of Mck1 and/or Kns1.
52. The method of any one of the preceding embodiments, wherein the host cell has increased expression and/or activity of Trm1.
53. The method of any one of the preceding embodiments, wherein the host cell has decreased activity of Maf1, e.g., by phosphorylation of Maf1, e.g., phosphorylation of a Serine in position 45 of Maf1.
54. The method of embodiment 53, wherein a decrease in the activity of Maf1 results in increased TREM production.
55. The method of embodiment 53 or 54, wherein the activity of Maf1 can be decreased by introducing a phosphomimetic Maf1 mutant, e.g., a mutant with a Serine to Aspartate mutation at position 45 (S45D); or by hyperactivating CK2/TORC1, e.g., which phosphorylates Maf1.
56. The method of any one of the preceding embodiments, further comprising measuring one or more of the following characteristics of the TREM composition (or an intermediate in the production of a TREM composition):
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) fragments of less than 0.1%, 1%, 2%, 3%, 4%, 5%, 6%, 7%, 8%, 9% or 10%;
(vi) low levels or absence of endotoxins, e.g., a negative result as measured by the Limulus amebocyte lysate (LAL) test;
(vii) in-vitro translation activity, e.g., as measured by an assay described in Example 15; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of or an undetectable level of viral contamination.
   57. The method of embodiment 56, further comprising, comparing the measured value with a reference value or a standard.
   58. The method of embodiment 57, further comprising, in response to the comparison, modulating the TREM composition to:
(i) increase the purity of the TREM composition;
(ii) decrease the amount of HCP in the composition;
(iii) decrease the amount of DNA in the composition;
(iv) decrease the amount of fragments in the composition;
(v) decrease the amount of endotoxins in the composition;
(vi) increase the in vitro translation activity of the composition;
(vii) increase the TREM concentration of the composition; or
(viii) increase the sterility of the composition.
   59. A method of making a TREM composition, comprising:

contacting a TREM containing a reaction mixture with a reagent, e.g., a capture reagent or a separation reagent, comprising a nucleic acid sequence complimentary with a TREM;

thereby making a TREM composition.

60. The method of embodiment 59, further comprising, denaturing a TREM, e.g., prior to hybridization with the capture reagent.
61. The method of embodiment 59, further comprising, renaturing a TREM, e.g., after hybridization and/or release from the capture reagent.
62. The method of any of embodiments 59-61, further wherein a single capture reagent is used, e.g., to make a TREM composition, wherein at least 50%, 55%, 60%, 65%, 70%, 75%, 80%, 85%, 90%, or 95% of the TREMs have a sequence complimentary with the capture reagent.
63. The method of any of embodiments 59-61, further wherein a plurality of capture reagents are used, e.g., to make a TREM composition having a plurality of different TREMs.
64. A method of making a pharmaceutical composition, comprising:

a) providing a purified TREM composition, e.g., a purified TREM composition made by culturing a mammalian host cell comprising DNA or RNA encoding a TREM under conditions sufficient to express the TREM, and purifying the expressed TREM from the host cell culture to produce a purified TREM composition,

b) providing a value, e.g., by evaluating or testing, for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of, or an undetectable level of viral contamination.

c) optionally, formulating the purified TREM composition as a pharmaceutical drug product (e.g., combining the TREM composition with a pharmaceutical excipient) if it meets a reference criteria for the one or more characteristics,

thereby making a pharmaceutical composition.

65. The method of embodiment 64, further comprising, comparing the measured value with a reference value or a standard.
66. The method of embodiment 65, further comprising, in response to the comparison, modulating the composition to:
(i) increase the purity of the TREM composition;
(ii) decrease the amount of HCP in the composition;
(iii) decrease the amount of DNA in the composition;
(iv) decrease the amount of fragments in the composition;
(v) decrease the amount of endotoxins in the composition;
(vi) increase the in vitro translation activity of the composition;
(vii) increase the TREM concentration of the composition; or
(viii) increase the sterility of the composition.
   67. A composition comprising a purified tRNA effector molecule (TREM) (e.g., a purified TREM composition made according to a method described herein), comprising:
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein, and optionally the RNA sequence is less than 100% identical to an RNA sequence encoded by a DNA sequence listed in Table 1.
   68. A GMP-grade, recombinant TREM composition (e.g., a TREM composition made in compliance with cGMP, and/or in accordance with similar requirements) comprising: (i) an RNA sequence at least 80% ((e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein, and optionally the RNA sequence is less than 100% identical to an RNA sequence encoded by a DNA sequence listed in Table 1.
   69. A pharmaceutical tRNA effector molecule (TREM) composition, comprising
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein, and optionally the RNA sequence is less than 100% identical to an RNA sequence encoded by a DNA sequence listed in Table 1.
   70. The pharmaceutical TREM composition of claim 69, comprising a purified tRNA effector molecule (TREM) (e.g., a purified TREM composition made according to a method described herein).
   71. The composition or pharmaceutical composition of any one of embodiments 67-70, wherein the TREM is made according to any one of embodiments 1-66.
   72. The composition or pharmaceutical composition of any one of embodiments 67-70, wherein the TREM comprises one or more post-transcriptional modifications listed in Table 2.
   73. The composition or pharmaceutical composition of embodiment 72, wherein the TREM comprises one or more post-transcriptional modifications listed in Table 2.
   74. A recombinant TREM composition of at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.
   75. A recombinant TREM composition of between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to 500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.
   76. A TREM composition comprising a consensus sequence of Formula I_{ZZZ},

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein:
R is a ribonucleotide residue;
(i) _{ZZZ} indicates any of the twenty amino acids;
(ii) Formula I corresponds to all species; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
   77. A TREM composition comprising a consensus sequence of Formula II_{ZZZ}, R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein:
R is a ribonucleotide residue;
(i) _{ZZZ} indicates any of the twenty amino acids;
(ii) Formula II corresponds to mammals; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
   78. A TREM composition comprising a consensus sequence of Formula III_{ZZZ},

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein:
R is a ribonucleotide residue;
(i) _{ZZZ} indicates any of the twenty amino acids;
(ii) Formula III corresponds to humans; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
   79. The composition or pharmaceutical composition of any one of embodiments 67-78, wherein the composition comprises one or more, e.g., a plurality, of TREMs.
   80. The composition or pharmaceutical composition of any one of embodiments 67-79, wherein the composition comprises one or more unique TREMs, e.g., one or more TREMs that comprise different anti-codon sequences.
   81. The composition or pharmaceutical composition of any one of embodiments 67-80, wherein the composition comprises one or more unique TREMs, e.g., TREMs that recognize different codons.
   82. The composition or pharmaceutical composition of any one of embodiments 67-81, wherein the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of, or an undetectable level of viral contamination.
   83. A method of modulating a tRNA pool in a cell comprising:

providing a purified TREM composition, and contacting the cell with the TREM composition,

thereby modulating the tRNA pool in the cell.

84. A method of contacting a cell, tissue, or subject with a TREM, comprising

contacting the cell, tissue or subject with a purified TREM composition, thereby contacting a cell, tissue, or subject with the TREM.

85. A method of presenting a TREM to a cell, tissue, or subject with a TREM, comprising

contacting the cell, tissue or subject with a purified TREM composition, thereby presenting the TREM to a cell, tissue, or subject.

86. A method of forming a TREM-contacted cell, tissue, or subject, comprising

contacting the cell, tissue or subject with a purified TREM composition, thereby forming a TREM-contacted cell, tissue, or subject.

87. A method of using a TREM comprising,

contacting the cell, tissue or subject with a purified TREM composition, thereby using the TREM.

88. A method of applying a TREM to a cell, tissue, or subject, comprising

contacting the cell, tissue or subject with a purified TREM composition, thereby applying a TREM to a cell, tissue, or subject.

89. A method of exposing a cell, tissue, or subject to a TREM, comprising

contacting the cell, tissue or subject with a purified TREM composition, thereby exposing a cell, tissue, or subject to a TREM.

90. A method of forming an admixture of a TREM and a cell, tissue, or subject, comprising

contacting the cell, tissue or subject with a TREM composition, thereby forming an admixture of a TREM and a cell, tissue, or subject.

91. A method of delivering a TREM to a cell, tissue, or subject, comprising:

providing a cell, tissue, or subject, and contacting the cell, tissue, or subject, with a TREM composition, e.g., a purified TREM composition, e.g., a pharmaceutical TREM composition.

92. A method, e.g., an ex vivo method, of modulating the metabolism, e.g., the translational capacity of an organelle, comprising:

providing a preparation of an organelle, e.g., mitochondria or chloroplasts, and contacting the organelle with a pharmaceutical TREM composition.

93. A method of treating a subject, e.g., modulating the metabolism, e.g., the translational capacity of a cell, in a subject, comprising:

providing, e.g., administering to the subject, an exogenous nucleic acid, e.g., a DNA or RNA, which encodes a TREM,

thereby treating the subject.

94. The method of any one of embodiments 83-93, wherein the TREM composition is made according to any one of embodiments 1-66, or the TREM comprises a composition provided in any one of embodiments 67-82.
95. The method of any one of embodiments 83-93, wherein the TREM composition is made by:

providing a mammalian host cell comprising an exogenous nucleic acid, e.g., a DNA or RNA, encoding the TREM;

maintaining the mammalian cell under conditions sufficient to express the TREM; and/or

purifying the TREM from the mammalian host cell, e.g., according to a method described herein.

96. The method of embodiment 95, wherein the mammalian host cell is chosen from: a non-human cell or cell line, or a human cell or cell line, e.g., a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.
97. The method of any one of embodiments 95-96, wherein the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity-based separation.
   98. The method of any one of embodiments 83-97, wherein the TREM comprises:
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein, and optionally the RNA sequence is less than 100% identical to an RNA sequence encoded by a DNA sequence listed in Table 1.
   99. The method of any one of embodiments 83-98, wherein the method is an in vitro method, e.g., a cell or tissue, is contacted with the TREM composition in vitro.
   100. The method of any one of embodiments 83-98, wherein the method is an ex vivo method, e.g., a cell or tissue, is contacted with the TREM composition ex vivo, and optionally, the contacted cell or tissue is introduced, e.g., administered, into a subject, e.g., the subject from which the cell or tissue came, or a different subject.
   101. The method of any one of embodiments 83-98, wherein the method is an in vivo method, e.g., a subject, or a tissue or cell of a subject, is contacted with the TREM composition in vivo.
   102. The method of any of embodiments 99-101, comprising contacting the TREM composition, e.g., a pharmaceutical TREM composition, with a cell.
   103. The method of any of embodiments 99-101, comprising contacting the TREM composition, e.g., a pharmaceutical TREM composition, with a tissue.
   104. The method of any of embodiments 99-100 or 102, comprising administering the TREM composition, e.g., a pharmaceutical TREM composition, to a subject.
   105. The method of any of embodiments 100 or 104, wherein the TREM composition is administered with a carrier or delivery agent, e.g., a liposome, a polymer (e.g., a polymer conjugate), a particle, a microsphere, microparticle, or a nanoparticle.
   106. The method of any of embodiments 99-105, wherein the cell is cancerous.
   107. The method of any of embodiments 99-105, wherein the cell is noncancerous.
   108. The method of any of embodiments 99-102, or 104-107, wherein the cell or tissue comprises:

a muscle cell or tissue (e.g., a skeletal muscle cell or tissue, a smooth muscle cell or tissue, or a cardiac muscle cell or tissue),

an epithelial cell or tissue;

a connective cell or tissue (e.g., adipose cell or tissue, bone cell or tissue, or blood cell), or

a nervous cell or tissue (e.g., a sensory neuron, a motor neuron, or an interneuron).

109. The method of any of embodiments 99-108, wherein the method comprises administering a cell that was contacted ex vivo or in vitro, with a TREM composition, to a subject.
110. A cell comprising a TREM made according to any one of embodiments 1-66.
111. A cell comprising a TREM of any one of embodiments 67-82.
112. A cell comprising an exogenous nucleic acid comprising:

a nucleic acid sequence, e.g., DNA or RNA, that encodes a TREM, wherein the nucleic acid sequence comprises:
(i) a control region sequence;
(ii) a sequence encoding a modified TREM;
(iii) a sequence encoding more than one TREM;
(iv) a sequence other than a tRNA^{Met} sequence; or
(v) a promoter sequence that comprises a Pol III recognition site, e.g., a U6 promoter, a 7SK promoter or a H1 promoter, or a fragment thereof.
   113. The method of any of embodiments 111-112, wherein the host cell is capable of a post-transcriptional modification, of the TREM.
   114. The method of any of embodiments 111-113, wherein the host cell is capable of a post-transcriptional modification, of the TREM, e.g., a post-transcriptional modification selected from Table 2.
   115. The method of any of embodiments 111-114, wherein the host cell has been modified to modulate, e.g., increase, its ability to provide a post-transcriptional modification, of the TREM, e.g., a post-transcriptional modification selected from Table 2, e.g., the host cell has been modified to provide for, an increase, or decrease in, the expression of a gene, e.g., a gene encoding an enzyme from Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC.
   116. The method of any of embodiments 111-115, wherein the host cell is a mammalian cell capable of a post-transcriptional modification, of the TREM, e.g., a post-transcriptional modification selected from Table 2.
   117. The method of any of embodiments 111-116, wherein the host cell comprises a cell or cell line chosen from: a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.
   118. The method of any of embodiments 111-117, wherein the host cell comprises a cell or cell line chosen from: a HeLa cell, a HEK293 cell, a HT-1080 cell, a PER.C6 cell, a HKB-11 cell, a CAP cell or a HuH-7 cell.
   119. The method of any of embodiments 111-1118, wherein the host cell has increased expression of an oncogene, e.g., Ras, c-myc or c-jun.
   120. The method of any of embodiments 111-119, wherein the host cell has decreased expression of a tumor suppressor, e.g., p53 or Rb.
   121. The method of any of embodiments 111-120, wherein the host cell has increased expression of RNA Polymerase III (RNA Pol III).
   122. The method of any of embodiments 111-121, wherein the host cell has increased expression of a tRNA^{Met}, e.g., tRNA^{iMet} or. tRNA^{eMet}.
   123. The method of any of embodiments 111-122, comprising culturing the host cell in a medium that promotes cell hyperproliferation (e.g., which promotes a signaling pathway amplified in cancer cells).
   124. The method of any of embodiments 111-123, comprising culturing the host cell in a medium that promotes growth, e.g., medium comprising or supplemented with one or a combination of growth factors, cytokines or hormones, e.g., one or a combination of serum (e.g., fetal bovine serum (FBS)), fibroblast growth factor (FGF), epidermal growth factors (EGF), insulin-like growth factors (IGF), transforming growth factor beta (TGFb), platelet derived growth factor (PDGF), hepatocyte growth factor (HGF), or tumor necrosis factor (TNF).
   125. The method of any of embodiments 111-124, comprising culturing the host cell in a medium that promotes post-transcriptional processing, e.g., of the TREM.
   126. The method of any of embodiments 111-125, comprising culturing the host cell under conditions, e.g., a medium that promotes overexpression or hyperactivation of enzymes involved in post-transcriptional processing, e.g., under conditions that promote:

a) removal of a 5′ leader sequence e.g., by RNase P;

b) 3′ trailer sequence exonuclease activity, e.g., RNase II, PNPase, RNase PH or RNase T activity;

c) CCA addition at a 3′ end, e.g., by a nucleotidyltransferase;

d) intron splicing, e.g., by one or more (e.g., all) of: a splicing endonuclease, a cyclic phosphodiesterase, an adenylyltransferase, a ligase, or a 2′ phosphotransferase;

e) a modification, e.g., by a modification enzyme, e.g., an enzyme that has one or more of the following enzymatic activities:
(i) adenosine A₃₄ to inosine I₃₄ deamination;
(ii) methylation of adenosine m¹A₅₈;
(iii) making a ncm⁵Um₃₄ or ncm⁵s²U₃₄ modification;
(iv) making a ct⁶A modification; isopentylation i⁶A₃₇ modification; A₃₇ to i⁶A₃₇ modification; or
(v) making a modification listed in Table 2; or

f) a synthetase involved in amino acid charging.

127. The method of any of embodiments 111-126, comprising culturing the host cell in a medium that has an excess of nutrients, e.g., is not nutrient limiting.
128. The method of any of embodiments 111-127, comprising culturing the host cell in a medium that promotes expression, e.g., increases expression and/or activity, of Mck1 and/or Kns1.
129. The method of any of embodiments 111-128, wherein the host cell has increased expression and/or activity of Trm1.
130. The method of any of embodiments 111-129, wherein the host cell has decreased activity of Maf1, e.g., by phosphorylation of Maf1, e.g., phosphorylation of a Serine in position 45 of Maf1.
131. The method of embodiment 130, wherein a decrease in the activity of Maf1 results in increased TREM production.
132. The method of embodiment 130 or 131, wherein the activity of Maf1 can be decreased by introducing a phosphomimetic Maf1 mutant, e.g., a mutant with a Serine to Aspartate mutation at position 45 (S45D); or by hyperactivating CK2/TORC1, e.g., which phosphorylates Maf1.
133. A reaction mixture comprising a TREM and a reagent, e.g., a capture reagent, or a separation reagent.
134. A bioreactor comprising a plurality of mammalian host cells described herein comprising exogenous DNA or RNA encoding a TREM.
135. The bioreactor of embodiment 134,
(i) comprising at least 1×10⁷, 1×10⁸, 1×10⁹, 1×10¹⁰, 1×10¹¹, 1×10¹², 1×10¹³, or 1×10¹⁴ host cells;
(ii) comprising between 100 mL and 100 liters of culture medium, e.g., at least 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, or 100 liters of culture medium;
(iii) wherein the bioreactor is selected from a continuous flow bioreactor, a batch process bioreactor, a perfusion bioreactor, and a fed batch bioreactor; or
(iv) wherein the bioreactor is held under conditions sufficient to express the TREM.
   136. A master cell bank comprising a host cell, e.g., as described herein.
   137. The master cell bank of embodiment 136, wherein the master cell bank comprises at least 1×10⁷, 1×10⁸, 1×10⁹, 1×10¹⁰, 1×10¹¹, 1×10¹², 1×10¹³, 1×10¹⁴, 1×10¹⁵, 1×10²⁰, 1×10²⁵, or 1×10³⁰ host cells.
   138. A method of evaluating a composition of TREM, e.g., a GMP-grade TREM (i.e., a TREM made in compliance with cGMP, and/or in accordance with similar requirements), comprising acquiring a value for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., the composition or preparation supports the growth of fewer than 100 viable microorganisms as tested under aseptic conditions, the composition or preparation meets the standard of USP <71>, and/or the composition or preparation meets the standard of USP <85> as described by cGMP guidelines for sterile drug products produced by aseptic processing; or
(x) viral contamination, e.g., the composition or preparation has an absence of, or an undetectable level of viral contamination.
   139. The method of making of any one of embodiments 1-66, the composition or pharmaceutical composition of any one of embodiments 67-82, the method of any one of embodiments 83-109, the cell of any one of embodiments 110-132, the reaction mixture of embodiment 133, the bioreactor of embodiment 134 or 135, the master cell bank of embodiment 136 or 137, or the method of evaluating of embodiment 138, wherein the TREM is encoded by, or expressed from, a nucleic acid sequence comprising:
(i) a control region sequence;
(ii) a sequence encoding a modified TREM;
(iii) a sequence encoding more than one TREM; or
(iv) a sequence other than a tRNA^{Met} sequence.
   140. The method, composition or pharmaceutical composition, cell, reaction mixture, bioreactor, or master cell bank of embodiment 74, wherein the nucleic acid sequence comprises a promoter sequence.
   141. The method, composition or pharmaceutical composition, cell, reaction mixture, bioreactor, or master cell bank of embodiment 139 or 140, wherein the nucleic acid sequence comprises a promoter sequence that comprises an RNA polymerase III (Pol III) recognition site, e.g., a Pol III binding site, e.g., a U6 promoter sequence or fragment thereof.
   142. The method, composition or pharmaceutical composition, cell, reaction mixture, bioreactor, or master cell bank of any one of embodiments 139-141, wherein the nucleic acid sequence comprises a promoter sequence that comprises a mutation, e.g., a promoter-up mutation, e.g., a mutation that increases transcription initiation, e.g., a mutation that increases TFIIIB binding.
   143. The method, composition or pharmaceutical composition, cell, reaction mixture, bioreactor, or master cell bank of any one of embodiments 139-142, wherein the nucleic acid sequence comprises a promoter sequence which increases Pol III binding and results in increased tRNA production, e.g., TREM production.
   144. The method of making of any one of embodiments 1-66 or 139-143, the composition or pharmaceutical composition of any one of embodiments 67-82 or 139-143, the method of any one of embodiments 83-109 or 139-143, the cell of any one of embodiments 110-132 or 139-143, the reaction mixture of embodiment 133 or 139-143, the bioreactor of embodiment 134-135 or 139-143, the master cell bank of embodiment 136-137 or 139-143, or the method of evaluating of embodiment 138 or 139-143, wherein the TREM enhances:

(a) the stability of a product, e.g., a protein, and/or

(b) ribosome occupancy of a product.

145. The method of making of any one of embodiments 1-66 or 139-144, the composition or pharmaceutical composition of any one of embodiments 67-82 or 139-144, the method of any one of embodiments 83-109 or 139-144, the cell of any one of embodiments 110-132 or 139-144, the reaction mixture of embodiment 133 or 139-144, the bioreactor of embodiment 134-135 or 139-144, the master cell bank of embodiment 136-137 or 139-144, or the method of evaluating of embodiment 138 or 139-144, wherein the TREM:

modulates ribosome occupancy;

modulates protein translation or stability;

modulates mRNA stability;

modulates protein folding or structure;

modulates protein transduction or compartmentalization;

modulates codon usage;

modulates cell fate; or

modulates a signaling pathway, e.g., a cellular signaling pathway.

146. The method of making of any one of embodiments 1-66 or 139-144, the composition or pharmaceutical composition of any one of embodiments 67-82 or 139-144, the method of any one of embodiments 83-109 or 139-144, the cell of any one of embodiments 110-132 or 139-144, the reaction mixture of embodiment 133 or 139-144, the bioreactor of embodiment 134-135 or 139-144, the master cell bank of embodiment 136-137 or 139-144, or the method of evaluating of embodiment 138 or 139-144, wherein the TREM comprises a post-transcriptional modification from Table 2.
147. The method of making of any one of embodiments 1-66 or 139-146, the composition or pharmaceutical composition of any one of embodiments 67-82 or 139-146, the method of any one of embodiments 83-109 or 139-146, the cell of any one of embodiments 110-132 or 139-146, the reaction mixture of embodiment 133 or 139-146, the bioreactor of embodiment 134-135 or 139-146, the master cell bank of embodiment 136-137 or 139-146, or the method of evaluating of embodiment 138 or 139-146, wherein the TREM comprises cognate adaptor function, and wherein the TREM mediates acceptance and incorporation of an amino acid associated in nature with the anti-codon of the TREM in the initiation or elongation of a peptide chain.
148. The method of making of any one of embodiments 1-66 or 139-147, the composition or pharmaceutical composition of any one of embodiments 67-82 or 139-147, the method of any one of embodiments 83-109 or 139-147, the cell of any one of embodiments 110-132 or 139-147, the reaction mixture of embodiment 133 or 139-147, the bioreactor of embodiment 134-135 or 139-147, the master cell bank of embodiment 136-137 or 139-147, or the method of evaluating of embodiment 138 or 139-147, wherein the TREM comprises non-cognate adaptor function, and wherein the TREM mediates acceptance and incorporation of an amino acid, e.g., a non-cognate amino acid, other than the amino acid associated in nature with the anti-codon of the TREM, in the initiation or elongation of a peptide chain, and the non-cognate amino acid residue is, e.g., a desired residue, e.g., a residue that does not mediate a disorder or unwanted trait, e.g., a wild type residue.
149. The method of making of any one of embodiments 1-66 or 139-148, the composition or pharmaceutical composition of any one of embodiments 67-82 or 139-148, the method of any one of embodiments 83-109 or 139-148, the cell of any one of embodiments 110-132 or 139-148, the reaction mixture of embodiment 133 or 139-148, the bioreactor of embodiment 134-135 or 139-148, the master cell bank of embodiment 136-137 or 139-148, or the method of evaluating of embodiment 138 or 139-148, wherein the TREM comprises an anti-codon sequence which is complimentary with a codon which

specifies a first amino acid residue, e.g., an unwanted or undesired codon, e.g., a codon associated with a disorder or unwanted trait, e.g., a mutant codon, and

the TREM mediates incorporation of a second amino acid residue, e.g., a desired codon, e.g., an amino acid not associated with a disorder or unwanted trait, e.g., a wild type amino acid.

150. The method of making of any one of embodiments 1-66 or 139-149, the composition or pharmaceutical composition of any one of embodiments 67-75, 79-82 or 139-149, the method of any one of embodiments 83-109 or 139-149, the cell of any one of embodiments 110-132 or 139-149, the reaction mixture of embodiment 133 or 139-149, the bioreactor of embodiment 134-135 or 139-149, the master cell bank of embodiment 136-137 or 139-149, or the method of evaluating of embodiment 138 or 139-149, wherein the TREM comprises an RNA sequence at least 80% (e.g., at least 85%, at least 90%, at least 95%, at least 97%, at least 98%, at least 99%) identical to an RNA sequence of a tRNA which occurs naturally.
151. The method of making of any one of embodiments 1-66 or 139-150, the composition or pharmaceutical composition of any one of embodiments 67-75, 79-82 or 139-150, the method of any one of embodiments 83-109 or 139-150, the cell of any one of embodiments 110-132 or 139-150, the reaction mixture of embodiment 133 or 139-150, the bioreactor of embodiment 134-135 or 139-150, the master cell bank of embodiment 136-137 or 139-150, or the method of evaluating of embodiment 138 or 139-150, wherein the TREM comprises an RNA sequence at least 80% (e.g., at least 85%, at least 90%, at least 95%, at least 97%, at least 98%, at least 99%) identical to an RNA encoded by a DNA sequence listed in Table 1, or a fragment or functional fragment thereof.
152. The method of making of any one of embodiments 1-66 or 139-151, the composition or pharmaceutical composition of any one of embodiments 67-75, 79-82 or 139-151, the method of any one of embodiments 83-109 or 139-151, the cell of any one of embodiments 110-132 or 139-151, the reaction mixture of embodiment 133 or 139-151, the bioreactor of embodiment 134-135 or 139-151, the master cell bank of embodiment 136-137 or 139-151, or the method of evaluating of embodiment 138 or 139-151, wherein the TREM comprises:

an RNA sequence encoded by a DNA sequence listed in Table 1, or a fragment thereof.

153. The method of making of any one of embodiments 1-66 or 139-152, the composition or pharmaceutical composition of any one of embodiments 67-75, 79-82 or 139-152, the method of any one of embodiments 83-109 or 139-152, the cell of any one of embodiments 110-132 or 139-152, the reaction mixture of embodiment 133 or 139-152, the bioreactor of embodiment 134-135 or 139-152, the master cell bank of embodiment 136-137 or 139-152, or the method of evaluating of embodiment 138 or 139-152, wherein the TREM comprises

an RNA sequence at least XX % identical to an RNA sequence encoded by a DNA sequence listed in Table 1, or a fragment thereof, wherein XX is selected from 80, 85, 90, 95, 96, 97, 98, or 99.

154. The method, composition or pharmaceutical composition, cell, reaction mixture, bioreactor, or master cell bank of embodiment 153, wherein XX is 80.
155. The method, composition or pharmaceutical composition, cell, reaction mixture, bioreactor, or master cell bank of embodiment 153, wherein XX is 85.
156. The method, composition or pharmaceutical composition, cell, reaction mixture, bioreactor, or master cell bank of embodiment 153, wherein XX is 90.
157. The method, composition or pharmaceutical composition, cell, reaction mixture, bioreactor, or master cell bank of embodiment 153, wherein XX is 95.
158. The method, composition or pharmaceutical composition, cell, reaction mixture, bioreactor, or master cell bank of embodiment 153, wherein XX is 97.
159. The method, composition or pharmaceutical composition, cell, reaction mixture, bioreactor, or master cell bank of embodiment 153, wherein XX is 98.
160. The method, composition or pharmaceutical composition, cell, reaction mixture, bioreactor, or master cell bank of embodiment 153, wherein XX is 99.
161. The method, composition or pharmaceutical composition, cell, reaction mixture, bioreactor, or master cell bank of any one of embodiments 153-160, wherein the DNA sequence is SEQ ID NO:1 or a fragment thereof, or SEQ ID NO:2 or a fragment thereof, or SEQ ID NO: 3 or a fragment thereof, or SEQ ID NO:4 or a fragment thereof, or SEQ ID NO: 5 or a fragment thereof, or SEQ ID NO: 6 or a fragment thereof, or SEQ ID NO: 7 or a fragment thereof, or SEQ ID NO:8 or a fragment thereof, or SEQ ID NO: 9 or a fragment thereof, or SEQ ID NO:10 or a fragment thereof, or SEQ ID NO: 11 or a fragment thereof, or SEQ ID NO:12 or a fragment thereof, or SEQ ID NO: 13 or a fragment thereof, or SEQ ID NO: 14 or a fragment thereof, or SEQ ID NO: 15 or a fragment thereof, or SEQ ID NO: 16 or a fragment thereof, or SEQ ID NO: 17 or a fragment thereof, or SEQ ID NO: 18 or a fragment thereof, or SEQ ID NO: 19 or a fragment thereof, or SEQ ID NO: 20 or a fragment thereof, or SEQ ID NO: 21 or a fragment thereof, or SEQ ID NO: 22 or a fragment thereof, or SEQ ID NO: 23 or a fragment thereof, or SEQ ID NO: 24 or a fragment thereof, or SEQ ID NO: 25 or a fragment thereof, or SEQ ID NO: 26 or a fragment thereof, or SEQ ID NO: 27 or a fragment thereof, or SEQ ID NO: 28 or a fragment thereof, or SEQ ID NO: 29 or a fragment thereof, or SEQ ID NO: 30 or a fragment thereof, or SEQ ID NO: 31 or a fragment thereof, or SEQ ID NO: 32 or a fragment thereof, or SEQ ID NO: 33 or a fragment thereof, or SEQ ID NO: 34 or a fragment thereof, or SEQ ID NO: 35 or a fragment thereof, or SEQ ID NO: 36 or a fragment thereof, or SEQ ID NO: 37 or a fragment thereof, or SEQ ID NO: 38 or a fragment thereof, or SEQ ID NO: 39 or a fragment thereof, or SEQ ID NO: 40 or a fragment thereof, or SEQ ID NO: 41 or a fragment thereof, or SEQ ID NO: 42 or a fragment thereof, or SEQ ID NO: 43 or a fragment thereof, or SEQ ID NO: 44 or a fragment thereof, or SEQ ID NO: 45 or a fragment thereof, or SEQ ID NO: 46 or a fragment thereof, or SEQ ID NO: 47 or a fragment thereof, or SEQ ID NO: 48 or a fragment thereof, or SEQ ID NO: 49 or a fragment thereof, or SEQ ID NO: 50 or a fragment thereof, or SEQ ID NO: 51 or a fragment thereof, or SEQ ID NO: 52 or a fragment thereof, or SEQ ID NO: 53 or a fragment thereof, or SEQ ID NO: 54 or a fragment thereof, or SEQ ID NO: 55 or a fragment thereof, or SEQ ID NO: 56 or a fragment thereof, or SEQ ID NO: 57 or a fragment thereof, or SEQ ID NO: 58 or a fragment thereof, or SEQ ID NO: 59 or a fragment thereof, or SEQ ID NO: 60 or a fragment thereof, or SEQ ID NO: 61 or a fragment thereof, or SEQ ID NO: 62 or a fragment thereof, or SEQ ID NO: 63 or a fragment thereof, or SEQ ID NO: 64 or a fragment thereof, or SEQ ID NO: 65 or a fragment thereof, or SEQ ID NO: 66 or a fragment thereof, or SEQ ID NO: 67 or a fragment thereof, or SEQ ID NO: 68 or a fragment thereof, or SEQ ID NO: 69 or a fragment thereof, or SEQ ID NO: 70 or a fragment thereof,} or SEQ ID NO: 71 or a fragment thereof, or SEQ ID NO: 72 or a fragment thereof, or SEQ ID NO: 73 or a fragment thereof, or SEQ ID NO: 74 or a fragment thereof, or SEQ ID NO: 75 or a fragment thereof, or SEQ ID NO: 76 or a fragment thereof, or SEQ ID NO: 77 or a fragment thereof, or SEQ ID NO: 78 or a fragment thereof, or SEQ ID NO: 79 or a fragment thereof, or SEQ ID NO: 80 or a fragment thereof, or SEQ ID NO: 81 or a fragment thereof, or SEQ ID NO: 82 or a fragment thereof, or SEQ ID NO: 83 or a fragment thereof, or SEQ ID NO: 84 or a fragment thereof, or SEQ ID NO: 85 or a fragment thereof, or SEQ ID NO: 86 or a fragment thereof, or SEQ ID NO: 87 or a fragment thereof, or SEQ ID NO: 88 or a fragment thereof, or SEQ ID NO: 89 or a fragment thereof, or SEQ ID NO: 90 or a fragment thereof, or SEQ ID NO: 91 or a fragment thereof, or SEQ ID NO: 92 or a fragment thereof, or SEQ ID NO: 93 or a fragment thereof, or SEQ ID NO: 94 or a fragment thereof, or SEQ ID NO: 95 or a fragment thereof, or SEQ ID NO: 96 or a fragment thereof, or SEQ ID NO: 97 or a fragment thereof, or SEQ ID NO: 98 or a fragment thereof, or SEQ ID NO: 99 or a fragment thereof, or SEQ ID NO: 100 or a fragment thereof, or SEQ ID NO: 101 or a fragment thereof, or SEQ ID NO: 102 or a fragment thereof, or SEQ ID NO: 103 or a fragment thereof, or SEQ ID NO: 104 or a fragment thereof, or SEQ ID NO: 105 or a fragment thereof, or SEQ ID NO: 106 or a fragment thereof, or SEQ ID NO: 107 or a fragment thereof, or SEQ ID NO: 108 or a fragment thereof, or SEQ ID NO:109 or a fragment thereof, or SEQ ID NO: 110 or a fragment thereof, or SEQ ID NO: 111 or a fragment thereof, or SEQ ID NO: 112 or a fragment thereof, or SEQ ID NO: 113 or a fragment thereof, or SEQ ID NO: 114 or a fragment thereof, or SEQ ID NO: 115 or a fragment thereof, or SEQ ID NO: 116 or a fragment thereof, or SEQ ID NO: 117 or a fragment thereof, or SEQ ID NO: 118 or a fragment thereof, or SEQ ID NO: 119 or a fragment thereof, or SEQ ID NO: 120 or a fragment thereof, or SEQ ID NO: 121 or a fragment thereof, or SEQ ID NO: 122 or a fragment thereof, or SEQ ID NO: 123 or a fragment thereof, or SEQ ID NO: 124 or a fragment thereof, or SEQ ID NO: 125 or a fragment thereof, or SEQ ID NO: 126 or a fragment thereof, or SEQ ID NO: 127 or a fragment thereof, or SEQ ID NO: 128 or a fragment thereof, or SEQ ID NO: 129 or a fragment thereof, or SEQ ID NO: 130 or a fragment thereof, or SEQ ID NO: 131 or a fragment thereof, or SEQ ID NO: 132 or a fragment thereof, or SEQ ID NO: 133 or a fragment thereof, or SEQ ID NO: 134 or a fragment thereof, or SEQ ID NO: 135 or a fragment thereof, or SEQ ID NO:136 or a fragment thereof, or SEQ ID NO: 137 or a fragment thereof, or SEQ ID NO: 138 or a fragment thereof, or SEQ ID NO: 139 or a fragment thereof, or SEQ ID NO: 140 or a fragment thereof, or SEQ ID NO: 141 or a fragment thereof, or SEQ ID NO: 142 or a fragment thereof, or SEQ ID NO: 143 or a fragment thereof, or SEQ ID NO: 144 or a fragment thereof, or SEQ ID NO: 145 or a fragment thereof, or SEQ ID NO: 146 or a fragment thereof, or SEQ ID NO: 147 or a fragment thereof, or SEQ ID NO: 148 or a fragment thereof, or SEQ ID NO: 149 or a fragment thereof, or SEQ ID NO: 150 or a fragment thereof, or SEQ ID NO: 151 or a fragment thereof, or SEQ ID NO: 152 or a fragment thereof, or SEQ ID NO: 153 or a fragment thereof, or SEQ ID NO: 154 or a fragment thereof, or SEQ ID NO: 155 or a fragment thereof, or SEQ ID NO: 156 or a fragment thereof, or SEQ ID NO: 157 or a fragment thereof, or SEQ ID NO: 158 or a fragment thereof, or SEQ ID NO: 159 or a fragment thereof, or SEQ ID NO: 160 or a fragment thereof, or SEQ ID NO: 161 or a fragment thereof, or SEQ ID NO: 162 or a fragment thereof, or SEQ ID NO: 163 or a fragment thereof, or SEQ ID NO: 164 or a fragment thereof, or SEQ ID NO: 165 or a fragment thereof, or SEQ ID NO: 166 or a fragment thereof, or SEQ ID NO: 167 or a fragment thereof, or SEQ ID NO: 168 or a fragment thereof, or SEQ ID NO: 169 or a fragment thereof, or SEQ ID NO: 170 or a fragment thereof, or SEQ ID NO: 171 or a fragment thereof, or SEQ ID NO: 172 or a fragment thereof, or SEQ ID NO: 173 or a fragment thereof, or SEQ ID NO: 174 or a fragment thereof, or SEQ ID NO: 175 or a fragment thereof, or SEQ ID NO: 176 or a fragment thereof, or SEQ ID NO: 177 or a fragment thereof, or SEQ ID NO: 178 or a fragment thereof, or SEQ ID NO: 179 or a fragment thereof, or SEQ ID NO: 180 or a fragment thereof, or SEQ ID NO: 181 or a fragment thereof, or SEQ ID NO: 182 or a fragment thereof, or SEQ ID NO: 183 or a fragment thereof, or SEQ ID NO: 184 or a fragment thereof, or SEQ ID NO: 185 or a fragment thereof, or SEQ ID NO: 186 or a fragment thereof, or SEQ ID NO: 187 or a fragment thereof, or SEQ ID NO: 188 or a fragment thereof, or SEQ ID NO: 189 or a fragment thereof, or SEQ ID NO: 190 or a fragment thereof, or SEQ ID NO: 191 or a fragment thereof, or SEQ ID NO: 192 or a fragment thereof, or SEQ ID NO: 193 or a fragment thereof, or SEQ ID NO: 194 or a fragment thereof, or SEQ ID NO: 195 or a fragment thereof, or SEQ ID NO: 196 or a fragment thereof, or SEQ ID NO: 197 or a fragment thereof, or SEQ ID NO: 198 or a fragment thereof, or SEQ ID NO: 199 or a fragment thereof, or SEQ ID NO: 200 or a fragment thereof, or SEQ ID NO: 201 or a fragment thereof, or SEQ ID NO: 202 or a fragment thereof, or SEQ ID NO: 203 or a fragment thereof, or SEQ ID NO: 204 or a fragment thereof, or SEQ ID NO: 205 or a fragment thereof, or SEQ ID NO: 206 or a fragment thereof, or SEQ ID NO: 207 or a fragment thereof, or SEQ ID NO: 208 or a fragment thereof, or SEQ ID NO: 209 or a fragment thereof, or SEQ ID NO: 210 or a fragment thereof, or SEQ ID NO: 211 or a fragment thereof, or SEQ ID NO: 212 or a fragment thereof, or SEQ ID NO: 213 or a fragment thereof, or SEQ ID NO: 214 or a fragment thereof, or SEQ ID NO: 215 or a fragment thereof, or SEQ ID NO: 216 or a fragment thereof, or SEQ ID NO: 217 or a fragment thereof, or SEQ ID NO: 218 or a fragment thereof, or SEQ ID NO: 219 or a fragment thereof, or SEQ ID NO: 220 or a fragment thereof, or SEQ ID NO: 221 or a fragment thereof, or SEQ ID NO: 222 or a fragment thereof, or SEQ ID NO: 223 or a fragment thereof, or SEQ ID NO: 224 or a fragment thereof, or SEQ ID NO: 225 or a fragment thereof, or SEQ ID NO: 226 or a fragment thereof, or SEQ ID NO: 227 or a fragment thereof, or SEQ ID NO: 228 or a fragment thereof, or SEQ ID NO: 229 or a fragment thereof, or SEQ ID NO: 230 or a fragment thereof, or SEQ ID NO: 231 or a fragment thereof, or SEQ ID NO: 232 or a fragment thereof, or SEQ ID NO: 233 or a fragment thereof, or SEQ ID NO: 234 or a fragment thereof, or SEQ ID NO: 235 or a fragment thereof, or SEQ ID NO: 236 or a fragment thereof, or SEQ ID NO: 237 or a fragment thereof, or SEQ ID NO: 238 or a fragment thereof, or SEQ ID NO: 239 or a fragment thereof, or SEQ ID NO: 240 or a fragment thereof, or SEQ ID NO: 241 or a fragment thereof, or SEQ ID NO: 242 or a fragment thereof, or SEQ ID NO: 243 or a fragment thereof, or SEQ ID NO: 244 or a fragment thereof, or SEQ ID NO: 245 or a fragment thereof, or SEQ ID NO: 246 or a fragment thereof, or SEQ ID NO: 247 or a fragment thereof, or SEQ ID NO: 248 or a fragment thereof, or SEQ ID NO: 249 or a fragment thereof, or SEQ ID NO: 250 or a fragment thereof, or SEQ ID NO: 251 or a fragment thereof, or SEQ ID NO: 252 or a fragment thereof, or SEQ ID NO: 253 or a fragment thereof, or SEQ ID NO: 254 or a fragment thereof, or SEQ ID NO: 255 or a fragment thereof, or SEQ ID NO: 256 or a fragment thereof, or SEQ ID NO: 257 or a fragment thereof, or SEQ ID NO: 258 or a fragment thereof, or SEQ ID NO: 259 or a fragment thereof, or SEQ ID NO: 260 or a fragment thereof, or SEQ ID NO: 261 or a fragment thereof, or SEQ ID NO: 262 or a fragment thereof, or SEQ ID NO: 263 or a fragment thereof, or SEQ ID NO: 264 or a fragment thereof, or SEQ ID NO: 265 or a fragment thereof, or SEQ ID NO: 266 or a fragment thereof, or SEQ ID NO: 267 or a fragment thereof, or SEQ ID NO: 268 or a fragment thereof, or SEQ ID NO: 269 or a fragment thereof, or SEQ ID NO: 270 or a fragment thereof, or SEQ ID NO: 271 or a fragment thereof, or SEQ ID NO: 272 or a fragment thereof, or SEQ ID NO: 273 or a fragment thereof, or SEQ ID NO: 274 or a fragment thereof, or SEQ ID NO: 275 or a fragment thereof, or SEQ ID NO: 276 or a fragment thereof, or SEQ ID NO: 277 or a fragment thereof, or SEQ ID NO: 278 or a fragment thereof, or SEQ ID NO: 279 or a fragment thereof, or SEQ ID NO: 280 or a fragment thereof, or SEQ ID NO: 281 or a fragment thereof, or SEQ ID NO: 282 or a fragment thereof, or SEQ ID NO: 283 or a fragment thereof, or SEQ ID NO: 284 or a fragment thereof, or SEQ ID NO: 285 or a fragment thereof, or SEQ ID NO: 286 or a fragment thereof, or SEQ ID NO: 287 or a fragment thereof, or SEQ ID NO: 288 or a fragment thereof, or SEQ ID NO: 289 or a fragment thereof, or SEQ ID NO: 290 or a fragment thereof, or SEQ ID NO: 291 or a fragment thereof, or SEQ ID NO: 292 or a fragment thereof, or SEQ ID NO: 293 or a fragment thereof, or SEQ ID NO: 294 or a fragment thereof, or SEQ ID NO: 295 or a fragment thereof, or SEQ ID NO: 296 or a fragment thereof, or SEQ ID NO: 297 or a fragment thereof, or SEQ ID NO: 298 or a fragment thereof, or SEQ ID NO: 299 or a fragment thereof, or SEQ ID NO: 300 or a fragment thereof, or SEQ ID NO: 301 or a fragment thereof, or SEQ ID NO: 302 or a fragment thereof, or SEQ ID NO: 303 or a fragment thereof, or SEQ ID NO: 304 or a fragment thereof, or SEQ ID NO: 305 or a fragment thereof, or SEQ ID NO: 306 or a fragment thereof, or SEQ ID NO: 307 or a fragment thereof, or SEQ ID NO: 308 or a fragment thereof, or SEQ ID NO: 309 or a fragment thereof, or SEQ ID NO: 310 or a fragment thereof, or SEQ ID NO: 311 or a fragment thereof, or SEQ ID NO: 312 or a fragment thereof, or SEQ ID NO: 313 or a fragment thereof, or SEQ ID NO: 314 or a fragment thereof, or SEQ ID NO: 315 or a fragment thereof, or SEQ ID NO: 316 or a fragment thereof, or SEQ ID NO: 317 or a fragment thereof, or SEQ ID NO: 318 or a fragment thereof, or SEQ ID NO: 319 or a fragment thereof, or SEQ ID NO: 320 or a fragment thereof, or SEQ ID NO: 321 or a fragment thereof, or SEQ ID NO: 322 or a fragment thereof, or SEQ ID NO: 323 or a fragment thereof, or SEQ ID NO: 324 or a fragment thereof, or SEQ ID NO: 325 or a fragment thereof, or SEQ ID NO: 326 or a fragment thereof, or SEQ ID NO: 327 or a fragment thereof, or SEQ ID NO: 328 or a fragment thereof, or SEQ ID NO: 329 or a fragment thereof, or SEQ ID NO: 330 or a fragment thereof, or SEQ ID NO: 331 or a fragment thereof, or SEQ ID NO: 332 or a fragment thereof, or SEQ ID NO: 333 or a fragment thereof, or SEQ ID NO: 334 or a fragment thereof, or SEQ ID NO: 335 or a fragment thereof, or SEQ ID NO: 336 or a fragment thereof, or SEQ ID NO: 337 or a fragment thereof, or SEQ ID NO: 338 or a fragment thereof, or SEQ ID NO: 339 or a fragment thereof, or SEQ ID NO: 340 or a fragment thereof, or SEQ ID NO: 341 or a fragment thereof, or SEQ ID NO: 342 or a fragment thereof, or SEQ ID NO: 343 or a fragment thereof, or SEQ ID NO: 344 or a fragment thereof, or SEQ ID NO: 345 or a fragment thereof, or SEQ ID NO: 346 or a fragment thereof, or SEQ ID NO: 347 or a fragment thereof, or SEQ ID NO: 348 or a fragment thereof, or SEQ ID NO: 349 or a fragment thereof, or SEQ ID NO: 350 or a fragment thereof, or SEQ ID NO: 351 or a fragment thereof, or SEQ ID NO: 352 or a fragment thereof, or SEQ ID NO: 353 or a fragment thereof, or SEQ ID NO: 354 or a fragment thereof, or SEQ ID NO: 355 or a fragment thereof, or SEQ ID NO: 356 or a fragment thereof, or SEQ ID NO: 357 or a fragment thereof, or SEQ ID NO: 358 or a fragment thereof, or SEQ ID NO: 359 or a fragment thereof, or SEQ ID NO: 360 or a fragment thereof, or SEQ ID NO: 361 or a fragment thereof, or SEQ ID NO: 362 or a fragment thereof, or SEQ ID NO: 363 or a fragment thereof, or SEQ ID NO: 364 or a fragment thereof, or SEQ ID NO: 365 or a fragment thereof, or SEQ ID NO: 366 or a fragment thereof, or SEQ ID NO: 367 or a fragment thereof, or SEQ ID NO: 368 or a fragment thereof, or SEQ ID NO: 369 or a fragment thereof, or SEQ ID NO: 370 or a fragment thereof, or SEQ ID NO: 371 or a fragment thereof, or SEQ ID NO: 372 or a fragment thereof, or SEQ ID NO: 373 or a fragment thereof, or SEQ ID NO: 374 or a fragment thereof, or SEQ ID NO: 375 or a fragment thereof, or SEQ ID NO: 376 or a fragment thereof, or SEQ ID NO: 377 or a fragment thereof, or SEQ ID NO: 378 or a fragment thereof, or SEQ ID NO: 379 or a fragment thereof, or SEQ ID NO: 380 or a fragment thereof, or SEQ ID NO: 381 or a fragment thereof, or SEQ ID NO: 382 or a fragment thereof, or SEQ ID NO: 383 or a fragment thereof, or SEQ ID NO: 384 or a fragment thereof, or SEQ ID NO: 385 or a fragment thereof, or SEQ ID NO: 386 or a fragment thereof, or SEQ ID NO: 387 or a fragment thereof, or SEQ ID NO: 388 or a fragment thereof, or SEQ ID NO: 389 or a fragment thereof, or SEQ ID NO: 390 or a fragment thereof, or SEQ ID NO: 391 or a fragment thereof, or SEQ ID NO: 392 or a fragment thereof, or SEQ ID NO: 393 or a fragment thereof, or SEQ ID NO: 394 or a fragment thereof, or SEQ ID NO: 395 or a fragment thereof, or SEQ ID NO: 396 or a fragment thereof, or SEQ ID NO: 397 or a fragment thereof, or SEQ ID NO: 398 or a fragment thereof, or SEQ ID NO: 399 or a fragment thereof, or SEQ ID NO: 400 or a fragment thereof, or SEQ ID NO: 401 or a fragment thereof, or SEQ ID NO: 402 or a fragment thereof, or SEQ ID NO: 403 or a fragment thereof, or SEQ ID NO: 404 or a fragment thereof, or SEQ ID NO: 405 or a fragment thereof, or SEQ ID NO: 406 or a fragment thereof, or SEQ ID NO: 407 or a fragment thereof, or SEQ ID NO: 408 or a fragment thereof, or SEQ ID NO: 409 or a fragment thereof, or SEQ ID NO: 410 or a fragment thereof, or SEQ ID NO: 411 or a fragment thereof, or SEQ ID NO: 412 or a fragment thereof, or SEQ ID NO: 413 or a fragment thereof, or SEQ ID NO: 414 or a fragment thereof, or SEQ ID NO: 415 or a fragment thereof, or SEQ ID NO: 416 or a fragment thereof, or SEQ ID NO: 417 or a fragment thereof, or SEQ ID NO: 418 or a fragment thereof, or SEQ ID NO: 419 or a fragment thereof, or SEQ ID NO: 420 or a fragment thereof, or SEQ ID NO: 421 or a fragment thereof, or SEQ ID NO: 422 or a fragment thereof, or SEQ ID NO: 423 or a fragment thereof, or SEQ ID NO: 424 or a fragment thereof, or SEQ ID NO: 425 or a fragment thereof, or SEQ ID NO: 426 or a fragment thereof, or SEQ ID NO: 427 or a fragment thereof, or SEQ ID NO:428 or a fragment thereof, or SEQ ID NO: 429 or a fragment thereof, or SEQ ID NO: 430 or a fragment thereof, or SEQ ID NO: 431 or a fragment thereof, or SEQ ID NO: 432 or a fragment thereof, or SEQ ID NO: 433 or a fragment thereof, or SEQ ID NO: 434 or a fragment thereof, or SEQ ID NO: 435 or a fragment thereof, or SEQ ID NO: 436 or a fragment thereof, or SEQ ID NO: 437 or a fragment thereof, or SEQ ID NO: 438 or a fragment thereof, or SEQ ID NO: 439 or a fragment thereof, or SEQ ID NO: 440 or a fragment thereof, or SEQ ID NO: 441 or a fragment thereof, or SEQ ID NO: 442 or a fragment thereof, or SEQ ID NO: 443 or a fragment thereof, or SEQ ID NO: 444 or a fragment thereof, or SEQ ID NO: 445 or a fragment thereof, or SEQ ID NO: 446 or a fragment thereof, or SEQ ID NO: 447 or a fragment thereof, or SEQ ID NO: 448 or a fragment thereof, or SEQ ID NO: 449 or a fragment thereof, or SEQ ID NO: 450 or a fragment thereof, or SEQ ID NO: 451 or a fragment thereof,

optionally wherein, a fragment comprises one or more, but not all, of: a Linker 1 region, an AStD stem region; a Linker 2 region; a stem-loop region, e.g., a D arm Region; a Linker 3 Region; a stem-loop region, e.g., an AC arm region; a variable region; a stem-loop region, e.g., a T arm Region; and a Linker 4 region, e.g., as these regions are described herein.

162. The composition or pharmaceutical composition of any one of embodiments 76-82, the methods of any one of embodiments 94-109, or the cell of any one of claims 110-132, wherein ZZZ indicates any of the following amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.
163. The method of making of any one of embodiments 1-66 or 139-161, the composition or pharmaceutical composition of any one of embodiments 67-75, 79-82, or 139-161, the method of any one of embodiments 83-109, or 139-161, the cell of any one of embodiments 110-132, or 139-161, the reaction mixture of embodiment 133 or 139-161, the bioreactor of embodiment 134-135 or 139-161, the master cell bank of embodiment 136-137 or 139-161, or the method of evaluating of embodiment 138 or 139-161, wherein the TREM comprises a property selected from the following (e.g., in a TREM having a structure R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂, wherein R is a ribonucleotide residue):

a) under physiological conditions residue R₀ forms a linker region, e.g., a Linker 1 region;

b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;

c) under physiological conditions residues R₈-R₉ forms a linker region, e.g., a Linker 2 region;

d) under physiological conditions residues -R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region; e) under physiological conditions residue -R₂₉ forms a linker region, e.g., a Linker 3 Region;

f) under physiological conditions residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;

g) under physiological conditions residue -[R₄₇]ₓ comprises a variable region;

h) under physiological conditions residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or

i) under physiological conditions residue R₇₂ forms a linker region, e.g., a Linker 4 region.

164. The method, composition or pharmaceutical composition, cell, reaction mixture, bioreactor, or master cell bank of embodiment 163, comprising any one of properties (a)-(i).
165. The method, composition or pharmaceutical composition, cell, reaction mixture, bioreactor, or master cell bank of embodiment 163, comprising any two of properties (a)-(i).
166. The method, composition or pharmaceutical composition, cell, reaction mixture, bioreactor, or master cell bank of embodiment 163, comprising any three of properties (a)-(i).
167. The method, composition or pharmaceutical composition, cell, reaction mixture, bioreactor, or master cell bank of embodiment 163, comprising any four of properties (a)-(i).
168. The method, composition or pharmaceutical composition, cell, reaction mixture, bioreactor, or master cell bank of embodiment 163, comprising any five of properties (a)-(i).
169. The method, composition or pharmaceutical composition, cell, reaction mixture, bioreactor, or master cell bank of embodiment 163, comprising any six of properties (a)-(i).
170. The composition or pharmaceutical composition, the methods, or the cell of embodiment 163, comprising any seven of properties (a)-(i).
171. The method, composition or pharmaceutical composition, cell, reaction mixture, bioreactor, or master cell bank of embodiment 163, comprising all of properties (a)-(i).
172. The method of making of any one of embodiments 1-66 or 139-171, the composition or pharmaceutical composition of any one of embodiments 67-82 or 139-171, the method of any one of embodiments 83-109 or 139-171, the cell of any one of embodiments 110-132 or 139-171, the reaction mixture of embodiment 133 or 139-171, the bioreactor of embodiment 134-135 or 139-171, the master cell bank of embodiment 136-137 or 139-171, or the method of evaluating of embodiment 138 or 139-171, wherein the TREM comprises a consensus sequence provided herein.
173. The method of making of any one of embodiments 1-66 or 139-171, the composition or pharmaceutical composition of any one of embodiments 67-82 or 139-171, the method of any one of embodiments 83-109 or 139-171, the cell of any one of embodiments 110-132 or 139-171, the reaction mixture of embodiment 133 or 139-171, the bioreactor of embodiment 134-135 or 139-171, the master cell bank of embodiment 136-137 or 139-171, or the method of evaluating of embodiment 138 or 139-171, wherein the TREM comprises a consensus sequence of Formula I_{ZZZ}, wherein _{ZZZ} indicates any of the twenty amino acids and Formula I corresponds to all species.
174. The method of making of any one of embodiments 1-66 or 139-171, the composition or pharmaceutical composition of any one of embodiments 67-82 or 139-171, the method of any one of embodiments 83-109 or 139-171, the cell of any one of embodiments 110-132 or 139-171, the reaction mixture of embodiment 133 or 139-171, the bioreactor of embodiment 134-135 or 139-171, the master cell bank of embodiment 136-137 or 139-171, or the method of evaluating of embodiment 138 or 139-171, wherein the TREM comprises a consensus sequence of Formula II_{ZZZ}, wherein _{ZZZ} indicates any of the twenty amino acids and Formula II corresponds to mammals.
175. The method of making of any one of embodiments 1-66 or 139-171, the composition or pharmaceutical composition of any one of embodiments 67-82 or 139-171, the method of any one of embodiments 83-109 or 139-171, the cell of any one of embodiments 110-132 or 139-171, the reaction mixture of embodiment 133 or 139-171, the bioreactor of embodiment 134-135 or 139-171, the master cell bank of embodiment 136-137 or 139-171, or the method of evaluating of embodiment 138 or 139-171, wherein the TREM comprises a consensus sequence of Formula III_{ZZZ}, wherein _{ZZZ} indicates any of the twenty amino acids and Formula III corresponds to humans.
176. The method of making of any one of embodiments 1-66 or 139-175, the composition or pharmaceutical composition of any one of embodiments 67-82 or 139-175, the method of any one of embodiments 83-109 or 139-175, the cell of any one of embodiments 110-132 or 139-175, the reaction mixture of embodiment 133 or 139-175, the bioreactor of embodiment 134-135 or 139-175, the master cell bank of embodiment 136-137 or 139-175, or the method of evaluating of embodiment 138 or 139-175, wherein the TREM comprises a variable region at position R₄₇.
177. The method, composition or pharmaceutical composition, cell, reaction mixture, bioreactor, or master cell bank of embodiment 176, wherein the variable region is 1-271 residues in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 residues).
178. The method, composition or pharmaceutical composition, cell, reaction mixture, bioreactor, or master cell bank of embodiment 176 or 177, wherein the variable region the variable region comprises any one, all or a combination of Adenine, Cytosine, Guanine or Uracil.
179. The method, composition or pharmaceutical composition, cell, reaction mixture, bioreactor, or master cell bank of any one of embodiments 176-178, wherein the variable region comprises a ribonucleic acid (RNA) sequence encoded by a deoxyribonucleic acid (DNA) sequence disclosed in Table 3, e.g., any one of SEQ ID NOs: 452-561 disclosed in Table 3.
180. The method of making of any one of embodiments 1-66 or 139-179, the composition or pharmaceutical composition of any one of embodiments 67-82 or 139-179, the method of any one of embodiments 83-109 or 139-179, the cell of any one of embodiments 110-132 or 139-179, the reaction mixture of embodiment 133 or 139-179, the bioreactor of embodiment 134-135 or 139-179, the master cell bank of embodiment 136-137 or 139-179, or the method of evaluating of embodiment 138 or 139-179, wherein the TREM comprises a property (e.g., one, two, three, four, five, six, seven, eight, nine or all of, or any combination thereof) from the following:

a) if the TREM, e.g., if the AC stem loop of the TREM, comprises an exogenous insert, the exogenous insert is no more than 5 consecutive ribonucleotide residues in length;

b) if the TREM, e.g., if the AC stem loop of the TREM, comprises an exogenous insert, the balance of the molecule comprises a non-naturally occurring sequence, e.g., a non-naturally occurring sequence of 1, 2, 3, 4, 5 or more ribonucleotide residues;

c) if the TREM, e.g., if the AC stem loop of the TREM, comprises an exogenous insert, the exogenous insert does not comprise an effector entity, e.g., an effector entity having a primary sequence, secondary or tertiary structure dependent biological function;

d) if the TREM, e.g., if the AC stem loop of the TREM, comprises an exogenous insert, the exogenous insert does not comprise: the epsilon domain of the human Hepatitis B virus; dimerization domain of HIV; or an aptamer that binds to malachite green, dextran, or streptavidin;

e) the TREM can be charged with an amino acid;

f) the TREM, is translationally competent, e.g., can modulate the extension of a nascent polypeptide;

g) the TREM is not a naturally occurring molecule;

h) the TREM is not a naturally occurring molecule having anti-angiogenic properties, e.g., as determined by inhibition of endothelial cell proliferation;

i) the TREM is not anti-angiogenic; and

j) the TREM, in a homologous cell, does not give rise to a naturally occurring anti-angiogenic fragment.

181. The method, composition or pharmaceutical composition, cell, reaction mixture, bioreactor, or master cell bank of embodiment 180, comprising property (f).
182. The method, composition or pharmaceutical composition, cell, reaction mixture, bioreactor, or master cell bank of embodiment 180, comprising a property selected from (a)-(f) and a property selected from (g)-(j).
183. The method, composition or pharmaceutical composition, cell, reaction mixture, bioreactor, or master cell bank of embodiment 180, comprising property (g) and/or (d).
184. The method, composition or pharmaceutical composition, cell, reaction mixture, bioreactor, or master cell bank of embodiment 183, further comprising property (h) or (i).
185. The method, composition or pharmaceutical composition, cell, reaction mixture, bioreactor, or master cell bank of any one of embodiments 180-184, comprising a property selected from:

a) the composition comprises at least 1, 2, 5, 10, or 1,000 grams of a TREM;

b) the composition does not comprise a full length tRNA and a naturally occurring anti-angiogenic fragment thereof; or

c) the composition comprises a TREM of any of embodiments 67-82.

186. The method, composition or pharmaceutical composition, cell, reaction mixture, bioreactor, or master cell bank of embodiment 180 or 181, comprising a property selected from (a)-(e) and a property selected from (g)-(j).
187. The method, composition or pharmaceutical composition, cell, reaction mixture, bioreactor, or master cell bank of embodiment 180, comprising any one of properties (a)-(f).
188. The method, composition or pharmaceutical composition, cell, reaction mixture, bioreactor, or master cell bank of embodiment 180, comprising any two of properties (a)-(f).
189. The method, composition or pharmaceutical composition, cell, reaction mixture, bioreactor, or master cell bank of embodiment 180, comprising any three of properties (a)-(f).
190. The method, composition or pharmaceutical composition, cell, reaction mixture, bioreactor, or master cell bank of embodiment 180, comprising any four of properties (a)-(f).
191. The method, composition or pharmaceutical composition, cell, reaction mixture, bioreactor, or master cell bank of embodiment 180, comprising any five of properties (a)-(f).
192. The method, composition or pharmaceutical composition, cell, reaction mixture, bioreactor, or master cell bank of embodiment 180, comprising all of properties (a)-(f).
193. The method, composition or pharmaceutical composition, cell, reaction mixture, bioreactor, or master cell bank of embodiment 180, comprising any one of properties (f)-(j).
194. The method, composition or pharmaceutical composition, cell, reaction mixture, bioreactor, or master cell bank of embodiment 180, comprising any two of properties (f)-(j).
195. The method, composition or pharmaceutical composition, cell, reaction mixture, bioreactor, 30 or master cell bank of embodiment 180, comprising any three of properties (f)-(j).
196. The method, composition or pharmaceutical composition, cell, reaction mixture, bioreactor, or master cell bank of embodiment 180, comprising any four of properties (f)-(j).
197. The method, composition or pharmaceutical composition, cell, reaction mixture, bioreactor, or master cell bank of embodiment 180, comprising all of properties (f)-(j).
198. The method, composition or pharmaceutical composition, cell, reaction mixture, bioreactor, or master cell bank of embodiment 180, further comprising any one, two, three or all of properties (g)-(j).
199. The composition or pharmaceutical composition of any one of embodiments 67-82 or 139-198, the method of any one of embodiments 83-109 or 139-198, the cell of any one of embodiments 110-132 or 139-198, the reaction mixture of embodiment 133 or 139-198, the bioreactor of embodiment 134-135 or 139-198, the master cell bank of embodiment 136-137 or 139-198, or the method of evaluating of embodiment 138 or 139-198, wherein the TREM recognizes a stop codon.
200. The composition or pharmaceutical composition, cell, reaction mixture, bioreactor, or master cell bank of embodiment 199, wherein the TREM mediates acceptance and incorporation of an amino acid.
201. The composition or pharmaceutical composition of any one of embodiments 67-82 or 139-198, the method of any one of embodiments 83-109 or 139-198, the cell of any one of embodiments 110-132 or 139-198, the reaction mixture of embodiment 133 or 139-198, the bioreactor of embodiment 134-135 or 139-198, the master cell bank of embodiment 136-137 or 139-198, or the method of evaluating of embodiment 138 or 139-198, wherein the TREM does not recognize a stop codon.
202. The method of making of any one of embodiments 1-66 or 139-198, the composition or pharmaceutical composition of any one of embodiments 67-82 or 139-201, the method of any one of embodiments 83-109 or 139-201, the cell of any one of embodiments 110-132 or 139-201, the reaction mixture of embodiment 133 or 139-201, the bioreactor of embodiment 134-135 or 139-201, the master cell bank of embodiment 136-137 or 139-201, or the method of evaluating of embodiment 138 or 139-201, wherein the TREM does not comprise a naturally occurring bacterial tRNA or fragment thereof (e.g., an E. coli tRNA or fragment thereof), or a naturally occurring yeast tRNA or fragment thereof.
203. The method of making of any one of embodiments 1-66 or 139-198, the composition or pharmaceutical composition of any one of embodiments 67-82 or 139-201, the method of any one of embodiments 83-109 or 139-201, the cell of any one of embodiments 110-132 or 139-201, the reaction mixture of embodiment 133 or 139-201, the bioreactor of embodiment 134-135 or 139-201, the master cell bank of embodiment 136-137 or 139-201, or the method of evaluating of embodiment 138 or 139-201, wherein the TREM is formulated as a lyophilized TREM composition.
204. The method of making of any one of embodiments 1-66 or 139-198, the composition or pharmaceutical composition of any one of embodiments 67-82 or 139-201, the method of any one of embodiments 83-109 or 139-201, the cell of any one of embodiments 110-132 or 139-201, the reaction mixture of embodiment 133 or 139-201, the bioreactor of embodiment 134-135 or 139-201, the master cell bank of embodiment 136-137 or 139-201, or the method of evaluating of embodiment 138 or 139-201, wherein the TREM is formulated as a liquid TREM composition.
205. The method of making of any one of embodiments 1-66 or 139-198, the composition or pharmaceutical composition of any one of embodiments 67-82 or 139-201, the method of any one of embodiments 83-109 or 139-201, the cell of any one of embodiments 110-132 or 139-201, the reaction mixture of embodiment 133 or 139-201, the bioreactor of embodiment 134-135 or 139-201, the master cell bank of embodiment 136-137 or 139-201, or the method of evaluating of embodiment 138 or 139-201, wherein the TREM is formulated as a frozen TREM composition.
206. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 1, or a fragment thereof.
207. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 2, or a fragment thereof.
208. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 3, or a fragment thereof.
209. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 4, or a fragment thereof.
210. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 5, or a fragment thereof.
211. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 6, or a fragment thereof.
212. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 7, or a fragment thereof.
213. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 8, or a fragment thereof.
214. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 9, or a fragment thereof.
215. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 10, or a fragment thereof.
216. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 11, or a fragment thereof.
217. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 12, or a fragment thereof.
218. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 13, or a fragment thereof.
219. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:14, or a fragment thereof.
220. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 15, or a fragment thereof.
221. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 16, or a fragment thereof.
222. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 17, or a fragment thereof.
223. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 18, or a fragment thereof.
224. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:19, or a fragment thereof.
225. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 20, or a fragment thereof.
226. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 21, or a fragment thereof.
227. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 22, or a fragment thereof.
228. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 23, or a fragment thereof.
229. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 24, or a fragment thereof.
230. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 25, or a fragment thereof.
231. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 26, or a fragment thereof.
232. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 27, or a fragment thereof.
233. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 28, or a fragment thereof.
234. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 29, or a fragment thereof.
235. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 30, or a fragment thereof.
236. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 31, or a fragment thereof.
237. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 32, or a fragment thereof.
238. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 33, or a fragment thereof.
239. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 34, or a fragment thereof.
240. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 35, or a fragment thereof.
241. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 36, or a fragment thereof.
242. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 37, or a fragment thereof.
243. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 38, or a fragment thereof.
244. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 39, or a fragment thereof.
245. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 40, or a fragment thereof.
246. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 41, or a fragment thereof.
247. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 42, or a fragment thereof.
248. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 43, or a fragment thereof.
249. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 44, or a fragment thereof.
250. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 45, or a fragment thereof.
251. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 46, or a fragment thereof.
252. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 47, or a fragment thereof.
253. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 48, or a fragment thereof.
254. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 49, or a fragment thereof.
255. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 50, or a fragment thereof.
256. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 51, or a fragment thereof.
257. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 52, or a fragment thereof.
258. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 53, or a fragment thereof.
259. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 54, or a fragment thereof.
260. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 55, or a fragment thereof.
261. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 56, or a fragment thereof.
262. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 57, or a fragment thereof.
263. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 58, or a fragment thereof.
264. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 59, or a fragment thereof.
265. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 60, or a fragment thereof.
266. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 61, or a fragment thereof.
267. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 62, or a fragment thereof.
268. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 63, or a fragment thereof.
269. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 64, or a fragment thereof.
270. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 65, or a fragment thereof.
271. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 66, or a fragment thereof.
272. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 67, or a fragment thereof.
273. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 68, or a fragment thereof.
274. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 69, or a fragment thereof.
275. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 70, or a fragment thereof.
276. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 71, or a fragment thereof.
277. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 72, or a fragment thereof.
278. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 73, or a fragment thereof.
279. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 74, or a fragment thereof.
280. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 75, or a fragment thereof.
281. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 76, or a fragment thereof.
282. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 77, or a fragment thereof.
283. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 78, or a fragment thereof.
284. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 79, or a fragment thereof.
285. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 80, or a fragment thereof.
286. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 81, or a fragment thereof.
287. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 82, or a fragment thereof.
288. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 83, or a fragment thereof.
289. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 84, or a fragment thereof.
290. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 85, or a fragment thereof.
291. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:86, or a fragment thereof.
292. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 87, or a fragment thereof.
293. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 88, or a fragment thereof.
294. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 89, or a fragment thereof.
295. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 90, or a fragment thereof.
296. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 91, or a fragment thereof.
297. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 92, or a fragment thereof.
298. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 93, or a fragment thereof.
299. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 94, or a fragment thereof.
300. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 95, or a fragment thereof.
301. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 96, or a fragment thereof.
302. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 97, or a fragment thereof.
303. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 98, or a fragment thereof.
304. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 99, or a fragment thereof.
305. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 100, or a fragment thereof.
306. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 101, or a fragment thereof.
307. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 102, or a fragment thereof.
308. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 103, or a fragment thereof.
309. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 104, or a fragment thereof.
310. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 105, or a fragment thereof.
311. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:106, or a fragment thereof.
312. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:107, or a fragment thereof.
313. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:108, or a fragment thereof.
314. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:109, or a fragment thereof.
315. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:110, or a fragment thereof.
316. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:111, or a fragment thereof.
317. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:112, or a fragment thereof.
318. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:113, or a fragment thereof.
319. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:114, or a fragment thereof.
320. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:115, or a fragment thereof.
321. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:116, or a fragment thereof.
322. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:117, or a fragment thereof.
323. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:118, or a fragment thereof.
324. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:119, or a fragment thereof.
325. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:120, or a fragment thereof.
326. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:121, or a fragment thereof.
327. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:122, or a fragment thereof.
328. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:123, or a fragment thereof.
329. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:124, or a fragment thereof.
330. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:125, or a fragment thereof.
331. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:126, or a fragment thereof.
332. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:127, or a fragment thereof.
333. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:128, or a fragment thereof.
334. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:129, or a fragment thereof.
335. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:130, or a fragment thereof.
336. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:131, or a fragment thereof.
337. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:132, or a fragment thereof.
338. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:133, or a fragment thereof.
339. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:134, or a fragment thereof.
340. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:135, or a fragment thereof.
341. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:136, or a fragment thereof.
342. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:137, or a fragment thereof.
343. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:138, or a fragment thereof.
344. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:139, or a fragment thereof.
345. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:140, or a fragment thereof.
346. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:141, or a fragment thereof.
347. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:142, or a fragment thereof.
348. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:143, or a fragment thereof.
349. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:144, or a fragment thereof.
350. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:145, or a fragment thereof.
351. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:146, or a fragment thereof.
352. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:147, or a fragment thereof.
353. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:148, or a fragment thereof.
354. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:149, or a fragment thereof.
355. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:150, or a fragment thereof.
356. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:151, or a fragment thereof.
357. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:152, or a fragment thereof.
358. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:153, or a fragment thereof.
359. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:154, or a fragment thereof.
360. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:155, or a fragment thereof.
361. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:156, or a fragment thereof.
362. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:157, or a fragment thereof.
363. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:158, or a fragment thereof.
364. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:159, or a fragment thereof.
365. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:160, or a fragment thereof.
366. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:161, or a fragment thereof.
367. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:162, or a fragment thereof.
368. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:163, or a fragment thereof.
369. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:164, or a fragment thereof.
370. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:165, or a fragment thereof.
371. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:166, or a fragment thereof.
372. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:167, or a fragment thereof.
373. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:168, or a fragment thereof.
374. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:169, or a fragment thereof.
375. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:170, or a fragment thereof.
376. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:171, or a fragment thereof.
377. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:172, or a fragment thereof.
378. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:173, or a fragment thereof.
379. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:174, or a fragment thereof.
380. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:175, or a fragment thereof.
381. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:176, or a fragment thereof.
382. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:177, or a fragment thereof.
383. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:178, or a fragment thereof.
384. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:179, or a fragment thereof.
385. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:180, or a fragment thereof.
386. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:181, or a fragment thereof.
387. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:182, or a fragment thereof.
388. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:183, or a fragment thereof.
389. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:184, or a fragment thereof.
390. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:185, or a fragment thereof.
391. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:186, or a fragment thereof.
392. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:187, or a fragment thereof.
393. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:188, or a fragment thereof.
394. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:189, or a fragment thereof.
395. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:190, or a fragment thereof.
396. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:191, or a fragment thereof.
397. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:192, or a fragment thereof.
398. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:193, or a fragment thereof.
399. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:194, or a fragment thereof.
400. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:195, or a fragment thereof.
401. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:196, or a fragment thereof.
402. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:197, or a fragment thereof.
403. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:198, or a fragment thereof.
404. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:199, or a fragment thereof.
405. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:200, or a fragment thereof.
406. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:201, or a fragment thereof.
407. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:202, or a fragment thereof.
408. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:203, or a fragment thereof.
409. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:204, or a fragment thereof.
410. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:205, or a fragment thereof.
411. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:206, or a fragment thereof.
412. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:207, or a fragment thereof.
413. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:208, or a fragment thereof.
414. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:209, or a fragment thereof.
415. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:210, or a fragment thereof.
416. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:211, or a fragment thereof.
417. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:212, or a fragment thereof.
418. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:213, or a fragment thereof.
419. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:214, or a fragment thereof.
420. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:215, or a fragment thereof.
421. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:216, or a fragment thereof.
422. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:217, or a fragment thereof.
423. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:218, or a fragment thereof.
424. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:219, or a fragment thereof.
425. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:220, or a fragment thereof.
426. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:221, or a fragment thereof.
427. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:222, or a fragment thereof.
428. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:223, or a fragment thereof.
429. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:224, or a fragment thereof.
430. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:225, or a fragment thereof.
431. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:226, or a fragment thereof.
432. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:227, or a fragment thereof.
433. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:228, or a fragment thereof.
434. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:229, or a fragment thereof.
435. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:230, or a fragment thereof.
436. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:231, or a fragment thereof.
437. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:232, or a fragment thereof.
438. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:233, or a fragment thereof.
439. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:234, or a fragment thereof.
440. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:235, or a fragment thereof.
441. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:236, or a fragment thereof.
442. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:237, or a fragment thereof.
443. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:238, or a fragment thereof.
444. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:239, or a fragment thereof.
445. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:240, or a fragment thereof.
446. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:241, or a fragment thereof.
447. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:242, or a fragment thereof.
448. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:243, or a fragment thereof.
449. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:244, or a fragment thereof.
450. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:245, or a fragment thereof.
451. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:246, or a fragment thereof.
452. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:247, or a fragment thereof.
453. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:248, or a fragment thereof.
454. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:249, or a fragment thereof.
455. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:250, or a fragment thereof.
456. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:251, or a fragment thereof.
457. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:252, or a fragment thereof.
458. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:253, or a fragment thereof.
459. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:254, or a fragment thereof.
460. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:255, or a fragment thereof.
461. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:256, or a fragment thereof.
462. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:257, or a fragment thereof.
463. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:258, or a fragment thereof.
464. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:259, or a fragment thereof.
465. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:260, or a fragment thereof.
466. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:261, or a fragment thereof.
467. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:262, or a fragment thereof.
468. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:263, or a fragment thereof.
469. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:264, or a fragment thereof.
470. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:265, or a fragment thereof.
471. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:266, or a fragment thereof.
472. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:267, or a fragment thereof.
473. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:268, or a fragment thereof.
474. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:269, or a fragment thereof.
475. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:270, or a fragment thereof.
476. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:271, or a fragment thereof.
477. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:272, or a fragment thereof.
478. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:273, or a fragment thereof.
479. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:274, or a fragment thereof.
480. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:275, or a fragment thereof.
481. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:276, or a fragment thereof.
482. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:277, or a fragment thereof.
483. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:278, or a fragment thereof.
484. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:279, or a fragment thereof.
485. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:280, or a fragment thereof.
486. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:281, or a fragment thereof.
487. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:282, or a fragment thereof.
488. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:283, or a fragment thereof.
489. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:284, or a fragment thereof.
490. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:285, or a fragment thereof.
491. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:286, or a fragment thereof.
492. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:287, or a fragment thereof.
493. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:288, or a fragment thereof.
494. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:289, or a fragment thereof.
495. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:290, or a fragment thereof.
496. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:291, or a fragment thereof.
497. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:292, or a fragment thereof.
498. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:293, or a fragment thereof.
499. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:294, or a fragment thereof.
500. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:295, or a fragment thereof.
501. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:296, or a fragment thereof.
502. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:297, or a fragment thereof.
503. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:298, or a fragment thereof.
504. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:299, or a fragment thereof.
505. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:300, or a fragment thereof.
506. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:301, or a fragment thereof.
507. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:302, or a fragment thereof.
508. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:303, or a fragment thereof.
509. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:304, or a fragment thereof.
510. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:305, or a fragment thereof.
511. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:306, or a fragment thereof.
512. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:307, or a fragment thereof.
513. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:308, or a fragment thereof.
514. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:309, or a fragment thereof.
515. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:310, or a fragment thereof.
516. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:311, or a fragment thereof.
517. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:312, or a fragment thereof.
518. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:313, or a fragment thereof.
519. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:314, or a fragment thereof.
520. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:315, or a fragment thereof.
521. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:316, or a fragment thereof.
522. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:317, or a fragment thereof.
523. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:318, or a fragment thereof.
524. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:319, or a fragment thereof.
525. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:320, or a fragment thereof.
526. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:321, or a fragment thereof.
527. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:322, or a fragment thereof.
528. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:323, or a fragment thereof.
529. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:324, or a fragment thereof.
530. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:325, or a fragment thereof.
531. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:326, or a fragment thereof.
532. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:327, or a fragment thereof.
533. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:328, or a fragment thereof.
534. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:329, or a fragment thereof.
535. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:330, or a fragment thereof.
536. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:331, or a fragment thereof.
537. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:332, or a fragment thereof.
538. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:333, or a fragment thereof.
539. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:334, or a fragment thereof.
540. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:335, or a fragment thereof.
541. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:336, or a fragment thereof.
542. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:337, or a fragment thereof.
543. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:338, or a fragment thereof.
544. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:339, or a fragment thereof.
545. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:340, or a fragment thereof.
546. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:341, or a fragment thereof.
547. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:342, or a fragment thereof.
548. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:343, or a fragment thereof.
549. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:344, or a fragment thereof.
550. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:345, or a fragment thereof.
551. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:346, or a fragment thereof.
552. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:347, or a fragment thereof.
553. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:348, or a fragment thereof.
554. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:349, or a fragment thereof.
555. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:350, or a fragment thereof.
556. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:351, or a fragment thereof.
557. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:352, or a fragment thereof.
558. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:353, or a fragment thereof.
559. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:354, or a fragment thereof.
560. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:355, or a fragment thereof.
561. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:356, or a fragment thereof.
562. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:357, or a fragment thereof.
563. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:358, or a fragment thereof.
564. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:359, or a fragment thereof.
565. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:360, or a fragment thereof.
566. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:361, or a fragment thereof.
567. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:362, or a fragment thereof.
568. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:363, or a fragment thereof.
569. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:364, or a fragment thereof.
570. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:365, or a fragment thereof.
571. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:366, or a fragment thereof.
572. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:367, or a fragment thereof.
573. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:368, or a fragment thereof.
574. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:369, or a fragment thereof.
575. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:370, or a fragment thereof.
576. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:371, or a fragment thereof.
577. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:372, or a fragment thereof.
578. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:373, or a fragment thereof.
579. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:374, or a fragment thereof.
580. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:375, or a fragment thereof.
581. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:376, or a fragment thereof.
582. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:377, or a fragment thereof.
583. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:378, or a fragment thereof.
584. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:379, or a fragment thereof.
585. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:380, or a fragment thereof.
586. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:381, or a fragment thereof.
587. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:382, or a fragment thereof.
588. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:383, or a fragment thereof.
589. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:384, or a fragment thereof.
590. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:385, or a fragment thereof.
591. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:386, or a fragment thereof.
592. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:387, or a fragment thereof.
593. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:388, or a fragment thereof.
594. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:389, or a fragment thereof.
595. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:390, or a fragment thereof.
596. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:391, or a fragment thereof.
597. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:392, or a fragment thereof.
598. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:393, or a fragment thereof.
599. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:394, or a fragment thereof.
600. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:395, or a fragment thereof.
601. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:396, or a fragment thereof.
602. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:397, or a fragment thereof.
603. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:398, or a fragment thereof.
604. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:399, or a fragment thereof.
605. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:400, or a fragment thereof.
606. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:401, or a fragment thereof.
607. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:402, or a fragment thereof.
608. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 403, or a fragment thereof.
609. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 404, or a fragment thereof.
610. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 405, or a fragment thereof.
611. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 406, or a fragment thereof.
612. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 407, or a fragment thereof.
613. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 408, or a fragment thereof.
614. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 409, or a fragment thereof.
615. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 410, or a fragment thereof.
616. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 411, or a fragment thereof.
617. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 412, or a fragment thereof.
618. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 413, or a fragment thereof.
619. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 414, or a fragment thereof.
620. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 415, or a fragment thereof.
621. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 416, or a fragment thereof.
622. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 417, or a fragment thereof.
623. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 418, or a fragment thereof.
624. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 419, or a fragment thereof.
625. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 420, or a fragment thereof.
626. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 421, or a fragment thereof.
627. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 422, or a fragment thereof.
628. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 423, or a fragment thereof.
629. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 424, or a fragment thereof.
630. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 425, or a fragment thereof.
631. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 426, or a fragment thereof.
632. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 427, or a fragment thereof.
633. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 428, or a fragment thereof.
634. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 429, or a fragment thereof.
635. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 430, or a fragment thereof.
636. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 431, or a fragment thereof.
637. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 432, or a fragment thereof.
638. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 433, or a fragment thereof.
639. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 434, or a fragment thereof.
640. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 435, or a fragment thereof.
641. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 436, or a fragment thereof.
642. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 437, or a fragment thereof.
643. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 438, or a fragment thereof.
644. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 439, or a fragment thereof.
645. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 440, or a fragment thereof.
646. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 441, or a fragment thereof.
647. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 442, or a fragment thereof.
648. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 443, or a fragment thereof.
649. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 444, or a fragment thereof.
650. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 445, or a fragment thereof.
651. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 446, or a fragment thereof.
652. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 447, or a fragment thereof.
653. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 448, or a fragment thereof.
654. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 449, or a fragment thereof.
655. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 450, or a fragment thereof.
656. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 451, or a fragment thereof.
657. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 562, or a fragment thereof.
658. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 563, or a fragment thereof.
659. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 564, or a fragment thereof.
660. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 565, or a fragment thereof.
661. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 566, or a fragment thereof.
662. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 567, or a fragment thereof.
663. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 568, or a fragment thereof.
664. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 569, or a fragment thereof.
665. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 570, or a fragment thereof.
666. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 571 or a fragment thereof.
667. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 572, or a fragment thereof.
668. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 573, or a fragment thereof.
669. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 574, or a fragment thereof.
670. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 575, or a fragment thereof.
671. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 576, or a fragment thereof.
672. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 577, or a fragment thereof.
673. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 578, or a fragment thereof.
674. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 579, or a fragment thereof.
675. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 580, or a fragment thereof.
676. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 581, or a fragment thereof.
677. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 582, or a fragment thereof.
678. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 583, or a fragment thereof.
679. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 584, or a fragment thereof.
680. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 585, or a fragment thereof.
681. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 586, or a fragment thereof.
682. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 587, or a fragment thereof.
683. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 588, or a fragment thereof.
684. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 589, or a fragment thereof.
685. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 590, or a fragment thereof.
686. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 591, or a fragment thereof.
687. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 592, or a fragment thereof.
688. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 593, or a fragment thereof.
689. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 594, or a fragment thereof.
690. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 595, or a fragment thereof.
691. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 596, or a fragment thereof.
692. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 597, or a fragment thereof.
693. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 598, or a fragment thereof.
694. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 599, or a fragment thereof.
695. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 600, or a fragment thereof.
696. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 601, or a fragment thereof.
697. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 602, or a fragment thereof.
698. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 603, or a fragment thereof.
699. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 604, or a fragment thereof.
700. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 605, or a fragment thereof.
701. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 606, or a fragment thereof.
702. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 607, or a fragment thereof.
703. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 608, or a fragment thereof.
704. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 609, or a fragment thereof.
705. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:610, or a fragment thereof.
706. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 611, or a fragment thereof.
707. The method, composition or pharmaceutical composition, cell, reaction mixture, bioreactor, or master cell bank of any one of embodiments 1-205, wherein the TREM comprises an RNA sequence encoded by the DNA sequence of SEQ ID NO:612, or a fragment thereof.
708. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 613, or a fragment thereof.
709. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 614, or a fragment thereof.
710. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 615, or a fragment thereof.
711. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 616, or a fragment thereof.
712. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 617, or a fragment thereof.
713. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 618, or a fragment thereof.
714. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 619, or a fragment thereof.
715. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 620, or a fragment thereof.
716. The method, composition or pharmaceutical composition, cell, reaction mixture, bioreactor, or master cell bank of any one of embodiments 1-205, wherein the TREM comprises an RNA sequence encoded by the DNA sequence of SEQ ID NO: 621, or a fragment thereof.
717. The method, composition or pharmaceutical composition, cell, reaction mixture, bioreactor, or master cell bank of any one of embodiments 206-716, wherein, a fragment comprises one or more, but not all, of: a Linker 1 region, an AStD stem region; a Linker 2 region; a stem-loop region, e.g., a D arm Region; a Linker 3 Region; a stem-loop region, e.g., an AC arm region; a variable region; a stem-loop region, e.g., a T arm Region; and a Linker 4 region, e.g., as these regions are described herein.
718. A method of making a purified tRNA effector molecule (TREM) pharmaceutical composition, comprising:

providing an insect host cell comprising an exogenous nucleic acid, e.g., a DNA or RNA, encoding the TREM;

maintaining the insect host cell under conditions sufficient to express the TREM;

purifying the TREM from the insect host cell, e.g., according to a method described herein; and

formulating the purified TREM as a pharmaceutical composition, e.g., by combining the TREM with a pharmaceutical excipient,

thereby making the TREM pharmaceutical composition.

719. The method of embodiment 718, wherein the insect host cell is chosen from: an insect cell or cell line, e.g., a Sf9 cell or cell line.
720. A method of making a purified tRNA effector molecule (TREM) pharmaceutical composition, comprising:

providing a yeast host cell comprising an exogenous nucleic acid, e.g., a DNA or RNA, encoding the TREM;

maintaining the yeast host cell under conditions sufficient to express the TREM;

purifying the TREM from the yeast host cell, e.g., according to a method described herein; and

formulating the purified TREM as a pharmaceutical composition, e.g., by combining the TREM with a pharmaceutical excipient,

thereby making the TREM pharmaceutical composition.

721. The method of embodiment 720, wherein the yeast host cell is chosen from: a yeast cell or cell line, e.g., a S. cerevisiae or S. pombe cell or cell line.
722. The method of any one of embodiments 718-721, wherein the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

Other features, objects, and advantages of the invention will be apparent from the description and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTIONS OF THE DRAWINGS

FIGS. 1A-1C are graphs showing an increase in cell growth in three cells lines after transfection with a TREM corresponding to the initiator methionine (iMet). FIG. 1A is a graph showing increased % cellular confluency (a measure of cell growth) of U20S cells transfected with Cy3-labeled iMet-CAT-TREM or transfected with a Cy3-labeled non-targeted control. FIG. 1B is a graph showing increased % cellular confluency (a measure of cell growth) of H1299 cells transfected with Cy3-labeled iMet-CAT-TREM or transfected with a Cy3-labeled non-targeted control. FIG. 1C is a graph showing increased % cellular confluency (a measure of cell growth) of Hela cells transfected with Cy3-labeled iMet-CAT-TREM or transfected with a Cy3-labeled non-targeted control.

FIG. 2 is a graph depicting an increase in NanoLuc reporter expression upon addition of iMET-TREM to a translational reaction with cell free lysate. As a control, a translational reaction with buffer was performed.

### DETAILED DESCRIPTION OF CERTAIN EMBODIMENTS

The present disclosure features tRNA-based effector molecules (TREMs) and methods relating thereto. As disclosed herein tRNA-based effector molecules (TREMs) are complex molecules which can mediate a variety of cellular processes. Pharmaceutical TREM compositions can be administered to a cell, a tissue, or to a subject to modulate these functions.

### Definitions

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

An “oncogene,” as that term is used herein, refers to a gene that modulates one or more cellular processes including: cell fate determination, cell survival and genome maintenance. In an embodiment, an oncogene provides a selective growth advantage to the cell in which it is present, e.g., deregulated, e.g., genetically deregulated (e.g., mutated or amplified) or epigenetically deregulated. Exemplary oncogenes include, Myc (e.g., c-Myc, N-Myc or L-Myc), c-Jun, Wnt, or RAS.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “synthetic TREM,” as that term is used herein, refers to a TREM which was synthesized other than in a cell having an endogenous nucleic acid encoding the TREM, e.g., by cell-free solid phase synthesis. A synthetic TREM can have the same, or a different, sequence, set of post-transcriptional modifications, or tertiary structure, as a native tRNA.

A “TREM expressed in a heterologous cell,” as that term is used herein, refers to a TREM made under non-native conditions. E.g., a TREM, i) made in a cell that, differs, e.g., genetically, metabolically (e.g., has a different profile of gene expression or has a different level of a cellular component, e.g., an absorbed nutrient), or epigenetically, from a naturally occurring cell; ii) made in a cell that, is cultured under conditions, e.g., nutrition, pH, temperature, cell density, or stress conditions, that are different from native conditions (native conditions are the conditions under which a cell makes a tRNA in nature); or iii) was made in a cell at a level, at a rate, or at a concentration, or was localized in a compartment or location, that differs from a reference, e.g., at a level, at a rate, or at a concentration, or was localized in a compartment or location, that differs from that which occurs under native conditions. A TREM expressed in a heterologous cell can have the same, or a different, sequence, set of post-transcriptional modifications, or tertiary structure, as a native tRNA.

A “tRNA”, as that term is used herein, refers to a naturally occurring transfer ribonucleic acid in its native state.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:

(a′) an optional linker region of a consensus sequence provided in the “Consensus Sequence” section, e.g., a Linker 1 region;

(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g., one of ordinary skill can determine the sequence which corresponds to an AStD from a tRNA sequence encoded by a nucleic acid in Table 1.)

In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I_{ZZZ}, wherein ZZZ indicates any of the twenty amino acids; In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II_{ZZZ}, wherein ZZZ indicates any of the twenty amino acids; In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III_{ZZZ}, wherein ZZZ indicates any of the twenty amino acids;

(a′-1) a linker comprising residues R₈-R₉ of a consensus sequence provided in the “Consensus Sequence” section, e.g., a Linker 2 region;

(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I_{ZZZ}, wherein ZZZ indicates any of the twenty amino acids; In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II_{ZZZ}, wherein ZZZ indicates any of the twenty amino acids; In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III_{ZZZ}, wherein ZZZ indicates any of the twenty amino acids;

(b′-1) a linker comprising residue R₂₉ of a consensus sequence provided in the “Consensus Sequence” section, e.g., a Linker 3 region;

(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I_{ZZZ}, wherein ZZZ indicates any of the twenty amino acids;

In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II_{ZZZ}, wherein ZZZ indicates any of the twenty amino acids; In an embodiment, the ACHD comprises residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III_{ZZZ}, wherein ZZZ indicates any of the twenty amino acids;

(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue -[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);

(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I_{ZZZ}, wherein ZZZ indicates any of the twenty amino acids; In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II_{ZZZ}, wherein ZZZ indicates any of the twenty amino acids; In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III_{ZZZ}, wherein ZZZ indicates any of the twenty amino acids;

(e′1) a linker comprising residue R₇₂ of a consensus sequence provided in the “Consensus Sequence” section, e.g., a Linker 4 region;

(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;

(g) a tertiary structure, e.g., an L-shaped tertiary structure;

(h) adaptor function, i.e., the TREM mediates acceptance of an amino acid, e.g., its cognate amino acid and transfer of the AA in the initiation or elongation of a polypeptide chain;

(i) cognate adaptor function wherein the TREM mediates acceptance and incorporation of an amino acid (e.g., cognate amino acid) associated in nature with the anti-codon of the TREM to initiate or elongate a polypeptide chain;

(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;

(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;

(l) a structure which allows for ribosome binding;

(m) a post-transcriptional modification, e.g., it comprises one or more modifications from Table 2, e.g., 1, 2, 3, 4, 5, 6, 7, 8, 9, 10, 11, 12, 13, 14, or 15 modifications listed in Table 2;

(n) the ability to inhibit a functional property of a tRNA, e.g., any of properties (h)-(k) possessed by a tRNA;

(o) the ability to modulate cell fate;

(p) the ability to modulate ribosome occupancy;

(q) the ability to modulate protein translation;

(r) the ability to modulate mRNA stability;

(s) the ability to modulate protein folding and structure;

(t) the ability to modulate protein transduction or compartmentalization;

(u) the ability to modulate protein stability; or

(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:

(i) an amino acid attachment domain that binds an amino acid (e.g., an AStD, as described in (a) herein; and

(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM composition,” as that term is used herein, refers to a composition comprising a plurality of TREMs. A TREM composition can comprise one or more species of TREMs. In an embodiment, the composition comprises only a single species of TREM. In an embodiment, the TREM composition comprises a first TREM species and a second TREM species. In an embodiment, the TREM composition comprises X TREM species, wherein X=2, 3, 4, 5, 6, 7, 8, 9, or 10. In an embodiment, the TREM has at least 70, 75, 80, 85, 90, or 95, or has 100%, identity with a sequence encoded by a nucleic acid in Table 1. A TREM composition can comprise one or more species of TREMs. In an embodiment, the TREM composition is purified from cell culture. In an embodiment the cell culture from which the TREM is purified comprises at least 1×10⁷ host cells, 1×10⁸ host cells, 1×10⁹ host cells, 1×10¹⁰ host cells, 1×10¹¹ host cells, 1×10¹² host cells, 1×10¹³ host cells, or 1×10¹⁴ host cells. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

A “tumor suppressor,” as that term is used herein, refers to a gene that modulates one or more cellular processes including: cell fate determination, cell survival and genome maintenance. In an embodiment, a tumor suppressor provides a selective growth advantage to the cell in which it is deregulated, e.g., genetically deregulated (e.g., mutated or deleted) or epigenetically deregulated. Exemplary tumor suppressors include p53 or Rb.

### Host Cells

A host cell is a cell (e.g., a cultured cell) that can be used for expression and/or purification of a TREM. In an embodiment, a host cell comprises a mammalian cell, e.g., a human cell. In an embodiment, a host cell comprises a non-mammalian cell, e.g., a yeast cell. In an embodiment, a host cell comprises a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell. In an embodiment, a host cell comprises a cancer cell, e.g., a solid tumor cell (e.g., a breast cancer cell (e.g., a MCF7 cell), a pancreatic cell line (e.g. a MIA PaCa-2 cell), a lung cancer cell, or a prostate cancer cell, or a hematological cancer cell). In an embodiment, a host cell comprises a cell that expresses one or more tissue-specific tRNAs. For example, a host cell can comprise a cell derived from a tissue associated with expression of a tRNA, e.g., a tissue-specific tRNA. In an embodiment, a host cell that expresses a tissue-specific tRNA is modified to express a TREM, or a fragment thereof.

In an embodiment, the host cell is not a bacterial cell, e.g., an E. coli cell.

In an embodiment, a host cell is a cell that can be maintained under conditions that allow for expression of a TREM.

In an embodiment, a host cell is capable of post-transcriptionally modifying the TREM, e.g., adding a post-transcriptional modification selected from Table 2. In an embodiment, a host cell expresses (e.g., naturally or heterologously) an enzyme listed in Table 2. In an embodiment, a host cell expresses (e.g., naturally or heterologously) an enzyme, e.g., an enzyme having nuclease activity (e.g., endonuclease activity or ribonuclease activity), e.g., or one or more of Dicer, Angiogenin, RNaseA, RNaseP, RNaseZ, Rny1 or PrrC.

### Method of Culturing Host Cell

A host cell can be cultured in a medium that promotes growth, e.g., proliferation or hyperproliferation of the host cell. A host cell can be cultured in a suitable media, e.g., any of the following media: DMEM, MEM, MEM alpha, RPMI, F-10 media, F-12 media, DMEM/F-12 media, IMDM, Medium 199, Leibovitz L-15, McCoys's 5A, MDCB media, or CMRL media. In an embodiment the media is supplemented with glutamine. In an embodiment, the media is not supplemented with glutamine. In an embodiment, a host cell is cultured in media that has an excess of nutrients, e.g., is not nutrient limiting. A host cell can be cultured in a medium comprising or supplemented with one or a combination of growth factors, cytokines or hormones, e.g., one or a combination of serum (e.g., fetal bovine serum (FBS)), HEPES, fibroblast growth factor (FGFs), epidermal growth factors (EGFs), insulin-like growth factors (IGFs), transforming growth factor beta (TGFb), platelet derived growth factor (PDGFs), hepatocyte growth factor (HGFs), or tumor necrosis factor (TNFs).

A host cell can also be cultured under conditions that induce stress, e.g., cellular stress, osmotic stress, translational stress, or oncogenic stress. In an embodiment, a host cell expressing a TREM, cultured under conditions that induce stress (e.g., as described herein) results in a fragment of the TREM, e.g., as described herein.

A host cell can be cultured under nutrient limiting conditions, e.g., the host cell is cultured in media that has a limited amount of one or more nutrients. Examples of nutrients that can be limiting are amino acids, lipids, carbohydrates, hormones, growth factors or vitamins. In an embodiment, a host cell expressing a TREM, cultured in media that has a limited amount of one or more nutrients, e.g., the media is nutrient starved, results in a fragment of the TREM, e.g., as described herein. In an embodiment, a host cell expressing a TREM, cultured in media that has a limited amount of one or more nutrients, e.g., the media is nutrient starved, results in a TREM that is uncharged (e.g. a uTREM).

A host cell can comprise an immortalized cell, e.g., a cell which expresses one or more enzymes involved in immortalization, e.g., TERT. In an embodiment, a host cell can be propagated indefinitely.

A host cell can be cultured in suspension or as a monolayer. Host cell cultures can be performed in a cell culture vessel or a bioreactor. Cell culture vessels include a cell culture dish, plate or flask. Exemplary cell culture vessels include 35 mm, 60 mm, 100 mm, or 150 mm dishes, multi-well plates (e.g., 6-well, 12-well, 24-well, 48-well or 96 well plates), or T-25, T-75 or T-160 flasks.

In an embodiment, a host cell can be cultured in a bioreactor. A bioreactor can be, e.g., a continuous flow batch bioreactor, a perfusion bioreactor, a batch process bioreactor or a fed batch bioreactor. A bioreactor can be maintained under conditions sufficient to express the TREM. The culture conditions can be modulated to optimize yield, purity or structure of the TREM. In an embodiment, a bioreactor comprises at least 1×10⁷, 1×10⁸, 1×10⁹, 1×10¹⁰, 1×10¹¹, 1×10¹², 1×10¹³, or 1×10¹⁴ host cells. In an embodiment, a bioreactor comprises between 1×10⁷ to 1×10¹⁴ host cells; between 1×10⁷ to 0.5×10¹⁴ host cells; between 1×10⁷ to 1×10¹³ host cells; between 1×10⁷ to 0.5×10¹³ host cells; between 1×10⁷ to 1×10¹² host cells; between 1×10⁷ to 0.5×10¹² host cells; between 1×10⁷ to 1×10¹¹ host cells; between 1×10⁷ to 0.5×10¹¹ host cells; between 1×10⁷ to 1×10¹⁰ host cells; between 1×10⁷ to 0.5×10¹⁰ host cells; between 1×10⁷ to 1×10⁹ host cells; between 1×10⁷ to 0.5×10⁹ host cells; between 1×10⁷ to 1×10⁸ host cells; between 1×10⁷ to 0.5×10⁸ host cells; between 0.5×10⁸ to 1×10¹⁴ host cells; between 1×10⁸ to 1×10¹⁴ host cells; between 0.5×10⁹ to 1×10¹⁴ host cells; between 1×10⁹ to 1×10¹⁴ host cells; between 0.5×10¹⁰ to 1×10¹⁴ host cells; between 1×10¹⁰ to 1×10¹⁴ host cells; between 0.5×10¹¹ to 1×10¹⁴ host cells; between 1×10¹¹ to 1×10¹⁴ host cells; between 0.5×10¹² to 1×10¹⁴ host cells; between 1×10¹² to 1×10¹⁴ host cells; between 0.5×10¹³ to 1×10¹⁴ host cells; between 1×10¹³ to 1×10¹⁴ host cells; or between 0.5×10¹³ to 1×10¹⁴ host cells.

In an embodiment, a bioreactor comprises at least 1×10⁵ host cells/mL, 2×10⁵ host cells/mL, 3×10⁵ host cells/mL, 4×10⁵ host cells/mL, 5×10⁵ host cells/mL, 6×10⁵ host cells/mL, 7×10⁵ host cells/mL, 8×10⁵ host cells/mL, 9×10⁵ host cells/mL, 1×10⁶ host cells/mL, 2×10⁶ host cells/mL, 3×10⁶ host cells/mL, 4×10⁶ host cells/mL, 5×10⁶ host cells/mL, 6×10⁶ host cells/mL, 7×10⁶ host cells/mL, 8×10⁶ host cells/mL, 9×10⁶ host cells/mL, 1×10⁷ host cells/mL, 2×10⁷ host cells/mL, 3×10⁷ host cells/mL, 4×10⁷ host cells/mL, 5×10⁷ host cells/mL, 6×10⁷ host cells/mL, 7×10⁷ host cells/mL, 8×10⁷ host cells/mL, 9×10⁷ host cells/mL, 1×10⁸ host cell/mL, 2×10⁸ host cells/mL, 3×10⁸ host cells/mL, 4×10⁸ host cells/mL, 5×10⁸ host cells/mL, 6×10⁸ host cells/mL, 7×10⁸ host cells/mL, 8×10⁸ host cells/mL, 9×10⁸ host cells/mL, or 1×10⁹ host cells/mL. In an embodiment, a bioreactor comprises between 1×10⁵ host cells/mL to 1×10⁹ host cells/mL, between 5×10⁵ host cells/mL to 1×10⁹ host cells/mL, between 1×10⁶ host cells/mL to 1×10⁹ host cells/mL; between 5×10⁶ host cells/mL to 1×10⁹ host cells/mL, between 1×10⁷ host cells/mL to 1×10⁹ host cells/mL, between 5×10⁷ host cells/mL to 1×10⁹ host cells/mL, between 1×10⁸ host cells/mL to 1×10⁹ host cells/mL, between 5×10⁸ host cells/mL to 1×10⁹ host cells/mL, between 1×10⁵ host cells/mL to 5×10⁸ host cells/mL, between 1×10⁵ host cells/mL to 1×10⁸ host cells/mL, between 1×10⁵ host cells/mL to 5×10⁷ host cells/mL, between 1×10⁵ host cells/mL to 1×10⁷ host cells/mL, between 1×10⁵ host cells/mL to 5×10⁶ host cells/mL, between 1×10⁵ host cells/mL to 1×10⁶ host cells/mL, or between 1×10⁵ host cells/mL to 5×10⁵ host cells/mL.

In an embodiment, a batch process bioreactor comprises 1×10⁶ to 1×10⁷ host cells/ml.

In an embodiment, a batch process bioreactor with a 100 mL volume comprises 1×10⁸ to 1×10⁹ host cells.

In an embodiment, a batch process bioreactor with a 100 L volume comprises 1×10¹¹ to 1×10¹² host cells.

In an embodiment, a fed batch bioreactor comprises 1×10⁷ to 3×10⁷ host cells/ml.

In an embodiment, a fed batch bioreactor with a 100 mL volume comprises 1×10⁹ to 3×10⁹ host cells.

In an embodiment, a fed batch bioreactor with a 100 L volume comprises 1×10¹² to 3×10¹² host cells.

In an embodiment, a perfusion bioreactor comprises 1×10⁸ host cells/ml.

In an embodiment, a perfusion bioreactor with a 100 mL volume comprises 1×10¹⁰ host cells.

In an embodiment, a perfusion bioreactor with a 100 L volume comprises 1×10¹³ host cells.

In an embodiment, a bioreactor is maintained under conditions that promote growth of the host cell, e.g., at a temperature (e.g., 37° C.) and gas concentration (e.g., 5% CO₂) that is permissive for growth of the host cell.

For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Exemplary bioreactor units, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. Any suitable bioreactor diameter can be used.

In an embodiment, the bioreactor can have a volume between about 100 mL and about 100 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

### Method of Modifying Host Cells

A host cell can be modified to optimize the production of a TREM, e.g., to have optimized TREM yield, purity, structure (e.g., folding), or stability. In an embodiment, a host cell can be modified (e.g., using a method described herein), to increase or decrease the expression of a desired molecule, e.g., gene, which optimizes production of the TREM, e.g., optimizes yield, purity, structure or stability of the TREM. In an embodiment, a host cell can be epigenetically modified, e.g., using a method described herein, to increase or decrease the expression of a desired gene, which optimizes production.

In an embodiment, a host cell can be modified to increase or decrease the expression of an oncogene (e.g., as described herein), a tumor suppressor (e.g., as described herein) or a molecule involved in tRNA or TREM modulation (e.g., a gene involved in tRNA or TREM transcription, processing, modification, stability or folding). Exemplary oncogenes include Myc (e.g., c-Myc, N-Myc or L-Myc), c-Jun, Wnt, or RAS. Exemplary tumor suppressors include p53 or Rb. Exemplary molecules involved in tRNA or TREM modulation include: RNA Polymerase III (Pol III) and Pol III accessory molecules (e.g., TFIIIB); Maf1, Trm1, Mck1 or Kns 1; enzymes involved in tRNA or TREM modification, e.g., genes listed in Table 2; or molecules with nuclease activity, e.g., or one or more of Dicer, Angiogenin, RNaseA, RNaseP, RNaseZ, Rny1 or PrrC.

In an embodiment, a host cell can be modified by: transfection (e.g., transient transfection or stable transfection); transduction (e.g., viral transduction, e.g., lentiviral, adenoviral or retroviral transduction); electroporation; lipid-based delivery of an agent (e.g., liposomes), nanoparticle based delivery of an agent; or other methods known in the art.

In an embodiment, a host cell can be modified to increase the expression of, e.g., overexpress, a desired molecule, e.g., a gene (e.g., an oncogene, or a gene involved in tRNA or TREM modulation (e.g., a gene encoding an enzyme listed in Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC. Exemplary methods of increasing the expression of a gene include: (a) contacting the host cell with a nucleic acid (e.g., DNA, or RNA) encoding the gene; (b) contacting the host cell with a peptide that expresses the target protein; (c) contacting the host cell with a molecule (e.g., a small RNA (e.g., a micro RNA, or a small interfering RNA) or a low molecular weight compound) that modulates, e.g., increases the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of a negative regulator of the target gene. In an embodiment, a nucleic acid encoding the gene, or a plasmid containing a nucleic acid encoding the gene can be introduced into the host cell by transfection or electroporation. In an embodiment, a nucleic acid encoding a gene can be introduced into the host cell by contacting the host cell with a virus (e.g., a lentivirus, adenovirus or retrovirus) expressing the gene.

In an embodiment, a host cell can be modified to decrease the expression of, e.g., minimize the expression, of a desired molecule, e.g., a gene (e.g., a tumor suppressor, or a gene involved in tRNA or TREM modulation). Exemplary methods of decreasing the expression of a gene include: (a) contacting the host cell with a nucleic acid (e.g., DNA, or RNA) encoding an inhibitor of the gene (e.g., a dominant negative variant or a negative regulator of the gene or protein encoded by the gene); (b) contacting the host cell with a peptide that inhibits the target protein; (c) contacting the host cell with a molecule (e.g., a small RNA (e.g., a micro RNA, or a small interfering RNA) or a low molecular weight compound) that modulates, e.g., inhibits the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of the target gene. In an embodiment, a nucleic acid encoding an inhibitor of the gene, or a plasmid containing a nucleic acid encoding an inhibitor of the gene can be introduced into the host cell by transfection or electroporation. In an embodiment, a nucleic acid encoding an inhibitor of the gene can be introduced into the host cell by contacting the host cell with a virus (e.g., a lentivirus, adenovirus or retrovirus) expressing the inhibitor of the gene.

In an embodiment, a host cell (e.g., a host cell described herein) is modified (e.g., by transfection with a nucleic acid), to express, e.g., overexpress, an oncogene, e.g., an oncogene described herein, e.g., c-Myc.

In an embodiment, a host cell (e.g., a host cell described herein) is modified (e.g., by transfection with a nucleic acid), to repress, e.g., downregulate, expression of a tumor suppressor, e.g., a tumor suppressor described herein, e.g., p53 or Rb.

In an embodiment, a host cell (e.g., a HEK293T cell) is modified (e.g., using a CRISPR/Cas9 molecule) to inhibit, e.g., knockout, expression of a gene that modulates a tRNA or TREM, e.g., Maf1. In an embodiment, a host cell (e.g., a HEK293T cell) is modified to overexpress a gene that modulates a tRNA or TREM, e.g., Trm1.

In an embodiment, a host cell (e.g., a HEK293T cell) is modified to overexpress a gene that modulates a tRNA or TREM, e.g., Trm1, and to overexpress an oncogene, e.g., an oncogene described herein, e.g., c-Myc.

### TREM

A “tRNA-based effector molecule” or “TREM” refers to an RNA molecule comprising one or more of the properties described herein. A TREM can be charged with an amino acid, e.g., a cognate amino acid; charged with a non-cognate amino acid (e.g., a mischarged TREM (mTREM); or not charged with an amino acid, e.g., an uncharged TREM (uTREM).

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM comprises at least 30 consecutive nucleotides of an RNA sequence encoded by a DNA sequence disclosed in Table 1, e.g., at least 30 consecutive nucleotides of an RNA sequence encoded by any one of SEQ ID NOs: 1-451 disclosed in Table 1. In an embodiment, a TREM comprises at least 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises at least 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

TABLE 1
List of tRNA sequences

| SEQ | | |
|---|---|---|
| ID | | |
| NO | tRNA name | tRNA sequence |
| 1 | Ala_AGC_chr6: 28763741-28763812 (−) | GGGGGTATAGCTCAGTGGTAGAGCGCGTGCTTAGCATGCACGAGGTCC |
| | | TGGGTTCGATCCCCAGTACCTCCA |
| 2 | Ala_AGC_chr6: 26687485-26687557 (+) | GGGGAATTAGCTCAAGTGGTAGAGCGCTTGCTTAGCACGCAAGAGGTA |
| | | GTGGGATCGATGCCCACATTCTCCA |
| 3 | Ala_AGC_chr6: 26572092-26572164 (−) | GGGGAATTAGCTCAAATGGTAGAGCGCTCGCTTAGCATGCGAGAGGTA |
| | | GCGGGATCGATGCCCGCATTCTCCA |
| 4 | Ala_AGC_chr6: 26682715-26682787 (+) | GGGGAATTAGCTCAAGTGGTAGAGCGCTTGCTTAGCATGCAAGAGGTA |
| | | GTGGGATCGATGCCCACATTCTCCA |
| 5 | Ala_AGC_chr6: 26705606-26705678 (+) | GGGGAATTAGCTCAAGCGGTAGAGCGCTTGCTTAGCATGCAAGAGGTA |
| | | GTGGGATCGATGCCCACATTCTCCA |
| 6 | Ala_AGC_chr6: 26673590-26673662 (+) | GGGGAATTAGCTCAAGTGGTAGAGCGCTTGCTTAGCATGCAAGAGGTA |
| | | GTGGGATCAATGCCCACATTCTCCA |
| 7 | Ala_AGC_chr14: 89445442-89445514 (+) | GGGGAATTAGCTCAAGTGGTAGAGCGCTCGCTTAGCATGCGAGAGGTA |
| | | GTGGGATCGATGCCCGCATTCTCCA |
| 8 | Ala_AGC_chr6: 58196623-58196695 (−) | GGGGAATTAGCCCAAGTGGTAGAGCGCTTGCTTAGCATGCAAGAGGTA |
| | | GTGGGATCGATGCCCACATTCTCCA |
| 9 | Ala_AGC_chr6: 28806221-28806292 (−) | GGGGGTGTAGCTCAGTGGTAGAGCGCGTGCTTAGCATGCACGAGGCCC |
| | | CGGGTTCAATCCCCGGCACCTCCA |
| 10 | Ala_AGC_chr6: 28574933-28575004 (+) | GGGGGTGTAGCTCAGTGGTAGAGCGCGTGCTTAGCATGTACGAGGTCC |
| | | CGGGTTCAATCCCCGGCACCTCCA |
| 11 | Ala_AGC_chr6: 28626014-28626085 (−) | GGGGATGTAGCTCAGTGGTAGAGCGCATGCTTAGCATGCATGAGGTCC |
| | | CGGGTTCGATCCCCAGCATCTCCA |
| 12 | Ala_AGC_chr6: 28678366-28678437 (+) | GGGGGTGTAGCTCAGTGGTAGAGCGCGTGCTTAGCATGCACGAGGCCC |
| | | TGGGTTCAATCCCCAGCACCTCCA |
| 13 | Ala_AGC_chr6: 28779849-28779920 (−) | GGGGGTATAGCTCAGCGGTAGAGCGCGTGCTTAGCATGCACGAGGTCC |
| | | TGGGTTCAATCCCCAATACCTCCA |
| 14 | Ala_AGC_chr6: 28687481-28687552 (+) | GGGGGTGTAGCTCAGTGGTAGAGCGCGTGCTTAGCATGCACGAGGCCC |
| | | CGGGTTCAATCCCTGGCACCTCCA |
| 15 | Ala_AGC_chr2: 27274082-27274154 (+) | GGGGGATTAGCTCAAATGGTAGAGCGCTCGCTTAGCATGCGAGAGGTA |
| | | GCGGGATCGATGCCCGCATCCTCCA |
| 16 | Ala_AGC_chr6: 26730737-26730809 (+) | GGGGAATTAGCTCAGGCGGTAGAGCGCTCGCTTAGCATGCGAGAGGTA |
| | | GCGGGATCGACGCCCGCATTCTCCA |
| 17 | Ala_CGC_chr6: 26553731-26553802 (+) | GGGGATGTAGCTCAGTGGTAGAGCGCATGCTTCGCATGTATGAGGTCC |
| | | CGGGTTCGATCCCCGGCATCTCCA |
| 18 | Ala_CGC_chr6: 28641613-28641684 (−) | GGGGATGTAGCTCAGTGGTAGAGCGCATGCTTCGCATGTATGAGGCCC |
| | | CGGGTTCGATCCCCGGCATCTCCA |
| 19 | Ala_CGC_chr2: 157257281-157257352 (+) | GGGGATGTAGCTCAGTGGTAGAGCGCGCGCTTCGCATGTGTGAGGTCC |
| | | CGGGTTCAATCCCCGGCATCTCCA |
| 20 | Ala_CGC_chr6: 28697092-28697163 (+) | GGGGGTGTAGCTCAGTGGTAGAGCGCGTGCTTCGCATGTACGAGGCCC |
| | | CGGGTTCGACCCCCGGCTCCTCCA |
| 21 | Ala_TGC_chr6: 28757547-28757618 (−) | GGGGGTGTAGCTCAGTGGTAGAGCGCATGCTTTGCATGTATGAGGTCC |
| | | CGGGTTCGATCCCCGGCACCTCCA |
| 22 | Ala_TGC_chr6: 28611222-28611293 (+) | GGGGATGTAGCTCAGTGGTAGAGCGCATGCTTTGCATGTATGAGGTCC |
| | | CGGGTTCGATCCCCGGCATCTCCA |
| 23 | Ala_TGC_chr5: 180633868-180633939 (+) | GGGGATGTAGCTCAGTGGTAGAGCGCATGCTTTGCATGTATGAGGCCC |
| | | CGGGTTCGATCCCCGGCATCTCCA |
| 24 | Ala_TGC_chr12: 125424512-125424583 (+) | GGGGATGTAGCTCAGTGGTAGAGCGCATGCTTTGCACGTATGAGGCCC |
| | | CGGGTTCAATCCCCGGCATCTCCA |
| 25 | Ala_TGC_chr6: 28785012-28785083 (−) | GGGGGTGTAGCTCAGTGGTAGAGCGCATGCTTTGCATGTATGAGGCCT |
| | | CGGGTTCGATCCCCGACACCTCCA |
| 26 | Ala_TGC_chr6: 28726141-28726212 (−) | GGGGGTGTAGCTCAGTGGTAGAGCACATGCTTTGCATGTGTGAGGCCC |
| | | CGGGTTCGATCCCCGGCACCTCCA |
| 27 | Ala_TGC_chr6: 28770577-28770647 (−) | GGGGGTGTAGCTCAGTGGTAGAGCGCATGCTTTGCATGTATGAGGCCT |
| | | CGGTTCGATCCCCGACACCTCCA |
| 28 | Arg_ACG_chr6: 26328368-26328440 (+) | GGGCCAGTGGCGCAATGGATAACGCGTCTGACTACGGATCAGAAGATT |
| | | CCAGGTTCGACTCCTGGCTGGCTCG |
| 29 | Arg_ACG_chr3: 45730491-45730563 (−) | GGGCCAGTGGCGCAATGGATAACGCGTCTGACTACGGATCAGAAGATT |
| | | CTAGGTTCGACTCCTGGCTGGCTCG |
| 30 | Arg_CCG_chr6: 28710729-28710801 (−) | GGCCGCGTGGCCTAATGGATAAGGCGTCTGATTCCGGATCAGAAGATT |
| | | GAGGGTTCGAGTCCCTTCGTGGTCG |
| 31 | Arg_CCG_chr17: 66016013-66016085 (−) | GACCCAGTGGCCTAATGGATAAGGCATCAGCCTCCGGAGCTGGGGATT |
| | | GTGGGTTCGAGTCCCATCTGGGTCG |
| 32 | Arg_CCT_chr17: 73030001-73030073 (+) | GCCCCAGTGGCCTAATGGATAAGGCACTGGCCTCCTAAGCCAGGGATT |
| | | GTGGGTTCGAGTCCCACCTGGGGTA |
| 33 | Arg_CCT_chr17: 73030526-73030598 (−) | GCCCCAGTGGCCTAATGGATAAGGCACTGGCCTCCTAAGCCAGGGATT |
| | | GTGGGTTCGAGTCCCACCTGGGGTG |
| 34 | Arg_CCT_chr16: 3202901-3202973 (+) | GCCCCGGTGGCCTAATGGATAAGGCATTGGCCTCCTAAGCCAGGGATT |
| | | GTGGGTTCGAGTCCCACCCGGGGTA |
| 35 | Arg_CCT_chr7: 139025446-139025518 (+) | GCCCCAGTGGCCTAATGGATAAGGCATTGGCCTCCTAAGCCAGGGATT |
| | | GTGGGTTCGAGTCCCATCTGGGGTG |
| 36 | Arg_CCT_chr16: 3243918-3243990 (+) | GCCCCAGTGGCCTGATGGATAAGGTACTGGCCTCCTAAGCCAGGGATT |
| | | GTGGGTTCGAGTTCCACCTGGGGTA |
| 37 | Arg_TCG_chr15: 89878304-89878376 (+) | GGCCGCGTGGCCTAATGGATAAGGCGTCTGACTTCGGATCAGAAGATT |
| | | GCAGGTTCGAGTCCTGCCGCGGTCG |
| 38 | Arg_TCG_chr6: 26323046-26323118 (+) | GACCACGTGGCCTAATGGATAAGGCGTCTGACTTCGGATCAGAAGATT |
| | | GAGGGTTCGAATCCCTCCGTGGTTA |
| 39 | Arg_TCG_chr17: 73031208-73031280 (+) | GACCGCGTGGCCTAATGGATAAGGCGTCTGACTTCGGATCAGAAGATT |
| | | GAGGGTTCGAGTCCCTTCGTGGTCG |
| 40 | Arg_TCG_chr6: 26299905-26299977 (+) | GACCACGTGGCCTAATGGATAAGGCGTCTGACTTCGGATCAGAAGATT |
| | | GAGGGTTCGAATCCCTTCGTGGTTA |
| 41 | Arg_TCG_chr6: 28510891-28510963 (−) | GACCACGTGGCCTAATGGATAAGGCGTCTGACTTCGGATCAGAAGATT |
| | | GAGGGTTCGAATCCCTTCGTGGTTG |
| 42 | Arg_TCG_chr9: 112960803-112960875 (+) | GGCCGTGTGGCCTAATGGATAAGGCGTCTGACTTCGGATCAAAAGATT |
| | | GCAGGTTTGAGTTCTGCCACGGTCG |
| 43 | Arg_TCT_chr1: 94313129-94313213 (+) | GGCTCCGTGGCGCAATGGATAGCGCATTGGACTTCTAGAGGCTGAAGG |
| | | CATTCAAAGGTTCCGGGTTCGAGTCCCGGCGGAGTCG |
| 44 | Arg_TCT_chr17: 8024243-8024330 (+) | GGCTCTGTGGCGCAATGGATAGCGCATTGGACTTCTAGTGACGAATAG |
| | | AGCAATTCAAAGGTTGTGGGTTCGAATCCCACCAGAGTCG |
| 45 | Arg_TCT_chr9: 131102355-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCG |
| 46 | Arg_TCT_chr11: 59318767-59318852 (+) | GGCTCTGTGGCGCAATGGATAGCGCATTGGACTTCTAGATAGTTAGAG |
| | | AAATTCAAAGGTTGTGGGTTCGAGTCCCACCAGAGTCG |
| 47 | Arg_TCT_chr1: 159111401-159111474 (−) | GTCTCTGTGGCGCAATGGACGAGCGCGCTGGACTTCTAATCCAGAGGT |
| | | TCCGGGTTCGAGTCCCGGCAGAGATG |
| 48 | Arg_TCT_chr6: 27529963-27530049 (+) | GGCTCTGTGGCGCAATGGATAGCGCATTGGACTTCTAGCCTAAATCAA |
| | | GAGATTCAAAGGTTGCGGGTTCGAGTCCCTCCAGAGTCG |
| 49 | Asn_GTT_chr1: 161510031-161510104 (+) | GTCTCTGTGGCGCAATCGGTTAGCGCGTTCGGCTGTTAACCGAAAGGT |
| | | TGGTGGTTCGATCCCACCCAGGGACG |
| 50 | Asn_GTT_chr1: 143879832-143879905 (−) | GTCTCTGTGGCGCAATCGGCTAGCGCGTTTGGCTGTTAACTAAAAGGTT |
| | | GGCGGTTCGAACCCACCCAGAGGCG |
| 51 | Asn_GTT_chr1: 144301611-144301684 (+) | GTCTCTGTGGTGCAATCGGTTAGCGCGTTCCGCTGTTAACCGAAAGCTT |
| | | GGTGGTTCGAGCCCACCCAGGGATG |
| 52 | Asn_GTT_chr1: 149326272-149326345 (−) | GTCTCTGTGGCGCAATCGGCTAGCGCGTTTGGCTGTTAACTAAAAAGTT |
| | | GGTGGTTCGAACACACCCAGAGGCG |
| 53 | Asn_GTT_chr1: 148248115-148248188 (+) | GTCTCTGTGGCGCAATCGGTTAGCGCGTTCGGCTGTTAACCGAAAGGT |
| | | TGGTGGTTCGAGCCCACCCAGGGACG |
| 54 | Asn_GTT_chr1: 148598314-148598387 (−) | GTCTCTGTGGCGCAATCGGTTAGCGCATTCGGCTGTTAACCGAAAGGT |
| | | TGGTGGTTCGAGCCCACCCAGGGACG |
| 55 | Asn_GTT_chr1: 17216172-17216245 (+) | GTCTCTGTGGCGCAATCGGTTAGCGCGTTCGGCTGTTAACCGAAAGAT |
| | | TGGTGGTTCGAGCCCACCCAGGGACG |
| 56 | Asn_GTT_chr1: 16847080-16847153 (−) | GTCTCTGTGGCGCAATCGGTTAGCGCGTTCGGCTGTTAACTGAAAGGTT |
| | | GGTGGTTCGAGCCCACCCAGGGACG |
| 57 | Asn_GTT_chr1: 149230570-149230643 (−) | GTCTCTGTGGCGCAATGGGTTAGCGCGTTCGGCTGTTAACCGAAAGGT |
| | | TGGTGGTTCGAGCCCATCCAGGGACG |
| 58 | Asn_GTT_chr1: 148000805-148000878 (+) | GTCTCTGTGGCGTAGTCGGTTAGCGCGTTCGGCTGTTAACCGAAAAGTT |
| | | GGTGGTTCGAGCCCACCCAGGAACG |
| 59 | Asn_GTT_chr1: 149711798-149711871 (−) | GTCTCTGTGGCGCAATCGGCTAGCGCGTTTGGCTGTTAACTAAAAGGTT |
| | | GGTGGTTCGAACCCACCCAGAGGCG |
| 60 | Asn_GTT_chr1: 145979034-145979107 (−) | GTCTCTGTGGCGCAATCGGTTAGCGCGTTCGGCTGTTAACTGAAAGGTT |
| | | AGTGGTTCGAGCCCACCCGGGGACG |
| 61 | Asp_GTC_chr12: 98897281-98897352 (+) | TCCTCGTTAGTATAGTGGTTAGTATCCCCGCCTGTCACGCGGGAGACCG |
| | | GGGTTCAATTCCCCGACGGGGAG |
| 62 | Asp_GTC_chr1: 161410615-161410686 (−) | TCCTCGTTAGTATAGTGGTGAGTATCCCCGCCTGTCACGCGGGAGACC |
| | | GGGGTTCGATTCCCCGACGGGGAG |
| 63 | Asp_GTC_chr6: 27551236-27551307 (−) | TCCTCGTTAGTATAGTGGTGAGTGTCCCCGTCTGTCACGCGGGAGACC |
| | | GGGGTTCGATTCCCCGACGGGGAG |
| 64 | Cys_GCA_chr7: 149007281-149007352 (+) | GGGGGCATAGCTCAGTGGTAGAGCATTTGACTGCAGATCAAGAGGTCC |
| | | CTGGTTCAAATCCAGGTGCCCCCT |
| 65 | Cys_GCA_chr7: 149074601-149074672 (−) | GGGGGTATAGCTCAGGGGTAGAGCATTTGACTGCAGATCAAGAGGTCC |
| | | CTGGTTCAAATCCAGGTGCCCCCC |
| 66 | Cys_GCA_chr7: 149112229-149112300 (−) | GGGGGTATAGCTTAGCGGTAGAGCATTTGACTGCAGATCAAGAGGTCC |
| | | CCGGTTCAAATCCGGGTGCCCCCT |
| 67 | Cys_GCA_chr7: 149344046-149344117 (−) | GGGGGTATAGCTTAGGGGTAGAGCATTTGACTGCAGATCAAAAGGTCC |
| | | CTGGTTCAAATCCAGGTGCCCCTT |
| 68 | Cys_GCA_chr7: 149052766-149052837 (−) | GGGGGTATAGCTCAGGGGTAGAGCATTTGACTGCAGATCAAGAGGTCC |
| | | CCAGTTCAAATCTGGGTGCCCCCT |
| 69 | Cys_GCA_chr17: 37017937-37018008 (−) | GGGGGTATAGCTCAGGGGTAGAGCATTTGACTGCAGATCAAGAAGTCC |
| | | CCGGTTCAAATCCGGGTGCCCCCT |
| 70 | Cys_GCA_chr7: 149281816-149281887 (+) | GGGGGTATAGCTCAGGGGTAGAGCATTTGACTGCAGATCAAGAGGTCT |
| | | CTGGTTCAAATCCAGGTGCCCCCT |
| 71 | Cys_GCA_chr7: 149243631-149243702 (+) | GGGGGTATAGCTCAGGGGTAGAGCACTTGACTGCAGATCAAGAAGTCC |
| | | TTGGTTCAAATCCAGGTGCCCCCT |
| 72 | Cys_GCA_chr7: 149388272-149388343 (−) | GGGGATATAGCTCAGGGGTAGAGCATTTGACTGCAGATCAAGAGGTCC |
| | | CCGGTTCAAATCCGGGTGCCCCCC |
| 73 | Cys_GCA_chr7: 149072850-149072921 (−) | GGGGGTATAGTTCAGGGGTAGAGCATTTGACTGCAGATCAAGAGGTCC |
| | | CTGGTTCAAATCCAGGTGCCCCCT |
| 74 | Cys_GCA_chr7: 149310156-149310227 (−) | GGGGGTATAGCTCAGGGGTAGAGCATTTGACTGCAAATCAAGAGGTCC |
| | | CTGATTCAAATCCAGGTGCCCCCT |
| 75 | Cys_GCA_chr4: 124430005-124430076 (−) | GGGGGTATAGCTCAGTGGTAGAGCATTTGACTGCAGATCAAGAGGTCC |
| | | CCGGTTCAAATCCGGGTGCCCCCT |
| 76 | Cys_GCA_chr7: 149295046-149295117 (+) | GGGCGTATAGCTCAGGGGTAGAGCATTTGACTGCAGATCAAGAGGTCC |
| | | CCAGTTCAAATCTGGGTGCCCCCT |
| 77 | Cys_GCA_chr7: 149361915-149361986 (+) | GGGGGTATAGCTCACAGGTAGAGCATTTGACTGCAGATCAAGAGGTCC |
| | | CCGGTTCAAATCTGGGTGCCCCCT |
| 78 | Cys_GCA_chr7: 149253802-149253871 (+) | GGGCGTATAGCTCAGGGGTAGAGCATTTGACTGCAGATCAAGAGGTCC |
| | | CCAGTTCAAATCTGGGTGCCCA |
| 79 | Cys_GCA_chr7: 149292305-149292376 (−) | GGGGGTATAGCTCACAGGTAGAGCATTTGACTGCAGATCAAGAGGTCC |
| | | CCGGTTCAAATCCGGTTACTCCCT |
| 80 | Cys_GCA_chr7: 149286164-149286235 (−) | GGGGGTATAGCTCAGGGGTAGAGCACTTGACTGCAGATCAAGAGGTCC |
| | | CTGGTTCAAATCCAGGTGCCCCCT |
| 81 | Cys_GCA_chr17: 37025545-37025616 (−) | GGGGGTATAGCTCAGTGGTAGAGCATTTGACTGCAGATCAAGAGGTCC |
| | | CTGGTTCAAATCCGGGTGCCCCCT |
| 82 | Cys_GCA_chr15: 80036997-80037069 (+) | GGGGGTATAGCTCAGTGGGTAGAGCATTTGACTGCAGATCAAGAGGTC |
| | | CCCGGTTCAAATCCGGGTGCCCCCT |
| 83 | Cys_GCA_chr3: 131947944-131948015 (−) | GGGGGTGTAGCTCAGTGGTAGAGCATTTGACTGCAGATCAAGAGGTCC |
| | | CTGGTTCAAATCCAGGTGCCCCCT |
| 84 | Cys_GCA_chr1: 93981834-93981906 (−) | GGGGGTATAGCTCAGGTGGTAGAGCATTTGACTGCAGATCAAGAGGTC |
| | | CCCGGTTCAAATCCGGGTGCCCCCT |
| 85 | Cys_GCA_chr14: 73429679-73429750 (+) | GGGGGTATAGCTCAGGGGTAGAGCATTTGACTGCAGATCAAGAGGTCC |
| | | CCGGTTCAAATCCGGGTGCCCCCT |
| 86 | Cys_GCA_chr3: 131950642-131950713 (−) | GGGGGTATAGCTCAGGGGTAGAGCATTTGACTGCAGATCAAGAGGTCC |
| | | CTGGTTCAAATCCAGGTGCCCCCT |
| 87 | Gln_CTG_chr6: 18836402-18836473 (+) | GGTTCCATGGTGTAATGGTTAGCACTCTGGACTCTGAATCCAGCGATCC |
| | | GAGTTCAAATCTCGGTGGAACCT |
| 88 | Gln_CTG_chr6: 27515531-27515602 (−) | GGTTCCATGGTGTAATGGTTAGCACTCTGGACTCTGAATCCAGCGATCC |
| | | GAGTTCAAGTCTCGGTGGAACCT |
| 89 | Gln_CTG_chr1: 145963304-145963375 (+) | GGTTCCATGGTGTAATGGTGAGCACTCTGGACTCTGAATCCAGCGATC |
| | | CGAGTTCGAGTCTCGGTGGAACCT |
| 90 | Gln_CTG_chr1: 147737382-147737453 (−) | GGTTCCATGGTGTAATGGTAAGCACTCTGGACTCTGAATCCAGCGATC |
| | | CGAGTTCGAGTCTCGGTGGAACCT |
| 91 | Gln_CTG_chr6: 27263212-27263283 (+) | GGTTCCATGGTGTAATGGTTAGCACTCTGGACTCTGAATCCGGTAATCC |
| | | GAGTTCAAATCTCGGTGGAACCT |
| 92 | Gln_CTG_chr6: 27759135-27759206 (−) | GGCCCCATGGTGTAATGGTCAGCACTCTGGACTCTGAATCCAGCGATC |
| | | CGAGTTCAAATCTCGGTGGGACCC |
| 93 | Gln_CTG_chr1: 147800937-147801008 (+) | GGTTCCATGGTGTAATGGTAAGCACTCTGGACTCTGAATCCAGCCATCT |
| | | GAGTTCGAGTCTCTGTGGAACCT |
| 94 | Gln_TTG_chr17: 47269890-47269961 (+) | GGTCCCATGGTGTAATGGTTAGCACTCTGGACTTTGAATCCAGCGATCC |
| | | GAGTTCAAATCTCGGTGGGACCT |
| 95 | Gln_TTG_chr6: 28557156-28557227 (+) | GGTCCCATGGTGTAATGGTTAGCACTCTGGACTTTGAATCCAGCAATCC |
| | | GAGTTCGAATCTCGGTGGGACCT |
| 96 | Gln_TTG_chr6: 26311424-26311495 (−) | GGCCCCATGGTGTAATGGTTAGCACTCTGGACTTTGAATCCAGCGATC |
| | | CGAGTTCAAATCTCGGTGGGACCT |
| 97 | Gln_TTG_chr6: 145503859-145503930 (+) | GGTCCCATGGTGTAATGGTTAGCACTCTGGGCTTTGAATCCAGCAATCC |
| | | GAGTTCGAATCTTGGTGGGACCT |
| 98 | Glu_CTC_chr1: 145399233-145399304 (−) | TCCCTGGTGGTCTAGTGGTTAGGATTCGGCGCTCTCACCGCCGCGGCCC |
| | | GGGTTCGATTCCCGGTCAGGGAA |
| 99 | Glu_CTC_chr1: 249168447-249168518 (+) | TCCCTGGTGGTCTAGTGGTTAGGATTCGGCGCTCTCACCGCCGCGGCCC |
| | | GGGTTCGATTCCCGGTCAGGAAA |
| 100 | Glu_TTC_chr2: 131094701-131094772 (−) | TCCCATATGGTCTAGCGGTTAGGATTCCTGGTTTTCACCCAGGTGGCCC |
| | | GGGTTCGACTCCCGGTATGGGAA |
| 101 | Glu_TTC_chr13 :45492062-45492133 (−) | TCCCACATGGTCTAGCGGTTAGGATTCCTGGTTTTCACCCAGGCGGCCC |
| | | GGGTTCGACTCCCGGTGTGGGAA |
| 102 | Glu_TTC_chr1: 17199078-17199149 (+) | TCCCTGGTGGTCTAGTGGCTAGGATTCGGCGCTTTCACCGCCGCGGCCC |
| | | GGGTTCGATTCCCGGCCAGGGAA |
| 103 | Glu_TTC_chr1: 16861774-16861845 (−) | TCCCTGGTGGTCTAGTGGCTAGGATTCGGCGCTTTCACCGCCGCGGCCC |
| | | GGGTTCGATTCCCGGTCAGGGAA |
| 104 | Gly_CCC_chr1 :16872434-16872504 (−) | GCATTGGTGGTTCAGTGGTAGAATTCTCGCCTCCCACGCGGGAGACCC |
| | | GGGTTCAATTCCCGGCCAATGCA |
| 105 | Gly_CCC_chr2:70476123-70476193 (−) | GCGCCGCTGGTGTAGTGGTATCATGCAAGATTCCCATTCTTGCGACCCG |
| | | GGTTCGATTCCCGGGCGGCGCA |
| 106 | Gly_CCC_chr17 : 19764175-19764245 (+) | GCATTGGTGGTTCAATGGTAGAATTCTCGCCTCCCACGCAGGAGACCC |
| | | AGGTTCGATTCCTGGCCAATGCA |
| 107 | Gly_GCC_chr1: 161413094-161413164 (+) | GCATGGGTGGTTCAGTGGTAGAATTCTCGCCTGCCACGCGGGAGGCCC |
| | | GGGTTCGATTCCCGGCCCATGCA |
| 108 | Gly_GCC_chr1: 161493637-161493707 (−) | GCATTGGTGGTTCAGTGGTAGAATTCTCGCCTGCCACGCGGGAGGCCC |
| | | GGGTTCGATTCCCGGCCAATGCA |
| 109 | Gly_GCC_chr16: 70812114-70812184 (−) | GCATTGGTGGTTCAGTGGTAGAATTCTCGCCTGCCACGCGGGAGGCCC |
| | | GGGTTTGATTCCCGGCCAGTGCA |
| 110 | Gly_GCC_chr1: 161450356-161450426 (+) | GCATAGGTGGTTCAGTGGTAGAATTCTTGCCTGCCACGCAGGAGGCCC |
| | | AGGTTTGATTCCTGGCCCATGCA |
| 111 | Gly_GCC_chr16:70822597-70822667 (+) | GCATTGGTGGTTCAGTGGTAGAATTCTCGCCTGCCATGCGGGCGGCCG |
| | | GGCTTCGATTCCTGGCCAATGCA |
| 112 | Gly_TCC_chr19: 4724082-4724153 (+) | GCGTTGGTGGTATAGTGGTTAGCATAGCTGCCTTCCAAGCAGTTGACC |
| | | CGGGTTCGATTCCCGGCCAACGCA |
| 113 | Gly_TCC_chr1: 145397864-145397935 (−) | GCGTTGGTGGTATAGTGGTGAGCATAGCTGCCTTCCAAGCAGTTGACC |
| | | CGGGTTCGATTCCCGGCCAACGCA |
| 114 | Gly_TCC_chr17: 8124866-8124937 (+) | GCGTTGGTGGTATAGTGGTAAGCATAGCTGCCTTCCAAGCAGTTGACC |
| | | CGGGTTCGATTCCCGGCCAACGCA |
| 115 | Gly_TCC_chr1: 161409961-161410032 (−) | GCGTTGGTGGTATAGTGGTGAGCATAGTTGCCTTCCAAGCAGTTGACC |
| | | CGGGCTCGATTCCCGCCCAACGCA |
| 116 | His_GTG_chr1: 145396881-145396952 (−) | GCCGTGATCGTATAGTGGTTAGTACTCTGCGTTGTGGCCGCAGCAACCT |
| | | CGGTTCGAATCCGAGTCACGGCA |
| 117 | His_GTG_chr1: 149155828-149155899 (−) | GCCATGATCGTATAGTGGTTAGTACTCTGCGCTGTGGCCGCAGCAACC |
| | | TCGGTTCGAATCCGAGTCACGGCA |
| 118 | Ile_AAT_chr6: 58149254-58149327 (+) | GGCCGGTTAGCTCAGTTGGTTAGAGCGTGGCGCTAATAACGCCAAGGT |
| | | CGCGGGTTCGATCCCCGTACGGGCCA |
| 119 | Ile_AAT_chr6: 27655967-27656040 (+) | GGCCGGTTAGCTCAGTTGGTTAGAGCGTGGTGCTAATAACGCCAAGGT |
| | | CGCGGGTTCGATCCCCGTACTGGCCA |
| 120 | Ile_AAT_chr6: 27242990-27243063 (−) | GGCTGGTTAGCTCAGTTGGTTAGAGCGTGGTGCTAATAACGCCAAGGT |
| | | CGCGGGTTCGATCCCCGTACTGGCCA |
| 121 | Ile_AAT_chr17: 8130309-8130382 (−) | GGCCGGTTAGCTCAGTTGGTTAGAGCGTGGTGCTAATAACGCCAAGGT |
| | | CGCGGGTTCGAACCCCGTACGGGCCA |
| 122 | Ile_AAT_chr6: 26554350-26554423 (+) | GGCCGGTTAGCTCAGTTGGTTAGAGCGTGGTGCTAATAACGCCAAGGT |
| | | CGCGGGTTCGATCCCCGTACGGGCCA |
| 123 | Ile_AAT_chr6: 26745255-26745328 (−) | GGCCGGTTAGCTCAGTTGGTTAGAGCGTGGTGCTAATAACGCTAAGGT |
| | | CGCGGGTTCGATCCCCGTACTGGCCA |
| 124 | Ile_AAT_chr6: 26721221-26721294 (−) | GGCCGGTTAGCTCAGTTGGTCAGAGCGTGGTGCTAATAACGCCAAGGT |
| | | CGCGGGTTCGATCCCCGTACGGGCCA |
| 125 | Ile_AAT_chr6: 27636362-27636435 (+) | GGCCGGTTAGCTCAGTCGGCTAGAGCGTGGTGCTAATAACGCCAAGGT |
| | | CGCGGGTTCGATCCCCGTACGGGCCA |
| 126 | Ile_AAT_chr6: 27241739-27241812 (+) | GGCTGGTTAGTTCAGTTGGTTAGAGCGTGGTGCTAATAACGCCAAGGT |
| | | CGTGGGTTCGATCCCCATATCGGCCA |
| 127 | Ile_GAT_chrX: 3756418-3756491 (−) | GGCCGGTTAGCTCAGTTGGTAAGAGCGTGGTGCTGATAACACCAAGGT |
| | | CGCGGGCTCGACTCCCGCACCGGCCA |
| 128 | Ile_TAT_chr19: 39902808-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCA |
| 129 | Ile_TAT_chr2: 43037676-43037768 (+) | GCTCCAGTGGCGCAATCGGTTAGCGCGCGGTACTTATACAGCAGTACA |
| | | TGCAGAGCAATGCCGAGGTTGTGAGTTCGAGCCTCACCTGGAGCA |
| 130 | Ile_TAT_chr6: 26988125-26988218 (+) | GCTCCAGTGGCGCAATCGGTTAGCGCGCGGTACTTATATGGCAGTATG |
| | | TGTGCGAGTGATGCCGAGGTTGTGAGTTCGAGCCTCACCTGGAGCA |
| 131 | Ile_TAT_chr6: 27599200-27599293 (+) | GCTCCAGTGGCGCAATCGGTTAGCGCGCGGTACTTATACAACAGTATA |
| | | TGTGCGGGTGATGCCGAGGTTGTGAGTTCGAGCCTCACCTGGAGCA |
| 132 | Ile_TAT_chr6: 28505367-28505460 (+) | GCTCCAGTGGCGCAATCGGTTAGCGCGCGGTACTTATAAGACAGTGCA |
| | | CCTGTGAGCAATGCCGAGGTTGTGAGTTCAAGCCTCACCTGGAGCA |
| 133 | Leu_AAG_chr5: 180524474-180524555 (−) | GGTAGCGTGGCCGAGCGGTCTAAGGCGCTGGATTAAGGCTCCAGTCTC |
| | | TTCGGAGGCGTGGGTTCGAATCCCACCGCTGCCA |
| 134 | Leu_AAG_chr5: 180614701-180614782 (+) | GGTAGCGTGGCCGAGCGGTCTAAGGCGCTGGATTAAGGCTCCAGTCTC |
| | | TTCGGGGGCGTGGGTTCGAATCCCACCGCTGCCA |
| 135 | Leu_AAG_chr6: 28956779-28956860 (+) | GGTAGCGTGGCCGAGCGGTCTAAGGCGCTGGATTAAGGCTCCAGTCTC |
| | | TTCGGGGGCGTGGGTTCAAATCCCACCGCTGCCA |
| 136 | Leu_AAG_chr6: 28446400-28446481 (−) | GGTAGCGTGGCCGAGTGGTCTAAGACGCTGGATTAAGGCTCCAGTCTC |
| | | TTCGGGGGCGTGGGTTTGAATCCCACCGCTGCCA |
| 137 | Leu_CAA_chr6: 28864000-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 138 | Leu_CAA_chr6: 28908830-28908934 (+) | GTCAGGATGGCCGAGTGGTCTAAGGCGCCAGACTCAAGCTTGGCTTCC |
| | | TCGTGTTGAGGATTCTGGTCTCCAATGGAGGCGTGGGTTCGAATCCCA |
| 139 | Leu_CAA_chr6: 27573417-27573524 (−) | GTCAGGATGGCCGAGTGGTCTAAGGCGCCAGACTCAAGCTTACTGCTT |
| | | CCTGTGTTCGGGTCTTCTGGTCTCCGTATGGAGGCGTGGGTTCGAATCC |
| 140 | Leu_CAA_chr6: 27570348-27570454 (−) | GTCAGGATGGCCGAGTGGTCTAAGGCGCCAGACTCAAGTTGCTACTTC |
| | | CCAGGTTTGGGGCTTCTGGTCTCCGCATGGAGGCGTGGGTTCGAATCC |
| 141 | Leu_CAA_chr1: 249168054-249168159 (+) | GTCAGGATGGCCGAGTGGTCTAAGGCGCCAGACTCAAGGTAAGCACCT |
| | | TGCCTGCGGGCTTTCTGGTCTCCGGATGGAGGCGTGGGTTCGAATCCC |
| 142 | Leu_CAA_chr11: 9296790-9296863 (+) | GCCTCCTTAGTGCAGTAGGTAGCGCATCAGTCTCAAAATCTGAATGGT |
| | | CCTGAGTTCAAGCCTCAGAGGGGGCA |
| 143 | Leu_CAA_chr1: 161581736-161581819 (−) | GTCAGGATGGCCGAGCAGTCTTAAGGCGCTGCGTTCAAATCGCACCCT |
| | | CCGCTGGAGGCGTGGGTTCGAATCCCACTTTTGACA |
| 144 | Leu_CAG_chr1: 161411323-161411405 (+) | GTCAGGATGGCCGAGCGGTCTAAGGCGCTGCGTTCAGGTCGCAGTCTC |
| | | CCCTGGAGGCGTGGGTTCGAATCCCACTCCTGACA |
| 145 | Leu_CAG_chr16: 57333863-57333945 (+) | GTCAGGATGGCCGAGCGGTCTAAGGCGCTGCGTTCAGGTCGCAGTCTC |
| | | CCCTGGAGGCGTGGGTTCGAATCCCACTTCTGACA |
| 146 | Leu_TAA_chr6: 144537684-144537766 (+) | ACCAGGATGGCCGAGTGGTTAAGGCGTTGGACTTAAGATCCAATGGAC |
| | | ATATGTCCGCGTGGGTTCGAACCCCACTCCTGGTA |
| 147 | Leu_TAA_chr6: 27688898-27688980 (−) | ACCGGGATGGCCGAGTGGTTAAGGCGTTGGACTTAAGATCCAATGGGC |
| | | TGGTGCCCGCGTGGGTTCGAACCCCACTCTCGGTA |
| 148 | Leu_TAA_chr11: 59319228-59319310 (+) | ACCAGAATGGCCGAGTGGTTAAGGCGTTGGACTTAAGATCCAATGGAT |
| | | TCATATCCGCGTGGGTTCGAACCCCACTTCTGGTA |
| 149 | Leu_TAA_chr6: 27198334-27198416 (−) | ACCGGGATGGCTGAGTGGTTAAGGCGTTGGACTTAAGATCCAATGGAC |
| | | AGGTGTCCGCGTGGGTTCGAGCCCCACTCCCGGTA |
| 150 | Leu_TAG_chr17: 8023632-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCA |
| 151 | Leu_TAG_chr14: 21093529-21093610 (+) | GGTAGTGTGGCCGAGCGGTCTAAGGCGCTGGATTTAGGCTCCAGTCTC |
| | | TTCGGGGGCGTGGGTTCGAATCCCACCACTGCCA |
| 152 | Leu_TAG_chr16: 22207032-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCA |
| 153 | Lys_CTT_chr14: 58706613-58706685 (−) | GCCCGGCTAGCTCAGTCGGTAGAGCATGGGACTCTTAATCCCAGGGTC |
| | | GTGGGTTCGAGCCCCACGTTGGGCG |
| 154 | Lys_CTT_chr19: 36066750-36066822 (+) | GCCCAGCTAGCTCAGTCGGTAGAGCATAAGACTCTTAATCTCAGGGTT |
| | | GTGGATTCGTGCCCCATGCTGGGTG |
| 155 | Lys_CTT_chr19: 52425393-52425466 (−) | GCAGCTAGCTCAGTCGGTAGAGCATGAGACTCTTAATCTCAGGGTCAT |
| | | GGGTTCGTGCCCCATGTTGGGTGCCA |
| 156 | Lys_CTT_chr1: 145395522-145395594 (−) | GCCCGGCTAGCTCAGTCGGTAGAGCATGAGACTCTTAATCTCAGGGTC |
| | | GTGGGTTCGAGCCCCACGTTGGGCG |
| 157 | Lys_CTT_chr16: 3207406-3207478 (−) | GCCCGGCTAGCTCAGTCGGTAGAGCATGAGACCCTTAATCTCAGGGTC |
| | | GTGGGTTCGAGCCCCACGTTGGGCG |
| 158 | Lys_CTT_chr16: 3241501-3241573 (+) | GCCCGGCTAGCTCAGTCGGTAGAGCATGGGACTCTTAATCTCAGGGTC |
| | | GTGGGTTCGAGCCCCACGTTGGGCG |
| 159 | Lys_CTT_chr16: 3230555-3230627 (−) | GCCCGGCTAGCTCAGTCGATAGAGCATGAGACTCTTAATCTCAGGGTC |
| | | GTGGGTTCGAGCCGCACGTTGGGCG |
| 160 | Lys_CTT_chr1: 55423542-55423614 (−) | GCCCAGCTAGCTCAGTCGGTAGAGCATGAGACTCTTAATCTCAGGGTC |
| | | ATGGGTTTGAGCCCCACGTTTGGTG |
| 161 | Lys_CTT_chr16: 3214939-3215011 (+) | GCCTGGCTAGCTCAGTCGGCAAAGCATGAGACTCTTAATCTCAGGGTC |
| | | GTGGGCTCGAGCTCCATGTTGGGCG |
| 162 | Lys_CTT_chr5: 26198539-26198611 (−) | GCCCGACTACCTCAGTCGGTGGAGCATGGGACTCTTCATCCCAGGGTT |
| | | GTGGGTTCGAGCCCCACATTGGGCA |
| 163 | Lys_TTT_chr16: 73512216-73512288 (−) | GCCTGGATAGCTCAGTTGGTAGAGCATCAGACTTTTAATCTGAGGGTC |
| | | CAGGGTTCAAGTCCCTGTTCAGGCA |
| 164 | Lys_TTT_chr12: 27843306-27843378 (+) | ACCCAGATAGCTCAGTCAGTAGAGCATCAGACTTTTAATCTGAGGGTC |
| | | CAAGGTTCATGTCCCTTTTTGGGTG |
| 165 | Lys_TTT_chr11: 122430655-122430727 (+) | GCCTGGATAGCTCAGTTGGTAGAGCATCAGACTTTTAATCTGAGGGTC |
| | | CAGGGTTCAAGTCCCTGTTCAGGCG |
| 166 | Lys_TTT_chr1: 204475655-204475727 (+) | GCCCGGATAGCTCAGTCGGTAGAGCATCAGACTTTTAATCTGAGGGTC |
| | | CAGGGTTCAAGTCCCTGTTCGGGCG |
| 167 | Lys_TTT_chr6: 27559593-27559665 (−) | GCCTGGATAGCTCAGTCGGTAGAGCATCAGACTTTTAATCTGAGGGTC |
| | | CAGGGTTCAAGTCCCTGTTCAGGCG |
| 168 | Lys_TTT_chr11: 59323902-59323974 (+) | GCCCGGATAGCTCAGTCGGTAGAGCATCAGACTTTTAATCTGAGGGTC |
| | | CGGGGTTCAAGTCCCTGTTCGGGCG |
| 169 | Lys_TTT_chr6: 27302769-27302841 (−) | GCCTGGGTAGCTCAGTCGGTAGAGCATCAGACTTTTAATCTGAGGGTC |
| | | CAGGGTTCAAGTCCCTGTCCAGGCG |
| 170 | Lys_TTT_chr6: 28715521-28715593 (+) | GCCTGGATAGCTCAGTTGGTAGAACATCAGACTTTTAATCTGACGGTG |
| | | CAGGGTTCAAGTCCCTGTTCAGGCG |
| 171 | Met_CAT_chr8: 124169470-124169542 (−) | GCCTCGTTAGCGCAGTAGGTAGCGCGTCAGTCTCATAATCTGAAGGTC |
| | | GTGAGTTCGATCCTCACACGGGGCA |
| 172 | Met_CAT_chr16: 71460396-71460468 (+) | GCCCTCTTAGCGCAGTGGGCAGCGCGTCAGTCTCATAATCTGAAGGTC |
| | | CTGAGTTCGAGCCTCAGAGAGGGCA |
| 173 | Met_CAT_chr6: 28912352-28912424 (+) | GCCTCCTTAGCGCAGTAGGCAGCGCGTCAGTCTCATAATCTGAAGGTC |
| | | CTGAGTTCGAACCTCAGAGGGGGCA |
| 174 | Met_CAT_chr6: 26735574-26735646 (−) | GCCCTCTTAGCGCAGCGGGCAGCGCGTCAGTCTCATAATCTGAAGGTC |
| | | CTGAGTTCGAGCCTCAGAGAGGGCA |
| 175 | Met_CAT_chr6: 26701712-26701784 (+) | GCCCTCTTAGCGCAGCTGGCAGCGCGTCAGTCTCATAATCTGAAGGTC |
| | | CTGAGTTCAAGCCTCAGAGAGGGCA |
| 176 | Met_CAT_chr16: 87417628-87417700 (−) | GCCTCGTTAGCGCAGTAGGCAGCGCGTCAGTCTCATAATCTGAAGGTC |
| | | GTGAGTTCGAGCCTCACACGGGGCA |
| 177 | Met_CAT_chr6: 58168492-58168564 (−) | GCCCTCTTAGTGCAGCTGGCAGCGCGTCAGTTTCATAATCTGAAAGTCC |
| | | TGAGTTCAAGCCTCAGAGAGGGCA |
| 178 | Phe_GAA_chr6: 28758499-28758571 (−) | GCCGAAATAGCTCAGTTGGGAGAGCGTTAGACTGAAGATCTAAAGGTC |
| | | CCTGGTTCGATCCCGGGTTTCGGCA |
| 179 | Phe_GAA_chr11: 59333853-59333925 (−) | GCCGAAATAGCTCAGTTGGGAGAGCGTTAGACTGAAGATCTAAAGGTC |
| | | CCTGGTTCAATCCCGGGTTTCGGCA |
| 180 | Phe_GAA_chr6: 28775610-28775682 (−) | GCCGAGATAGCTCAGTTGGGAGAGCGTTAGACTGAAGATCTAAAGGTC |
| | | CCTGGTTCAATCCCGGGTTTCGGCA |
| 181 | Phe_GAA_chr6: 28791093-28791166 (−) | GCCGAAATAGCTCAGTTGGGAGAGCGTTAGACCGAAGATCTTAAAGGT |
| | | CCCTGGTTCAATCCCGGGTTTCGGCA |
| 182 | Phe_GAA_chr6: 28731374-28731447 (−) | GCTGAAATAGCTCAGTTGGGAGAGCGTTAGACTGAAGATCTTAAAGTT |
| | | CCCTGGTTCAACCCTGGGTTTCAGCC |
| 183 | Pro_AGG_chr16: 3241989-3242060 (+) | GGCTCGTTGGTCTAGGGGTATGATTCTCGCTTAGGATGCGAGAGGTCC |
| | | CGGGTTCAAATCCCGGACGAGCCC |
| 184 | Pro_AGG_chr1: 167684725-167684796 (−) | GGCTCGTTGGTCTAGGGGTATGATTCTCGCTTAGGGTGCGAGAGGTCC |
| | | CGGGTTCAAATCCCGGACGAGCCC |
| 185 | Pro_CGG_chr1: 167683962-167684033 (+) | GGCTCGTTGGTCTAGGGGTATGATTCTCGCTTCGGGTGCGAGAGGTCC |
| | | CGGGTTCAAATCCCGGACGAGCCC |
| 186 | Pro_CGG_chr6: 27059521-27059592 (+) | GGCTCGTTGGTCTAGGGGTATGATTCTCGCTTCGGGTGTGAGAGGTCCC |
| | | GGGTTCAAATCCCGGACGAGCCC |
| 187 | Pro_TGG_chr14: 21101165-21101236 (+) | GGCTCGTTGGTCTAGTGGTATGATTCTCGCTTTGGGTGCGAGAGGTCCC |
| | | GGGTTCAAATCCCGGACGAGCCC |
| 188 | Pro_TGG_chr11: 75946869-75946940 (−) | GGCTCGTTGGTCTAGGGGTATGATTCTCGGTTTGGGTCCGAGAGGTCCC |
| | | GGGTTCAAATCCCGGACGAGCCC |
| 189 | Pro_TGG_chr5: 180615854-180615925 (−) | GGCTCGTTGGTCTAGGGGTATGATTCTCGCTTTGGGTGCGAGAGGTCCC |
| | | GGGTTCAAATCCCGGACGAGCCC |
| 190 | SeC_TCA_chr19: 45981859-45981945 (−) | GCCCGGATGATCCTCAGTGGTCTGGGGTGCAGGCTTCAAACCTGTAGC |
| | | TGTCTAGCGACAGAGTGGTTCAATTCCACCTTTCGGGCG |
| 191 | SeC_TCA_chr22: 44546537-44546620 (+) | GCTCGGATGATCCTCAGTGGTCTGGGGTGCAGGCTTCAAACCTGTAGC |
| | | TGTCTAGTGACAGAGTGGTTCAATTCCACCTTTGTA |
| 192 | Ser_AGA_chr6: 27509554-27509635 (−) | GTAGTCGTGGCCGAGTGGTTAAGGCGATGGACTAGAAATCCATTGGGG |
| | | TTTCCCCGCGCAGGTTCGAATCCTGCCGACTACG |
| 193 | Ser_AGA_chr6: 26327817-26327898 (+) | GTAGTCGTGGCCGAGTGGTTAAGGCGATGGACTAGAAATCCATTGGGG |
| | | TCTCCCCGCGCAGGTTCGAATCCTGCCGACTACG |
| 194 | Ser_AGA_chr6: 27499987-27500068 (+) | GTAGTCGTGGCCGAGTGGTTAAGGCGATGGACTAGAAATCCATTGGGG |
| | | TTTCCCCACGCAGGTTCGAATCCTGCCGACTACG |
| 195 | Ser_AGA_chr6: 27521192-27521273 (−) | GTAGTCGTGGCCGAGTGGTTAAGGTGATGGACTAGAAACCCATTGGGG |
| | | TCTCCCCGCGCAGGTTCGAATCCTGCCGACTACG |
| 196 | Ser_CGA_chr17: 8042199-8042280 (−) | GCTGTGATGGCCGAGTGGTTAAGGCGTTGGACTCGAAATCCAATGGGG |
| | | TCTCCCCGCGCAGGTTCGAATCCTGCTCACAGCG |
| 197 | Ser_CGA_chr6: 27177628-27177709 (+) | GCTGTGATGGCCGAGTGGTTAAGGCGTTGGACTCGAAATCCAATGGGG |
| | | TCTCCCCGCGCAGGTTCAAATCCTGCTCACAGCG |
| 198 | Ser_CGA_chr6: 27640229-27640310 (−) | GCTGTGATGGCCGAGTGGTTAAGGTGTTGGACTCGAAATCCAATGGGG |
| | | GTTCCCCGCGCAGGTTCAAATCCTGCTCACAGCG |
| 199 | Ser_CGA_chr12: 56584148-56584229 (+) | GTCACGGTGGCCGAGTGGTTAAGGCGTTGGACTCGAAATCCAATGGGG |
| | | TTTCCCCGCACAGGTTCGAATCCTGTTCGTGACG |
| 200 | Ser_GCT_chr6: 27065085-27065166 (+) | GACGAGGTGGCCGAGTGGTTAAGGCGATGGACTGCTAATCCATTGTGC |
| | | TCTGCACGCGTGGGTTCGAATCCCACCCTCGTCG |
| 201 | Ser_GCT_chr6: 27265775-27265856 (+) | GACGAGGTGGCCGAGTGGTTAAGGCGATGGACTGCTAATCCATTGTGC |
| | | TCTGCACGCGTGGGTTCGAATCCCACCTTCGTCG |
| 202 | Ser_GCT_chr11: 66115591-66115672 (+) | GACGAGGTGGCCGAGTGGTTAAGGCGATGGACTGCTAATCCATTGTGC |
| | | TTTGCACGCGTGGGTTCGAATCCCATCCTCGTCG |
| 203 | Ser_GCT_chr6: 28565117-28565198 (−) | GACGAGGTGGCCGAGTGGTTAAGGCGATGGACTGCTAATCCATTGTGC |
| | | TCTGCACGCGTGGGTTCGAATCCCATCCTCGTCG |
| 204 | Ser_GCT_chr6: 28180815-28180896 (+) | GACGAGGTGGCCGAGTGGTTAAGGCGATGGACTGCTAATCCATTGTGC |
| | | TCTGCACACGTGGGTTCGAATCCCATCCTCGTCG |
| 205 | Ser_GCT_chr6: 26305718-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCG |
| 206 | Ser_TGA_chr10: 69524261-69524342 (+) | GCAGCGATGGCCGAGTGGTTAAGGCGTTGGACTTGAAATCCAATGGGG |
| | | TCTCCCCGCGCAGGTTCGAACCCTGCTCGCTGCG |
| 207 | Ser_TGA_chr6: 27513468-27513549 (+) | GTAGTCGTGGCCGAGTGGTTAAGGCGATGGACTTGAAATCCATTGGGG |
| | | TTTCCCCGCGCAGGTTCGAATCCTGCCGACTACG |
| 208 | Ser_TGA_chr6: 26312824-26312905 (−) | GTAGTCGTGGCCGAGTGGTTAAGGCGATGGACTTGAAATCCATTGGGG |
| | | TCTCCCCGCGCAGGTTCGAATCCTGCCGACTACG |
| 209 | Ser_TGA_chr6: 27473607-27473688 (−) | GTAGTCGTGGCCGAGTGGTTAAGGCGATGGACTTGAAATCCATTGGGG |
| | | TTTCCCCGCGCAGGTTCGAATCCTGTCGGCTACG |
| 210 | Thr_AGT_chr17: 8090478-8090551 (+) | GGCGCCGTGGCTTAGTTGGTTAAAGCGCCTGTCTAGTAAACAGGAGAT |
| | | CCTGGGTTCGAATCCCAGCGGTGCCT |
| 211 | Thr_AGT_chr6: 26533145-26533218 (−) | GGCTCCGTGGCTTAGCTGGTTAAAGCGCCTGTCTAGTAAACAGGAGAT |
| | | CCTGGGTTCGAATCCCAGCGGGGCCT |
| 212 | Thr_AGT_chr6: 28693795-28693868 (+) | GGCTCCGTAGCTTAGTTGGTTAAAGCGCCTGTCTAGTAAACAGGAGAT |
| | | CCTGGGTTCGACTCCCAGCGGGGCCT |
| 213 | Thr_AGT_chr6: 27694473-27694546 (+) | GGCTTCGTGGCTTAGCTGGTTAAAGCGCCTGTCTAGTAAACAGGAGAT |
| | | CCTGGGTTCGAATCCCAGCGAGGCCT |
| 214 | Thr_AGT_chr17: 8042770-8042843 (−) | GGCGCCGTGGCTTAGCTGGTTAAAGCGCCTGTCTAGTAAACAGGAGAT |
| | | CCTGGGTTCGAATCCCAGCGGTGCCT |
| 215 | Thr_AGT_chr6: 27130050-27130123 (+) | GGCCCTGTGGCTTAGCTGGTCAAAGCGCCTGTCTAGTAAACAGGAGAT |
| | | CCTGGGTTCGAATCCCAGCGGGGCCT |
| 216 | Thr_CGT_chr6: 28456770-28456843 (−) | GGCTCTATGGCTTAGTTGGTTAAAGCGCCTGTCTCGTAAACAGGAGAT |
| | | CCTGGGTTCGACTCCCAGTGGGGCCT |
| 217 | Thr_CGT_chr16: 14379750-14379821 (+) | GGCGCGGTGGCCAAGTGGTAAGGCGTCGGTCTCGTAAACCGAAGATCA |
| | | CGGGTTCGAACCCCGTCCGTGCCT |
| 218 | Thr_CGT_chr6: 28615984-28616057 (−) | GGCTCTGTGGCTTAGTTGGCTAAAGCGCCTGTCTCGTAAACAGGAGAT |
| | | CCTGGGTTCGAATCCCAGCGGGGCCT |
| 219 | Thr_CGT_chr17: 29877093-29877164 (+) | GGCGCGGTGGCCAAGTGGTAAGGCGTCGGTCTCGTAAACCGAAGATCG |
| | | CGGGTTCGAACCCCGTCCGTGCCT |
| 220 | Thr_CGT_chr6: 27586135-27586208 (+) | GGCCCTGTAGCTCAGCGGTTGGAGCGCTGGTCTCGTAAACCTAGGGGT |
| | | CGTGAGTTCAAATCTCACCAGGGCCT |
| 221 | Thr_TGT_chr6: 28442329-28442402 (−) | GGCTCTATGGCTTAGTTGGTTAAAGCGCCTGTCTTGTAAACAGGAGAT |
| | | CCTGGGTTCGAATCCCAGTAGAGCCT |
| 222 | Thr_TGT_chr1: 222638347-222638419 (+) | GGCTCCATAGCTCAGTGGTTAGAGCACTGGTCTTGTAAACCAGGGGTC |
| | | GCGAGTTCGATCCTCGCTGGGGCCT |
| 223 | Thr_TGT_chr14: 21081949-21082021 (−) | GGCTCCATAGCTCAGGGGTTAGAGCGCTGGTCTTGTAAACCAGGGGTC |
| | | GCGAGTTCAATTCTCGCTGGGGCCT |
| 224 | Thr_TGT_chr14: 21099319-21099391 (−) | GGCTCCATAGCTCAGGGGTTAGAGCACTGGTCTTGTAAACCAGGGGTC |
| | | GCGAGTTCAAATCTCGCTGGGGCCT |
| 225 | Thr_TGT_chr14: 21149849-21149921 (+) | GGCCCTATAGCTCAGGGGTTAGAGCACTGGTCTTGTAAACCAGGGGTC |
| | | GCGAGTTCAAATCTCGCTGGGGCCT |
| 226 | Thr_TGT_chr5: 180618687-180618758 (−) | GGCTCCATAGCTCAGGGGTTAGAGCACTGGTCTTGTAAACCAGGGTCG |
| | | CGAGTTCAAATCTCGCTGGGGCCT |
| 227 | Trp_CCA_chr17: 8124187-8124258 (−) | GGCCTCGTGGCGCAACGGTAGCGCGTCTGACTCCAGATCAGAAGGTTG |
| | | CGTGTTCAAATCACGTCGGGGTCA |
| 228 | Trp_CCA_chr17: 19411494-19411565 (+) | GACCTCGTGGCGCAATGGTAGCGCGTCTGACTCCAGATCAGAAGGTTG |
| | | CGTGTTCAAGTCACGTCGGGGTCA |
| 229 | Trp_CCA_chr6: 26319330-26319401 (−) | GACCTCGTGGCGCAACGGTAGCGCGTCTGACTCCAGATCAGAAGGTTG |
| | | CGTGTTCAAATCACGTCGGGGTCA |
| 230 | Trp_CCA_chr12: 98898030-98898101 (+) | GACCTCGTGGCGCAACGGTAGCGCGTCTGACTCCAGATCAGAAGGCTG |
| | | CGTGTTCGAATCACGTCGGGGTCA |
| 231 | Trp_CCA_chr7: 99067307-99067378 (+) | GACCTCGTGGCGCAACGGCAGCGCGTCTGACTCCAGATCAGAAGGTTG |
| | | CGTGTTCAAATCACGTCGGGGTCA |
| 232 | Tyr_ATA_chr2: 219110549-219110641 (+) | CCTTCAATAGTTCAGCTGGTAGAGCAGAGGACTATAGCTACTTCCTCA |
| | | GTAGGAGACGTCCTTAGGTTGCTGGTTCGATTCCAGCTTGAAGGA |
| 233 | Tyr_GTA_chr6: 26569086-26569176 (+) | CCTTCGATAGCTCAGTTGGTAGAGCGGAGGACTGTAGTTGGCTGTGTC |
| | | CTTAGACATCCTTAGGTCGCTGGTTCGAATCCGGCTCGAAGGA |
| 234 | Tyr_GTA_chr2: 27273650-27273738 (+) | CCTTCGATAGCTCAGTTGGTAGAGCGGAGGACTGTAGTGGATAGGGCG |
| | | TGGCAATCCTTAGGTCGCTGGTTCGATTCCGGCTCGAAGGA |
| 235 | Tyr_GTA_chr6: 26577332-26577420 (+) | CCTTCGATAGCTCAGTTGGTAGAGCGGAGGACTGTAGGCTCATTAAGC |
| | | AAGGTATCCTTAGGTCGCTGGTTCGAATCCGGCTCGGAGGA |
| 236 | Tyr_GTA_chr14: 21125623-21125716 (−) | CCTTCGATAGCTCAGCTGGTAGAGCGGAGGACTGTAGATTGTATAGAC |
| | | ATTTGCGGACATCCTTAGGTCGCTGGTTCGATTCCAGCTCGAAGGA |
| 237 | Tyr_GTA_chr8: 67025602-67025694 (+) | CCTTCGATAGCTCAGCTGGTAGAGCGGAGGACTGTAGCTACTTCCTCA |
| | | GCAGGAGACATCCTTAGGTCGCTGGTTCGATTCCGGCTCGAAGGA |
| 238 | Tyr_GTA_chr8: 67026223-67026311 (+) | CCTTCGATAGCTCAGCTGGTAGAGCGGAGGACTGTAGGCGCGCGCCCG |
| | | TGGCCATCCTTAGGTCGCTGGTTCGATTCCGGCTCGAAGGA |
| 239 | Tyr_GTA_chr14: 21121258-21121351 (−) | CCTTCGATAGCTCAGCTGGTAGAGCGGAGGACTGTAGCCTGTAGAAAC |
| | | ATTTGTGGACATCCTTAGGTCGCTGGTTCGATTCCGGCTCGAAGGA |
| 240 | Tyr_GTA_chr14: 21131351-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGA |
| 241 | Tyr_GTA_chr14: 21151432-21151520 (+) | CCTTCGATAGCTCAGCTGGTAGAGCGGAGGACTGTAGTACTTAATGTG |
| | | TGGTCATCCTTAGGTCGCTGGTTCGATTCCGGCTCGAAGGA |
| 242 | Tyr_GTA_chr6: 26595102-26595190 (+) | CCTTCGATAGCTCAGCTGGTAGAGCGGAGGACTGTAGGGGTTTGAATG |
| | | TGGTCATCCTTAGGTCGCTGGTTCGAATCCGGCTCGGAGGA |
| 243 | Tyr_GTA_chr14: 21128117-21128210 (−) | CCTTCGATAGCTCAGCTGGTAGAGCGGAGGACTGTAGACTGCGGAAAC |
| | | GTTTGTGGACATCCTTAGGTCGCTGGTTCAATTCCGGCTCGAAGGA |
| 244 | Tyr_GTA_chr6: 26575798-26575887 (+) | CTTTCGATAGCTCAGTTGGTAGAGCGGAGGACTGTAGGTTCATTAAAC |
| | | TAAGGCATCCTTAGGTCGCTGGTTCGAATCCGGCTCGAAGGA |
| 245 | Tyr_GTA_chr8: 66609532-66609619 (−) | TCTTCAATAGCTCAGCTGGTAGAGCGGAGGACTGTAGGTGCACGCCCG |
| | | TGGCCATTCTTAGGTGCTGGTTTGATTCCGACTTGGAGAG |
| 246 | Val_AAC_chr3: 169490018-169490090 (+) | GTTTCCGTAGTGTAGTGGTTATCACGTTCGCCTAACACGCGAAAGGTCC |
| | | CCGGTTCGAAACCGGGCGGAAACA |
| 247 | Val_AAC_chr5: 180615416-180615488 (−) | GTTTCCGTAGTGTAGTGGTCATCACGTTCGCCTAACACGCGAAAGGTC |
| | | CCCGGTTCGAAACCGGGCGGAAACA |
| 248 | Val_AAC_chr6: 27618707-27618779 (−) | GTTTCCGTAGTGTAGTGGTTATCACGTTCGCCTAACACGCGAAAGGTCC |
| | | CTGGATCAAAACCAGGCGGAAACA |
| 249 | Val_AAC_chr6: 27648885-27648957 (−) | GTTTCCGTAGTGTAGTGGTTATCACGTTCGCCTAACACGCGAAAGGTCC |
| | | GCGGTTCGAAACCGGGCGGAAACA |
| 250 | Val_AAC_chr6: 27203288-27203360 (+) | GTTTCCGTAGTGTAGTGGTTATCACGTTTGCCTAACACGCGAAAGGTCC |
| | | CCGGTTCGAAACCGGGCAGAAACA |
| 251 | Val_AAC_chr6: 28703206-28703277 (−) | GGGGGTGTAGCTCAGTGGTAGAGCGTATGCTTAACATTCATGAGGCTC |
| | | TGGGTTCGATCCCCAGCACTTCCA |
| 252 | Val_CAC_chr1: 161369490-161369562 (−) | GTTTCCGTAGTGTAGTGGTTATCACGTTCGCCTCACACGCGAAAGGTCC |
| | | CCGGTTCGAAACCGGGCGGAAACA |
| 253 | Val_CAC_chr6: 27248049-27248121 (−) | GCTTCTGTAGTGTAGTGGTTATCACGTTCGCCTCACACGCGAAAGGTCC |
| | | CCGGTTCGAAACCGGGCAGAAGCA |
| 254 | Val_CAC_chr19: 4724647-4724719 (−) | GTTTCCGTAGTGTAGCGGTTATCACATTCGCCTCACACGCGAAAGGTCC |
| | | CCGGTTCGATCCCGGGCGGAAACA |
| 255 | Val_CAC_chr1: 149298555-149298627 (−) | GTTTCCGTAGTGTAGTGGTTATCACGTTCGCCTCACACGCGAAAGGTCC |
| | | CCGGTTCGAAACTGGGCGGAAACA |
| 256 | Val_CAC_chr1: 149684088-149684161 (−) | GTTTCCGTAGTGTAGTGGTTATCACGTTCGCCTCACACGCGTAAAGGTC |
| | | CCCGGTTCGAAACCGGGCGGAAACA |
| 257 | Val_CAC_chr6: 27173867-27173939 (−) | GTTTCCGTAGTGGAGTGGTTATCACGTTCGCCTCACACGCGAAAGGTC |
| | | CCCGGTTTGAAACCAGGCGGAAACA |
| 258 | Val_TAC_chr11: 59318102-59318174 (−) | GGTTCCATAGTGTAGTGGTTATCACGTCTGCTTTACACGCAGAAGGTCC |
| | | TGGGTTCGAGCCCCAGTGGAACCA |
| 259 | Val_TAC_chr11: 59318460-59318532 (−) | GGTTCCATAGTGTAGCGGTTATCACGTCTGCTTTACACGCAGAAGGTCC |
| | | TGGGTTCGAGCCCCAGTGGAACCA |
| 260 | Val_TAC_chr10: 5895674-5895746 (−) | GGTTCCATAGTGTAGTGGTTATCACATCTGCTTTACACGCAGAAGGTCC |
| | | TGGGTTCAAGCCCCAGTGGAACCA |
| 261 | Val_TAC_chr6: 27258405-27258477 (+) | GTTTCCGTGGTGTAGTGGTTATCACATTCGCCTTACACGCGAAAGGTCC |
| | | TCGGGTCGAAACCGAGCGGAAACA |
| 262 | iMet_CAT_chr1: 153643726-153643797 (+) | AGCAGAGTGGCGCAGCGGAAGCGTGCTGGGCCCATAACCCAGAGGTC |
| | | GATGGATCGAAACCATCCTCTGCTA |
| 263 | iMet_CAT_chr6: 27745664-27745735 (+) | AGCAGAGTGGCGCAGCGGAAGCGTGCTGGGCCCATAACCCAGAGGTC |
| | | GATGGATCTAAACCATCCTCTGCTA |
| 264 | Glu_TTC_chr1: 16861773-16861845 (−) | TCCCTGGTGGTCTAGTGGCTAGGATTCGGCGCTTTCACCGCCGCGGCCC |
| | | GGGTTCGATTCCCGGTCAGGGAAT |
| 265 | Gly_CCC_chr1: 17004765-17004836 (−) | GCGTTGGTGGTTTAGTGGTAGAATTCTCGCCTCCCATGCGGGAGACCC |
| | | GGGTTCAATTCCCGGCCACTGCAC |
| 266 | Gly_CCC_chr1: 17053779-17053850 (+) | GGCCTTGGTGGTGCAGTGGTAGAATTCTCGCCTCCCACGTGGGAGACC |
| | | CGGGTTCAATTCCCGGCCAATGCA |
| 267 | Glu_TTC_chr1: 17199077-17199149 (+) | GTCCCTGGTGGTCTAGTGGCTAGGATTCGGCGCTTTCACCGCCGCGGCC |
| | | CGGGTTCGATTCCCGGCCAGGGAA |
| 268 | Asn_GTT_chr1: 17216171-17216245 (+) | TGTCTCTGTGGCGCAATCGGTTAGCGCGTTCGGCTGTTAACCGAAAGA |
| | | TTGGTGGTTCGAGCCCACCCAGGGACG |
| 269 | Arg_TCT_chr1: 94313128-94313213 (+) | TGGCTCCGTGGCGCAATGGATAGCGCATTGGACTTCTAGAGGCTGAAG |
| | | GCATTCAAAGGTTCCGGGTTCGAGTCCCGGCGGAGTCG |
| 270 | Lys_CTT_chr1: 145395521-145395594 (−) | GCCCGGCTAGCTCAGTCGGTAGAGCATGAGACTCTTAATCTCAGGGTC |
| | | GTGGGTTCGAGCCCCACGTTGGGCGC |
| 271 | His_GTG_chr1: 145396880-145396952 (−) | GCCGTGATCGTATAGTGGTTAGTACTCTGCGTTGTGGCCGCAGCAACCT |
| | | CGGTTCGAATCCGAGTCACGGCAG |
| 272 | Gly_TCC_chr1: 145397863-145397935 (−) | GCGTTGGTGGTATAGTGGTGAGCATAGCTGCCTTCCAAGCAGTTGACC |
| | | CGGGTTCGATTCCCGGCCAACGCAG |
| 273 | Glu_CTC_chr1: 145399232-145399304 (−) | TCCCTGGTGGTCTAGTGGTTAGGATTCGGCGCTCTCACCGCCGCGGCCC |
| | | GGGTTCGATTCCCGGTCAGGGAAA |
| 274 | Gln_CTG_chr1: 145963303-145963375 (+) | AGGTTCCATGGTGTAATGGTGAGCACTCTGGACTCTGAATCCAGCGAT |
| | | CCGAGTTCGAGTCTCGGTGGAACCT |
| 275 | Asn_GTT_chr1: 148000804-148000878 (+) | TGTCTCTGTGGCGTAGTCGGTTAGCGCGTTCGGCTGTTAACCGAAAAGT |
| | | TGGTGGTTCGAGCCCACCCAGGAACG |
| 276 | Asn_GTT_chr1: 148248114-148248188 (+) | TGTCTCTGTGGCGCAATCGGTTAGCGCGTTCGGCTGTTAACCGAAAGG |
| | | TTGGTGGTTCGAGCCCACCCAGGGACG |
| 277 | Asn_GTT_chr1: 148598313-148598387 (−) | GTCTCTGTGGCGCAATCGGTTAGCGCATTCGGCTGTTAACCGAAAGGT |
| | | TGGTGGTTCGAGCCCACCCAGGGACGC |
| 278 | Asn_GTT_chr1: 149230569-149230643 (−) | GTCTCTGTGGCGCAATGGGTTAGCGCGTTCGGCTGTTAACCGAAAGGT |
| | | TGGTGGTTCGAGCCCATCCAGGGACGC |
| 279 | Val_CAC_chr1: 149294665-149294736 (−) | GCACTGGTGGTTCAGTGGTAGAATTCTCGCCTCACACGCGGGACACCC |
| | | GGGTTCAATTCCCGGTCAAGGCAA |
| 280 | Val_CAC_chr1: 149298554-149298627 (−) | GTTTCCGTAGTGTAGTGGTTATCACGTTCGCCTCACACGCGAAAGGTCC |
| | | CCGGTTCGAAACTGGGCGGAAACAG |
| 281 | Gly_CCC_chr1: 149680209-149680280 (−) | GCACTGGTGGTTCAGTGGTAGAATTCTCGCCTCCCACGCGGGAGACCC |
| | | GGGTTTAATTCCCGGTCAAGATAA |
| 282 | Val_CAC_chr1: 149684087-149684161 (−) | GTTTCCGTAGTGTAGTGGTTATCACGTTCGCCTCACACGCGTAAAGGTC |
| | | CCCGGTTCGAAACCGGGCGGAAACAT |
| 283 | Met_CAT_chr1: 153643725-153643797 (+) | TAGCAGAGTGGCGCAGCGGAAGCGTGCTGGGCCCATAACCCAGAGGT |
| | | CGATGGATCGAAACCATCCTCTGCTA |
| 284 | Val_CAC_chr1: 161369489-161369562 (−) | GTTTCCGTAGTGTAGTGGTTATCACGTTCGCCTCACACGCGAAAGGTCC |
| | | CCGGTTCGAAACCGGGCGGAAACAA |
| 285 | Asp_GTC_chr1: 161410614-161410686 (−) | TCCTCGTTAGTATAGTGGTGAGTATCCCCGCCTGTCACGCGGGAGACC |
| | | GGGGTTCGATTCCCCGACGGGGAGG |
| 286 | Gly_GCC_chr1: 161413093-161413164 (+) | TGCATGGGTGGTTCAGTGGTAGAATTCTCGCCTGCCACGCGGGAGGCC |
| | | CGGGTTCGATTCCCGGCCCATGCA |
| 287 | Glu_CTC_chr1: 161417017-161417089 (−) | TCCCTGGTGGTCTAGTGGTTAGGATTCGGCGCTCTCACCGCCGCGGCCC |
| | | GGGTTCGATTCCCGGTCAGGGAAG |
| 288 | Asp_GTC_chr1: 161492934-161493006 (+) | ATCCTTGTTACTATAGTGGTGAGTATCTCTGCCTGTCATGCGTGAGAGA |
| | | GGGGGTCGATTCCCCGACGGGGAG |
| 289 | Gly_GCC_chr1: 161493636-161493707 (−) | GCATTGGTGGTTCAGTGGTAGAATTCTCGCCTGCCACGCGGGAGGCCC |
| | | GGGTTCGATTCCCGGCCAATGCAC |
| 290 | Leu_CAG_chr1: 161500131-161500214 (−) | GTCAGGATGGCCGAGCGGTCTAAGGCGCTGCGTTCAGGTCGCAGTCTC |
| | | CCCTGGAGGCGTGGGTTCGAATCCCACTCCTGACAA |
| 291 | Gly_TCC_chr1: 161500902-161500974 (+) | CGCGTTGGTGGTATAGTGGTGAGCATAGCTGCCTTCCAAGCAGTTGAC |
| | | CCGGGTTCGATTCCCGGCCAACGCA |
| 292 | Asn_GTT_chr1: 161510030-161510104 (+) | CGTCTCTGTGGCGCAATCGGTTAGCGCGTTCGGCTGTTAACCGAAAGG |
| | | TTGGTGGTTCGATCCCACCCAGGGACG |
| 293 | Glu_TTC_chr1: 161582507-161582579 (+) | CGCGTTGGTGGTGTAGTGGTGAGCACAGCTGCCTTTCAAGCAGTTAAC |
| | | GCGGGTTCGATTCCCGGGTAACGAA |
| 294 | Pro_CGG_chr1: 167683961-167684033 (+) | CGGCTCGTTGGTCTAGGGGTATGATTCTCGCTTCGGGTGCGAGAGGTC |
| | | CCGGGTTCAAATCCCGGACGAGCCC |
| 295 | Pro_AGG_chr1: 167684724-167684796 (−) | GGCTCGTTGGTCTAGGGGTATGATTCTCGCTTAGGGTGCGAGAGGTCC |
| | | CGGGTTCAAATCCCGGACGAGCCCT |
| 296 | Lys_TTT_chr1: 204475654-204475727 (+) | CGCCCGGATAGCTCAGTCGGTAGAGCATCAGACTTTTAATCTGAGGGT |
| | | CCAGGGTTCAAGTCCCTGTTCGGGCG |
| 297 | Lys_TTT_chr1: 204476157-204476230 (−) | GCCCGGATAGCTCAGTCGGTAGAGCATCAGACTTTTAATCTGAGGGTC |
| | | CAGGGTTCAAGTCCCTGTTCGGGCGT |
| 298 | Leu_CAA_chr1: 249168053-249168159 (+) | TGTCAGGATGGCCGAGTGGTCTAAGGCGCCAGACTCAAGGTAAGCACC |
| | | TTGCCTGCGGGCTTTCTGGTCTCCGGATGGAGGCGTGGGTTCGAATCCC |
| 299 | Glu_CTC_chr1: 249168446-249168518 (+) | TTCCCTGGTGGTCTAGTGGTTAGGATTCGGCGCTCTCACCGCCGCGGCC |
| | | CGGGTTCGATTCCCGGTCAGGAAA |
| 300 | Tyr_GTA_chr2: 27273649-27273738 (+) | GCCTTCGATAGCTCAGTTGGTAGAGCGGAGGACTGTAGTGGATAGGGC |
| | | GTGGCAATCCTTAGGTCGCTGGTTCGATTCCGGCTCGAAGGA |
| 301 | Ala_AGC_chr2: 27274081-27274154 (+) | CGGGGGATTAGCTCAAATGGTAGAGCGCTCGCTTAGCATGCGAGAGGT |
| | | AGCGGGATCGATGCCCGCATCCTCCA |
| 302 | Ile_TAT_chr2: 43037675-43037768 (+) | AGCTCCAGTGGCGCAATCGGTTAGCGCGCGGTACTTATACAGCAGTAC |
| | | ATGCAGAGCAATGCCGAGGTTGTGAGTTCGAGCCTCACCTGGAGCA |
| 303 | Gly_CCC_chr2: 70476122-70476193 (−) | GCGCCGCTGGTGTAGTGGTATCATGCAAGATTCCCATTCTTGCGACCCG |
| | | GGTTCGATTCCCGGGCGGCGCAT |
| 304 | Glu_TTC_chr2: 131094700-131094772 (−) | TCCCATATGGTCTAGCGGTTAGGATTCCTGGTTTTCACCCAGGTGGCCC |
| | | GGGTTCGACTCCCGGTATGGGAAC |
| 305 | Ala_CGC_chr2: 157257280-157257352 (+) | GGGGGATGTAGCTCAGTGGTAGAGCGCGCGCTTCGCATGTGTGAGGTC |
| | | CCGGGTTCAATCCCCGGCATCTCCA |
| 306 | Gly_GCC_chr2: 157257658-157257729 (−) | GCATTGGTGGTTCAGTGGTAGAATTCTCGCCTGCCACGCGGGAGGCCC |
| | | GGGTTCGATTCCCGGCCAATGCAA |
| 307 | Arg_ACG_chr3: 45730490-45730563 (−) | GGGCCAGTGGCGCAATGGATAACGCGTCTGACTACGGATCAGAAGATT |
| | | CTAGGTTCGACTCCTGGCTGGCTCGC |
| 308 | Val_AAC_chr3: 169490017-169490090 (+) | GGTTTCCGTAGTGTAGTGGTTATCACGTTCGCCTAACACGCGAAAGGT |
| | | CCCCGGTTCGAAACCGGGCGGAAACA |
| 309 | Val_AAC_chr5: 180596609-180596682 (+) | AGTTTCCGTAGTGTAGTGGTTATCACGTTCGCCTAACACGCGAAAGGT |
| | | CCCCGGTTCGAAACCGGGCGGAAACA |
| 310 | Leu_AAG_chr5: 180614700-180614782 (+) | AGGTAGCGTGGCCGAGCGGTCTAAGGCGCTGGATTAAGGCTCCAGTCT |
| | | CTTCGGGGGCGTGGGTTCGAATCCCACCGCTGCCA |
| 311 | Val_AAC_chr5: 180615415-180615488 (−) | GTTTCCGTAGTGTAGTGGTCATCACGTTCGCCTAACACGCGAAAGGTC |
| | | CCCGGTTCGAAACCGGGCGGAAACAT |
| 312 | Pro_TGG_chr5: 180615853-180615925 (−) | GGCTCGTTGGTCTAGGGGTATGATTCTCGCTTTGGGTGCGAGAGGTCCC |
| | | GGGTTCAAATCCCGGACGAGCCCA |
| 313 | Thr_TGT_chr5: 180618686-180618758 (−) | GGCTCCATAGCTCAGGGGTTAGAGCACTGGTCTTGTAAACCAGGGTCG |
| | | CGAGTTCAAATCTCGCTGGGGCCTG |
| 314 | Ala_TGC_chr5: 180633867-180633939 (+) | TGGGGATGTAGCTCAGTGGTAGAGCGCATGCTTTGCATGTATGAGGCC |
| | | CCGGGTTCGATCCCCGGCATCTCCA |
| 315 | Lys_CTT_chr5: 180634754-180634827 (+) | CGCCCGGCTAGCTCAGTCGGTAGAGCATGAGACTCTTAATCTCAGGGT |
| | | CGTGGGTTCGAGCCCCACGTTGGGCG |
| 316 | Val_AAC_chr5: 180645269-180645342 (−) | GTTTCCGTAGTGTAGTGGTTATCACGTTCGCCTAACACGCGAAAGGTCC |
| | | CCGGTTCGAAACCGGGCGGAAACAA |
| 317 | Lys_CTT_chr5: 180648978-180649051 (−) | GCCCGGCTAGCTCAGTCGGTAGAGCATGAGACTCTTAATCTCAGGGTC |
| | | GTGGGTTCGAGCCCCACGTTGGGCGT |
| 318 | Val_CAC_chr5: 180649394-180649467 (−) | GTTTCCGTAGTGTAGTGGTTATCACGTTCGCCTCACACGCGAAAGGTCC |
| | | CCGGTTCGAAACCGGGCGGAAACAC |
| 319 | Met_CAT_chr6: 26286753-26286825 (+) | CAGCAGAGTGGCGCAGCGGAAGCGTGCTGGGCCCATAACCCAGAGGT |
| | | CGATGGATCGAAACCATCCTCTGCTA |
| 320 | Ser_GCT_chr6: 26305717-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCGC |
| 321 | Gln_TTG_chr6: 26311423-26311495 (−) | GGCCCCATGGTGTAATGGTTAGCACTCTGGACTTTGAATCCAGCGATC |
| | | CGAGTTCAAATCTCGGTGGGACCTG |
| 322 | Gln_TTG_chr6: 26311974-26312046 (−) | GGCCCCATGGTGTAATGGTTAGCACTCTGGACTTTGAATCCAGCGATC |
| | | CGAGTTCAAATCTCGGTGGGACCTA |
| 323 | Ser_TGA_chr6: 26312823-26312905 (−) | GTAGTCGTGGCCGAGTGGTTAAGGCGATGGACTTGAAATCCATTGGGG |
| | | TCTCCCCGCGCAGGTTCGAATCCTGCCGACTACGG |
| 324 | Met_CAT_chr6: 26313351-26313423 (−) | AGCAGAGTGGCGCAGCGGAAGCGTGCTGGGCCCATAACCCAGAGGTC |
| | | GATGGATCGAAACCATCCTCTGCTAT |
| 325 | Arg_TCG_chr6: 26323045-26323118 (+) | GGACCACGTGGCCTAATGGATAAGGCGTCTGACTTCGGATCAGAAGAT |
| | | TGAGGGTTCGAATCCCTCCGTGGTTA |
| 326 | Ser_AGA_chr6: 26327816-26327898 (+) | TGTAGTCGTGGCCGAGTGGTTAAGGCGATGGACTAGAAATCCATTGGG |
| | | GTCTCCCCGCGCAGGTTCGAATCCTGCCGACTACG |
| 327 | Met_CAT_chr6: 26330528-26330600 (−) | AGCAGAGTGGCGCAGCGGAAGCGTGCTGGGCCCATAACCCAGAGGTC |
| | | GATGGATCGAAACCATCCTCTGCTAG |
| 328 | Leu_CAG_chr6: 26521435-26521518 (+) | CGTCAGGATGGCCGAGCGGTCTAAGGCGCTGCGTTCAGGTCGCAGTCT |
| | | CCCCTGGAGGCGTGGGTTCGAATCCCACTCCTGACA |
| 329 | Thr_AGT_chr6: 26533144-26533218 (−) | GGCTCCGTGGCTTAGCTGGTTAAAGCGCCTGTCTAGTAAACAGGAGAT |
| | | CCTGGGTTCGAATCCCAGCGGGGCCTG |
| 330 | Arg_ACG_chr6: 26537725-26537798 (+) | AGGGCCAGTGGCGCAATGGATAACGCGTCTGACTACGGATCAGAAGA |
| | | TTCCAGGTTCGACTCCTGGCTGGCTCG |
| 331 | Val_CAC_chr6: 26538281-26538354 (+) | GGTTTCCGTAGTGTAGTGGTTATCACGTTCGCCTCACACGCGAAAGGTC |
| | | CCCGGTTCGAAACCGGGCGGAAACA |
| 332 | Ala_CGC_chr6: 26553730-26553802 (+) | AGGGGATGTAGCTCAGTGGTAGAGCGCATGCTTCGCATGTATGAGGTC |
| | | CCGGGTTCGATCCCCGGCATCTCCA |
| 333 | Ile_AAT_chr6: 26554349-26554423 (+) | TGGCCGGTTAGCTCAGTTGGTTAGAGCGTGGTGCTAATAACGCCAAGG |
| | | TCGCGGGTTCGATCCCCGTACGGGCCA |
| 334 | Pro_AGG_chr6: 26555497-26555569 (+) | CGGCTCGTTGGTCTAGGGGTATGATTCTCGCTTAGGGTGCGAGAGGTC |
| | | CCGGGTTCAAATCCCGGACGAGCCC |
| 335 | Lys_CTT_chr6: 26556773-26556846 (+) | AGCCCGGCTAGCTCAGTCGGTAGAGCATGAGACTCTTAATCTCAGGGT |
| | | CGTGGGTTCGAGCCCCACGTTGGGCG |
| 336 | Tyr_GTA_chr6: 26569085-26569176 (+) | TCCTTCGATAGCTCAGTTGGTAGAGCGGAGGACTGTAGTTGGCTGTGT |
| | | CCTTAGACATCCTTAGGTCGCTGGTTCGAATCCGGCTCGAAGGA |
| 337 | Ala_AGC_chr6: 26572091-26572164 (−) | GGGGAATTAGCTCAAATGGTAGAGCGCTCGCTTAGCATGCGAGAGGTA |
| | | GCGGGATCGATGCCCGCATTCTCCAG |
| 338 | Met_CAT_chr6: 26766443-26766516 (+) | CGCCCTCTTAGCGCAGCGGGCAGCGCGTCAGTCTCATAATCTGAAGGT |
| | | CCTGAGTTCGAGCCTCAGAGAGGGCA |
| 339 | Ile_TAT_chr6: 26988124-26988218 (+) | TGCTCCAGTGGCGCAATCGGTTAGCGCGCGGTACTTATATGGCAGTAT |
| | | GTGTGCGAGTGATGCCGAGGTTGTGAGTTCGAGCCTCACCTGGAGCA |
| 340 | His_GTG_chr6: 27125905-27125977 (+) | TGCCGTGATCGTATAGTGGTTAGTACTCTGCGTTGTGGCCGCAGCAACC |
| | | TCGGTTCGAATCCGAGTCACGGCA |
| 341 | Ile_AAT_chr6: 27144993-27145067 (−) | GGCCGGTTAGCTCAGTTGGTTAGAGCGTGGTGCTAATAACGCCAAGGT |
| | | CGCGGGTTCGATCCCCGTACGGGCCAC |
| 342 | Val_AAC_chr6: 27203287-27203360 (+) | AGTTTCCGTAGTGTAGTGGTTATCACGTTTGCCTAACACGCGAAAGGTC |
| | | CCCGGTTCGAAACCGGGCAGAAACA |
| 343 | Val_CAC_chr6: 27248048-27248121 (−) | GCTTCTGTAGTGTAGTGGTTATCACGTTCGCCTCACACGCGAAAGGTCC |
| | | CCGGTTCGAAACCGGGCAGAAGCAA |
| 344 | Asp_GTC_chr6: 27447452-27447524 (+) | TTCCTCGTTAGTATAGTGGTGAGTATCCCCGCCTGTCACGCGGGAGACC |
| | | GGGGTTCGATTCCCCGACGGGGAG |
| 345 | Ser_TGA_chr6: 27473606-27473688 (−) | GTAGTCGTGGCCGAGTGGTTAAGGCGATGGACTTGAAATCCATTGGGG |
| | | TTTCCCCGCGCAGGTTCGAATCCTGTCGGCTACGG |
| 346 | Gln_CTG_chr6: 27487307-27487379 (+) | AGGTTCCATGGTGTAATGGTTAGCACTCTGGACTCTGAATCCAGCGAT |
| | | CCGAGTTCAAATCTCGGTGGAACCT |
| 347 | Asp_GTC_chr6: 27551235-27551307 (−) | TCCTCGTTAGTATAGTGGTGAGTGTCCCCGTCTGTCACGCGGGAGACC |
| | | GGGGTTCGATTCCCCGACGGGGAGA |
| 348 | Val_AAC_chr6: 27618706-27618779 (−) | GTTTCCGTAGTGTAGTGGTTATCACGTTCGCCTAACACGCGAAAGGTCC |
| | | CTGGATCAAAACCAGGCGGAAACAA |
| 349 | Ile_AAT_chr6: 27655966-27656040 (+) | CGGCCGGTTAGCTCAGTTGGTTAGAGCGTGGTGCTAATAACGCCAAGG |
| | | TCGCGGGTTCGATCCCCGTACTGGCCA |
| 350 | Gln_CTG_chr6: 27759134-27759206 (−) | GGCCCCATGGTGTAATGGTCAGCACTCTGGACTCTGAATCCAGCGATC |
| | | CGAGTTCAAATCTCGGTGGGACCCA |
| 351 | Gln_TTG_chr6: 27763639-27763711 (−) | GGCCCCATGGTGTAATGGTTAGCACTCTGGACTTTGAATCCAGCGATC |
| | | CGAGTTCAAATCTCGGTGGGACCTT |
| 352 | Ala_AGC_chr6: 28574932-28575004 (+) | TGGGGGTGTAGCTCAGTGGTAGAGCGCGTGCTTAGCATGTACGAGGTC |
| | | CCGGGTTCAATCCCCGGCACCTCCA |
| 353 | Ala_AGC_chr6: 28626013-28626085 (−) | GGGGATGTAGCTCAGTGGTAGAGCGCATGCTTAGCATGCATGAGGTCC |
| | | CGGGTTCGATCCCCAGCATCTCCAG |
| 354 | Ala_CGC_chr6: 28697091-28697163 (+) | AGGGGGTGTAGCTCAGTGGTAGAGCGCGTGCTTCGCATGTACGAGGCC |
| | | CCGGGTTCGACCCCCGGCTCCTCCA |
| 355 | Ala_AGC_chr6: 28806220-28806292 (−) | GGGGGTGTAGCTCAGTGGTAGAGCGCGTGCTTAGCATGCACGAGGCCC |
| | | CGGGTTCAATCCCCGGCACCTCCAT |
| 356 | Ala_AGC_chr6: 28831461-28831533 (−) | GGGGGTGTAGCTCAGTGGTAGAGCGCGTGCTTAGCATGCACGAGGCCC |
| | | CGGGTTCAATCCCCGGCACCTCCAG |
| 357 | Leu_CAA_chr6: 28863999-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 358 | Leu_CAA_chr6: 28908829-28908934 (+) | TGTCAGGATGGCCGAGTGGTCTAAGGCGCCAGACTCAAGCTTGGCTTC |
| | | CTCGTGTTGAGGATTCTGGTCTCCAATGGAGGCGTGGGTTCGAATCCC |
| 359 | Gln_CTG_chr6: 28909377-28909449 (−) | GGTTCCATGGTGTAATGGTTAGCACTCTGGACTCTGAATCCAGCGATCC |
| | | GAGTTCAAATCTCGGTGGAACCTT |
| 360 | Leu_AAG_chr6: 28911398-28911480 (−) | GGTAGCGTGGCCGAGCGGTCTAAGGCGCTGGATTAAGGCTCCAGTCTC |
| | | TTCGGGGGCGTGGGTTCGAATCCCACCGCTGCCAG |
| 361 | Met_CAT_chr6: 28912351-28912424 (+) | TGCCTCCTTAGCGCAGTAGGCAGCGCGTCAGTCTCATAATCTGAAGGT |
| | | CCTGAGTTCGAACCTCAGAGGGGGCA |
| 362 | Lys_TTT_chr6: 28918805-28918878 (+) | AGCCCGGATAGCTCAGTCGGTAGAGCATCAGACTTTTAATCTGAGGGT |
| | | CCAGGGTTCAAGTCCCTGTTCGGGCG |
| 363 | Met_CAT_chr6: 28921041-28921114 (−) | GCCTCCTTAGCGCAGTAGGCAGCGCGTCAGTCTCATAATCTGAAGGTC |
| | | CTGAGTTCGAACCTCAGAGGGGGCAG |
| 364 | Glu_CTC_chr6: 28949975-28950047 (+) | TTCCCTGGTGGTCTAGTGGTTAGGATTCGGCGCTCTCACCGCCGCGGCC |
| | | CGGGTTCGATTCCCGGTCAGGGAA |
| 365 | Leu_TAA_chr6: 144537683-144537766 (+) | CACCAGGATGGCCGAGTGGTTAAGGCGTTGGACTTAAGATCCAATGGA |
| | | CATATGTCCGCGTGGGTTCGAACCCCACTCCTGGTA |
| 366 | Pro_AGG_chr7: 128423503-128423575 (+) | TGGCTCGTTGGTCTAGGGGTATGATTCTCGCTTAGGGTGCGAGAGGTC |
| | | CCGGGTTCAAATCCCGGACGAGCCC |
| 367 | Arg_CCT_chr7: 139025445-139025518 (+) | AGCCCCAGTGGCCTAATGGATAAGGCATTGGCCTCCTAAGCCAGGGAT |
| | | TGTGGGTTCGAGTCCCATCTGGGGTG |
| 368 | Cys_GCA_chr7: 149388271-149388343 (−) | GGGGATATAGCTCAGGGGTAGAGCATTTGACTGCAGATCAAGAGGTCC |
| | | CCGGTTCAAATCCGGGTGCCCCCCC |
| 369 | Tyr_GTA_chr8: 67025601-67025694 (+) | CCCTTCGATAGCTCAGCTGGTAGAGCGGAGGACTGTAGCTACTTCCTC |
| | | AGCAGGAGACATCCTTAGGTCGCTGGTTCGATTCCGGCTCGAAGGA |
| 370 | Tyr_GTA_chr8: 67026222-67026311 (+) | CCCTTCGATAGCTCAGCTGGTAGAGCGGAGGACTGTAGGCGCGCGCCC |
| | | GTGGCCATCCTTAGGTCGCTGGTTCGATTCCGGCTCGAAGGA |
| 371 | Ala_AGC_chr8: 67026423-67026496 (+) | TGGGGGATTAGCTCAAATGGTAGAGCGCTCGCTTAGCATGCGAGAGGT |
| | | AGCGGGATCGATGCCCGCATCCTCCA |
| 372 | Ser_AGA_chr8: 96281884-96281966 (−) | GTAGTCGTGGCCGAGTGGTTAAGGCGATGGACTAGAAATCCATTGGGG |
| | | TCTCCCCGCGCAGGTTCGAATCCTGCCGACTACGG |
| 373 | Met_CAT_chr8: 124169469-124169542 (−) | GCCTCGTTAGCGCAGTAGGTAGCGCGTCAGTCTCATAATCTGAAGGTC |
| | | GTGAGTTCGATCCTCACACGGGGCAC |
| 374 | Arg_TCT_chr9: 131102354-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCGA |
| 375 | Asn_GTT_chr10: 22518437-22518511 (−) | GTCTCTGTGGCGCAATCGGTTAGCGCGTTCGGCTGTTAACCGAAAGGT |
| | | TGGTGGTTCGAGCCCACCCAGGGACGC |
| 376 | Ser_TGA_chr10: 69524260-69524342 (+) | GGCAGCGATGGCCGAGTGGTTAAGGCGTTGGACTTGAAATCCAATGGG |
| | | GTCTCCCCGCGCAGGTTCGAACCCTGCTCGCTGCG |
| 377 | Val_TAC_chr11: 59318101-59318174 (−) | GGTTCCATAGTGTAGTGGTTATCACGTCTGCTTTACACGCAGAAGGTCC |
| | | TGGGTTCGAGCCCCAGTGGAACCAT |
| 378 | Val_TAC_chr11: 59318459-59318532 (−) | GGTTCCATAGTGTAGCGGTTATCACGTCTGCTTTACACGCAGAAGGTCC |
| | | TGGGTTCGAGCCCCAGTGGAACCAC |
| 379 | Arg_TCT_chr11: 59318766-59318852 (+) | TGGCTCTGTGGCGCAATGGATAGCGCATTGGACTTCTAGATAGTTAGA |
| | | GAAATTCAAAGGTTGTGGGTTCGAGTCCCACCAGAGTCG |
| 380 | Leu_TAA_chr11: 59319227-59319310 (+) | TACCAGAATGGCCGAGTGGTTAAGGCGTTGGACTTAAGATCCAATGGA |
| | | TTCATATCCGCGTGGGTTCGAACCCCACTTCTGGTA |
| 381 | Lys_TTT_chr11: 59323901-59323974 (+) | GGCCCGGATAGCTCAGTCGGTAGAGCATCAGACTTTTAATCTGAGGGT |
| | | CCGGGGTTCAAGTCCCTGTTCGGGCG |
| 382 | Phe_GAA_chr11: 59324969-59325042 (−) | GCCGAAATAGCTCAGTTGGGAGAGCGTTAGACTGAAGATCTAAAGGTC |
| | | CCTGGTTCGATCCCGGGTTTCGGCAG |
| 383 | Lys_TTT_chr11:59327807-59327880 (−) | GCCCGGATAGCTCAGTCGGTAGAGCATCAGACTTTTAATCTGAGGGTC |
| | | CAGGGTTCAAGTCCCTGTTCGGGCGG |
| 384 | Phe_GAA_chr11:59333852-59333925 (−) | GCCGAAATAGCTCAGTTGGGAGAGCGTTAGACTGAAGATCTAAAGGTC |
| | | CCTGGTTCAATCCCGGGTTTCGGCAG |
| 385 | Ser_GCT_chr11: 66115590-66115672 (+) | GGACGAGGTGGCCGAGTGGTTAAGGCGATGGACTGCTAATCCATTGTG |
| | | CTTTGCACGCGTGGGTTCGAATCCCATCCTCGTCG |
| 386 | Pro_TGG_chr11: 75946868-75946940 (−) | GGCTCGTTGGTCTAGGGGTATGATTCTCGGTTTGGGTCCGAGAGGTCCC |
| | | GGGTTCAAATCCCGGACGAGCCCC |
| 387 | Ser_CGA_chr12: 56584147-56584229 (+) | AGTCACGGTGGCCGAGTGGTTAAGGCGTTGGACTCGAAATCCAATGGG |
| | | GTTTCCCCGCACAGGTTCGAATCCTGTTCGTGACG |
| 388 | Asp_GTC_chr12: 98897280-98897352 (+) | CTCCTCGTTAGTATAGTGGTTAGTATCCCCGCCTGTCACGCGGGAGACC |
| | | GGGGTTCAATTCCCCGACGGGGAG |
| 389 | Trp_CCA_chr12: 98898029-98898101 (+) | GGACCTCGTGGCGCAACGGTAGCGCGTCTGACTCCAGATCAGAAGGCT |
| | | GCGTGTTCGAATCACGTCGGGGTCA |
| 390 | Ala_TGC_chr12: 125406300-125406372 (−) | GGGGATGTAGCTCAGTGGTAGAGCGCATGCTTTGCATGTATGAGGCCC |
| | | CGGGTTCGATCCCCGGCATCTCCAT |
| 391 | Phe_GAA_chr12: 125412388-125412461 (−) | GCCGAAATAGCTCAGTTGGGAGAGCGTTAGACTGAAGATCTAAAGGTC |
| | | CCTGGTTCGATCCCGGGTTTCGGCAC |
| 392 | Ala_TGC_chr12: 125424511-125424583 (+) | AGGGGATGTAGCTCAGTGGTAGAGCGCATGCTTTGCACGTATGAGGCC |
| | | CCGGGTTCAATCCCCGGCATCTCCA |
| 393 | Asn_GTT_chr13: 31248100-31248174 (−) | GTCTCTGTGGCGCAATCGGTTAGCGCGTTCGGCTGTTAACCGAAAGGT |
| | | TGGTGGTTCGAGCCCACCCAGGGACGG |
| 394 | Glu_TTC_chr13: 45492061-45492133 (−) | TCCCACATGGTCTAGCGGTTAGGATTCCTGGTTTTCACCCAGGCGGCCC |
| | | GGGTTCGACTCCCGGTGTGGGAAC |
| 395 | Thr_TGT_chr14: 21081948-21082021 (−) | GGCTCCATAGCTCAGGGGTTAGAGCGCTGGTCTTGTAAACCAGGGGTC |
| | | GCGAGTTCAATTCTCGCTGGGGCCTG |
| 396 | Leu_TAG_chr14: 21093528-21093610 (+) | TGGTAGTGTGGCCGAGCGGTCTAAGGCGCTGGATTTAGGCTCCAGTCT |
| | | CTTCGGGGGCGTGGGTTCGAATCCCACCACTGCCA |
| 397 | Thr_TGT_chr14: 21099318-21099391 (−) | GGCTCCATAGCTCAGGGGTTAGAGCACTGGTCTTGTAAACCAGGGGTC |
| | | GCGAGTTCAAATCTCGCTGGGGCCTC |
| 398 | Pro_TGG_chr14: 21101164-21101236 (+) | TGGCTCGTTGGTCTAGTGGTATGATTCTCGCTTTGGGTGCGAGAGGTCC |
| | | CGGGTTCAAATCCCGGACGAGCCC |
| 399 | Tyr_GTA_chr14: 21131350-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGAA |
| 400 | Thr_TGT_chr14: 21149848-21149921 (+) | AGGCCCTATAGCTCAGGGGTTAGAGCACTGGTCTTGTAAACCAGGGGT |
| | | CGCGAGTTCAAATCTCGCTGGGGCCT |
| 401 | Tyr_GTA_chr14: 21151431-21151520 (+) | TCCTTCGATAGCTCAGCTGGTAGAGCGGAGGACTGTAGTACTTAATGT |
| | | GTGGTCATCCTTAGGTCGCTGGTTCGATTCCGGCTCGAAGGA |
| 402 | Pro_TGG_chr14: 21152174-21152246 (+) | TGGCTCGTTGGTCTAGGGGTATGATTCTCGCTTTGGGTGCGAGAGGTCC |
| | | CGGGTTCAAATCCCGGACGAGCCC |
| 403 | Lys_CTT_chr14: 58706612-58706685 (−) | GCCCGGCTAGCTCAGTCGGTAGAGCATGGGACTCTTAATCCCAGGGTC |
| | | GTGGGTTCGAGCCCCACGTTGGGCGC |
| 404 | Ile_AAT_chr14: 102783428-102783502 (+) | CGGCCGGTTAGCTCAGTTGGTTAGAGCGTGGTGCTAATAACGCCAAGG |
| | | TCGCGGGTTCGATCCCCGTACGGGCCA |
| 405 | Glu_TTC_chr15: 26327380-26327452 (−) | TCCCACATGGTCTAGCGGTTAGGATTCCTGGTTTTCACCCAGGCGGCCC |
| | | GGGTTCGACTCCCGGTGTGGGAAT |
| 406 | Ser_GCT_chr15: 40886022-40886104 (−) | GACGAGGTGGCCGAGTGGTTAAGGCGATGGACTGCTAATCCATTGTGC |
| | | TCTGCACGCGTGGGTTCGAATCCCATCCTCGTCGA |
| 407 | His_GTG_chr15: 45490803-45490875 (−) | GCCGTGATCGTATAGTGGTTAGTACTCTGCGTTGTGGCCGCAGCAACCT |
| | | CGGTTCGAATCCGAGTCACGGCAT |
| 408 | His_GTG_chr15: 45493348-45493420 (+) | CGCCGTGATCGTATAGTGGTTAGTACTCTGCGTTGTGGCCGCAGCAAC |
| | | CTCGGTTCGAATCCGAGTCACGGCA |
| 409 | Gln_CTG_chr15: 66161399-66161471 (−) | GGTTCCATGGTGTAATGGTTAGCACTCTGGACTCTGAATCCAGCGATCC |
| | | GAGTTCAAATCTCGGTGGAACCTG |
| 410 | Lys_CTT_chr15: 79152903-79152976 (+) | TGCCCGGCTAGCTCAGTCGGTAGAGCATGGGACTCTTAATCCCAGGGT |
| | | CGTGGGTTCGAGCCCCACGTTGGGCG |
| 411 | Arg_TCG_chr15: 89878303-89878376 (+) | GGGCCGCGTGGCCTAATGGATAAGGCGTCTGACTTCGGATCAGAAGAT |
| | | TGCAGGTTCGAGTCCTGCCGCGGTCG |
| 412 | Gly_CCC_chr16: 686735-686806 (−) | GCGCCGCTGGTGTAGTGGTATCATGCAAGATTCCCATTCTTGCGACCCG |
| | | GGTTCGATTCCCGGGCGGCGCAC |
| 413 | Arg_CCG_chr16: 3200674-3200747 (+) | GGGCCGCGTGGCCTAATGGATAAGGCGTCTGATTCCGGATCAGAAGAT |
| | | TGAGGGTTCGAGTCCCTTCGTGGTCG |
| 414 | Arg_CCT_chr16: 3202900-3202973 (+) | CGCCCCGGTGGCCTAATGGATAAGGCATTGGCCTCCTAAGCCAGGGAT |
| | | TGTGGGTTCGAGTCCCACCCGGGGTA |
| 415 | Lys_CTT_chr16: 3207405-3207478 (−) | GCCCGGCTAGCTCAGTCGGTAGAGCATGAGACCCTTAATCTCAGGGTC |
| | | GTGGGTTCGAGCCCCACGTTGGGCGT |
| 416 | Thr_CGT_chr16: 14379749-14379821 (+) | AGGCGCGGTGGCCAAGTGGTAAGGCGTCGGTCTCGTAAACCGAAGATC |
| | | ACGGGTTCGAACCCCGTCCGTGCCT |
| 417 | Leu_TAG_chr16: 22207031-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCAC |
| 418 | Leu_AAG_chr16: 22308460-22308542 (+) | GGGTAGCGTGGCCGAGCGGTCTAAGGCGCTGGATTAAGGCTCCAGTCT |
| | | CTTCGGGGGCGTGGGTTCGAATCCCACCGCTGCCA |
| 419 | Leu_CAG_chr16: 57333862-57333945 (+) | AGTCAGGATGGCCGAGCGGTCTAAGGCGCTGCGTTCAGGTCGCAGTCT |
| | | CCCCTGGAGGCGTGGGTTCGAATCCCACTTCTGACA |
| 420 | Leu_CAG_chr16: 57334391-57334474 (−) | GTCAGGATGGCCGAGCGGTCTAAGGCGCTGCGTTCAGGTCGCAGTCTC |
| | | CCCTGGAGGCGTGGGTTCGAATCCCACTTCTGACAG |
| 421 | Met_CAT_chr16: 87417627-87417700 (−) | GCCTCGTTAGCGCAGTAGGCAGCGCGTCAGTCTCATAATCTGAAGGTC |
| | | GTGAGTTCGAGCCTCACACGGGGCAG |
| 422 | Leu_TAG_chr17: 8023631-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCAG |
| 423 | Arg_TCT_chr17: 8024242-8024330 (+) | TGGCTCTGTGGCGCAATGGATAGCGCATTGGACTTCTAGTGACGAATA |
| | | GAGCAATTCAAAGGTTGTGGGTTCGAATCCCACCAGAGTCG |
| 424 | Gly_GCC_chr17: 8029063-8029134 (+) | CGCATTGGTGGTTCAGTGGTAGAATTCTCGCCTGCCACGCGGGAGGCC |
| | | CGGGTTCGATTCCCGGCCAATGCA |
| 425 | Ser_CGA_chr17: 8042198-8042280 (−) | GCTGTGATGGCCGAGTGGTTAAGGCGTTGGACTCGAAATCCAATGGGG |
| | | TCTCCCCGCGCAGGTTCGAATCCTGCTCACAGCGT |
| 426 | Thr_AGT_chr17: 8042769-8042843 (−) | GGCGCCGTGGCTTAGCTGGTTAAAGCGCCTGTCTAGTAAACAGGAGAT |
| | | CCTGGGTTCGAATCCCAGCGGTGCCTG |
| 427 | Trp_CCA_chr17: 8089675-8089747 (+) | CGACCTCGTGGCGCAACGGTAGCGCGTCTGACTCCAGATCAGAAGGTT |
| | | GCGTGTTCAAATCACGTCGGGGTCA |
| 428 | Ser_GCT_chr17: 8090183-8090265 (+) | AGACGAGGTGGCCGAGTGGTTAAGGCGATGGACTGCTAATCCATTGTG |
| | | CTCTGCACGCGTGGGTTCGAATCCCATCCTCGTCG |
| 429 | Thr_AGT_chr17: 8090477-8090551 (+) | CGGCGCCGTGGCTTAGTTGGTTAAAGCGCCTGTCTAGTAAACAGGAGA |
| | | TCCTGGGTTCGAATCCCAGCGGTGCCT |
| 430 | Trp_CCA_chr17: 8124186-8124258 (−) | GGCCTCGTGGCGCAACGGTAGCGCGTCTGACTCCAGATCAGAAGGTTG |
| | | CGTGTTCAAATCACGTCGGGGTCAA |
| 431 | Gly_TCC_chr17: 8124865-8124937 (+) | AGCGTTGGTGGTATAGTGGTAAGCATAGCTGCCTTCCAAGCAGTTGAC |
| | | CCGGGTTCGATTCCCGGCCAACGCA |
| 432 | Asp_GTC_chr17: 8125555-8125627 (−) | TCCTCGTTAGTATAGTGGTGAGTATCCCCGCCTGTCACGCGGGAGACC |
| | | GGGGTTCGATTCCCCGACGGGGAGA |
| 433 | Pro_CGG_chr17: 8126150-8126222 (−) | GGCTCGTTGGTCTAGGGGTATGATTCTCGCTTCGGGTGCGAGAGGTCC |
| | | CGGGTTCAAATCCCGGACGAGCCCT |
| 434 | Thr_AGT_chr17: 8129552-8129626 (−) | GGCGCCGTGGCTTAGTTGGTTAAAGCGCCTGTCTAGTAAACAGGAGAT |
| | | CCTGGGTTCGAATCCCAGCGGTGCCTT |
| 435 | Ser_AGA_chr17: 8129927-8130009 (−) | GTAGTCGTGGCCGAGTGGTTAAGGCGATGGACTAGAAATCCATTGGGG |
| | | TCTCCCCGCGCAGGTTCGAATCCTGCCGACTACGT |
| 436 | Trp_CCA_chr17: 19411493-19411565 (+) | TGACCTCGTGGCGCAATGGTAGCGCGTCTGACTCCAGATCAGAAGGTT |
| | | GCGTGTTCAAGTCACGTCGGGGTCA |
| 437 | Thr_CGT_chr17: 29877092-29877164 (+) | AGGCGCGGTGGCCAAGTGGTAAGGCGTCGGTCTCGTAAACCGAAGATC |
| | | GCGGGTTCGAACCCCGTCCGTGCCT |
| 438 | Cys_GCA_chr17: 37023897-37023969 (+) | AGGGGGTATAGCTCAGTGGTAGAGCATTTGACTGCAGATCAAGAGGTC |
| | | CCCGGTTCAAATCCGGGTGCCCCCT |
| 439 | Cys_GCA_chr17: 37025544-37025616 (−) | GGGGGTATAGCTCAGTGGTAGAGCATTTGACTGCAGATCAAGAGGTCC |
| | | CTGGTTCAAATCCGGGTGCCCCCTC |
| 440 | Cys_GCA_chr17: 37309986-37310058 (−) | GGGGGTATAGCTCAGTGGTAGAGCATTTGACTGCAGATCAAGAGGTCC |
| | | CCGGTTCAAATCCGGGTGCCCCCTC |
| 441 | Gln_TTG_chr17: 47269889-47269961 (+) | AGGTCCCATGGTGTAATGGTTAGCACTCTGGACTTTGAATCCAGCGAT |
| | | CCGAGTTCAAATCTCGGTGGGACCT |
| 442 | Arg_CCG_chr17: 66016012-66016085 (−) | GACCCAGTGGCCTAATGGATAAGGCATCAGCCTCCGGAGCTGGGGATT |
| | | GTGGGTTCGAGTCCCATCTGGGTCGC |
| 443 | Arg_CCT_chr17: 73030000-73030073 (+) | AGCCCCAGTGGCCTAATGGATAAGGCACTGGCCTCCTAAGCCAGGGAT |
| | | TGTGGGTTCGAGTCCCACCTGGGGTA |
| 444 | Arg_CCT_chr17: 73030525-73030598 (−) | GCCCCAGTGGCCTAATGGATAAGGCACTGGCCTCCTAAGCCAGGGATT |
| | | GTGGGTTCGAGTCCCACCTGGGGTGT |
| 445 | Arg_TCG_chr17: 73031207-73031280 (+) | AGACCGCGTGGCCTAATGGATAAGGCGTCTGACTTCGGATCAGAAGAT |
| | | TGAGGGTTCGAGTCCCTTCGTGGTCG |
| 446 | Asn_GTT_chr19: 1383561-1383635 (+) | CGTCTCTGTGGCGCAATCGGTTAGCGCGTTCGGCTGTTAACCGAAAGG |
| | | TTGGTGGTTCGAGCCCACCCAGGGACG |
| 447 | Gly_TCC_chr19: 4724081-4724153 (+) | GGCGTTGGTGGTATAGTGGTTAGCATAGCTGCCTTCCAAGCAGTTGAC |
| | | CCGGGTTCGATTCCCGGCCAACGCA |
| 448 | Val_CAC_chr19: 4724646-4724719 (−) | GTTTCCGTAGTGTAGCGGTTATCACATTCGCCTCACACGCGAAAGGTCC |
| | | CCGGTTCGATCCCGGGCGGAAACAG |
| 449 | Thr_AGT_chr19: 33667962-33668036 (+) | TGGCGCCGTGGCTTAGTTGGTTAAAGCGCCTGTCTAGTAAACAGGAGA |
| | | TCCTGGGTTCGAATCCCAGCGGTGCCT |
| 450 | Ile_TAT_chr19: 39902807-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCAC |
| 451 | Gly_GCC_chr21: 18827106-18827177 (−) | GCATGGGTGGTTCAGTGGTAGAATTCTCGCCTGCCACGCGGGAGGCCC |
| | | GGGTTCGATTCCCGGCCCATGCAG |

In an embodiment, a TREM, e.g., an exogenous TREM, comprises 1, 2, 3, or 4 of the following properties:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype.

In an embodiment, the expression profile can be mediated by a change introduced into a nucleic acid that modulates expression, or by addition of an agent that modulates expression of the RNA molecule.

In an embodiment, a TREM, e.g., an exogenous TREM comprises (a), (b), (c) and (d).

In an embodiment, a TREM, e.g., an exogenous TREM comprises (a), (b) and (c).

In an embodiment, a TREM, e.g., an exogenous TREM comprises (a), (b) and (d).

In an embodiment, a TREM, e.g., an exogenous TREM comprises (a), (c) and (d).

In an embodiment, a TREM, e.g., an exogenous TREM comprises (b), (c) and (d).

In an embodiment, a TREM, e.g., an exogenous TREM comprises (a) and (d).

In an embodiment, a TREM, e.g., an exogenous TREM comprises (c) and (d).

### TREM Fragments

In an embodiment, a TREM comprises a fragment (sometimes referred to herein as a TREM fragment), e.g., a fragment of a RNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., Dicer, Angiogenin, RNaseP, RNaseZ, Rny1, or PrrC.

In an embodiment, a TREM fragment can be produced in vivo, ex vivo or in vitro. In an embodiment, a TREM fragment is produced in vivo, in the host cell. In an embodiment, a TREM fragment is produced ex vivo. In an embodiment, a TREM fragment is produced in vitro, e.g., as described in Example 12. In an embodiment, the TREM fragment is produced by fragmenting an expressed TREM after production of the TREM by the cell, e.g., a TREM produced by the host cell is fragmented after release or purification from the host cell, e.g., the TREM is fragmented ex vivo or in vitro.

Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

In an embodiment, a TREM fragment comprises a TREM structure, domain, or activity, e.g., as described herein above. In an embodiment, a TREM fragment comprises adaptor function, e.g., as described herein. In an embodiment, a TREM fragment comprises cognate adaptor function, e.g., as described herein. In an embodiment, a TREM fragment comprises non-cognate adaptor function, e.g., as described herein. In an embodiment, a TREM fragment comprises regulatory function, e.g., as described herein.

In an embodiment, a TREM fragment comprises translation inhibition function, e.g., displacement of an initiation factor, e.g., eIF4G.

In an embodiment, a TREM fragment comprises epigenetic function, e.g., epigenetic inheritance of a disorder, e.g., a metabolic disorder. In some embodiments, an epigenetic inheritance function can have a generational impact, e.g., as compared to somatic epigenetic regulation.

In an embodiment, a TREM fragment comprises retroviral regulation function, e.g., regulation of retroviral reverse transcription, e.g., HERV regulation.

In an embodiment, a TREM fragment comprises gene silencing function, e.g., by binding to AGO and/or PIWI.

In an embodiment, a TREM fragment comprises neuroprotectant function, e.g., by the sequestration of a translation initiation factor, e.g., in stress granules, to promote, e.g., motor neuron survival under cellular stress.

In an embodiment, a TREM fragment comprises anti-cancer function, e.g., by preventing cancer progression through the binding and/or sequestration of, e.g., metastatic transcript-stabilizing proteins.

In an embodiment, a TREM fragment comprises cell survival function, e.g., increased cell survival, by binding to, e.g., cytochrome c and/or cyt c ribonucleoprotein complex.

In an embodiment, a TREM fragment comprises ribosome biogenesis function, e.g., a TREM fragment can regulate ribosome biogenesis by, e.g., regulation of, e.g., binding to, an mRNA coding for ribosomal proteins.

### TREM Modifications

A TREM described herein can comprise a moiety, often referred to herein as a modification, e.g., a moiety described in Table 2. While the term modification as used herein should not generally be construed to be the product of any particular process, in embodiments, the formation of a modification can be mediated by an enzyme in Table 2. In embodiments, the modification is formed post-transcriptionally. In embodiments, the modification is formed co-transcriptionally. In an embodiment, the modification occurs in vivo, e.g., in the host cell.

In an embodiment, the modification is a modification listed in any of rows 1-62 of Table 2. In an embodiment, the modification is a modification listed in any of rows 1-62 of Table 2, and the formation of the modification is mediated by an enzyme in Table 2. In an embodiment the modification is selected from a row in Table 2 and the formation of the modification is mediated by an enzyme from the same row in Table 2.

TABLE 2
List of tRNA modifications and associated enzymes.

| | Short | | |
|---|---|---|---|
| | Name | Modification | Enzyme list |
| 1 | m1Am | 1,2′-O-dimethyladenosine | METTL3 |
| 2 | imG | wyosine | Trm5, Tyw1, Tyw2, Tyw3, and Tyw4 |
| 3 | m5s2U | 5-methyl-2-thiouridine | TrmU |
| 4 | m6t6A | N6-methyl-N6- | TRMO, TrmO |
| | | threonylcarbamoyladenosine | |
| 5 | QtRNA | queuosine | TGTase |
| 6 | OHyW | hydroxywybutosine | Trm5, TYW1 , TYW2, TYW3 , TYW4 |
| 7 | io6A | N6-(cis-hydroxyisopentenyl)adenosine | TRIT1 |
| 8 | Gr(p) | 2′-O-ribosylguanosine (phosphate) | |
| 9 | ho5U | 5-hydroxyuridine | |
| 10 | ncm5Um | 5-carbamoylmethyl-2′-O-methyluridine | ELP1, ELP2, ELP3, ELP4, ELP5, ELP6, |
| | | | KTI111, KTI112, KTI113, Uba4, Urm1, |
| | | | Tum1, Ncs6, Ncs2, Trm9, Sit4, Isu1, Isu2, |
| | | | Sap185, Sap190 |
| 11 | OHyW* | hydroxywybutosine | wybutosine hydroxylases |
| 12 | acp3U | 3-(3-amino-3-carboxypropyl)uridine | |
| 13 | mcm5s2U | 5-methoxycarbonylmethyl-2-thiouridine | ALKBH8, Ncs6, Trm9, Ncs2, TrmU, |
| | | | CTU1, CTU2, ELP1, ELP2, ELP3, ELP4, |
| | | | ELP5, ELP6 |
| 14 | m5U | 5-methyluridine | Trm2 |
| 15 | D | dihydrouridine | DUS1, DUS2, DUS3, DUS4 |
| 16 | mcm5Um | 5-methoxycarbonylmethyl-2′-O- | ELP1, ELP2, ELP3, ELP4, ELP5, |
| | | methyluridine | ELP6, Trm9, ALKBH-MT,? |
| 17 | m5C | 5-methylcytidine | Dnmt2, Dnmt2, EfmM, Nop2, Rcm1, RlmI, |
| | | | RlmO, RsmB, RsmF, Trm4, nsun2 |
| 18 | ac4C | N4-acetylcytidine | NAT10, Rra1, TmcA |
| 19 | m1A | 1-methyladenosine | Bmt2, KamB, NpmA, Rrp8, TRMT10C, |
| | | | Trm61, TrmI, TrmK, Trmt61A, Trmt61B |
| 20 | tm5U | 5-taurinomethyluridine | MTU1 |
| 21 | m1G | 1-methylguanosine | AviRa, RImA(I), RlmA(II), TRM5, |
| | | | TRMT10A, TRMT10B, TRMT10C, Taw22, |
| | | | Trm10, Trm5, Trmb, TrmD |
| 22 | Cm | 2-O-methylcytidine | |
| 23 | m1I | 1-methylinosine | |
| 24 | Ar(p) | 2′O-ribosyladenosine (phosphate) | |
| 25 | galQtRNA | galactosyl-queuosine | |
| 26 | mcm5U | 5-methoxycarbonylmethyluridine | ALKBH8, Trm9, ELP1, ELP2, ELP3, |
| | | | ELP4, ELP5, ELP6 |
| 27 | m1Y | 1-methylpseudouridine | |
| 28 | Gm | 2′O-methylguanosine | MRM1, Mrm1, Nop1, RNMTL1, RlmB, |
| | | | Spb1, Trm3, Trm7, TrmH |
| 29 | manQtRNA | mannosyl-queuosine | Man/Gal-Q-transferase |
| 30 | yW | wybutosine | TYW1, 2, 3, 4 |
| 31 | f5C | 5-formylcytidine | MTU1 |
| 32 | tm5s2U | 5-taurinomethyl-2-thiouridine | TrmU |
| 33 | m2, 2G | N2,N2-dimethylguanosine | Trm1 |
| 34 | chm5U | 5-carboxyhydroxymethyluridine | |
| 35 | s2U | 2-thiouridine | MnmA, Mtu1, Ncs2, Ncs6, TrmU |
| 36 | mnm5s2U | 5-methylaminomethyl-2-thiouridine | MnmCD, MnmD, MnmA, Mtu1, TrmU |
| 37 | m6A | N6-methyladenosine | ErmAM, ErmBC, ErmC′, Ime4, METTL14, |
| | | | METTL3, RlmF, RlmJ, RsmA, TrmM |
| 38 | mchm5U | 5-(carboxyhydroxymethyl)uridine methyl | ALKBH8 |
| | | ester | |
| 39 | m2G | N2-methylguanosine | Trm112, Trm11 |
| 40 | cmnm5U | 5-carboxymethylaminomethyluridine | tRNA (cytidine(34)-2′-O)-methyltransferase |
| 41 | Ym | 2′O-methylpseudouridine | NEP1 |
| 42 | f5Cm | 5-formyl-2′-O-methylcytidine | |
| 43 | ncm5U | 5-carbamoylmethyluridine | ELP1, ELP2, ELP3, ELP4, ELP5, ELP6 |
| 44 | I | inosine | Tad1, Tad2, Tad3, TadA |
| 45 | g6A | N6-glycinylcarbamoyladenosine | METTL8 |
| 46 | cmnm5s2U | 5-carboxymethylaminomethyl-2- | MnmA, Mtu1, TrmU, MnmE, MnmG, |
| | | thiouridine | Mss1, Mto1 |
| 47 | Um | 2′O-methyluridine | AviRb, MRM2, Mrm2, Nop1, RlmE, Spb1, |
| | | | Trm44, TrmJ, TrmL, aTrm56 |
| 48 | Y | pseudouridine | Cbf5, Pus1, Pus10, Pus2, Pus3, Pus4, Pus5, |
| | | | Pus6, Pus7, Pus8, Pus9, RluA, RluB, RluC, |
| | | | RluD, RluE, RluF, TruA, TruB, TruC, TruD |
| 49 | ms2i6A | 2-methylthio-N6-isopentenyladenosine | MiaA |
| 50 | m3C | 3 -methylcytidine | Trm140, METTL2 and METTE6 |
| 51 | o2yW | peroxywybutosine | TRM5, TYW1, TYW2, TYW3, TYW4, |
| | | | TYW5, TRM4 |
| 52 | m5Um | 5,2′O-dimethyluridine | |
| 53 | ms2t6A | 2-methylthio-N6- | Yrdc/Sua5, MtaB/e-MtaB, SAM, “S” |
| | | threonylcarbamoyladenosine | |
| 54 | i6A | N6-isopentenyladenosine | MiaA, Mod5 |
| 55 | ms2io6A | 2-methylthio-N6-(cis- | MiaE |
| | | hydroxyisopentenyl) adenosine | |
| 56 | Am | 2_-O-methyladenosine | (2′-O-methyladenosine-N6-)- |
| | | | methyltransferase |
| 57 | m7G | 7-methylguanosine | Abd1, ArmA, Bud23, RlmKL, RmtB, |
| | | | RsmG, Sgm, TRMB, Trm8, TrmB, WDR4 |
| 58 | t6A | N6-threonylcarbamoyladenosine | Bud32, Gon7, Cgi121 |
| 59 | | N1-methylguanine | Trm10 |
| 60 | | N7-methylguanine | Trm8, Trm82 |
| 61 | | 2′-O methylribose | Trm3, Trm13, Trm44, Trm7, Trm732, |
| | | | Rtt10 |
| 62 | | Ribose 2′-O-ribosyl phosphate | Rit1 |

### TREM Fusion

In an embodiment, a TREM disclosed herein comprises an additional moiety, e.g., a fusion moiety. In an embodiment, the fusion moiety can be used for purification, to alter folding of the TREM, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM. In an embodiment, the fusion moiety can be encoded by the same or different nucleic acid molecule that encodes the TREM.

### TREM Consensus Sequence

In an embodiment, a TREM disclosed herein comprises a consensus sequence provided herein.

In an embodiment, a TREM disclosed herein comprises a consensus sequence of Formula I_{ZZZ}, wherein _{ZZZ} indicates any of the twenty amino acids and Formula I corresponds to all species.

In an embodiment, a TREM disclosed herein comprises a consensus sequence of Formula II_{ZZZ}, wherein _{ZZZ} indicates any of the twenty amino acids and Formula II corresponds to mammals.

In an embodiment, a TREM disclosed herein comprises a consensus sequence of Formula III_{ZZZ}, wherein _{ZZZ} indicates any of the twenty amino acids and Formula III corresponds to humans.

In an embodiment, _{ZZZ} indicates any of the twenty amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an embodiment, a TREM disclosed herein comprises a property selected from the following:

a) under physiological conditions residue R₀ forms a linker region, e.g., a Linker 1 region;

b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;

c) under physiological conditions residues R₈-R₉ forms a linker region, e.g., a Linker 2 region;

d) under physiological conditions residues -R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region; e) under physiological conditions residue -R₂₉ forms a linker region, e.g., a Linker 3 Region;

f) under physiological conditions residues -R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;

g) under physiological conditions residue -[R₄₇]ₓ comprises a variable region, e.g., as described herein;

h) under physiological conditions residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or

i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein R is a ribonucleotide residue and the consensus for Ala is:
R₀=absent;
R₁₄, R₅₇=are independently A or absent;
R₂₆=A, C, G or absent;
R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉,
R₆₃, R₆₄, R₆₆, R₆₇=are independently N or absent;
R₁₁, R₃₅, R₆₅=are independently A, C, U or absent;
R₁, R₉, R₂₀, R₃₈, R₄₀, R₅₁, R₅₂, R₅₆=are independently A, G or absent;
R₇, R₂₂, R₂₅, R₂₇, R₂₉, R₄₆, R₅₃, R₇₂=are independently A, G, U or absent;
R₂₄, R₆₉=are independently A, U or absent;
R₇₀, R₇₁=are independently C or absent;
R₃, R₄=are independently C, G or absent;
R₁₂, R₃₃, R₃₆, R₆₂, R₆₈=are independently C, G, U or absent;
R₁₃, R₁₇, R₂₈, R₃₉, R₅₅, R₆₀, R₆₁=are independently C, U or absent;
R₁₀, R₁₉, R₂₃=are independently G or absent;
R₂=G, U or absent;
R₈, R₁₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA} (SEQ ID NO: 564),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I A_{R}G (SEQ ID NO: 565),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₅₇=A or absent;
R₉,R₂₇=are independently A, C, G or absent;
R₁,R₂,R₃,R₄,R₅,R₆,R₇,R₁₁,R₁₂,R₁₆,R₂₁,R₂₂,R₂₃,R₂₅,R₂₆,R₂₉,R₃₀,R₃₁,R₃₂,R₃₃,R₃₄,R₃₇,R₄₂,R₄₄,R₄₅, R₄₆,R₄₈,R₄₉,R₅₀,R₅₁,R₅₈,R₆₂,R₆₃,R₆₄,R₆₈,R₆₆,R₆₇,R₆₈,R₆₉,R₇₀,R₇₁=are independently N or absent;
R₁₃,R₁₇,R₄₁=are independently A, C, U or absent;
R₁₉,R₂₀,R₂₄,R₄₀,R₅₆=are independently A, G or absent;
R₁₄,R₁₅,R₇₂=are independently A, G, U or absent;
R₁₈=A, U or absent;
R₃₈=C or absent;
R₃₅,R₄₃,R₆₁=are independently C, G, U or absent;
R₂₈,R₅₅,R₅₉,R₆₀=are independently C, U or absent;
R₀,R₁₀,R₅₂=are independently G or absent;
R₈,R₃₉=are independently G, U or absent;
R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II A_{R}G (SEQ ID NO: 566),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=absent;
R₂₄,R₅₇=are independently A or absent;
R₄₁=A, C or absent;
R₃,R₇,R₃₄,R₅₀=are independently A, C, G or absent;
R₂,R₅,R₆,R₁₂,R₂₆,R₃₂,R₃₇,R₄₄,R₅₈,R₆₆,R₆₇,R₆₈,R₇₀=are independently N or absent;
R₄₉,R₇₁=are independently A, C, U or absent;
R₁,R₁₅,R₁₉,R₂₅,R₂₇,R₄₀,R₄₅,R₄₆,R₅₆,R₇₂=are independently A, G or absent;
R₁₄,R₂₉,R₆₃=are independently A, G, U or absent;
R₁₆,R₂₁=are independently A, U or absent;
R₃₈,R₆₁=are independently C or absent;
R₃₃,R₄₈=are independently C, G or absent;
R₄,R₉,R₁₁,R₄₃,R₆₂,R₆₄,R₆₉=are independently C, G, U or absent;
R₁₃,R₂₂,R₂₈,R₃₀,R₃₁,R₃₅,R₅₅,R₆₀,R₆₅=are independently C, U or absent;
R₀,R₁₀,R₂₀,R₂₃,R₅₁,R₅₂=are independently G or absent;
R₈,R₃₉,R₄₂=are independently G, U or absent;
R₁₇,R₃₆,R₅₃,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III A_{R}G (SEQ ID NO: 567),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=is absent;
R₁₅,R₂₁,R₂₄,R₄₁,R₅₇=are independently A or absent;
R₃₄,R₄₄=are independently A, C or absent;
R₃,R₅,R₅₈=are independently A, C, G or absent;
R₂,R₆,R₆₆,R₇₀=are independently N or absent;
R₃₇,R₄₉=are independently A, C, U or absent;
R₁,R₂₅,R₂₉,R₄₀,R₄₅,R₄₆,R₅₀=are independently A, G or absent;
R₁₄,R₆₃,R₆₈=are independently A, G, U or absent;
R₁₆=A, U or absent;
R₃₈,R₆₁=are independently C or absent;
R₇,R₁₁,R₁₂,R₂₆,R₄₈=are independently C, G or absent;
R₆₄,R₆₇,R₆₉=are independently C, G, U or absent;
R₄,R₁₃,R₂₂,R₂₈,R₃₀,R₃₁,R₃₅,R₄₃,R₅₈,R₆₀,R₆₂,R₆₈,R₇₁=are independently C, U or absent;
R₀,R₁₀,R₁₉,R₂₀,R₂₃,R₂₇,R₃₃,R₅₁,R₅₂,R₅₆,R₇₂=are independently G or absent;
R₈,R₉,R₃₂,R₃₉,R₄₂=are independently G, U or absent;
R₁₇,R₃₆,R₅₃,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I_{ASN} (SEQ ID NO: 568),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀,R₁₈=are absent;
R₄₁=A or absent;
R₁₄,R₄₈,R₅₆=are independently A, C, G or absent;
R₂,R₄,R₅,R₆,R₁₂,R₁₇,R₂₆,R₂₉,R₃₀,R₃₁,R₄₄,R₄₅,R₄₆,R₄₉,R₅₀,R₅₈,R₆₂,R₆₃,R₆₅,R₆₆,R₆₇,R₆₈,R₇₀,R₇₁=are independently N or absent;
R₁₁,R₁₃,R₂₂,R₄₂,R₅₅,R₅₉=are independently A, C, U or absent;
R₉,R₁₅,R₂₄,R₂₇,R₃₄,R₃₇,R₅₁,R₇₂=are independently A, G or absent;
R₁,R₇,R₂₅,R₆₉=are independently A, G, U or absent;
R₄₀,R₅₇=are independently A, U or absent;
R₆₀=C or absent;
R₃₃=C, G or absent;
R₂₁,R₃₂,R₄₃,R₆₄=are independently C, G, U or absent;
R₃,R₁₆,R₂₈,R₃₅,R₃₆,R₆₁=are independently C, U or absent;
R₁₀,R₁₉,R₂₀,R₅₂=are independently G or absent;
R₅₄=G, U or absent;
R₈,R₂₃,R₃₈,R₃₉,R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASN} (SEQ ID NO: 569),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀,R₁₈=are absent
R₂₄,R₄₁,R₄₆,R₆₂=are independently A or absent;
R₅₉=A, C or absent;
R₁₄,R₅₆,R₆₆=are independently A, C, G or absent;
R₁₇,R₂₉=are independently N or absent;
R₁₁,R₂₆,R₄₂,R₅₅=are independently A, C, U or absent;
R₁,R₉,R₁₂,R₁₅,R₂₅,R₃₄,R₃₇,R₄₈,R₅₁,R₆₇,R₆₈,R₆₉,R₇₀,R₇₂=are independently A, G or absent;
R₄₄,R₄₅,R₅₈=are independently A, G, U or absent;
R₄₀,R₅₇=are independently A, U or absent;
R₅,R₂₈,R₆₀=are independently C or absent;
R₃₃,R₆₅=are independently C, G or absent;
R₂₁,R₄₃,R₇₁=are independently C, G, U or absent;
R₃,R₆,R₁₃,R₂₂,R₃₂,R₃₅,R₃₆,R₆₁,R₆₃,R₆₄=are independently C, U or absent;
R₇,R₁₀,R₁₉,R₂₀,R₂₇,R₄₉,R₅₂=are independently G or absent;
R₅₄=G, U or absent;
R₂,R₄,R₅,R₁₆,R₂₃,R₃₀,R₃₁,R₃₈,R₃₉,R₅₀,R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASN} (SEQ ID NO: 570),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀,R₁₈=are absent
R₂₄,R₄₀,R₄₁,R₄₆,R₆₂=are independently A or absent;
R₅₉=A, C or absent;
R₁₄,R₅₆,R₆₆=are independently A, C, G or absent;
R₁₁,R₂₆,R₄₂,R₅₅=are independently A, C, U or absent;
R₁,R₉,R₁₂,R₁₅,R₃₄,R₃₇,R₄₈,R₅₁,R₆₇,R₆₈,R₆₉,R₇₀=are independently A, G or absent;
R₄₄,R₄₅,R₅₈=are independently A, G, U or absent;
R₅₇=A, U or absent;
R₅,R₂₈,R₆₀=are independently C or absent;
R₃₃,R₆₅=are independently C, G or absent;
R₁₇,R₂₁,R₂₉=are independently C, G, U or absent;
R₃,R₆,R₁₃,R₂₂,R₃₂,R₃₅,R₃₆,R₄₃,R₆₁,R₆₃,R₆₄,R₇₁=are independently C, U or absent;
R₇,R₁₀,R₁₉,R₂₀,R₂₅,R₂₇,R₄₉,R₅₂,R₇₂=are independently G or absent;
R₅₄=G, U or absent;
R₂,R₄,R₈,R₁₆,R₂₃,R₃₀,R₃₁,R₃₈,R₃₉,R₅₀,R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I ASP (SEQ ID NO: 571),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀=absent
R₂₄,R₇₁=are independently A, C or absent;
R₃₃,R₄₆=are independently A, C, G or absent;
R₂,R₃,R₄,R₅,R₆,R₁₂,R₁₆,R₂₂,R₂₆,R₂₉,R₃₁,R₃₂,R₄₄,R₄₈,R₄₉,R₅₈,R₆₃,R₆₄,R₆₆,R₆₇,R₆₈,R₆₉=are independently N or absent;
R₁₃,R₂₁,R₃₄,R₄₁,R₅₇,R₆₅=are independently A, C, U or absent;
R₉,R₁₀,R₁₄,R₁₅,R₂₀,R₂₇,R₃₇,R₄₀,R₅₁,R₅₆,R₇₂=are independently A, G or absent;
R₇,R₂₅,R₄₂=are independently A, G, U or absent;
R₃₉=C or absent;
R₅₀,R₆₂=are independently C, G or absent;
R₃₀,R₄₃,R₄₅,R₅₅,R₇₀=are independently C, G, U or absent;
R₈,R₁₁,R₁₇,R₁₈,R₂₈,R₃₅,R₅₃,R₅₉,R₆₀,R₆₁=are independently C, U or absent;
R₁₉,R₅₂=are independently G or absent;
R₁=G, U or absent;
R₂₃,R₃₆,R₃₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ASP (SEQ ID NO: 572),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀,R₁₇,R₁₈,R₂₃=are independently absent;
R₉,R₄₀=are independently A or absent;
R₂₄,R₇₁=are independently A, C or absent;
R₆₇,R₆₈=are independently A, C, G or absent;
R₂,R₆,R₆₆=are independently N or absent;
R₅₇,R₆₃=are independently A, C, U or absent;
R₁₀,R₁₄,R₂₇,R₃₃,R₃₇,R₄₄,R₄₆,R₅₁,R₅₆,R₆₄,R₇₂=are independently A, G or absent;
R₇,R₁₂,R₂₆,R₆₅=are independently A, U or absent;
R₃₉,R₆₁,R₆₂=are independently C or absent;
R₃,R₃₁,R₄₅,R₇₀=are independently C, G or absent;
R₄,R₅,R₂₉,R₄₃,R₅₅=are independently C, G, U or absent;
R₈,R₁₁,R₁₃,R₃₀,R₃₂,R₃₄,R₃₅,R₄₁,R₄₈,R₅₃,R₅₉,R₆₀=are independently C, U or absent;
R₁₅,R₁₉,R₂₀,R₂₅,R₄₂,R₅₀,R₅₂=are independently G or absent;
R₁,R₂₂,R₄₉,R₅₈,R₆₉=are independently G, U or absent;
R₁₆,R₂₁,R₂₈,R₃₆,R₃₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASP (SEQ ID NO: 573),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀,R₁₇,R₁₈,R₂₃=are absent
R₉,R₁₂,R₄₀,R₆₅,R₇₁=are independently A or absent;
R₂,R₂₄,R₅₇=are independently A, C or absent;
R₆,R₁₄,R₂₇,R₄₆,R₅₁,R₅₆,R₆₄,R₆₇,R₆₈=are independently A, G or absent;
R₃,R₃₁,R₃₅,R₃₉,R₆₁,R₆₂=are independently C or absent;
R₆₆=C, G or absent;
R₅,R₈,R₂₉,R₃₀,R₃₂,R₃₄,R₄₁,R₄₃,R₄₈,R₅₅,R₅₉,R₆₀,R₆₃=are independently C, U or absent;
R₁₀,R₁₅,R₁₉,R₂₀,R₂₅,R₃₃,R₃₇,R₄₂,R₄₄,R₄₅,R₄₉,R₅₀,R₅₂,R₆₉,R₇₀,R₇₂=are independently G or absent;
R₂₂,R₅₈=are independently G, U or absent;
R₁,R₄,R₇,R₁₁,R₁₃,R₁₆,R₂₁,R₂₆,R₂₈,R₃₆,R₃₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I_{CYS} (SEQ ID NO: 574),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀=absent
R₁₄,R₃₉,R₅₇=are independently A or absent;
R₄₁=A, C or absent;
R₁₀,R₁₅,R₂₇,R₃₃,R₆₂=are independently A, C, G or absent;
R₃,R₄,R₅,R₆,R₁₂,R₁₃,R₁₆,R₂₄,R₂₆,R₂₉,R₃₀,R₃₁,R₃₂,R₃₄,R₄₂,R₄₄,R₄₅,R₄₆,R₄₈,R₄₉,R₅₈,R₆₃,R₆₄,R₆₆,
R₆₇,R₆₈,R₆₉,R₇₀=are independently N or absent;
R₆₅=A, C, U or absent;
R₉,R₂₅,R₃₇,R₄₀,R₅₂,R₅₆=are independently A, G or absent;
R₇,R₂₀,R₅₁=are independently A, G, U or absent;
R₁₈,R₃₈,R₅₅=are independently C or absent;
R₂=C, G or absent;
R₂₁,R₂₈,R₄₃,R₅₀=are independently C, G, U or absent;
R₁₁,R₂₂,R₂₃,R₃₅,R₃₆,R₅₉,R₆₀,R₆₁,R₇₁,R₇₂=are independently C, U or absent;
R₁,R₁₉=are independently G or absent;
R₁₇=G, U or absent;
R₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{CYS} (SEQ ID NO: 575),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein R is a ribonucleotide residue and the consensus for Cys is:
R₀,R₁₈,R₂₃=are absent;
R₁₄,R₂₄,R₂₆,R₂₉,R₃₉,R₄₁,R₄₅,R₅₇=are independently A or absent;
R₄₄=A, C or absent;
R₂₇,R₆₂=are independently A, C, G or absent;
R₁₆=A, C, G, U or absent;
R₃₀,R₇₀=are independently A, C, U or absent;
R₅,R₇,R₉,R₂₅,R₃₄,R₃₇,R₄₀,R₄₆,R₅₂,R₅₆,R₅₈,R₆₆=are independently A, G or absent;
R₂₀,R₅₁=are independently A, G, U or absent;
R₃₅,R₃₈,R₄₃,R₅₅,R₆₉=are independently C or absent;
R₂,R₄,R₁₅=are independently C, G or absent;
R₁₃=C, G, U or absent;
R₆,R₁₁,R₂₈,R₃₆,R₄₈,R₄₉,R₅₀,R₆₀,R₆₁,R₆₇,R₆₈,R₇₁,R₇₂=are independently C, U or absent;
R₁,R₃,R₁₀,R₁₉,R₃₃,R₆₃=are independently G or absent;
R₈,R₁₇,R₂₁,R₆₄=are independently G, U or absent;
R₁₂,R₂₂,R₃₁,R₃₂,R₄₂,R₅₃,R₅₄,R₆₅=are independently U or absent;
R₅₉=U, or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{CYS} (SEQ ID NO: 576),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀,R₁₈,R₂₃=are absent
R₁₄,R₂₄,R₂₆,R₂₉,R₃₄,R₃₉,R₄₁,R₄₅,R₅₇,R₅₈=are independently A or absent;
R₄₄,R₇₀=are independently A, C or absent;
R₆₂=A, C, G or absent;
R₁₆=N or absent;
R₅,R₇,R₉,R₂₀,R₄₀,R₄₆,R₅₁,R₅₂,R₅₆,R₆₆=are independently A, G or absent;
R₂₈,R₃₅,R₃₈,R₄₃,R₅₅,R₆₇,R₆₉=are independently C or absent;
R₄,R₁₅=are independently C, G or absent;
R₆,R₁₁,R₃,R₃₀,R₄₈,R₄₉,R₅₀,R₆₀,R₆₁,R₆₈,R₇₁,R₇₂=are independently C, U or absent;
R₁,R₂,R₃,R₁₀,R₁₉,R₂₅,R₂₇,R₃₃,R₃₇,R₆₃=are independently G or absent;
R₈,R₂₁,R₆₄=are independently G, U or absent;
R₁₂,R₁₇,R₂₂,R₃₁,R₃₂,R₃₆,R₄₂,R₅₃,R₅₄, R₅₉,R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I_{GLN}(SEQ ID NO: 577),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀,R₁₈=are absent;
R₁₄,R₂₄,R₅₇=are independently A or absent;
R₉,R₂₆,R₂₇,R₃₃,R₅₆=are independently A, C, G or absent;
R₂,R₄,R₅,R₆,R₁₂,R₁₃,R₁₆,R₂₁,R₂₂,R₂,R₂₉,R₃₀,R₃₁,R₃₂,R₃₄,R₄₁,R₄₂,R₄₄,R₄₅,R₄₆,R₄₈,R₄₉,R₅₀,R₅₈,R₆₂,R₆₃,R₆₆,R₆₇,R₆₈,R₆₉,R₇₀=are independently N or absent;
R₁₇,R₂₃,R₄₃,R₆₅,R₇₁=are independently A, C, U or absent;
R₁₅,R₄₀,R₅₁,R₅₂=are independently A, G or absent;
R₁,R₇,R₇₂=are independently A, G, U or absent;
R₃,R₁₁,R₃₇,R₆₀,R₆₄=are independently C, G, U or absent;
R₂₈,R₃₅,R₅₅,R₅₉,R₆₁=are independently C, U or absent;
R₁₀,R₁₉,R₂₀=are independently G or absent;
R₃₉=G, U or absent;
R₈,R₃₆,R₃₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLN} (SEQ ID NO: 578),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀,R₁₈,R₂₃=are absent
R₁₄,R₂₄,R₅₇=are independently A or absent;
R₁₇,R₇₁=are independently A, C or absent;
R₂₅,R₂₆,R₃₃,R₄₄,R₄₆,R₅₆,R₆₉=are independently A, C, G or absent;
R₄,R₅,R₁₂,R₂₂,R₂₉,R₃₀,R₄₈,R₄₉,R₆₃,R₆₇,R₆₈=are independently N or absent;
R₃₁,R₄₃,R₆₂,R₆₅,R₇₀=are independently A, C, U or absent;
R₁₅,R₂₇,R₃₄,R₄₀,R₄₁,R₅₁,R₅₂=are independently A, G or absent;
R₂,R₇,R₂₁,R₄₅,R₅₀,R₅₈,R₆₆,R₇₂=are independently A, G, U or absent;
R₃,R₁₃,R₃₂,R₃₇,R₄₂,R₆₀,R₆₄=are independently C, G, U or absent;
R₆,R₁₁,R₂₈,R₃₅,R₅₅,R₅₉,R₆₁=are independently C, U or absent;
R₉,R₁₀,R₁₉,R₂₀=are independently G or absent;
R₁,R₁₆,R₃₉=are independently G, U or absent;
R₈,R₃₆,R₃₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLN} (SEQ ID NO: 579),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀,R₁₈,R₂₃=are absent
R₁₄,R₂₄,R₄₁,R₅₇=are independently A or absent;
R₁₇,R₇₁=are independently A, C or absent;
R₅,R₂₅,R₂₆,R₄₆,R₅₆,R₆₉=are independently A, C, G or absent;
R₄,R₂₂,R₂₉,R₃₀,R₄₈,R₄₉,R₆₃,R₆₈=are independently N or absent;
R₄₃,R₆₂,R₆₅,R₇₀=are independently A, C, U or absent;
R₁₅,R₂₇,R₃₃,R₃₄,R₄₀,R₅₁,R₅₂=are independently A, G or absent;
R₂,R₇,R₁₂,R₄₅,R₅₀,R₅₈,R₆₆=are independently A, G, U or absent;
R₃₁=A, U or absent;
R₃₂,R₄₄,R₆₀=are independently C, G or absent;
R₃,R₁₃,R₃₇,R₄₂,R₆₄,R₆₇=are independently C, G, U or absent;
R₆,R₁₁,R₂₈,R₃₅,R₅₅,R₅₉,R₆₁=are independently C, U or absent;
R₉,R₁₀,R₁₉,R₂₀=are independently G or absent;
R₁,R₂₁,R₃₉,R₇₂=are independently G, U or absent;
R₈,R₁₆,R₃₆,R₃₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I_{GLU} (SEQ ID NO: 580),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀=absent;
R₃₄,R₄₃,R₆₈,R₆₉=are independently A, C, G or absent;
R₁,R₂,R₅,R₆,R₉,R₁₂,R₁₆,R₂₀,R₂₁,R₂₆,R₂₇,R₂₉,R₃,R₃₁,R₃₂,R₃₃,R₄₁,R₄₄,R₄₅,R₄₆,R₄₈,R₅₀,R₅₁,R₅₅,R₆₃,R₆₄,R₆₅,R₆₆,R₇₀,R₇₁=are independently N or absent;
R₁₃,R₁₇,R₂₃,R₆₁=are independently A, C, U or absent;
R₁₀,R₁₄,R₂₄,R₄₀,R₅₂,R₅₆=are independently A, G or absent;
R₇,R₁₅,R₂₅,R₆₇,R₇₂=are independently A, G, U or absent;
R₁₁,R₅₇=are independently A, U or absent;
R₃₉=C, G or absent;
R₃,R₄,R₂₂,R₄₂,R₄₉,R₅₅,R₆₂=are independently C, G, U or absent;
R₁₈,R₂₈,R₃₅,R₃₇,R₅₃,R₅₉,R₆₀=are independently C, U or absent; R₁₉=G or absent;
R₈,R₃₆,R₃₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLU} (SEQ ID NO: 581),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀,R₁₈,R₂₃=are absent
R₁₇,R₄₀=are independently A or absent;
R₂₆,R₂₇,R₃₄,R₄₃,R₆₈,R₆₉,R₇₁=are independently A, C, G or absent;
R₁,R₂,R₅,R₁₂,R₂₁,R₃₁,R₃₃,R₄₁,R₄₅,R₄₈,R₅₁,R₅₈,R₆₆,R₇₀=are independently N or absent;
R₄₄,R₆₁=are independently A, C, U or absent;
R₉,R₁₄,R₂₄,R₂₅,R₅₂,R₅₆,R₆₃=are independently A, G or absent;
R₇,R₁₅,R₄₆,R₅₀,R₆₇,R₇₂=are independently A, G, U or absent;
R₂₉,R₅₇=are independently A, U or absent;
R₆₀=C or absent;
R₃₉=C, G or absent;
R₃,R₆,R₂₀,R₃₀,R₃₂,R₄₂,R₅₅,R₆₂,R₆₅=are independently C, G, U or absent;
R₄,R₈,R₁₆,R₂₈,R₃₅,R₃₇,R₄₉,R₅₃,R₅₉=are independently C, U or absent;
R₁₀,R₁₉=are independently G or absent;
R₂₂,R₆₄=are independently G, U or absent;
R₁₁,R₁₃,R₃₆,R₃₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLU} (SEQ ID NO: 582),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀,R₁₇,R₁₈,R₂₃=are absent
R₁₄,R₂₇,R₄₀,R₇₁=are independently A or absent;
R₄₄=A, C or absent;
R₄₃=A, C, G or absent;
R₁,R₃₁,R₃₃,R₄₅,R₅₁,R₆₆=are independently N or absent;
R₂₁,R₄₁=are independently A, C, U or absent;
R₇,R₂₄,R₂₅,R₅₀,R₅₂,R₅₆,R₆₃,R₆₈,R₇₀=are independently A, G or absent;
R₅,R₄₆=are independently A, G, U or absent;
R₂₉,R₅₇,R₆₇,R₇₂=are independently A, U or absent;
R₂,R₃₉,R₆₀=are independently C or absent;
R₃,R₁₂,R₂₀,R₂₆,R₃₄,R₆₉=are independently C, G or absent;
R₆,R₃₀,R₄₂,R₄₈,R₆₉=are independently C, G, U or absent;
R₄,R₁₆,R₂₈,R₃₅,R₃₇,R₄₉,R₅₃,R₅₅,R₅₈,R₆₁,R₆₂=are independently C, U or absent;
R₉,R₁₀,R₁₉,R₆₄=are independently G or absent;
R₁₅,R₂₂,R₃₂=are independently G, U or absent;
R₈,R₁₁,R₁₃,R₃₆,R₃₈,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I_{GLY}(SEQ ID NO: 583),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀=absent;
R₂₄=A or absent;
R₃,R₉,R₄₀,R₅₀,R₅₁=are independently A, C, G or absent;
R₄,R₅,R₆,R₇,R₁₂,R₁₆,R₂₁,R₂₂,R₂₆,R₂₉,R₃₀,R₃₁,R₃₂,R₃₃,R₃₄,R₄₁,R₄₂,R₄₃,R₄₄,R₄₅,R₄₆,R₄₈,R₄₉,R₅₈,R₆₃,R₆₄,R₆₅,R₆₆,R₆₇,R₆₈=are independently N or absent;
R₅₉=A, C, U or absent;
R₁,R₁₀,R₁₄,R₁₅,R₂₇,R₅₆=are independently A, G or absent;
R₂₀,R₂₅=are independently A, G, U or absent;
R₅₇,R₇₂=are independently A, U or absent;
R₃₈,R₃₉,R₆₀=are independently C or absent;
R₅₂=C, G or absent;
R₂,R₁₉,R₃₇,R₅₄,R₅₅,R₆₁,R₆₂,R₆₉,R₇₀=are independently C, G, U or absent;
R₁₁,R₁₃,R₁₇,R₂₈,R₃₅,R₃₆,R₇₁=are independently C, U or absent;
R₈,R₁₈,R₂₃,R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLY} (SEQ ID NO: 584),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for Gly is:
R₀,R₁₈,R₂₃=are absent
R₂₄,R₂₇,R₄₀,R₇₂=are independently A or absent;
R₂₆=A, C or absent;
R₃,R₇,R₆₈=are independently A, C, G or absent;
R₅,R₃₀,R₄₁,R₄₂,R₄₄,R₄₉,R₆₇=are independently A, C, G, U or absent;
R₃₁,R₃₂,R₃₄=are independently A, C, U or absent;
R₉,R₁₀,R₁₄,R₁₅,R₃₃,R₅₀,R₅₆=are independently A, G or absent;
R₁₂,R₁₆,R₂₂,R₂₅,R₂₉,R₄₆=are independently A, G, U or absent;
R₅₇=A, U or absent;
R₁₇,R₃₈,R₃₉,R₆₀,R₆₁,R₇₁=are independently C or absent;
R₆,R₅₂,R₆₄,R₆₆=are independently C, G or absent;
R₂,R₄,R₃₇,R₄₈,R₅₅,R₆₅=are independently C, G, U or absent;
R₁₃,R₃₅,R₄₃,R₆₂,R₆₉=are independently C, U or absent;
R₁,R₁₉,R₂₀,R₅₁,R₇₀=are independently G or absent;
R₂₁,R₄₅,R₆₃=are independently G, U or absent;
R₈,R₁₁,R₂₈,R₃₆,R₅₃,R₅₄,R₅₈,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLY} (SEQ ID NO: 585),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀,R₁₈,R₂₃=are absent
R₂₄,R₂₇,R₄₀,R₇₂=are independently A or absent;
R₂₆=A, C or absent;
R₃,R₇,R₄₉,R₆₈=are independently A, C, G or absent;
R₅,R₃₀,R₄₁,R₄₄,R₆₇=are independently N or absent;
R₃₁,R₃₂,R₃₄=are independently A, C, U or absent;
R₉,R₁₀,R₁₄,R₁₅,R₃₃,R₅₀,R₅₆=are independently A, G or absent;
R₁₂,R₂₅,R₂₉,R₄₂,R₄₆=are independently A, G, U or absent;
R₁₆,R₅₇=are independently A, U or absent;
R₁₇,R₃₈,R₃₉,R₆₀,R₆₁,R₇₁=are independently C or absent;
R₆,R₅₂,R₆₄,R₆₆=are independently C, G or absent;
R₃₇,R₄₈,R₆₅=are independently C, G, U or absent;
R₂,R₄,R₁₃,R₃₅,R₄₃,R₅₅,R₆₂,R₆₉=are independently C, U or absent;
R₁,R₁₉,R₂₀,R₅₁,R₇₀=are independently G or absent;
R₂₁,R₂₂,R₄₅,R₆₃=are independently G, U or absent;
R₈,R₁₁,R₂₈,R₃₆,R₅₃,R₅₄,R₅₈,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I_{HIS} (SEQ ID NO: 586),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₂₃=absent;
R₁₄,R₂₄,R₅₇=are independently A or absent;
R₇₂=A, C or absent;
R₉,R₂₇,R₄₃,R₄₈,R₆₉=are independently A, C, G or absent;
R₃,R₄,R₅,R₆,R₁₂,R₂₅,R₂₆,R₂₉,R₃₀,R₃₁,R₃₄,R₄₂,R₄₅,R₄₆,R₄₉,R₅₀,R₅₈,R₆₂,R₆₃,R₆₆,R₆₇,R₆₈=are independently N or absent;
R₁₃,R₂₁,R₄₁,R₄₄,R₆₅=are independently A, C, U or absent;
R₄₀,R₅₁,R₅₆,R₇₀=are independently A, G or absent;
R₇,R₃₂=are independently A, G, U or absent;
R₅₅,R₆₀=are independently C or absent;
R₁₁,R₁₆,R₃₃,R₆₄=are independently C, G, U or absent;
R₂,R₁₇,R₂₂,R₂₈,R₃₅,R₅₃,R₅₉,R₆₁,R₇₁=are independently C, U or absent;
R₁,R₁₀,R₁₅,R₁₉,R₂,R₃₇,R₃₉,R₅₂=are independently G or absent; R₀=G, U or absent;
R₈,R₁₈,R₃₆,R₃₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{HIS} (SEQ ID NO: 587),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀,R₁₇,R₁₈,R₂₃=are absent;
R₇,R₁₂,R₁₄,R₂₄,R₂₇,R₄₅,R₅₇,R₅₈,R₆₃,R₆₇,R₇₂=are independently A or absent;
R₃=A, C, U or absent;
R₄,R₄₃,R₅₆,R₇₀=are independently A, G or absent;
R₄₉=A, U or absent;
R₂,R₂₈,R₃₀,R₄₁,R₄₂,R₄₄,R₄₈,R₅₅,R₆₀,R₆₆,R₇₁=are independently C or absent;
R₂₅=C, G or absent;
R₉=C, G, U or absent;
R₈,R₁₃,R₂₆,R₃₃,R₃₅,R₅₀,R₅₃,R₆₁,R₆₈=are independently C, U or absent;
R₁,R₆,R₁₀,R₁₅,R₁₉,R₂₀,R₃₂,R₃₄,R₃₇,R₃₉,R₄₀,R₄₆,R₅₁,R₅₂,R₆₂,R₆₄,R₆₉=are independently G or absent;
R₁₆=G, U or absent;
R₅,R₁₁,R₂₁,R₂₂,R₂₉,R₃₁,R₃₆,R₃₈,R₅₄,R₅₉,R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{HIS} (SEQ ID NO: 588),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀,R₁₇,R₁₈,R₂₃=are absent
R₇,R₁₂,R₁₄,R₂₄,R₂₇,R₄₅,R₅₇,R₅₈,R₆₃,R₆₇,R₇₂=are independently A or absent;
R₃=A, C or absent;
R₄,R₄₃,R₅₆,R₇₀=are independently A, G or absent;
R₄₉=A, U or absent;
R₂,R₂₈,R₃₀,R₄₁,R₄₂,R₄₄,R₄₈,R₅₅,R₆₀,R₆₆,R₇₁=are independently C or absent;
R₈,R₉,R₂₆,R₃₃,R₃₅,R₅₀,R₆₁,R₆₈=are independently C, U or absent;
R₁,R₆,R₁₀,R₁₅,R₁₉,R₂₀,R₂₅,R₃₂,R₃₄,R₃₇,R₃₉,R₄₀,R₄₆,R₅₁,R₅₂,R₆₂,R₆₄,R₆₉=are independently G or absent;
R₅,R₁₁,R₁₃,R₁₆,R₂₁,R₂₂,R₂₉,R₃₁,R₃₆,R₃₈,R₅₃,R₅₄,R₅₉,R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I_{ILE} (SEQ ID NO: 589),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₂₃=absent;
R₃₈,R₄₁,R₅₇,R₇₂=are independently A or absent;
R₁,R₂₆=are independently A, C, G or absent; R₀,R₃,R₄,R₆,R₁₆,R₃₁,R₃₂,R₃₄,R₃₇,R₄₂,R₄₃,R₄₄,R₄₅,R₄₆,R₄₈,R₄₉,R₅₀,R₅₅,R₅₉,R₆₂,R₆₃,R₆₄,R₆₆,R₆₇,R₆₈,R₆₉=are independently N or absent;
R₂₂,R₆₁,R₆₅=are independently A, C, U or absent;
R₉,R₁₄,R₁₅,R₂₄,R₂₇,R₄₀=are independently A, G or absent;
R₇,R₂₅,R₂₉,R₅₁,R₅₆=are independently A, G, U or absent;
R₁₈,R₅₄=are independently A, U or absent;
R₆₀=C or absent;
R₂,R₅₂,R₇₀=are independently C, G or absent;
R₅,R₁₂,R₂₁,R₃₀,R₃₃,R₇₁=are independently C, G, U or absent;
R₁₁,R₁₃,R₁₇,R₂₈,R₃₅,R₅₃,R₅₅=are independently C, U or absent;
R₁₀,R₁₉,R₂₀=are independently G or absent;
R₈,R₃₆,R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ILE (SEQ ID NO: 590),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀,R₁₈,R₂₃=are absent
R₂₄,R₃₈,R₄₀,R₄₁,R₅₇,R₇₂=are independently A or absent;
R₂₆,R₆₅=are independently A, C or absent;
R₅₈,R₅₉,R₆₇=are independently N or absent;
R₂₂=A, C, U or absent;
R₆,R₉,R₁₄,R₁₅,R₂₉,R₃₄,R₄₃,R₄₆,R₄₈,R₅₀,R₅₁,R₆₃,R₆₉=are independently A, G or absent;
R₃₇,R₅₆=are independently A, G, U or absent;
R₅₄=A, U or absent;
R₂₈,R₃₅,R₆₀,R₆₂,R₇₁=are independently C or absent;
R₂,R₅₂,R₇₀=are independently C, G or absent;
R₅=C, G, U or absent;
R₃,R₄,R₁₁,R₁₃,R₁₇,R₂₁,R₃₀,R₄₂,R₄₄,R₄₅,R₄₉,R₅₃,R₅₅,R₆₁,R₆₄,R₆₆=are independently C, U or absent;
R₁,R₁₀,R₁₉,R₂₀,R₂₅,R₂₇,R₃₁,R₆₈=are independently G or absent;
R₇,R₁₂,R₃₂=are independently G, U or absent;
R₈,R₁₆,R₃₃,R₃₆,R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ILE (SEQ ID NO: 591),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀,R₁₈,R₂₃=are absent
R₁₄,R₂₄,R₃₈,R₄₀,R₄₁,R₅₇,R₇₂=are independently A or absent;
R₂₆,R₆₅=are independently A, C or absent;
R₂₂,R₅₉=are independently A, C, U or absent;
R₆,R₉,R₁₅,R₃₄,R₄₃,R₄₆,R₅₁,R₅₆,R₆₃,R₆₉=are independently A, G or absent;
R₃₇=A, G, U or absent;
R₁₃,R₂₈,R₃₅,R₄₄,R₅₅,R₆₀,R₆₂,R₇₁=are independently C or absent;
R₂,R₅,R₇₀=are independently C, G or absent;
R₅₈,R₆₇=are independently C, G, U or absent;
R₃,R₄,R₁₁,R₁₇,R₂₁,R₃₀,R₄₂,R₄₅,R₄₉,R₅₃,R₆₁,R₆₄,R₆₆=are independently C, U or absent;
R₁,R₁₀,R₁₉,R₂₀,R₂₅,R₂₇,R₂₉,R₃₁,R₃₂,R₄₈,R₅₀,R₅₂,R₆₈=are independently G or absent;
R₇,R₁₂=are independently G, U or absent;
R₈,R₁₆,R₃₃,R₃₆,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I_{MET} (SEQ ID NO: 592),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀,R₂₃=are absent;
R₁₄,R₃₈,R₄₀,R₅₇=are independently A or absent;
R₆₀=A, C or absent;
R₃₃,R₄₈,R₇₀=are independently A, C, G or absent;
R₁,R₃,R₄,R₅,R₆,R₁,R₁₂,R₁₆,R₁₇,R₂₁,R₂₂,R₂₆,R₂₇,R₂₉,R₃,R₃₁,R₃₂,R₄₂,R₄₄,R₄₅,R₄₆,R₄₉,R₅₀,R₅₈,R₆ 2,R₆₃,R₆₆,R₆₇,R₆₈,R₆₉,R₇₁=are independently N or absent;
R₁₈,R₃₅,R₄₁,R₅₉,R₆₅=are independently A, C, U or absent;
R₉,R₁₅,R₅₁=are independently A, G or absent;
R₇,R₂₄,R₂₅,R₃₄,R₅₃,R₅₆=are independently A, G, U or absent;
R₇₂=A, U or absent;
R₃₇=C or absent;
R₁₀,R₅₅=are independently C, G or absent;
R₂,R₁₃,R₂₈,R₄₃,R₆₄=are independently C, G, U or absent;
R₃₆,R₆₁=are independently C, U or absent;
R₁₉,R₂₀,R₅₂=are independently G or absent;
R₈,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{MET} (SEQ ID NO: 593),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀,R₁₈,R₂₂,R₂₃=are absent
R₁₄,R₂₄,R₃₈,R₄₀,R₄₁,R₅₇,R₇₂=are independently A or absent;
R₅₉,R₆₀,R₆₂,R₆₅=are independently A, C or absent;
R₆,R₄₅,R₆₇=are independently A, C, G or absent;
R₄=N or absent;
R₂₁,R₄₂=are independently A, C, U or absent;
R₁,R₉,R₂₇,R₂₉,R₃₂,R₄₆,R₅₁=are independently A, G or absent;
R₁₇,R₄₉,R₅₃,R₅₆,R₅₈=are independently A, G, U or absent;
R₆₃=A, U or absent;
R₃,R₁₃,R₃₇=are independently C or absent;
R₄₈,R₅₅,R₆₄,R₇₀=are independently C, G or absent;
R₂,R₅,R₆₆,R₆₈=are independently C, G, U or absent;
R₁₁,R₁₆,R₂₆,R₂₈,R₃₀,R₃₁,R₃₅,R₃₆,R₄₃,R₄₄,R₆₁,R₇₁=are independently C, U or absent;
R₁₀,R₁₂,R₁₅,R₁₉,R₂₀,R₂₅,R₃₃,R₅₂,R₆₉=are independently G or absent;
R₇,R₃₄,R₅₀=are independently G, U or absent;
R₈,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{MET} (SEQ ID NO: 594),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀,R₁₈,R₂₂,R₂₃=are absent
R₁₄,R₂₄,R₃₈,R₄₀,R₄₁,R₅₇,R₇₂=are independently A or absent;
R₅₉,R₆₂,R₆₅=are independently A, C or absent;
   R₆,R₆₇=are independently A, C, G or absent;
   R₄,R₂₁=are independently A, C, U or absent;
   R₁,R₉,R₂₇,R₂₉,R₃₂,R₄₅,R₄₆,R₅₁=are independently A, G or absent;
   R₁₇,R₅₆,R₅₈=are independently A, G, U or absent;
   R₄₉,R₅₃,R₆₃=are independently A, U or absent;
   R₃,R₁₃,R₂₆,R₃₇,R₄₃,R₆₀=are independently C or absent;
   R₂,R₄₈,R₅₅,R₆₄,R₇₀=are independently C, G or absent;
   R₅,R₆₆=are independently C, G, U or absent;
   R₁₁,R₁₆,R₂₈,R₃₀,R₃₁,R₃₅,R₃₆,R₄₂,R₄₄,R₆₁,R₇₁=are independently C, U or absent;
   R₁₀,R₁₂,R₁₅,R₁₉,R₂₀,R₂₅,R₃₃,R₅₂,R₆₉=are independently G or absent;
   R₇,R₃₄,R₅₀,R₆₈=are independently G, U or absent;
   R₈,R₃₉,R₅₄=are independently U or absent;
   [R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I_{LEU} (SEQ ID NO: 595),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent;
R₃₈,R₅₇=are independently A or absent;
R₆₀=A, C or absent;
R₁,R₁₃,R₂₇,R₄₈,R₅₁,R₅₆=are independently A, C, G or absent;
R₂,R₃,R₄,R₅,R₆,R₇,R₉,R₁₀,R₁₁,R₁₂,R₁₆,R₂₃,R₂₆,R₂₈,R₂₉,R₃₀,R₃₁,R₃₂,R₃₃,R₃₄,R₃₇,R₄₁,R₄₂,R₄₃,R₄₄,
R₄₅,R₄₆,R₄₉,R₅₀,R₅₈,R₆₂,R₆₃,R₆₅,R₆₆,R₆₇,R₆₈,R₆₉,R₇₀=are independently N or absent;
R₁₇,R₁₈,R₂₁,R₂₂,R₂₅,R₃₅,R₅₅=are independently A, C, U or absent;
R₁₄,R₁₅,R₃₉,R₇₂=are independently A, G or absent;
R₂₄,R₄₀=are independently A, G, U or absent;
R₅₂,R₆₁,R₆₄,R₇₁=are independently C, G, U or absent;
R₃₆,R₅₃,R₅₉=are independently C, U or absent;
R₁₉=G or absent;
R₂₀=G, U or absent;
R₈,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LEU} (SEQ ID NO: 596),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein R is a ribonucleotide residue and the consensus for Leu is:
R₀=absent
R₃₈,R₅₇,R₇₂=are independently A or absent;
R₆₀=A, C or absent;
R₄,R₅,R₄₈,R₅₀,R₅₆,R₆₉=are independently A, C, G or absent;
R₆,R₃₃,R₄₁,R₄₃,R₄₆,R₄₉,R₅₈,R₆₃,R₆₆,R₇₀=are independently N or absent;
R₁₁,R₁₂,R₁₇,R₂₁,R₂₂,R₂₈,R₃₁,R₃₇,R₄₄,R₅₅=are independently A, C, U or absent;
R₁,R₉,R₁₄,R₁₅,R₂₄,R₂₇,R₃₄,R₃₉=are independently A, G or absent;
R₇,R₂₉,R₃₂,R₄₀,R₄₅=are independently A, G, U or absent;
R₂₅=A, U or absent;
R₁₃=C, G or absent;
R₂,R₃,R₁₆,R₂₆,R₃₀,R₅₂,R₆₂,R₆₄,R₆₅,R₆₇,R₆₈=are independently C, G, U or absent;
R₁₈,R₃₅,R₄₂,R₅₃,R₅₉,R₆₁,R₇₁=are independently C, U or absent;
R₁₉,R₅₁=are independently G or absent;
R₁₀,R₂₀=are independently G, U or absent;
R₈,R₂₃,R₃₆,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LEU} (SEQ ID NO: 597),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₈,R₅₇,R₇₂=are independently A or absent;
R₆₀=A, C or absent;
R₄,R₅,R₄₈,R₅₀,R₅₆,R₅₈,R₆₉=are independently A, C, G or absent;
R₆,R₃₃,R₄₃,R₄₆,R₄₉,R₆₃,R₆₆,R₇₀=are independently N or absent;
R₁₁,R₁₂,R₁₇,R₂₁,R₂₂,R₂₈,R₃₁,R₃₇,R₄₁,R₄₄,R₅₅=are independently A, C, U or absent;
R₁,R₉,R₁₄,R₁₅,R₂₄,R₂₇,R₃₄,R₃₉=are independently A, G or absent;
R₇,R₂₉,R₃₂,R₄₀,R₄₅=are independently A, G, U or absent;
R₂₅=A, U or absent;
R₁₃=C, G or absent;
R₂,R₃,R₁₆,R₃₀,R₅₂,R₆₂,R₆₄,R₆₇,R₆₈=are independently C, G, U or absent;
R₁₈,R₃₅,R₄₂,R₅₃,R₅₉,R₆₁,R₆₅,R₇₁=are independently C, U or absent;
R₁₉,R₅₁=are independently G or absent;
R₁₀,R₂₀,R₂₆=are independently G, U or absent;
R₈,R₂₃,R₃₆,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I_{LYS} (SEQ ID NO: 598),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀=absent
R₁₄=A or absent;
R₄₀,R₄₁=are independently A, C or absent;
R₃₄,R₄₃,R₅₁=are independently A, C, G or absent;
R₁,R₂,R₃,R₄,R₅,R₆,R₇,R₁₁,R₁₂,R₁₆,R₂₁,R₂₆,R₃₀,R₃₁,R₃₂,R₄₄,R₄₅,R₄₆,R₄₈,R₄₉,R₅₀,R₅₈,R₆₂,R₆₃,R₆₅,
R₆₆,R₆₇,R₆₈,R₆₉,R₇₀=are independently N or absent;
R₁₃,R₁₇,R₅₉,R₇₁=are independently A, C, U or absent;
R₉,R₁₅,R₁₉,R₂₀,R₂₅,R₂₇,R₅₂,R₅₆=are independently A, G or absent;
R₂₄,R₂₉,R₇₂=are independently A, G, U or absent;
R₁₈,R₅₇=are independently A, U or absent;
R₁₀,R₃₃=are independently C, G or absent;
R₄₂,R₆₁,R₆₄=are independently C, G, U or absent;
R₂₈,R₃₅,R₃₆,R₃₇,R₅₃,R₅₅,R₆₀=are independently C, U or absent;
R₈,R₂₂,R₂₃,R₃₈,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LYS} (SEQ ID NO: 599),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀,R₁₈,R₂₃=are absent
R₁₄=A or absent;
R₄₀,R₄₁,R₄₃=are independently A, C or absent;
R₃,R₇=are independently A, C, G or absent;
R₁,R₆,R₁₁,R₃₁,R₄₅,R₄₈,R₄₉,R₆₃,R₆₅,R₆₆,R₆₈=are independently N or absent;
R₂,R₁₂,R₁₃,R₁₇,R₄₄,R₆₇,R₇₁=are independently A, C, U or absent;
R₉,R₁₅,R₁₉,R₂₀,R₂₅,R₂₇,R₃₄,R₅₀,R₅₂,R₅₆,R₇₀,R₇₂=are independently A, G or absent;
R₅,R₂₄,R₂₆,R₂₉,R₃₂,R₄₆,R₆₉=are independently A, G, U or absent;
R₅₇=A, U or absent;
R₁₀,R₆₁=are independently C, G or absent;
R₄,R₁₆,R₂₁,R₃₀,R₅₈,R₆₄=are independently C, G, U or absent;
R₂₈,R₃₅,R₃₆,R₃₇,R₄₂,R₅₃,R₅₅,R₅₉,R₆₀,R₆₂=are independently C, U or absent;
R₃₃,R₅₁=are independently G or absent;
R₈=G, U or absent;
R₂₂,R₃₈,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LYS} (SEQ ID NO: 600),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀,R₁₈,R₂₃=absent
R₉,R₁₄,R₃₄,R₄₁=are independently A or absent;
R₄₀=A, C or absent;
R₁,R₃,R₇,R₃₁=are independently A, C, G or absent;
R₄₈,R₆₅,R₆₈=are independently N or absent;
R₂,R₁₃,R₁₇,R₄₄,R₆₃,R₆₆=are independently A, C, U or absent;
R₅,R₁₅,R₁₉,R₂₀,R₂₅,R₂₇,R₂₉,R₅₀,R₅₂,R₅₆,R₇₀,R₇₂=are independently A, G or absent;
R₆,R₂₄,R₃₂,R₄₉=are independently A, G, U or absent;
R₁₂,R₂₆,R₄₆,R₅₇=are independently A, U or absent;
R₁₁,R₂₈,R₃₅,R₄₃=are independently C or absent;
R₁₀,R₄₅,R₆₁=are independently C, G or absent;
R₄,R₂₁,R₆₄=are independently C, G, U or absent;
R₃₇,R₅₃,R₅₅,R₅₉,R₆₀,R₆₂,R₆₇,R₇₁=are independently C, U or absent;
R₃₃,R₅₁=are independently G or absent;
R₈,R₃₀,R₅₈,R₆₉=are independently G, U or absent;
R₁₆,R₂₂,R₃₆,R₃₈,R₃₉,R₄₂,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I PHE (SEQ ID NO: 601),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀,R₂₃=are absent
R₉,R₁₄,R₃₈,R₃₉,R₅₇,R₇₂=are independently A or absent;
R₇₁=A, C or absent;
R₄₁,R₇₀=are independently A, C, G or absent;
R₄,R₅,R₆,R₃₀,R₃₁,R₃₂,R₃₄,R₄₂,R₄₄,R₄₅,R₄₆,R₄₈,R₄₉,R₅₈,R₆₂,R₆₃,R₆₆,R₆₇,R₆₈,R₆₉=are independently N or absent;
R₁₆,R₆₁,R₆₅=are independently A, C, U or absent;
R₁₅,R₂₆,R₂₇,R₂₉,R₄₀,R₅₆=are independently A, G or absent;
R₇,R₅₁=are independently A, G, U or absent;
R₂₂,R₂₄=are independently A, U or absent;
R₅₅,R₆₀=are independently C or absent;
R₂,R₃,R₂₁,R₃₃,R₄₃,R₅₀,R₆₄=are independently C, G, U or absent;
R₁₁,R₁₂,R₁₃,R₁₇,R₂₈,R₃₅,R₃₆,R₅₉=are independently C, U or absent;
R₁₀,R₁₉,R₂₀,R₂₅,R₃₇,R₅₂=are independently G or absent;
R₁=G, U or absent;
R₈,R₁₈,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PHE} (SEQ ID NO: 602),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀,R₁₈,R₂₃=absent
R₁₄,R₂₄,R₃₈,R₃₉,R₅₇,R₇₂=are independently A or absent;
R₄₆,R₇₁=are independently A, C or absent;
R₄,R₇₀=are independently A, C, G or absent;
R₄₅=A, C, U or absent;
R₆,R₇,R₁₅,R₂₆,R₂₇,R₃₂,R₃₄,R₄₀,R₄₁,R₅₆,R₆₉=are independently A, G or absent;
R₂₉=A, G, U or absent;
R₅,R₉,R₆₇=are independently A, U or absent;
R₃₅,R₄₉,R₅₅,R₆₀=are independently C or absent;
R₂₁,R₄₃,R₆₂=are independently C, G or absent;
R₂,R₃₃,R₆₈=are independently C, G, U or absent;
R₃,R₁₁,R₁₂,R₁₃,R₂₈,R₃₀,R₃₆,R₄₂,R₄₄,R₄₈,R₅₈,R₅₉,R₆₁,R₆₆=are independently C, U or absent;
R₁₀,R₁₉,R₂₀,R₂₅,R₃₇,R₅₁,R₅₂,R₆₃,R₆₄=are independently G or absent;
R₁,R₃₁,R₅₀=are independently G, U or absent;
R₈,R₁₆,R₁₇,R₂₂,R₅₃,R₅₄,R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀,R₁₈,R₂₂,R₂₃=absent
R₅,R₇,R₁₄,R₂₄,R₂₆,R₃₂,R₃₄,R₃₈,R₃₉,R₄₁,R₅₇,R₇₂=are independently A or absent;
R₄₆=A, C or absent;
R₇₀=A, C, G or absent;
R₄,R₆,R₁₅,R₅₆,R₆₉=are independently A, G or absent;
R₉,R₄₅=are independently A, U or absent;
R₂,R₁₁,R₁₃,R₃₅,R₄₃,R₄₉,R₅₅,R₆₀,R₆₈,R₇₁=are independently C or absent;
R₃₃=C, G or absent;
R₃,R₂₈,R₃₆,R₄₈,R₅₈,R₅₉,R₆₁=are independently C, U or absent;
R₁,R₁₀,R₁₉,R₂₀,R₂₁,R₂₅,R₂₇,R₂₉,R₃₇,R₄₀,R₅₁,R₅₂,R₆₂,R₆₃,R₆₄=are independently G or absent;
R₈,R₁₂,R₁₆,R₁₇,R₃₀,R₃₁,R₄₂,R₄₄,R₅₀,R₅₃,R₅₄,R₆₅,R₆₆,R₆₇=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I_{PRO} (SEQ ID NO: 604),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀=absent
R₁₄,R₅₇=are independently A or absent;
R₇₀,R₇₂=are independently A, C or absent;
R₉,R₂₆,R₂₇=are independently A, C, G or absent;
R₄,R₅,R₆,R₁₆,R₂₁,R₂₉,R₃₀,R₃₁,R₃₂,R₃₃,R₃₄,R₃₇,R₄₁,R₄₂,R₄₃,R₄₄,R₄₅,R₄₆,R₄₈,R₄₉,R₅₀,R₅₈,R₆₁,R₆₂,
R₆₃,R₆₄,R₆₆,R₆₇,R₆₈=are independently N or absent;
R₃₅,R₆₅=are independently A, C, U or absent;
R₂₄,R₄₀,R₅₆=are independently A, G or absent;
R₇,R₂₅,R₅₁=are independently A, G, U or absent;
R₅₅,R₆₀=are independently C or absent;
R₁,R₃,R₇₁=are independently C, G or absent;
R₁₁,R₁₂,R₂₀,R₆₉=are independently C, G, U or absent;
R₁₃,R₁₇,R₁₈,R₂₂,R₂₃,R₂₈,R₅₉=are independently C, U or absent;
R₁₀,R₁₅,R₁₉,R₃₈,R₃₉,R₅₂=are independently G or absent;
R₂=are independently G, U or absent;
R₈,R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PRO} (SEQ ID NO: 605),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀,R₁₇,R₁₈,R₂₂,R₂₃=absent;
R₁₄,R₄₅,R₅₆,R₅₇,R₅₈,R₆₅,R₆₈=are independently A or absent;
R₆₁=A, C, G or absent;
R₄₃=N or absent;
R₃₇=A, C, U or absent;
R₂₄,R₂₇,R₃₃,R₄₀,R₄₄,R₆₃=are independently A, G or absent;
R₃,R₁₂,R₃₀,R₃₂,R₄₈,R₅₅,R₆₀,R₇₀,R₇₁,R₇₂=are independently C or absent;
R₅,R₃₄,R₄₂,R₆₆=are independently C, G or absent;
R₂₀=C, G, U or absent;
R₃₅,R₄₁,R₄₉,R₆₂=are independently C, U or absent;
R₁,R₂,R₆,R₉,R₁₀,R₁₅,R₁₉,R₂₆,R₃₈,R₃₉,R₄₆,R₅₀,R₅₁,R₅₂,R₆₄,R₆₇,R₆₉=are independently G or absent;
R₁₁,R₁₆=are independently G, U or absent;
R₄,R₇,R₈,R₁₃,R₂₁,R₂₅,R₂₈,R₂₉,R₃₁,R₃₆,R₅₃,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{PRO} (SEQ ID NO: 606),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀,R₁₇,R₁₈,R₂₂,R₂₃=absent
R₁₄,R₄₅,R₅₆,R₅₇,R₅₈,R₆₅,R₆₈=are independently A or absent;
R₃₇=A, C, U or absent;
R₂₄,R₂₇,R₄₀=are independently A, G or absent;
R₃,R₅,R₁₂,R₃₀,R₃₂,R₄₈,R₄₉,R₅₅,R₆₀,R₆₁,R₆₂,R₆₆,R₇₀,R₇₁,R₇₂=are independently C or absent;
R₃₄,R₄₂=are independently C, G or absent;
R₄₃=C, G, U or absent;
R₄₁=C, U or absent;
R₁,R₂,R₆,R₉,R₁₀,R₁₅,R₁₉,R₂₀,R₂₆,R₃₃,R₃₈,R₃₉,R₄₄,R₄₆,R₅₀,R₅₁,R₅₂,R₆₃,R₆₄,R₆₇,R₆₉=are independently G or absent;
R₁₆=G, U or absent;
R₄,R₇,R₈,R₁₁,R₁₃,R₂₁,R₂₅,R₂₈,R₂₉,R₃₁,R₃₅,R₃₆,R₅₃,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I_{SER} (SEQ ID NO: 607),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀=absent;
R₁₄,R₂₄,R₅₇=are independently A or absent;
R₄₁=A, C or absent;
R₂,R₃,R₄,R₅,R₆,R₇,R₉,R₁₀,R₁₁,R₁₂,R₁₃,R₁₆,R₂₁,R₂₅,R₂₆,R₂₇,R₂₈,R₃,R₃₁,R₃₂,R₃₃,R₃₄,R₃₇,R₄₂,R₄₃, R₄₄,R₄₅,R₄₆,R₄₈,R₄₉,R₅₀,R₆₂,R₆₃,R₆₄,R₆₅,R₆₆,R₆₇,R₆₈,R₆₉,R₇₀=are independently N or absent;
R₁₈=A, C, U or absent;
R₁₅,R₄₀,R₅₁,R₅₆=are independently A, G or absent;
R₁,R₂₉,R₅₈,R₇₂=are independently A, G, U or absent;
R₃₉=A, U or absent;
R₆₀=C or absent;
R₃₈=C, G or absent;
R₁₇,R₂₂,R₂₃,R₇₁=are independently C, G, U or absent;
R₈,R₃₅,R₃₆,R₅₅,R₅₉,R₆₁=are independently C, U or absent;
R₁₉,R₂₀=are independently G or absent;
R₅₂=G, U or absent;
R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{SER} (SEQ ID NO: 608),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀,R₂₃=absent
R₁₄,R₂₄,R₄₁,R₅₇=are independently A or absent;
R₄₄=A, C or absent;
R₂₅,R₄₅,R₄₈=are independently A, C, G or absent;
R₂,R₃,R₄,R₅,R₃₇,R₅₀,R₆₂,R₆₆,R₆₇,R₆₉,R₇₀=are independently N or absent;
R₁₂,R₂₈,R₆₅=are independently A, C, U or absent;
R₉,R₁₅,R₂₉,R₃₄,R₄₀,R₅₆,R₆₃=are independently A, G or absent;
R₇,R₂₆,R₃₀,R₃₃,R₄₆,R₅₈,R₇₂=are independently A, G, U or absent;
R₃₉=A, U or absent;
R₁₁,R₃₅,R₆₀,R₆₁=are independently C or absent;
R₁₃,R₃₈=are independently C, G or absent;
R₆,R₁₇,R₃₁,R₄₃,R₆₄,R₆₈=are independently C, G, U or absent;
R₃₆,R₄₂,R₄₉,R₅₅,R₅₉,R₇₁=are independently C, U or absent;
R₁₀,R₁₉,R₂₀,R₂₇,R₅₁=are independently G or absent;
R₁,R₁₆,R₃₂,R₅₂=are independently G, U or absent;
R₈,R₁₈,R₂₁,R₂₂,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{SER} (SEQ ID NO: 609),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀,R₂₃=absent
R₁₄,R₂₄,R₄₁,R₅₇,R₅₈=are independently A or absent;
R₄₄=A, C or absent;
R₂₅,R₄₈=are independently A, C, G or absent;
R₂,R₃,R₅,R₃₇,R₆₆,R₆₇,R₆₉,R₇₀=are independently N or absent;
R₁₂,R₂₈,R₆₂=are independently A, C, U or absent;
R₇,R₉,R₁₅,R₂₉,R₃₃,R₃₄,R₄₀,R₄₅,R₅₆,R₆₃=are independently A, G or absent;
R₄,R₂₆,R₄₆,R₅₀=are independently A, G, U or absent;
R₃₀,R₃₉=are independently A, U or absent;
R₁₁,R₁₇,R₃₅,R₆₀,R₆₁=are independently C or absent;
R₁₃,R₃₈=are independently C, G or absent;
R₆,R₆₄=are independently C, G, U or absent;
R₃₁,R₄₂,R₄₃,R₄₉,R₅₅,R₅₉,R₆₅,R₆₈,R₇₁=are independently C, U or absent;
R₁₀,R₁₉,R₂₀,R₂₇,R₅₁,R₅₂=are independently G or absent;
R₁,R₁₆,R₃₂,R₇₂=are independently G, U or absent;
R₈,R₁₈,R₂₁,R₂₂,R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I_{THR} (SEQ ID NO: 610),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀,R₂₃=absent
R₁₄,R₄₁,R₅₇=are independently A or absent;
R₅₆,R₇₀=are independently A, C, G or absent;
R₄,R₅,R₆,R₇,R₁₂,R₁₆,R₂₆,R₃₀,R₃₁,R₃₂,R₃₄,R₃₇,R₄₂,R₄₄,R₄₅,R₄₆,R₄₈,R₄₉,R₅₀,R₅₈,R₆₂,R₆₃,R₆₄,R₆₅,R₆₆,R₆₇,R₆₈,R₇₂=are independently N or absent;
R₁₃,R₁₇,R₂₁,R₃₅,R₆₁=are independently A, C, U or absent;
R₁,R₉,R₂₄,R₂₇,R₂₉,R₆₉=are independently A, G or absent;
R₁₅,R₂₅,R₅₁=are independently A, G, U or absent;
R₄₀,R₅₃=are independently A, U or absent;
R₃₃,R₄₃=are independently C, G or absent;
R₂,R₃,R₅₉=are independently C, G, U or absent;
R₁₁,R₁₈,R₂₂,R₂₈,R₃₆,R₅₄,R₅₅,R₆₀,R₇₁=are independently C, U or absent;
R₁₀,R₂₀,R₃₈,R₅₂=are independently G or absent;
R₁₉=G, U or absent;
R₈,R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II THR (SEQ ID NO: 611),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀,R₁₈,R₂₃=absent
R₁₄,R₄₁,R₅₇=are independently A or absent;
R₉,R₄₂,R₄₄,R₄₈,R₅₆,R₇₀=are independently A, C, G or absent;
R₄,R₆,R₁₂,R₂₆,R₄₉,R₅₈,R₆₃,R₆₄,R₆₆,R₆₈=are independently N or absent;
R₁₃,R₂₁,R₃₁,R₃₇,R₆₂=are independently A, C, U or absent;
R₁,R₁₅,R₂₄,R₂₇,R₂₉,R₄₆,R₅₁,R₆₉=are independently A, G or absent;
R₇,R₂₅,R₄₅,R₅₀,R₆₇=are independently A, G, U or absent;
R₄₀,R₅₃=are independently A, U or absent;
R₃₅=C or absent;
R₃₃,R₄₃=are independently C, G or absent;
R₂,R₃,R₅,R₁₆,R₃₂,R₃₄,R₅₉,R₆₅,R₇₂=are independently C, G, U or absent;
R₁₁,R₁₇,R₂₂,R₂₈,R₃₀,R₃₆,R₅₅,R₆₀,R₆₁,R₇₁=are independently C, U or absent;
R₁₀,R₁₉,R₂₀,R₃₈,R₅₂=are independently G or absent;
R₈,R₃₉,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{THR} (SEQ ID NO: 612),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀,R₁₈,R₂₃=absent
R₁₄,R₄₀,R₄₁,R₅₇=are independently A or absent;
R₄₄=A, C or absent;
R₉,R₄₂,R₄₈,R₅₆=are independently A, C, G or absent;
R₄,R₆,R₁₂,R₂₆,R₅₈,R₆₄,R₆₆,R₆₈=are independently N or absent;
R₁₃,R₂₁,R₃₁,R₃₇,R₄₉,R₆₂=are independently A, C, U or absent;
R₁,R₁₅,R₂₄,R₂₇,R₂₉,R₄₆,R₅₁,R₆₉=are independently A, G or absent;
R₇,R₂₅,R₄₅,R₅₀,R₆₃,R₆₇=are independently A, G, U or absent;
R₅₃=A, U or absent;
R₃₅=C or absent;
R₂,R₃₃,R₄₃,R₇₀=are independently C, G or absent;
R₅,R₁₆,R₃₄,R₅₉,R₆₅=are independently C, G, U or absent;
R₃,R₁₁,R₂₂,R₂₈,R₃₀,R₃₆,R₅₅,R₆₀,R₆₁,R₇₁=are independently C, U or absent;
R₁₀,R₁₉,R₂₀,R₃₈,R₅₂=are independently G or absent;
R₃₂=G, U or absent;
R₈,R₁₇,R₃₉,R₅₄,R₇₂=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I_{TRP} (SEQ ID NO: 613),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀=absent;
R₂₄,R₃₉,R₄₁,R₅₇=are independently A or absent;
R₂,R₃,R₂₆,R₂₇,R₄₀,R₄₈=are independently A, C, G or absent;
R₄,R₅,R₆,R₂₉,R₃₀,R₃₁,R₃₂,R₃₄,R₄₂,R₄₄,R₄₅,R₄₆,R₄₉,R₅₁,R₅₈,R₆₃,R₆₆,R₆₇,R₆₈=are independently N or absent;
R₁₃,R₁₄,R₁₆,R₁₈,R₂₁,R₆₁,R₆₅,R₇₁=are independently A, C, U or absent;
R₁,R₉,R₁₀,R₁₅,R₃₃,R₅₀,R₅₆=are independently A, G or absent;
R₇,R₂₅,R₇₂=are independently A, G, U or absent;
R₃₇,R₃₈,R₅₅,R₆₀=are independently C or absent;
R₁₂,R₃₅,R₄₃,R₆₄,R₆₉,R₇₀=are independently C, G, U or absent;
R₁₁,R₁₇,R₂₂,R₂₈,R₅₉,R₆₂=are independently C, U or absent;
R₁₉,R₂₀,R₅₂=are independently G or absent;
R₈,R₂₃,R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II TRP (SEQ ID NO: 614),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀,R₁₈,R₂₂,R₂₃=absent
R₁₄,R₂₄,R₃₉,R₄₁,R₅₇,R₇₂=are independently A or absent;
R₃,R₄,R₁₃,R₆₁,R₇₁=are independently A, C or absent;
R₆,R₄₄=are independently A, C, G or absent;
R₂₁=A, C, U or absent;
R₂,R₇,R₁₅,R₂₅,R₃₃,R₃₄,R₄₅,R₅₆,R₆₃=are independently A, G or absent;
R₅₈=A, G, U or absent;
R₄₆=A, U or absent;
R₃₇,R₃₈,R₅₅,R₆₀,R₆₂=are independently C or absent;
R₁₂,R₂₆,R₂₇,R₃₅,R₄₀,R₄₈,R₆₇=are independently C, G or absent;
R₃₂,R₄₃,R₆₈=are independently C, G, U or absent;
R₁₁,R₁₆,R₂₈,R₃₁,R₄₉,R₅₉,R₆₅,R₇₀=are independently C, U or absent;
R₁,R₉,R₁₀,R₁₉,R₂₀,R₅₀,R₅₂,R₆₉=are independently G or absent;
R₅,R₈,R₂₉,R₃₀,R₄₂,R₅₁,R₆₄,R₆₆=are independently G, U or absent;
R₁₇,R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TRP} (SEQ ID NO: 615),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀,R₁₈,R₂₂,R₂₃=absent
R₁₄,R₂₄,R₃₉,R₄₁,R₅₇,R₇₂=are independently A or absent;
R₃,R₄,R₁₃,R₆₁,R₇₁=are independently A, C or absent;
R₆,R₄₄=are independently A, C, G or absent;
R₂₁=A, C, U or absent;
R₂,R₇,R₁₅,R₂₅,R₃₃,R₃₄,R₄₅,R₅₆,R₆₃=are independently A, G or absent;
R₅₈=A, G, U or absent;
R₄₆=A, U or absent;
R₃₇,R₃₈,R₅₅,R₆₀,R₆₂=are independently C or absent;
R₁₂,R₂₆,R₂₇,R₃₅,R₄₀,R₄₈,R₆₇=are independently C, G or absent;
R₃₂,R₄₃,R₆₈=are independently C, G, U or absent;
R₁₁,R₁₆,R₂₈,R₃₁,R₄₉,R₅₉,R₆₅,R₇₀=are independently C, U or absent;
R₁,R₉,R₁₀,R₁₉,R₂₀,R₅₀,R₅₂,R₆₉=are independently G or absent;
R₅,R₈,R₂₉,R₃₀,R₄₂,R₅₁,R₆₄,R₆₆=are independently G, U or absent;
R₁₇,R₃₆,R₅₃,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I_{TYR} (SEQ ID NO: 616),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀=absent
R₁₄,R₃₉,R₅₇=are independently A or absent;
R₄₁,R₄₈,R₅₁,R₇₁=are independently A, C, G or absent;
R₃,R₄,R₅,R₆,R₉,R₁₀,R₁₂,R₁₃,R₁₆,R₂₅,R₂₆,R₃,R₃₁,R₃₂,R₄₂,R₄₄,R₄₅,R₄₆,R₄₉,R₅₀,R₅₈,R₆₂,R₆₃,R₆₆,
R₆₇,R₆₈,R₆₉,R₇₀=are independently N or absent;
R₂₂,R₆₅=are independently A, C, U or absent;
R₁₅,R₂₄,R₂₇,R₃₃,R₃₇,R₄₀,R₅₆=are independently A, G or absent;
R₇,R₂₉,R₃₄,R₇₂=are independently A, G, U or absent;
R₂₃,R₅₃=are independently A, U or absent;
R₃₅,R₆₀=are independently C or absent;
R₂₀=C, G or absent;
R₁,R₂,R₂₈,R₆₁,R₆₄=are independently C, G, U or absent;
R₁₁,R₁₇,R₂₁,R₄₃,R₅₅=are independently C, U or absent;
R₁₉,R₅₂=are independently G or absent;
R₈,R₁₈,R₃₆,R₃₈,R₅₄,R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TYR} (SEQ ID NO: 617),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀,R₁₈,R₂₃=absent
R₇,R₉,R₁₄,R₂₄,R₂₆,R₃₄,R₃₉,R₅₇=are independently A or absent;
R₄₄,R₆₉=are independently A, C or absent;
R₇₁=A, C, G or absent;
R₆₈=N or absent;
R₅₈=A, C, U or absent;
R₃₃,R₃₇,R₄₁,R₅₆,R₆₂,R₆₃=are independently A, G or absent;
R₆,R₂₉,R₇₂=are independently A, G, U or absent;
R₃₁,R₄₅,R₅₃=are independently A, U or absent;
R₁₃,R₃₅,R₄₉,R₆₀=are independently C or absent;
R₂₀,R₄₈,R₆₄,R₆₇,R₇₀=are independently C, G or absent;
R₁,R₂,R₅,R₁₆,R₆₆=are independently C, G, U or absent;
R₁₁,R₂₁,R₂₈,R₄₃,R₅₅,R₆₁=are independently C, U or absent;
R₁₀,R₁₅,R₁₉,R₂₅,R₂₇,R₄₀,R₅₁,R₅₂=are independently G or absent;
R₃,R₄,R₃₀,R₃₂,R₄₂,R₄₆=are independently G, U or absent;
R₈,R₁₂,R₁₇,R₂₂,R₃₆,R₃₈,R₅₀,R₅₄,R₅₉,R₆₅=are independently U or absent;
[R₄₇],x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TYR} (SEQ ID NO: 618),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀,R₁₈,R₂₃=absent
R₇,R₉,R₁₄,R₂₄,R₂₆,R₃₄,R₃₉,R₅₇,R₇₂=are independently A or absent;
R₄₄,R₆₉=are independently A, C or absent;
R₇₁=A, C, G or absent;
R₃₇,R₄₁,R₅₆,R₆₂,R₆₃=are independently A, G or absent;
R₆,R₂₉,R₆₈=are independently A, G, U or absent;
R₃₁,R₄₅,R₅₈=are independently A, U or absent;
R₁₃,R₂₈,R₃₅,R₄₉,R₆₀,R₆₁=are independently C or absent;
R₅,R₄₈,R₆₄,R₆₇,R₇₀=are independently C, G or absent;
R₁,R₂=are independently C, G, U or absent;
R₁₁,R₁₆,R₂₁,R₄₃,R₅₅,R₆₆=are independently C, U or absent;
R₁₀,R₁₅,R₁₉,R₂₀,R₂₅,R₂₇,R₃₃,R₄₀,R₅₁,R₅₂=are independently G or absent;
R₃,R₄,R₃₀,R₃₂,R₄₂,R₄₆=are independently G, U or absent;
R₈,R₁₂,R₁₇,R₂₂,R₃₆,R₃₈,R₅₀,R₅₃,R₅₄,R₅₉,R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I_{VAL} (SEQ ID NO: 619),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀,R₂₃=absent;
R₂₄,R₃₈,R₅₇=are independently A or absent;
R₉,R₇₂=are independently A, C, G or absent;
R₂,R₄,R₅,R₆,R₇,R₁₂,R₁₅,R₁₆,R₂₁,R₂₅,R₂₆,R₂₉,R₃₁,R₃₂,R₃₃,R₃₄,R₃₇,R₄₁,R₄₂,R₄₃,R₄₄,R₄₅,R₄₆,R₄₈,R₄₉,R₅₀,R₅₈,R₆₁,R₆₂,R₆₃,R₆₄,R₆₅,R₆₆,R₆₇,R₆₈,R₆₉,R₇₀=are independently N or absent;
R₁₇,R₃₅,R₅₉=are independently A, C, U or absent;
R₁₀,R₁₄,R₂₇,R₄₀,R₅₂,R₅₆=are independently A, G or absent;
R₁,R₃,R₅₁,R₅₃=are independently A, G, U or absent;
R₃₉=C or absent;
R₁₃,R₃₀,R₅₅=are independently C, G, U or absent;
R₁₁,R₂₂,R₂₈,R₆₀,R₇₁=are independently C, U or absent;
R₁₉=G or absent;
R₂₀=G, U or absent;
R₈,R₁₈,R₃₆,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{VAL} (SEQ ID NO: 620),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀,R₁₈,R₂₃=absent;
R₂₄,R₃₈,R₅₇=are independently A or absent;
R₆₄,R₇₀,R₇₂=are independently A, C, G or absent;
R₁₅,R₁₆,R₂₆,R₂₉,R₃₁,R₃₂,R₄₃,R₄₄,R₄₅,R₄₉,R₅₀,R₅₈,R₆₂,R₆₅=are independently N or absent;
R₆,R₁₇,R₃₄,R₃₇,R₄₁,R₅₉=are independently A, C, U or absent;
R₉,R₁₀,R₁₄,R₂₇,R₄₀,R₄₆,R₅₁,R₅₂,R₅₆=are independently A, G or absent;
R₇,R₁₂,R₂₅,R₃₃,R₅₃,R₆₃,R₆₆,R₆₈=are independently A, G, U or absent;
R₆₉=A, U or absent;
R₃₉=C or absent;
R₅,R₆₇=are independently C, G or absent;
R₂,R₄,R₁₃,R₄₈,R₅₅,R₆₁=are independently C, G, U or absent;
R₁₁,R₂₂,R₂₈,R₃₀,R₃₅,R₆₀,R₇₁=are independently C, U or absent;
R₁₉=G or absent;
R₁,R₃,R₂₀,R₄₂=are independently G, U or absent;
R₈,R₂₁,R₃₆,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{VAL} (SEQ ID NO: 621),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀,R₁₈,R₂₃=absent
R₂₄,R₃₈,R₄₀,R₅₇,R₇₂=are independently A or absent;
R₂₉,R₆₄,R₇₀=are independently A, C, G or absent;
R₄₉,R₅₀,R₆₂=are independently N or absent;
R₁₆,R₂₆,R₃₁,R₃₂,R₃₇,R₄₁,R₄₃,R₅₉,R₆₅=are independently A, C, U or absent;
R₉,R₁₄,R₂₇,R₄₆,R₅₂,R₅₆,R₆₆=are independently A, G or absent;
R₇,R₁₂,R₂₅,R₃₃,R₄₄,R₄₅,R₅₃,R₅₈,R₆₃,R₆₈=are independently A, G, U or absent;
R₆₉=A, U or absent;
R₃₉=C or absent;
R₅,R₆₇=are independently C, G or absent;
R₂,R₄,R₁₃,R₁₅,R₄₈,R₅₅=are independently C, G, U or absent;
R₆,R₁₁,R₂₂,R₂₈,R₃₀,R₃₄,R₃₅,R₆₀,R₆₁,R₇₁=are independently C, U or absent;
R₁₀,R₁₉,R₅₁=are independently G or absent;
R₁,R₃,R₂₀,R₄₂=are independently G, U or absent;
R₈,R₁₇,R₂₁,R₃₆,R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 3, e.g., any one of SEQ ID NOs: 452-561 disclosed in Table 3.

TABLE 3
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Method of Making TREMs

Methods for designing and constructing expression vectors and modifying a host cell for production of a target (e.g., a TREM or an enzyme disclosed herein) use techniques known in the art. For example, a cell is genetically modified to express an exogenous TREM using cultured mammalian cells (e.g., cultured human cells), insect cells, yeast, bacteria, or other cells under the control of appropriate promoters. Generally, recombinant methods may be used. See, in general, Pharmaceutical Biotechnology: Fundamentals and Applications, Springer (2013); Green and Sambrook (Eds.), Molecular Cloning: A Laboratory Manual (Fourth Edition), Cold Spring Harbor Laboratory Press (2012). For example, mammalian expression vectors may comprise non-transcribed elements such as an origin of replication, a suitable promoter and enhancer, and other 5′ or 3′ flanking non-transcribed sequences. DNA sequences derived from the SV40 viral genome, for example, SV40 origin, early promoter, enhancer, splice, and polyadenylation sites may be used to provide the other genetic elements required for expression of a heterologous DNA sequence.

A method of making a TREM or TREM composition disclosed herein comprises use of a host cell, e.g., a modified host cell, expressing a TREM.

The modified host cell is cultured under conditions that allow for expression of the TREM. In an embodiment, the culture conditions can be modulated to increase expression of the TREM. The method of making a TREM further comprises purifying the expressed TREM from the host cell culture to produce a TREM composition. In an embodiment the TREM is a TREM fragment, e.g., a fragment of a tRNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., RNase A, Dicer, Angiogenin, RNaseP, RNaseZ, Rny1 or PrrC.

In an embodiment, a method of making a TREM described herein comprises contacting (e.g., transducing or transfecting) a host cell (e.g., as described herein, e.g., a modified host cell) with an exogenous nucleic acid described herein, e.g., a DNA or RNA, encoding a TREM under conditions sufficient to express the TREM. In an embodiment, the exogenous nucleic acid comprises an RNA (or DNA encoding an RNA) that comprises a ribonucleic acid (RNA) sequence of an RNA encoded by a DNA sequence disclosed in Table 1. In an embodiment, the exogenous nucleic acid comprises an RNA sequence (or DNA encoding an RNA sequence) that is at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1. In an embodiment, the exogenous nucleic acid comprises an RNA sequence (or DNA encoding an RNA sequence) that comprises at least 30 consecutive nucleotides of a ribonucleic acid (RNA) sequence encoded by a deoxyribonucleic acid (DNA) sequence disclosed in Table 1. In an embodiment, the exogenous nucleic acid comprises an RNA sequence (or DNA encoding an RNA sequence) that comprises at least 30 consecutive nucleotides of an RNA sequence at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1.

In an embodiment, the host cell is transduced with a virus (e.g., a lentivirus, adenovirus or retrovirus) expressing a TREM, e.g., as described in Example 8.

The expressed TREM can be purified from the host cell or host cell culture to produce a TREM composition, e.g., as described herein. Purification of the TREM can be performed by affinity purification, e.g., as described in the MACS Isolation of specific tRNA molecules protocol, or other methods known in the art. In an embodiment, a TREM is purified by a method described in Example 7.

In an embodiment, a method of making a TREM, e.g., a TREM composition, comprises contacting a TREM with a reagent, e.g., a capture reagent comprising a nucleic acid sequence complimentary with a TREM. A single capture reagent or a plurality of capture reagents can be used to make a TREM, e.g., a TREM composition. When a single capture reagent is used, the capture reagent can have at least 50%, 55%, 60%, 65%, 70%, 75%, 80%, 85%, 90%, or 95% complimentary sequence with the TREM. When a plurality of capture reagents is used, a composition of TREMs having a plurality of different TREMs can be made. In an embodiment, the capture reagent can be conjugated to an agent, e.g., biotin.

In an embodiment, the method comprises denaturing the TREM, e.g., prior to hybridization with the capture reagent. In an embodiment, the method comprises, renaturing the TREM, after hybridization and/or release from the capture reagent.

In an embodiment, a method of making a TREM, e.g., a TREM composition, comprises contacting a TREM with a reagent, e.g., a separation reagent, e.g., a chromatography reagent. In an embodiment, a chromatography reagent includes a column chromatography reagent, a planar chromatography reagent, a displacement chromatography reagent, a gas chromatography reagent, a liquid chromatography reagent, an affinity chromatography reagent, an ion-exchange chromatography reagent, or a size-exclusion chromatography reagent.

In an embodiment, a TREM made by any of the methods described herein can be: (i) charged with an amino acid, e.g., a cognate amino acid; (ii) charged with a non-cognate amino acid (e.g., a mischarged TREM (mTREM); or (iii) not charged with an amino acid, e.g., an uncharged TREM (uTREM).

In an embodiment, a TREM made by any of the methods described herein is an uncharged TREM (uTREM). In an embodiment, a method of making a uTREM comprises culturing the host cell in media that has a limited amount of one or more nutrients, e.g., the media is nutrient starved.

In an embodiment, a charged TREM, e.g., a TREM charged with a cognate AA or a non-cognate AA, can be uncharged, e.g., by dissociating the AA, e.g., by incubating the TREM at a high temperature.

### Exogenous Nucleic Acid Encoding a TREM or a TREM Fragment

In an embodiment, an exogenous nucleic acid, e.g., a DNA or RNA, encoding a TREM comprises a nucleic acid sequence comprising a nucleic acid sequence of one or a plurality of RNA sequences encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, or 99% identical to an RNA sequence encoded by a DNA sequence disclosed in Table 1, e.g., any one of SEQ ID NOs: 1-451 as disclosed in Table 1. In one embodiment, the exogenous nucleic acid, e.g., a DNA or RNA, encoding a TREM comprises a nucleic acid sequence less than 100% identical to an RNA sequence encoded by a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM comprises the nucleic acid sequence of an RNA sequence encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, 99%, or 100% identical to a plurality of RNA sequences encoded by a DNA sequence disclosed in Table 1, e.g., any one of SEQ ID NOs: 1-451 as disclosed in Table 1. In an embodiment, an exogenous nucleic acid encoding a TREM comprises an RNA sequence encoded by a DNA sequence at least 60%, 65%, 70%, 75%, 80%, 85%, 90%, 95%, 96%, 97%, 98%, or 99% identical to a DNA sequence disclosed in Table 1, e.g., any one of SEQ ID NOs: 1-451 as disclosed in Table 1. In an embodiment, the exogenous nucleic acid encoding a TREM comprises an RNA sequence encoded by a DNA sequence less than 100% identical to a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM comprises an RNA sequence of one or a plurality of TREM fragments, e.g., a fragment of an RNA encoded by a DNA sequence disclosed in Table 1, e.g., as described herein, e.g., a fragment of any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence of an RNA encoded by a DNA sequence provided in Table 1, e.g., any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence at least 60%, 65%, 70%, 75%, 80%, 85%, 90%, 95%, 96%, 97%, 98%, or 99% identical to an RNA encoded by a DNA sequence provided in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence encoded by a DNA sequence at least 60%, 65%, 70%, 75%, 80%, 85%, 90%, 95%, 96%, 97%, 98%, or 99% identical to a DNA sequence provided in Table 1, e.g., any one of SEQ ID NOs: 1-451 as disclosed in Table 1.

In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence disclosed in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1 e.g., any one of SEQ ID NOs: 1-451 as disclosed in Table 1.

In an embodiment, the exogenous nucleic acid comprises a DNA, which upon transcription, expresses a TREM.

In an embodiment, the exogenous nucleic acid comprises an RNA, which upon reverse transcription, results in a DNA which can be transcribed to provide the TREM.

In an embodiment, the exogenous nucleic acid encoding a TREM comprises: (i) a control region sequence; (ii) a sequence encoding a modified TREM; (iii) a sequence encoding more than one TREM; or (iv) a sequence other than a tRNA^{Met} sequence.

In an embodiment, the exogenous nucleic acid encoding a TREM comprises a promoter sequence. In an embodiment, the exogenous nucleic acid comprises an RNA Polymerase III (Pol III) recognition sequence, e.g., a Pol III binding sequence. In an embodiment, the promoter sequence comprises a U6 promoter sequence or fragment thereof. In an embodiment, the nucleic acid sequence comprises a promoter sequence that comprises a mutation, e.g., a promoter-up mutation, e.g., a mutation that increases transcription initiation, e.g., a mutation that increases TFIIIB binding. In an embodiment, the nucleic acid sequence comprises a promoter sequence which increases Pol III binding and results in increased tRNA production, e.g., TREM production.

Also disclosed herein is a plasmid comprising an exogenous nucleic acid encoding a TREM. In an embodiment, the plasmid comprises a promoter sequence, e.g., as described herein.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index.Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM.

In an embodiment, a TREM composition, e.g., a TREM pharmaceutical composition, is at least 10, 20, 30, 40, 50, 60, 70, 80, 90, 95 or 99% dry weight TREMs.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition produced by any of the methods of making disclosed herein can be charged with an amino acid using an in vitro charging reaction as disclosed in Example 11, or as known in the art.

In an embodiment, a TREM composition comprise one or more species of TREMs. In an embodiment, a TREM composition comprises a single species of TREMs. In an embodiment, a TREM composition comprises a first TREM species and a second TREM species. In an embodiment, the TREM composition comprises X TREM species, wherein X=2, 3, 4, 5, 6, 7, 8, 9, or 10.

In an embodiment, the TREM has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, e.g., a separation reagent described herein.

In some embodiments, a TREM composition can be formulated with water for injection. In some embodiments, a TREM composition formulated with water for injection is suitable for pharmaceutical use, e.g., comprises a pharmaceutical TREM composition.

### TREM Purification

A TREM composition, e.g., a TREM pharmaceutical composition, may be purified from host cells by nucleotide purification techniques. In one embodiment, a TREM composition is purified by affinity purification, e.g., as described in the MACS Isolation of specific tRNA molecules protocol, or by a method described in Example 1-3 or 7. In one embodiment, a TREM composition is purified by liquid chromatography, e.g., reverse-phase ion-pair chromatography (IP-RP), ion-exchange chromatography (IE), affinity chromatography (AC), size-exclusion chromatography (SEC), and combinations thereof. See, e.g., Baronti et al. Analytical and Bioanalytical Chemistry (2018) 410:3239-3252.

In an embodiment, a TREM composition can be purified with a purification method comprising one, two or all of the following steps, e.g., in the order recited: (i) separating nucleic acids from protein to provide and RNA preparation; (ii) separating RNA with of less than 200 nt from larger RNA species; and/or (iii) separating a TREM from other RNA species by affinity-based separation, e.g., sequence affinity.

In an embodiment, steps (i)-(iii) are performed in the order recited.

In an embodiment, the purification method comprises step (i). In an embodiment, step (i) comprises extracting nucleic acids from protein in a sample, e.g., as described in Example 1. In an embodiment, the extraction method comprises a phenol chloroform extraction, In an embodiment, the purification method comprises step (ii). In an embodiment, step (ii) is performed on a sample, after step (i). In an embodiment, step (ii) comprises separating RNA of less than a threshold size, e.g., less than 500 nt, 400 nt, 300 nt, 250 nt, or 200 nt in size from larger RNAs, e.g., using a miRNeasy kit as described in Example 1. In an embodiment, step (ii) comprises performing a salt precipitation, e.g., LiCl precipitation, to enrich for small RNAs (e.g., remove large RNAs), as described in Example 1. In an embodiment, separation of the RNA of less than a threshold size from larger RNAs, e.g., using a miRNeasy kit, is performed prior to the salt precipitation, e.g., LiCl precipitation. In an embodiment, step (ii) further comprises performing a desalting or buffer exchange step, e.g., with a G25 column.

In an embodiment, the purification method comprises step (iii). In an embodiment, step (iii) comprises performing an affinity-based separation to enrich for a TREM. In an embodiment, step (iii) is performed on a sample after step (i) and/or step (ii). In an embodiment, the affinity based separation comprises a sequence based separation, e.g., using a probe (e.g., oligo) comprising a sequence that binds to a TREM, e.g., as described in Example 1. In an embodiment, the probe (e.g., oligo) comprises one or more tags, e.g., a biotin tag and/or a fluorescent tag.

In an embodiment, the TREM purification method comprising steps (i), (ii) and (iii) results in a purified TREM composition. In an embodiment, a TREM composition purified according to a method described herein results in lesser RNA contaminants, e.g., as compared to a Trizol RNA extraction purification method.

### TREM Quality Control and Production Assessment

A TREM or a TREM composition, e.g., a pharmaceutical TREM composition, produced by any of the methods disclosed herein can be assessed for a characteristic associated with the TREM or the TREM preparation, such as purity, host cell protein or DNA content, endotoxin level, sterility, TREM concentration, TREM structure, or functional activity of the TREM. Any of the above-mentioned characteristics can be evaluated by providing a value for the characteristic, e.g., by evaluating or testing the TREM, the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of HCP in the composition; (iii) decrease the amount of DNA in the composition; (iv) decrease the amount of fragments in the composition; (v) decrease the amount of endotoxins in the composition; (vi) increase the in vitro translation activity of the composition; (vii) increase the TREM concentration of the composition; or (viii) inactivate or remove any viral contaminants present in the composition, e.g., by reducing the pH of the composition or by filtration.

In an embodiment, the TREM (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has a host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., TREM composition or an intermediate in the production of the TREM composition) has a host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the TREM composition.

In an embodiment, the TREM (e.g., TREM composition or an intermediate in the production of the TREM composition) has a DNA content, e.g., host cell DNA content, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., TREM composition or an intermediate in the production of the TREM composition) has less than 0.1%, 0,5%, 1%, 2%, 3%, 4%, 5%, 6%, 7%, 8%, 9%, 10%, 15%, 20%, 25% TREM fragments relative to full length TREMs.

In an embodiment, the TREM (e.g., TREM composition or an intermediate in the production of the TREM composition) has low levels or absence of endotoxins, e.g., a negative result as measured by the Limulus amebocyte lysate (LAL) test; In an embodiment, the TREM (e.g., TREM composition or an intermediate in the production of the TREM composition) has in-vitro translation activity, e.g., as measured by an assay described in Example 15.

In an embodiment, the TREM (e.g., TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

An TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments the TREM, or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM). The viral vector may be administered to a cell or to a subject (e.g., a human subject or animal model) to deliver a TREM, a TREM composition or a pharmaceutical TREM composition. A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM composition or a pharmaceutical TREM composition described herein can be administered to a cell in a vesicle or other membrane-based carrier.

In embodiments, a TREM or TREM composition, or pharmaceutical TREM composition described herein is administered in or via a cell, vesicle or other membrane-based carrier. In one embodiment, the TREM or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM or TREM compositions or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the TREM or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the TREM or TREM composition, or pharmaceutical TREM composition described herein.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%. 50%. 60%. 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

Table of Contents for Examples
Manufacture and preparation of TREMs

| | |
|---|---|
| Example 1 | Manufacture of a TREM in a mammalian production host cell from |
| | transient transfection |
| Example 2 | Manufacture of a TREM in a mammalian production host cell from |
| | stable cell lines |
| Example 3 | Manufacture of a TREM in a mammalian production host cell from |
| | stable cell lines |

Delivery of TREMs

| | |
|---|---|
| Example 4 | Delivery of TREMs to mammalian cells |

Assays to analyze TREM activity

| | |
|---|---|
| Example 5 | TREM functional activity assay in mammalian cells |
| Example 6 | TREM translational activity assay in Human Cell Extract Cell-Free |
| | Protein Synthesis (hCFPS) lysate |

Manufacture and preparation of TREMs

| | |
|---|---|
| Example 7 | Manufacture of a TREM in a mammalian production host cell, and use |
| | thereof to modulate a cellular function −1 |
| Example 8 | Manufacture of a TREM in a mammalian production host cell, and use |
| | thereof to modulate a cellular function −2 |
| Example 9 | Manufacture of a TREM in modified mammalian production host cell |
| | expressing an oncogene |
| Example 10 | Preparation of a TREM production host cell modified to inhibit a |
| | repressor of tRNA synthesis |
| Example 11 | Manufacture of a TREM in modified mammalian production host cell |
| | overexpressing an oncogene and a tRNA modifying enzyme |

Production of TREMs

| | |
|---|---|
| Example 12 | Production of a mischarged TREM |
| Example 13 | Production of a TREM fragment (in vitro) |
| Example 14 | Production of a TREM fragment in a cell expression system |

Assays to analyze TREM activity

| | |
|---|---|
| Example 15 | TREM translational activity assay |
| Example 16 | Assay for modulation of cell state |
| Example 17 | Assay for the activity of an uncharged TREM to modulate autophagy |
| Example 18 | Assay for activity of a mischarged TREM (mTREM) |

### Example 1: Manufacture of a TREM in a Mammalian Production Host Cell from Transient Transfection

This example describes the manufacture of a TREM produced in mammalian host cells which transiently express a TREM.

### Plasmid Generation

To generate a plasmid comprising a sequence encoding a TREM, in this example, iMet-CAT TREM, a DNA fragment containing one copy of the sequence AGCAGAGTGGCGCAGCGGAAGCGTGCTGGGCCCATAACCCAGAGGTCGATGGATCG AAACCATCCTCTGCTA (SEQ ID NO: 262) was synthesized and cloned into the pLKO.1-puro-mCherry backbone plasmid with a U6 promoter following the manufacturer's instructions and standard molecular cloning techniques.

### Transfection

Three (3) μg of plasmid described above was used to transfect a T175 flask of HEK293T cells plated at 80% confluency using 9 uL of lipofectamine RNAiMax reagents according to the manufacturer's instructions. Cells were harvested at 48 hours post-transfection for purification.

### Purification Using a Small RNA Isolation Kit

The iMet-overexpressing cells were lysed. To generate a small RNA (sRNA) fraction, a small RNA isolation kit, such as the Qiagen miRNeasy kit, was used to separate RNAs smaller than 200 nucleotides from the rest of the total RNA pool in the lysate, per manufacturer's instructions. To further exclude larger RNAs, a LiCl precipitation was performed to remove remaining large RNAs in the sRNA fraction. Finally, the sRNA fraction was added to a G50 column to remove RNAs smaller than 10 nucleotides from the sRNA fraction and for buffer exchange.

To isolate the TREM from the sRNA fraction, a probe binding method was used. A biotinylated capture probe corresponding to a DNA probe or a 2′-OMe nucleic acid that is complementary to a unique region of the target TREM being purified, in this example, a probe conjugated to biotin at the 5′ end with the sequence TAGCAGAGGATGGTTTCGATCCATCA (SEQ ID NO: 267), was used to bind and purify the iMet-CAT-TREM. The sRNA fraction was incubated with annealing buffer and the biotinylated capture probe at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times with wash buffer. The TREM retained on the beads was eluted by adding elution buffer with or without a DNase enzyme to ensure complete removal of the DNA capture probe and then admixed with a pharmaceutically acceptable excipient to make a test TREM product.

### Example 2: Manufacture of a TREM in a Mammalian Production Host Cell from Stable Cell Lines

This example describes the manufacture of a TREM produced in mammalian host cells stably expressing a TREM.

### Preparation of TREM Expressing Lentivirus

To prepare a TREM expressing lentivirus in a 10 mm dish, packaging cells, such as HEK293T cells (293T cells (ATCC® CRL-3216™), were forward transfected with 9 μg of a plasmid comprising a sequence encoding a TREM as described in Example 1, and 9 μg ViraPower lentiviral packaging mix using TransIT-LT1 transfection reagents according to the manufacturer's instructions.

After 18 hours, the media was replaced with fresh antibiotic-free high-FBS (30% FBS) media and 24 hours later, the media containing the virus was harvested and stored at 4° C. Another 15 mL of high-FBS media was added to the plate and harvested 24 hours later. Both virus-containing media harvests were pooled and filtered through a 0.45-micron filter. The viral copy number was assessed using the Lenti-X qRT-PCR Titration Kit according to the manufacturer's protocol.

Transduction of Host Cells with TREM Expressing Lentivirus

To transduce the cells with TREM expressing lentivirus, the lentivirus-containing media was diluted with complete cell media at a 1:4 ratio, in the presence of 10 μg/mL polybrene, and added to the cells. In this example 293T cells were used. The plate was spun for 2 hours at 1000×g to spin infect the cells. After 18 hours, the media was replaced to allow the cells to recover. Forty-eight hours after transduction, puromycin (at 2 μg/mL) antibiotic selection was performed for 5-7 days alongside a population of untransduced control cells.

The TREMs were isolated, purified, and formulated as described in Example 1 to result in a TREM preparation.

### Purification Using Phenol Chloroform Extraction

The total RNA pool from cells was recovered from cells by guanidinium thiocyanate-phenol-chloroform extraction and concentrated by ethanol precipitation as described in J. Sambrook and D. Russell (2001) Molecular Cloning: A Laboratory Manual, vol. 2, Cold Spring Harbor Laboratory Press, New York, N.Y., USA, 3rd edition 2. The total tRNA pool in the precipitate was then separated from larger nucleic acids (including rRNA and DNA) by precipitation under high lithium salt conditions as described in Cathala, G. et al., DNA, 1983; 2(4):329-35. The elution fraction containing the TREM was further purified through probe binding.

The TREM fraction was incubated with annealing buffer and the biotinylated capture probe corresponding to a DNA probe or a 2′-OMe nucleic acid that is complementary to a unique region of the target TREM being purified. In this example, a probe conjugated to biotin at the 5′ end with the sequence TAGCAGAGGATGGTTTCGATCCATCA (SEQ ID NO: 267), was used to purify the TREM comprising iMet-CAT. The mixture was incubated at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times. The TREM retained on the beads were eluted by adding elution buffer with or without a DNase enzyme to ensure complete removal the DNA capture probe and then admixed with a pharmaceutically acceptable excipient to make a test TREM product.

### Example 3: Manufacture of a TREM in a Mammalian Production Host Cell from Stable Cell Lines

This example describes the manufacture of a TREM from crude cell lysate, produced from mammalian host cells.

### Generation of Stable Cells Expressing TREM

In this example, a plasmid comprising a sequence encoding a TREM is generated as described in Example 1 or 2. Preparation of TREM expressing lentivirus and transduction of host cells with TREM-expressing lentivirus was performed as described in Example 2.

Purification from Crude Cell Lysate

The TREM-overexpressing cells, in this example the iMet-CAT-TREM overexpressing cells, were lysed and the lysed material was incubated with annealing buffer and the biotinylated capture probe corresponding to a DNA probe or a 2′-OMe nucleic acid that is complementary to a unique region of the target TREM being purified. In this example, a probe conjugated to biotin at the 5′ end with the sequence TAGCAGAGGATGGTTTCGATCCATCA (SEQ ID NO: 267), was used to purify the TREM comprising iMet-CAT. The mixture was incubated at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times. The TREM retained on the beads were eluted by adding elution buffer with or without a DNase enzyme to ensure complete removal the DNA capture probe and then admixed with a pharmaceutically acceptable excipient to make a test TREM product.

### Example 4: Delivery of TREMs to Mammalian Cells

This example describes the delivery of a TREM to mammalian cells.

To ensure proper folding, the TREM was heated at 85° C. for 2 minutes and then snap cooled at 4° C. for 5 minutes. To deliver the TREM to mammalian cells, 100 nM of two TREM preparations labeled with Cy3 at different positions (Cy3-iMET-1 and Cy3-iMET-2) were transfected in U2OS (U-2 OS (ATCC® HTB-96™)), H1299 (NCI-H1299 (ATCC® CRL-5803™)), and HeLa (HeLa (ATCC® CCL-2™)) cells using RNAiMax reagents according to the manufacturer's instructions. After 18 hours, the transfection media was removed and replaced with fresh complete media (U2OS: McCoy's 5A, 10% FBS, 1% PenStrep; H1299: RPMI1640, 10% FBS, 1% PenStrep; HeLa: EMEM, 10% FBS, 1% PenStrep).

To observe TREM delivery to cells, the cells were monitored in a live cell analysis system. In this example, the IncuCyte (from Essen Bioscience) was used to monitor cells. The cells were monitored for 4 days (20×, red 550 ms).

Cy3 fluorescence signal was readily detected from cells that had been delivered the Cy3-labeled TREMs. The Cy3 fluorescence signal was observed for over 48 hours from the cells in which the TREMs had been delivered. Detection of Cy-3 fluorescence from the cells confirmed delivery of the Cy3-labeled TREM to the cells.

### Example 5: Increased Cell Growth in Mammalian Cells with TREM

This example describes increased cell growth of a mammalian cell upon TREM delivery.

To ensure proper folding, the iMet TREM was heated at 85° C. for 2 minutes and then snap cooled at 4° C. for 5 minutes. To deliver the iMet TREM to mammalian cells, 100 nM of Cy3-labeled iMet TREM was transfected in U2OS (U-2 OS (ATCC® HTB-96™)), H1299 (NCI-H1299 (ATCC® CRL-5803™)), and HeLa (HeLa (ATCC® CCL-2™)) cells using RNAiMax reagents according to the manufacturer's instructions. As a control, a Cy3-labeled non targeted control siRNA was delivered to cells. After 18 hours, the transfection media was removed and replaced with fresh complete media (U2OS: McCoy's 5A, 10% FBS, 1% PenStrep; H1299: RPMI1640, 10% FBS, 1% PenStrep; HeLa: EMEM, 10% FBS, 1% PenStrep). To observe changes in cell growth, the cells were monitored in a live cell analysis system, in this example in the IncuCyte (from Essen Bioscience), for 4 days (20×, phase contrast).

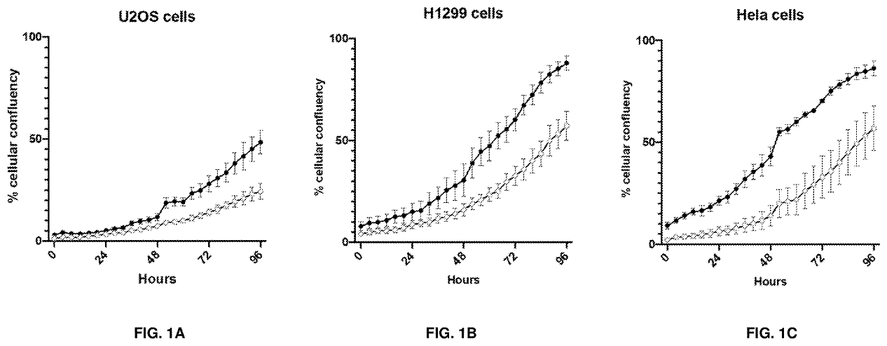

Delivery of iMet TREM to U2OS cells (FIG. 1A), H1299 (FIG. 1B) or Hela cells (FIG. 1C) led to a substantial increase in cell growth in all of the cell lines that were tested. The increase in cell growth was compared to cell growth observed with delivery of a Cy3-labeled non-targeted control (Cy3-NTC). The data demonstrates that delivery of a TREM to cells results in increased proliferation and growth.

### Example 6: TREM Translational Activity Assay in Human Cell Extract Cell-Free Protein Synthesis (hCFPS) Lysate

This example describes a TREM mediated increase in translational activity in a cell-free lysate system.

### Preparing Human Cell Extracts

HEK293T cells were grown to ˜80% confluency in 40×150 mm culture dishes. The cells were harvested, washed in PBS, resuspended 1:1 in ice-cold hypotonic lysis buffer (20 mM HEPES pH 7.6, 10 mM KAc, 1.5 mM MgAc, 5 mM DTT and 5× complete EDTA-free proteinase inhibitor cocktail) and incubated on ice for 30 minutes. Cells were lysed using a Dounce homogenizer or by passing the lysate through a 27 G needle, until >95% of the cells were disrupted. The lysate was centrifuged at 14,000 g for 10 mins at 4° C., the supernatant was collected and diluted with the hypotonic lysis buffer to get a ˜15 mg/ml protein solution.

Transcribing mRNAs

mRNA transcription templates were designed to have a T7 polymerase promoter, a beta-globin 3′UTR, a nanoLuc ORF, and a short artificial 3′UTR. The templates were PCR amplified and used to transcribe capped and poly-adenylated mRNAs with a HiScribe T7 ARCA mRNA kit with tailing (New England Biolabs) following the manufacturer's recommended protocol.

Performing the TREM Translational Activity Assay in hCFPS Lysate

Translation reactions were set up in translation buffer (16 mM HEPES pH 7.6, 2.2 mM MgAc, 60 mM KCl, 0.02 mM complete amino acid mix, 1 mM ATP, 0.5 mM GTP, 20 mM creatine phosphate, 0.1 μg/L creatine kinase, 0.1 mM spermidine, 2 U/μl RiboLock RNase Inhibitor) with 35% HEK293T lysate, 0.02 μM capped and poly-adenylated nanoLuc mRNA and 2 μM cell-purified TREM (purified according to Example 2). The reactions were performed in 10 μl triplicates at 37° C. for 30 minutes. For the control reactions, one control reaction was performed with no TREM addition to the reaction and one control reaction was performed with no mRNA addition to the reaction. Then, the NanoLuc activity was detected by mixing each reaction with 40 μl of room temperature Nano-Glo Luciferase assay system (Promega) and reading the luminescence in a plate reader.

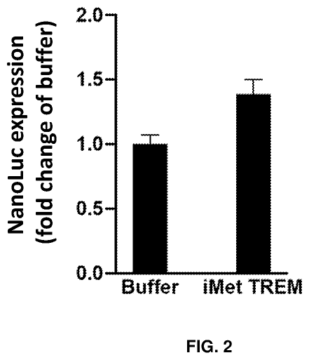

As shown in FIG. 2, the iMET TREM reaction resulted in about a 1.5 fold increase in NanoLuc expression as compared to the control reaction (buffer). The data shows that delivery of the TREM results in an increase in nanoLuc mRNA translation as reflected by an increase in luminescence.

### Example 7: Manufacture of a TREM in a Mammalian Production Host Cell, and Use Thereof to Modulate a Cellular Function-1

This example describes the manufacture of a TREM produced in mammalian host cells.

### Plasmid Generation

To generate a plasmid comprising a sequence encoding a TREM, in this example, iMet-CAT TREM, a DNA fragment with genomic location 6p22.2 and sequence AGCAGAGTGGCGCAGCGGAAGCGTGCTGGGCCCATAACCCAGAGGTCGATGGATCG AAACCATCCTCTGCTA (SEQ ID NO: 622)) is PCR-amplified from human genomic DNA using the following primer pairs: 5′-TGAGTTGGCAACCTGTGGTA (SEQ ID NO: 623) and 5′-TTGGGTGTCCATGAAAATCA (SEQ ID NO: 624). This fragment is cloned into the pLKO.1 puro backbone plasmid with a U6 promoter (or any other RNA polymerase III recruiting promoter) following the manufacturer's instructions.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR).

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe or a 2′-OMe nucleic acid that is complementary to a unique region of the target TREM being purified, in this example, a probe conjugated to biotin at the 3′ end with the sequence UAGCAGAGGAUGGUUUCGAUCCAUCA (SEQ ID NO: 625), is used to purify the iMet-CAT-TREM. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-TREM complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Use

One microgram of the test TREM preparation and a control agent are contacted by transfection, electroporation or liposomal delivery, with a cultured cell line, such as a HEP-3B or HEK293T, a tissue or a subject, for a time sufficient for the TREM preparation to modulate a translation level or activity of the cell, relative to the control agent.

### Example 8: Manufacture of a TREM in a Mammalian Production Host Cell, and Use Thereof to Modulate a Cellular Function-2

This example describes the manufacture of a TREM produced in mammalian host cells.

### Plasmid Generation

To generate a plasmid comprising a sequence encoding a TREM, in this example, iMet-CAT-TREM, a DNA fragment containing at least one copy of the sequence AGCAGAGTGGCGCAGCGGAAGCGTGCTGGGCCCATAACCCAGAGGTCGATGGATCG AAACCATCCTCTGCTA (SEQ ID NO: 626) is synthesized and cloned into the pLKO.1 puro backbone plasmid with a U6 promoter (or any other RNA polymerase III recruiting promoter) following the manufacturer's instructions and standard molecular cloning techniques.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR) or Nanopore sequencing.

### Purification

At the optimized harvest timepoint, the cells are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe or a 2′-OMe nucleic acid that is complementary to a unique region of the target TREM being purified. In this example, a probe with the sequence 5′biotin-TAGCAGAGGATGGTTTCGATCCATCA (SEQ ID NO: 627) is used to purify the -iMet-CAT-TREM. The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. Alternatively, the beads are washed three times and the final wash is examined by UV spectroscopy to measure the amount of nucleic acid present in the final wash. The TREM retained on the beads are eluted three times using RNase-free water which can be pre-heated to 80° C., and then admixed with a pharmaceutically acceptable excipient to make a test TREM product.

### Use

One microgram of the test TREM preparation and a control agent are contacted by transfection, electroporation or liposomal delivery, with a cultured cell line, such as HeLa, HEP-3B or HEK293T, a tissue or a subject, for a time sufficient for the TREM preparation to modulate a translation level or activity of the cell, relative to the control agent.

### Example 9: Manufacture of a TREM in Modified Mammalian Production Host Cell Expressing an Oncogene

This example describes the manufacture of a TREM in mammalian host cells modified to overexpress Myc.

### Plasmid Generation and Host Cell Modification

To make the production host cells for this example, HeLa cells (ATCC® CCL-2™) or HEP-3B cells (ATCC® HB-8064™) are transfected with a plasmid containing the gene sequence coding for the c-myc oncogene protein (e.g., pcDNA3-cmyc (Addgene plasmid #16011)) using routine molecular biology techniques. The resulting cell line is referred to herein as HeLamyc+ host cells or HEP-3Bmyc+ host cells.

### Preparation of TREM Expressing Lentivirus

To prepare a TREM expressing lentivirus, HEK293T cells are co-transfected with 3 μg of each packaging vector (pRSV-Rev, pCMV-VSVG-G and pCgpV) and 9 μg of the plasmid comprising a sequence encoding a TREM as described in Example 7, using Lipofectamine 2000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 m filter.

Transduction of Host Cells with TREM Expressing Lentivirus

Two (2) mL of virus prepared as described above is used to transduce 100,000 HeLamyc+ host cells or HEP-3Bmyc+ host cells, in the presence of 8 μg/mL polybrene. Forty-eight hours after transduction, puromycin (at 2 μg/mL) antibiotic selection is performed for 2-7 days alongside a population of untransduced control cells.

The TREMs are isolated, purified, and formulated as described in Example 7 or 8 to result in a TREM composition or preparation.

### Example 10: Preparation of a TREM Production Host Cell Modified to Inhibit a Repressor of tRNA Synthesis

This example describes the preparation of Hek293Maf-/TRM1 cells for the production of a TREM.

Maf1 is a repressor of tRNA synthesis. A Maf1 knockout HEK293T cell line is generated using standard CRISPR/Cas knockout techniques, e.g., a CRISPR/Cas system can be designed to introduce a frameshift mutation in a coding exon of Maf1 to reduce the expression of Maf1 or knockout Maf1 expression, to generate a Hek293Maf-cell line that has reduced expression level and/or activity of Maf1. This cell line is then transfected with an expression plasmid for modifying enzyme Trm1 (tRNA (guanine26-N2)-dimethyltransferase) such as pCMV6-XL4-Trm1, and selected with a selection marker, e.g., neomycin, to generate a stable cell line overexpressing Trm1 (Hek293Maf-/TRM1 cells).

Hek293Maf-/TRM1 cells can be used as production host cells for the preparation of a TREM as described in any of Examples 7-9.

### Example 11: Manufacture of a TREM in Modified Mammalian Production Host Cells Overexpressing an Oncogene and a tRNA Modifying Enzyme

This Example describes the manufacture of a TREM in mammalian host cells modified to overexpress Myc and Trm1.

### Plasmid Generation

In this example, a plasmid comprising a TREM is generated as described in Example 7 or 8.

### Host Cell Modification, Transduction and Purification

A human cell line, such as HEK293T, stably overexpressing Myc oncogene is generated by transduction of retrovirus expressing the myc oncogene from the pBABEpuro-c-myc^{T58A} plasmid into HEK293T cells. To generate myc-expressing retrovirus, HEK293T cells are transfected using the calcium phosphate method with the human c-myc retroviral vector, pBABEpuro-c-myc^{T58A} and the packaging vector, ψ2 vector. After 6 hours, transfection media is removed and replaced with fresh media. After a 24-hour incubation, media is collected and filtered through a 0.45 um filter. For the retroviral infection, HEK293T cells are infected with retrovirus and polybrene (8 ug/ml) using spin infection at 18° C. for 1 hour at 2500 rpm. After 24 hours, the cell culture medium is replaced with fresh medium and 24 hours later, the cells are selected with 2 μg/mL puromycin. Once cells stably overexpressing the oncogene myc are established, they are transfected with a Trm1 plasmid, such as the pCMV6-XL4-Trm1 plasmid, and selected with a selection marker, in this case with neomycin, to generate a stable cell line overexpressing Trm1, in addition to Myc. In parallel, lentivirus to overexpress TREM is generated as described in Example 9 with HEK293T cells and PLKO.1-TREM vectors.

One hundred thousand (1×10⁵) cells overexpressing Myc and Trm1 are transduced with the TREM virus in the presence of 8 μg/mL polybrene. Media is replaced 24 hours later. Forty-eight hours after transduction, antibiotic selection is performed with 2 μg/mL puromycin for 2-7 days alongside a population of untransduced control cells. The TREMs are isolated, purified and formulated using the method described in Example 7 or 8 to produce a TREM preparation.

### Example 12: Production of a Mischarged TREM

This example describes the production of a TREM charged with an amino acid that does not correspond to its natural anticodon.

A TREM is produced as described in any of Examples 7-11. The TREM product is charged with a heterologous amino acid using an in vitro charging reaction known in the art (see, e.g., Walker & Fredrick (2008) Methods (San Diego, Calif.) 44(2):81-6). Briefly, the purified TREM, for example a TREM comprising tRNA-Val(GTG), is placed in a buffer with the heterologous amino acid of interest (for example glutamic acid), and the corresponding aminoacyl-tRNA synthetase (for example a Valyl-tRNA synthetase mutated to enhance tRNA mischarging), to induce TREM charging.

To isolate the aminoacyl-TREM, the in vitro charging reaction is passed through a spin column and the concentration based on the A₂₆₀ absorbance is determined as is the extent of aminoacylation using acid gel electrophoresis. Aminoacylated TREM can also be isolated by binding to His6-tagged EF-Tu (“His6” disclosed as SEQ ID NO: 628), followed by affinity chromatography on Ni-NTA agarose, phenol-chloroform extraction and subsequent precipitation of the nucleic acids as described in Rezgui et al., 2013, PNAS 110:12289-12294.

### Example 13: Production of a TREM Fragment (In Vitro)

This example describes the production of a TREM fragment in vitro, from a TREM manufactured in mammalian host cells.

A TREM is made as described in any one of Examples 7-13 above. An enzymatic cleavage assay with enzymes known to generate tRNA fragments, such as RNase A or angiogenin, is used to produce fragments for administration to a cell, tissue or subject.

Briefly, a TREM manufactured as describe above is incubated in one of: 0.1M Hepes/NaOH, pH 7.4 with 10 nM final concentration of RNase A for 10 min at 30° C., or 0.1M MES, 0.1M NaCl, pH 6.0, with an effective amount of angiogenin, and BSA for 6 hours at 37° C.

To isolate a target TREM fragment after enzymatic treatment, a sequence affinity purification procedure is performed, as described above.

### Example 14: Production of a TREM Fragment in a Cell Expression System

This example describes the production of a TREM fragment in a cell expression system.

A cell line stably overexpressing a TREM is generated as described in any of Examples 7-9 or 11. Hek293T cells overexpressing the TREM are treated with 0.5 pg/ml recombinant angiogenin for 90 min before total RNA is extracted with Trizol. Size selection of RNAs smaller than 200 nucleotides is performed using a small RNA isolation kit per manufacturer's instructions. Streptavidin-conjugated RNase-free magnetic beads are incubated at room temperature for 30 min with 200 mM of biotinylated oligonucleotides corresponding to a probe or a DNA probe that is complementary to a unique region of the TREM fragment being purified. The beads are washed and heated for 10 min at 75° C. The size-selected RNA eluate is also heated for 10 min at 75° C. and then mixed with the beads. The TREM-bead mixture is incubated at room temperature for 3 hours to allow binding of the TREMs to the bead-bound DNA probe. The beads are then washed until the wash solution at 260 nm is close to zero (0). Alternatively, the beads are washed three times and the final wash is examined by UV spectroscopy to measure the amount of nucleic acid present in the final wash. The TREM retained on the beads are eluted 3 times using RNase-free water pre-heated to 80° C. or elution buffer pre-heated to 80° C.

### Example 15: TREM Translational Activity Assays

This example describes assays to evaluate the ability of a TREM to be incorporated into a nascent polypeptide chain.

### Translation of the FLAG-AA-his Peptide Sequence

A test TREM is assayed in an in-vitro translation reaction with an mRNA encoding the peptide FLAG-XXX-His6× (“His6” disclosed as SEQ ID NO: 628), where XXX are 3 consecutive codons corresponding to the test TREM anticodon.

A tRNA-depleted rabbit reticulocyte lysate (Jackson et al. 2001. RNA 7:765-773) is incubated 1 hour at 30° C. with 10-25 ug/mL of the test TREM in addition to 10-25 ug/mL of the tRNAs required for the FLAG and His tag translation. In this example, the TREM used is Ile-GAT-TREM, therefore the peptide used is FLAG-LLL-His6× (“His6” disclosed as SEQ ID NO: 628) and the TREM added is TREM-Ile-GAT, in addition to the following, which are added to translate the peptide FLAG and HIS tags: tRNA-Asp-GAC, tRNA-Tyr-TAC, tRNA-Lys-AAA, tRNA-Lys-AAAG, tRNA-Asp-GAT, tRNA-His-CAT. To determine if the test TREM is functionally able to be incorporated into a nascent peptide, an ELISA capture assay is performed. Briefly, an immobilized anti-His6× antibody (“His6” disclosed as SEQ ID NO: 628) is used to capture the FLAG-LLL-His6× peptide (“His6” disclosed as SEQ ID NO: 628) from the reaction mixture. The reaction mixture is then washed off and the peptide is detected with an enzyme-conjugated anti-FLAG antibody, which reacts to a substrate in the ELISA detection step. If the TREM produced is functional, the FLAG-LLL-His6 peptide (“His6” disclosed as SEQ ID NO: 628) is produced and detection occurs by the ELISA capture assay.

If the TREM produced is not functional, the FLAG-LLL-His6 peptide (“His6” disclosed as SEQ ID NO: 628) is not produced and no detection occurs by the ELISA capture assay.

### Translational Suppression Assay

This assay describes a test TREM having translational adaptor molecule function by rescuing a suppression mutation and allowing the full protein to be translated. The test TREM, in this example Ile-CUA-TREM, is produced such that it contains the sequence of the Ile-GAT-TREM body but with the anticodon sequence corresponding to CUA instead of GAT. HeLa cells are co-transfected with 50 ng of TREM and with 200 ng of a DNA plasmid encoding a mutant GFP containing a TAG stop codon at the S29 position as described in Geslain et al. 2010. J Mol Biol. 396:821-831. HeLa cells transfected with the GFP plasmid alone serve as a negative control. After 24 hours, cells are collected and analyzed for fluorescence recovery by flow cytometry. The fluorescence is read out with an emission peak at 509 nm (excitation at 395 nm). It is expected that if the test TREM is functional, it can or will be sufficient to rescue the stop mutation in the GFP molecule and can produce the full-length fluorescent protein, which is detected by flow cytometry. If the test TREM is not functional or is less functional, the stop mutation is likely not to be rescued, and no fluorescence is emitted from the GFP molecule and accordingly a reduced GFP signal or no GFP signal is detected by flow cytometry.

### In Vitro Translational Assay

This assay describes a test TREM having translational adaptor molecule function by successfully being incorporated into a nascent polypeptide chain in an in vitro translation reaction. First, a rabbit reticulocyte lysate that is depleted of the endogenous tRNA using an antisense or complimentary oligonucleotide which (i) targets the sequence between the anticodon and variable loop; or (ii) binds the region between the anticodon and variable loop is generated (see, e.g., Cui et al. 2018. Nucleic Acids Res. 46(12):6387-6400). 10-25 ug/mL of the test TREM is added in addition to 2 ug/uL of a GFP-encoding mRNA to the depleted lysate. A non-depleted lysate with the GFP mRNA, with or without the test TREM added are used as a positive control. A depleted lysate with the GFP mRNA but without the test TREM added is used as a negative control. The progress of GFP mRNA translation is monitored by fluorescence increase on a microplate reader at 37° C. for 3-5 h using λₑₓ485/λₑₘ528. It is expected for the experimental sample to be able to produce similar levels of fluorescence over time as the positive control and to be able to produce higher levels of fluorescence over time compared to the negative control. If so, these results would likely indicate that the test TREM is sufficient to, or can complement the depleted lysate and is thus likely functional.

### Example 16: Assay for Modulation of Cell State

This example describes an assay for detecting activity of a TREM in modulating cell status, e.g., cell death.

TREM fragments are produced as described in Example 13. One (1) uM of TREM fragments are transfected into HEK293T cells with Lipofectamine 3000 and incubated for 1-6 hours in hour-long intervals followed by cell lysis. Cell lysates are analyzed by Western blotting and blots are probed with antibodies against total and cleaved caspase 3 and 9 as readouts of apoptosis. To measure cellular viability, cells are washed and fixed with 4% paraformaldehyde in PBS for 15 minutes at room temperature. Fixed and washed cells are then treated with 0.1% Triton X-100 for 10 minutes at room temperature and washed with PBS three times. Finally, cells are treated with TUNEL assay reaction mixture at 37° C. for 1 hour in the dark. Samples are analyzed by flow cytometry.

### Example 17: Assay for the Activity of an Uncharged TREM to Modulate Autophagy

This example describes an assay to test an uncharged TREM for ability to modulate, e.g., induce, autophagy, e.g., the ability to activate GCN2-dependent stress response (starvation) pathway signaling, inhibit mTOR or activate autophagy.

A test uncharged TREM (uTREM) preparation is delivered to HEK293T or HeLa cells through transfection or liposomal delivery. Once the uTREM is delivered, a time course is performed ranging from 30 minutes to 6 hours with hour-long interval time points. Cells are then trypsinized, washed and lysed. The same procedure is executed with a charged control TREM as well as random RNA oligos as controls. Cell lysates are analyzed by Western blotting and blots are probed with antibodies against known readouts of GCN2 pathway activation, mTOR pathway inhibition or autophagy induction, including but not limited to phospho-eIF2a, ATF4, phospho-ULK1, phospho-4EBP1, phospho-eIF2a, phospho-Akt and phospho-p70S6K. A total protein loading control, such as GAPDH, actin or tubulin, as well as the non-modified (i.e. non-phosphorylated) signaling protein, i.e. using eIF2a as a control for phospho-eIF2a, are probed as loading controls. Delivery of the uTREM, compared to controls, is or can be expected to show activation of GCN2 starvation signaling pathway, autophagy pathway and/or inhibition of the mTOR pathway as determined by Western blot analysis.

### Example 18: Assay for Activity of a Mischarged TREM (mTREM)

This example describes an assay to test the functionality of a mTREM produced in a cell system using plasmid transfection followed by in vitro mischarging.

In this example, an mTREM can translate a mutant mRNA into a wild type (WT) protein by incorporation of the WT amino acid in the protein despite an mRNA containing a mutated codon. GFP mRNA molecules with either a T203I or E222G mutation, which prevent GFP excitation at the 470 nm and 390 nm wavelengths, respectively, are used for this example. GFP mutants which prevent GFP fluorescence could also be used as reporter proteins in this assay. Briefly, an in vitro translation assay is used, using a rabbit reticulocyte lysate containing the GFP E222G mutated mRNA (GAG→GGG mutation) and an excess of the mTREM, in this case Glu-CCC-TREM. As a negative control, no mischarged TREM is added to the reaction. If the mTREM is functional, it is or can be expected that the GFP protein produced fluoresces when illuminated with a 390 nm excitation wavelength using a fluorimeter. If the mTREM is not functional or is less functional, the GFP protein produced fluoresces only when excited with a 470 nm wavelength, as is observed in the negative control.

What is claimed is:

1. A method of making a purified tRNA effector molecule (TREM) pharmaceutical composition, comprising:
providing a mammalian host cell comprising an exogenous nucleic acid, e.g., a DNA or RNA, encoding the TREM;
maintaining the mammalian cell under conditions sufficient to express the TREM;
purifying the TREM from the mammalian host cell, e.g., according to a method described herein; and
formulating the purified TREM as a pharmaceutical composition, e.g., by combining the TREM with a pharmaceutical excipient,
thereby making the TREM pharmaceutical composition.

2. The method of claim 1, wherein the nucleic acid comprises an RNA, which upon reverse transcription, results in a DNA which can be transcribed into the TREM.

3. The method of claim 1 or 2, wherein the nucleic acid comprises an RNA sequence at least 90% identical to an RNA sequence encoded by a DNA sequence listed in Table 1, or a fragment or functional fragment thereof.

4. The method of claim 1 or 2, wherein the nucleic acid comprises an RNA sequence comprising a consensus sequence provided herein.

5. The method of any one of the preceding claims, wherein the mammalian host cell is chosen from: a non-human cell or cell line, or a human cell or cell line, e.g., a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.

6. The method of any one of the preceding claims, wherein the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity-based separation.

7. A composition comprising a purified tRNA effector molecule (TREM) (e.g., a purified TREM composition made according to a method described herein), comprising:
(i) an RNA sequence at least 90% identical to an RNA sequence encoded by a DNA sequence listed in Table 1, or a fragment or functional fragment thereof; or
(ii) an RNA sequence comprising a consensus sequence provided herein.

8. A GMP-grade, recombinant TREM composition (e.g., a TREM composition made in compliance with cGMP, and/or in accordance with similar requirements) comprising:
(i) an RNA sequence at least 90% identical to an RNA sequence encoded by a DNA sequence listed in Table 1, or a fragment or functional fragment thereof; or
(ii) an RNA sequence comprising a consensus sequence provided herein.

9. The TREM composition of claim 7 or 8, wherein the composition comprises one or more, e.g., a plurality, of TREMs.

10. The TREM composition of any one of claims 7 to 9, wherein the composition comprises at least 1, 2, 3, 4, 5, 6, 7, 8, 9 or 10 species of TREMs.

11. The TREM composition of any one of claims 7 to 10, wherein the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of, or an undetectable level of viral contamination.

12. A method of modulating a tRNA pool in a cell comprising:
providing a purified TREM composition, and contacting the cell with the TREM composition,
thereby modulating the tRNA pool in the cell.

13. The method of claim 12, wherein the TREM composition is made by:
providing a mammalian host cell comprising an exogenous nucleic acid, e.g., a DNA or RNA, encoding the TREM;
maintaining the mammalian cell under conditions sufficient to express the TREM; and/or
purifying the TREM from the mammalian host cell, e.g., according to a method described herein.

14. The method of claim 12 or 13, wherein the mammalian host cell is chosen from: a non-human cell or cell line, or a human cell or cell line, e.g., a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.

15. The method of any one of claims 12 to 14, wherein the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity-based separation.

16. The method of any one of claims 12 to 15, wherein the TREM comprises:
(i) an RNA sequence at least 80% identical to an RNA sequence encoded by a DNA sequence listed in Table 1, or a fragment or functional fragment thereof; or
(ii) an RNA sequence comprising a consensus sequence provided herein.

17. A method of making a tRNA effector molecule (TREM) composition, comprising:
(a) providing a mammalian host cell comprising exogenous nucleic acid, e.g., a DNA or
RNA, encoding a TREM under conditions sufficient to express the TREM, and
(b) purifying the expressed TREM from the mammalian host cell to produce a TREM composition,
thereby making the TREM composition.

18. A method of making a pharmaceutical TREM composition comprising:
combining
a) a TREM, e.g., a purified TREM composition, e.g., a TREM composition made by a method described herein; and
b) a pharmaceutically acceptable component, e.g., an excipient,
thereby making a pharmaceutical TREM composition.

19. A method of making a purified tRNA effector molecule (TREM) pharmaceutical composition, comprising:
purifying the TREM from a mammalian host cell;
formulating the purified TREM as a pharmaceutical composition, e.g., by combining the TREM with a pharmaceutical excipient,
thereby making the TREM pharmaceutical composition.

20. A method of making a TREM composition, comprising:
contacting a TREM containing a reaction mixture with a reagent, e.g., a capture reagent or a separation reagent, comprising a nucleic acid sequence complimentary with a TREM;
thereby making a TREM composition.

21. A method of making a pharmaceutical composition, comprising:
a) providing a purified TREM composition, e.g., a purified TREM composition made by culturing a mammalian host cell comprising DNA or RNA encoding a TREM under conditions sufficient to express the TREM, and purifying the expressed TREM from the host cell culture to produce a purified TREM composition,
b) providing a value, e.g., by evaluating or testing, for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of, or an undetectable level of viral contamination.
c) optionally, formulating the purified TREM composition as a pharmaceutical drug product (e.g., combining the TREM composition with a pharmaceutical excipient) if it meets a reference criteria for the one or more characteristics,
thereby making a pharmaceutical composition.

22. A pharmaceutical tRNA effector molecule (TREM) composition, comprising
(i) an RNA sequence at least 80% identical to an RNA sequence encoded by a DNA sequence listed in Table 1, or a fragment or functional fragment thereof; or
(ii) an RNA sequence comprising a consensus sequence provided herein.

23. A recombinant TREM composition of at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.

24. A recombinant TREM composition of between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to 500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.

25. A TREM composition comprising a consensus sequence of Formula I_{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) _{ZZZ} indicates any of the twenty amino acids;
(ii) Formula I corresponds to all species; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

26. A TREM composition comprising a consensus sequence of Formula II_{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) _{ZZZ} indicates any of the twenty amino acids;
(ii) Formula II corresponds to mammals; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

27. A TREM composition comprising a consensus sequence of Formula III_{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) _{ZZZ} indicates any of the twenty amino acids;
(ii) Formula III corresponds to humans; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

28. A method of contacting a cell, tissue, or subject with a TREM, comprising
contacting the cell, tissue or subject with a purified TREM composition,
thereby contacting a cell, tissue, or subject with the TREM.

29. A method of presenting a TREM to a cell, tissue, or subject with a TREM, comprising
contacting the cell, tissue or subject with a purified TREM composition,
thereby presenting the TREM to a cell, tissue, or subject.

30. A method of forming a TREM-contacted cell, tissue, or subject, comprising
contacting the cell, tissue or subject with a purified TREM composition,
thereby forming a TREM-contacted cell, tissue, or subject.

31. A method of using a TREM comprising,
contacting the cell, tissue or subject with a purified TREM composition,
thereby using the TREM.

32. A method of applying a TREM to a cell, tissue, or subject, comprising
contacting the cell, tissue or subject with a purified TREM composition,
thereby applying a TREM to a cell, tissue, or subject.

33. A method of exposing a cell, tissue, or subject to a TREM, comprising
contacting the cell, tissue or subject with a purified TREM composition,
thereby exposing a cell, tissue, or subject to a TREM.

34. A method of forming an admixture of a TREM and a cell, tissue, or subject, comprising
contacting the cell, tissue or subject with a TREM composition,
thereby forming an admixture of a TREM and a cell, tissue, or subject.

35. A method of delivering a TREM to a cell, tissue, or subject, comprising:
providing a cell, tissue, or subject, and contacting the cell, tissue, or subject, with a TREM composition, e.g., a purified TREM composition, e.g., a pharmaceutical TREM composition.

36. A method, e.g., an ex vivo method, of modulating the metabolism, e.g., the translational capacity of an organelle, comprising:
providing a preparation of an organelle, e.g., mitochondria or chloroplasts, and contacting the organelle with a pharmaceutical TREM composition.

37. A method of treating a subject, e.g., modulating the metabolism, e.g., the translational capacity of a cell, in a subject, comprising:
providing, e.g., administering to the subject, an exogenous nucleic acid, e.g., a DNA or RNA, which encodes a TREM,
thereby treating the subject.

38. A cell comprising a TREM made according to a method of making a TREM disclosed herein.

39. A cell comprising a TREM disclosed herein.

40. A cell comprising an exogenous nucleic acid comprising:
a nucleic acid sequence, e.g., DNA or RNA, that encodes a TREM, wherein the nucleic acid sequence comprises:
(i) a control region sequence;
(ii) a sequence encoding a modified TREM;
(iii) a sequence encoding more than one TREM;
(iv) a sequence other than a tRNA^{Met} sequence; or
(v) a promoter sequence that comprises a Pol III recognition site, e.g., a U6 promoter, a 7SK promoter or a H1 promoter, or a fragment thereof.

41. A reaction mixture comprising a TREM and a reagent, e.g., a capture reagent, or a separation reagent.

42. A bioreactor comprising a plurality of mammalian host cells described herein comprising exogenous DNA or RNA.

43. A master cell bank comprising a host cell, e.g., as described herein.

44. A method of evaluating a composition of TREM, e.g., a GMP-grade TREM (i.e., a TREM made in compliance with cGMP, and/or in accordance with similar requirements), comprising acquiring a value for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 15;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., the composition or preparation supports the growth of fewer than 100 viable microorganisms as tested under aseptic conditions, the composition or preparation meets the standard of USP <71>, and/or the composition or preparation meets the standard of USP <85> as described by cGMP guidelines for sterile drug products produced by aseptic processing; or
(x) viral contamination, e.g., the composition or preparation has an absence of, or an undetectable level of viral contamination.


SEQUENCE LISTING (628 sequences)

SEQ ID NO: 1 - DNA, 72 bp, Homo sapiens
gggggtatagctcagtggtagagcgcgtgcttagcatgcacgaggtcctgggttcgatccccagtacctcca

SEQ ID NO: 2 - DNA, 73 bp, Homo sapiens
ggggaattagctcaagtggtagagcgcttgcttagcacgcaagaggtagtgggatcgatgcccacattctcca

SEQ ID NO: 3 - DNA, 73 bp, Homo sapiens
ggggaattagctcaaatggtagagcgctcgcttagcatgcgagaggtagcgggatcgatgcccgcattctcca

SEQ ID NO: 4 - DNA, 73 bp, Homo sapiens
ggggaattagctcaagtggtagagcgcttgcttagcatgcaagaggtagtgggatcgatgcccacattctcca

SEQ ID NO: 5 - DNA, 73 bp, Homo sapiens
ggggaattagctcaagcggtagagcgcttgcttagcatgcaagaggtagtgggatcgatgcccacattctcca

SEQ ID NO: 6 - DNA, 73 bp, Homo sapiens
ggggaattagctcaagtggtagagcgcttgcttagcatgcaagaggtagtgggatcaatgcccacattctcca

SEQ ID NO: 7 - DNA, 73 bp, Homo sapiens
ggggaattagctcaagtggtagagcgctcgcttagcatgcgagaggtagtgggatcgatgcccgcattctcca

SEQ ID NO: 8 - DNA, 73 bp, Homo sapiens
ggggaattagcccaagtggtagagcgcttgcttagcatgcaagaggtagtgggatcgatgcccacattctcca

SEQ ID NO: 9 - DNA, 72 bp, Homo sapiens
gggggtgtagctcagtggtagagcgcgtgcttagcatgcacgaggccccgggttcaatccccggcacctcca

SEQ ID NO: 10 - DNA, 72 bp, Homo sapiens
gggggtgtagctcagtggtagagcgcgtgcttagcatgtacgaggtcccgggttcaatccccggcacctcca

SEQ ID NO: 11 - DNA, 72 bp, Homo sapiens
ggggatgtagctcagtggtagagcgcatgcttagcatgcatgaggtcccgggttcgatccccagcatctcca

SEQ ID NO: 12 - DNA, 72 bp, Homo sapiens
gggggtgtagctcagtggtagagcgcgtgcttagcatgcacgaggccctgggttcaatccccagcacctcca

SEQ ID NO: 13 - DNA, 72 bp, Homo sapiens
gggggtatagctcagcggtagagcgcgtgcttagcatgcacgaggtcctgggttcaatccccaatacctcca

SEQ ID NO: 14 - DNA, 72 bp, Homo sapiens
gggggtgtagctcagtggtagagcgcgtgcttagcatgcacgaggccccgggttcaatccctggcacctcca

SEQ ID NO: 15 - DNA, 73 bp, Homo sapiens
gggggattagctcaaatggtagagcgctcgcttagcatgcgagaggtagcgggatcgatgcccgcatcctcca

SEQ ID NO: 16 - DNA, 73 bp, Homo sapiens
ggggaattagctcaggcggtagagcgctcgcttagcatgcgagaggtagcgggatcgacgcccgcattctcca

SEQ ID NO: 17 - DNA, 72 bp, Homo sapiens
ggggatgtagctcagtggtagagcgcatgcttcgcatgtatgaggtcccgggttcgatccccggcatctcca

SEQ ID NO: 18 - DNA, 72 bp, Homo sapiens
ggggatgtagctcagtggtagagcgcatgcttcgcatgtatgaggccccgggttcgatccccggcatctcca

SEQ ID NO: 19 - DNA, 72 bp, Homo sapiens
ggggatgtagctcagtggtagagcgcgcgcttcgcatgtgtgaggtcccgggttcaatccccggcatctcca

SEQ ID NO: 20 - DNA, 72 bp, Homo sapiens
gggggtgtagctcagtggtagagcgcgtgcttcgcatgtacgaggccccgggttcgacccccggctcctcca

SEQ ID NO: 21 - DNA, 72 bp, Homo sapiens
gggggtgtagctcagtggtagagcgcatgctttgcatgtatgaggtcccgggttcgatccccggcacctcca

SEQ ID NO: 22 - DNA, 72 bp, Homo sapiens
ggggatgtagctcagtggtagagcgcatgctttgcatgtatgaggtcccgggttcgatccccggcatctcca

SEQ ID NO: 23 - DNA, 72 bp, Homo sapiens
ggggatgtagctcagtggtagagcgcatgctttgcatgtatgaggccccgggttcgatccccggcatctcca

SEQ ID NO: 24 - DNA, 72 bp, Homo sapiens
ggggatgtagctcagtggtagagcgcatgctttgcacgtatgaggccccgggttcaatccccggcatctcca

SEQ ID NO: 25 - DNA, 72 bp, Homo sapiens
gggggtgtagctcagtggtagagcgcatgctttgcatgtatgaggcctcgggttcgatccccgacacctcca

SEQ ID NO: 26 - DNA, 72 bp, Homo sapiens
gggggtgtagctcagtggtagagcacatgctttgcatgtgtgaggccccgggttcgatccccggcacctcca

SEQ ID NO: 27 - DNA, 71 bp, Homo sapiens
gggggtgtagctcagtggtagagcgcatgctttgcatgtatgaggcctcggttcgatccccgacacctcca

SEQ ID NO: 28 - DNA, 73 bp, Homo sapiens
gggccagtggcgcaatggataacgcgtctgactacggatcagaagattccaggttcgactcctggctggctcg

SEQ ID NO: 29 - DNA, 73 bp, Homo sapiens
gggccagtggcgcaatggataacgcgtctgactacggatcagaagattctaggttcgactcctggctggctcg

SEQ ID NO: 30 - DNA, 73 bp, Homo sapiens
ggccgcgtggcctaatggataaggcgtctgattccggatcagaagattgagggttcgagtcccttcgtggtcg

SEQ ID NO: 31 - DNA, 73 bp, Homo sapiens
gacccagtggcctaatggataaggcatcagcctccggagctggggattgtgggttcgagtcccatctgggtcg

SEQ ID NO: 32 - DNA, 73 bp, Homo sapiens
gccccagtggcctaatggataaggcactggcctcctaagccagggattgtgggttcgagtcccacctggggta

SEQ ID NO: 33 - DNA, 73 bp, Homo sapiens
gccccagtggcctaatggataaggcactggcctcctaagccagggattgtgggttcgagtcccacctggggtg

SEQ ID NO: 34 - DNA, 73 bp, Homo sapiens
gccccggtggcctaatggataaggcattggcctcctaagccagggattgtgggttcgagtcccacccggggta

SEQ ID NO: 35 - DNA, 73 bp, Homo sapiens
gccccagtggcctaatggataaggcattggcctcctaagccagggattgtgggttcgagtcccatctggggtg

SEQ ID NO: 36 - DNA, 73 bp, Homo sapiens
gccccagtggcctgatggataaggtactggcctcctaagccagggattgtgggttcgagttccacctggggta

SEQ ID NO: 37 - DNA, 73 bp, Homo sapiens
ggccgcgtggcctaatggataaggcgtctgacttcggatcagaagattgcaggttcgagtcctgccgcggtcg

SEQ ID NO: 38 - DNA, 73 bp, Homo sapiens
gaccacgtggcctaatggataaggcgtctgacttcggatcagaagattgagggttcgaatccctccgtggtta

SEQ ID NO: 39 - DNA, 73 bp, Homo sapiens
gaccgcgtggcctaatggataaggcgtctgacttcggatcagaagattgagggttcgagtcccttcgtggtcg

SEQ ID NO: 40 - DNA, 73 bp, Homo sapiens
gaccacgtggcctaatggataaggcgtctgacttcggatcagaagattgagggttcgaatcccttcgtggtta

SEQ ID NO: 41 - DNA, 73 bp, Homo sapiens
gaccacgtggcctaatggataaggcgtctgacttcggatcagaagattgagggttcgaatcccttcgtggttg

SEQ ID NO: 42 - DNA, 73 bp, Homo sapiens
ggccgtgtggcctaatggataaggcgtctgacttcggatcaaaagattgcaggtttgagttctgccacggtcg

SEQ ID NO: 43 - DNA, 85 bp, Homo sapiens
ggctccgtggcgcaatggatagcgcattggacttctagaggctgaaggcattcaaaggttccgggttcgagtcccggcggagtcg

SEQ ID NO: 44 - DNA, 88 bp, Homo sapiens
ggctctgtggcgcaatggatagcgcattggacttctagtgacgaatagagcaattcaaaggttgtgggttcgaatcccaccagagtcg

SEQ ID NO: 45 - DNA, 91 bp, Homo sapiens
ggctctgtggcgcaatggatagcgcattggacttctagctgagcctagtgtggtcattcaaaggttgtgggttcgagtcccaccagagtcg

SEQ ID NO: 46 - DNA, 86 bp, Homo sapiens
ggctctgtggcgcaatggatagcgcattggacttctagatagttagagaaattcaaaggttgtgggttcgagtcccaccagagtcg

SEQ ID NO: 47 - DNA, 74 bp, Homo sapiens
gtctctgtggcgcaatggacgagcgcgctggacttctaatccagaggttccgggttcgagtcccggcagagatg

SEQ ID NO: 48 - DNA, 87 bp, Homo sapiens
ggctctgtggcgcaatggatagcgcattggacttctagcctaaatcaagagattcaaaggttgcgggttcgagtccctccagagtcg

SEQ ID NO: 49 - DNA, 74 bp, Homo sapiens
gtctctgtggcgcaatcggttagcgcgttcggctgttaaccgaaaggttggtggttcgatcccacccagggacg

SEQ ID NO: 50 - DNA, 74 bp, Homo sapiens
gtctctgtggcgcaatcggctagcgcgtttggctgttaactaaaaggttggcggttcgaacccacccagaggcg

SEQ ID NO: 51 - DNA, 74 bp, Homo sapiens
gtctctgtggtgcaatcggttagcgcgttccgctgttaaccgaaagcttggtggttcgagcccacccagggatg

SEQ ID NO: 52 - DNA, 74 bp, Homo sapiens
gtctctgtggcgcaatcggctagcgcgtttggctgttaactaaaaagttggtggttcgaacacacccagaggcg

SEQ ID NO: 53 - DNA, 74 bp, Homo sapiens
gtctctgtggcgcaatcggttagcgcgttcggctgttaaccgaaaggttggtggttcgagcccacccagggacg

SEQ ID NO: 54 - DNA, 74 bp, Homo sapiens
gtctctgtggcgcaatcggttagcgcattcggctgttaaccgaaaggttggtggttcgagcccacccagggacg

SEQ ID NO: 55 - DNA, 74 bp, Homo sapiens
gtctctgtggcgcaatcggttagcgcgttcggctgttaaccgaaagattggtggttcgagcccacccagggacg

SEQ ID NO: 56 - DNA, 74 bp, Homo sapiens
gtctctgtggcgcaatcggttagcgcgttcggctgttaactgaaaggttggtggttcgagcccacccagggacg

SEQ ID NO: 57 - DNA, 74 bp, Homo sapiens
gtctctgtggcgcaatgggttagcgcgttcggctgttaaccgaaaggttggtggttcgagcccatccagggacg

SEQ ID NO: 58 - DNA, 74 bp, Homo sapiens
gtctctgtggcgtagtcggttagcgcgttcggctgttaaccgaaaagttggtggttcgagcccacccaggaacg

SEQ ID NO: 59 - DNA, 74 bp, Homo sapiens
gtctctgtggcgcaatcggctagcgcgtttggctgttaactaaaaggttggtggttcgaacccacccagaggcg

SEQ ID NO: 60 - DNA, 74 bp, Homo sapiens
gtctctgtggcgcaatcggttagcgcgttcggctgttaactgaaaggttagtggttcgagcccacccggggacg

SEQ ID NO: 61 - DNA, 72 bp, Homo sapiens
tcctcgttagtatagtggttagtatccccgcctgtcacgcgggagaccggggttcaattccccgacggggag

SEQ ID NO: 62 - DNA, 72 bp, Homo sapiens
tcctcgttagtatagtggtgagtatccccgcctgtcacgcgggagaccggggttcgattccccgacggggag

SEQ ID NO: 63 - DNA, 72 bp, Homo sapiens
tcctcgttagtatagtggtgagtgtccccgtctgtcacgcgggagaccggggttcgattccccgacggggag

SEQ ID NO: 64 - DNA, 72 bp, Homo sapiens
gggggcatagctcagtggtagagcatttgactgcagatcaagaggtccctggttcaaatccaggtgccccct

SEQ ID NO: 65 - DNA, 72 bp, Homo sapiens
gggggtatagctcaggggtagagcatttgactgcagatcaagaggtccctggttcaaatccaggtgcccccc

SEQ ID NO: 66 - DNA, 72 bp, Homo sapiens
gggggtatagcttagcggtagagcatttgactgcagatcaagaggtccccggttcaaatccgggtgccccct

SEQ ID NO: 67 - DNA, 72 bp, Homo sapiens
gggggtatagcttaggggtagagcatttgactgcagatcaaaaggtccctggttcaaatccaggtgcccctt

SEQ ID NO: 68 - DNA, 72 bp, Homo sapiens
gggggtatagctcaggggtagagcatttgactgcagatcaagaggtccccagttcaaatctgggtgccccct

SEQ ID NO: 69 - DNA, 72 bp, Homo sapiens
gggggtatagctcaggggtagagcatttgactgcagatcaagaagtccccggttcaaatccgggtgccccct

SEQ ID NO: 70 - DNA, 72 bp, Homo sapiens
gggggtatagctcaggggtagagcatttgactgcagatcaagaggtctctggttcaaatccaggtgccccct

SEQ ID NO: 71 - DNA, 72 bp, Homo sapiens
gggggtatagctcaggggtagagcacttgactgcagatcaagaagtccttggttcaaatccaggtgccccct

SEQ ID NO: 72 - DNA, 72 bp, Homo sapiens
ggggatatagctcaggggtagagcatttgactgcagatcaagaggtccccggttcaaatccgggtgcccccc

SEQ ID NO: 73 - DNA, 72 bp, Homo sapiens
gggggtatagttcaggggtagagcatttgactgcagatcaagaggtccctggttcaaatccaggtgccccct

SEQ ID NO: 74 - DNA, 72 bp, Homo sapiens
gggggtatagctcaggggtagagcatttgactgcaaatcaagaggtccctgattcaaatccaggtgccccct

SEQ ID NO: 75 - DNA, 72 bp, Homo sapiens
gggggtatagctcagtggtagagcatttgactgcagatcaagaggtccccggttcaaatccgggtgccccct

SEQ ID NO: 76 - DNA, 72 bp, Homo sapiens
gggcgtatagctcaggggtagagcatttgactgcagatcaagaggtccccagttcaaatctgggtgccccct

SEQ ID NO: 77 - DNA, 72 bp, Homo sapiens
gggggtatagctcacaggtagagcatttgactgcagatcaagaggtccccggttcaaatctgggtgccccct

SEQ ID NO: 78 - DNA, 70 bp, Homo sapiens
gggcgtatagctcaggggtagagcatttgactgcagatcaagaggtccccagttcaaatctgggtgccca

SEQ ID NO: 79 - DNA, 72 bp, Homo sapiens
gggggtatagctcacaggtagagcatttgactgcagatcaagaggtccccggttcaaatccggttactccct

SEQ ID NO: 80 - DNA, 72 bp, Homo sapiens
gggggtatagctcaggggtagagcacttgactgcagatcaagaggtccctggttcaaatccaggtgccccct

SEQ ID NO: 81 - DNA, 72 bp, Homo sapiens
gggggtatagctcagtggtagagcatttgactgcagatcaagaggtccctggttcaaatccgggtgccccct

SEQ ID NO: 82 - DNA, 73 bp, Homo sapiens
gggggtatagctcagtgggtagagcatttgactgcagatcaagaggtccccggttcaaatccgggtgccccct

SEQ ID NO: 83 - DNA, 72 bp, Homo sapiens
gggggtgtagctcagtggtagagcatttgactgcagatcaagaggtccctggttcaaatccaggtgccccct

SEQ ID NO: 84 - DNA, 73 bp, Homo sapiens
gggggtatagctcaggtggtagagcatttgactgcagatcaagaggtccccggttcaaatccgggtgccccct

SEQ ID NO: 85 - DNA, 72 bp, Homo sapiens
gggggtatagctcaggggtagagcatttgactgcagatcaagaggtccccggttcaaatccgggtgccccct

SEQ ID NO: 86 - DNA, 72 bp, Homo sapiens
gggggtatagctcaggggtagagcatttgactgcagatcaagaggtccctggttcaaatccaggtgccccct

SEQ ID NO: 87 - DNA, 72 bp, Homo sapiens
ggttccatggtgtaatggttagcactctggactctgaatccagcgatccgagttcaaatctcggtggaacct

SEQ ID NO: 88 - DNA, 72 bp, Homo sapiens
ggttccatggtgtaatggttagcactctggactctgaatccagcgatccgagttcaagtctcggtggaacct

SEQ ID NO: 89 - DNA, 72 bp, Homo sapiens
ggttccatggtgtaatggtgagcactctggactctgaatccagcgatccgagttcgagtctcggtggaacct

SEQ ID NO: 90 - DNA, 72 bp, Homo sapiens
ggttccatggtgtaatggtaagcactctggactctgaatccagcgatccgagttcgagtctcggtggaacct

SEQ ID NO: 91 - DNA, 72 bp, Homo sapiens
ggttccatggtgtaatggttagcactctggactctgaatccggtaatccgagttcaaatctcggtggaacct

SEQ ID NO: 92 - DNA, 72 bp, Homo sapiens
ggccccatggtgtaatggtcagcactctggactctgaatccagcgatccgagttcaaatctcggtgggaccc

SEQ ID NO: 93 - DNA, 72 bp, Homo sapiens
ggttccatggtgtaatggtaagcactctggactctgaatccagccatctgagttcgagtctctgtggaacct

SEQ ID NO: 94 - DNA, 72 bp, Homo sapiens
ggtcccatggtgtaatggttagcactctggactttgaatccagcgatccgagttcaaatctcggtgggacct

SEQ ID NO: 95 - DNA, 72 bp, Homo sapiens
ggtcccatggtgtaatggttagcactctggactttgaatccagcaatccgagttcgaatctcggtgggacct

SEQ ID NO: 96 - DNA, 72 bp, Homo sapiens
ggccccatggtgtaatggttagcactctggactttgaatccagcgatccgagttcaaatctcggtgggacct

SEQ ID NO: 97 - DNA, 72 bp, Homo sapiens
ggtcccatggtgtaatggttagcactctgggctttgaatccagcaatccgagttcgaatcttggtgggacct

SEQ ID NO: 98 - DNA, 72 bp, Homo sapiens
tccctggtggtctagtggttaggattcggcgctctcaccgccgcggcccgggttcgattcccggtcagggaa

SEQ ID NO: 99 - DNA, 72 bp, Homo sapiens
tccctggtggtctagtggttaggattcggcgctctcaccgccgcggcccgggttcgattcccggtcaggaaa

SEQ ID NO: 100 - DNA, 72 bp, Homo sapiens
tcccatatggtctagcggttaggattcctggttttcacccaggtggcccgggttcgactcccggtatgggaa

SEQ ID NO: 101 - DNA, 72 bp, Homo sapiens
tcccacatggtctagcggttaggattcctggttttcacccaggcggcccgggttcgactcccggtgtgggaa

SEQ ID NO: 102 - DNA, 72 bp, Homo sapiens
tccctggtggtctagtggctaggattcggcgctttcaccgccgcggcccgggttcgattcccggccagggaa

SEQ ID NO: 103 - DNA, 72 bp, Homo sapiens
tccctggtggtctagtggctaggattcggcgctttcaccgccgcggcccgggttcgattcccggtcagggaa

SEQ ID NO: 104 - DNA, 71 bp, Homo sapiens
gcattggtggttcagtggtagaattctcgcctcccacgcgggagacccgggttcaattcccggccaatgca

SEQ ID NO: 105 - DNA, 71 bp, Homo sapiens
gcgccgctggtgtagtggtatcatgcaagattcccattcttgcgacccgggttcgattcccgggcggcgca

SEQ ID NO: 106 - DNA, 71 bp, Homo sapiens
gcattggtggttcaatggtagaattctcgcctcccacgcaggagacccaggttcgattcctggccaatgca

SEQ ID NO: 107 - DNA, 71 bp, Homo sapiens
gcatgggtggttcagtggtagaattctcgcctgccacgcgggaggcccgggttcgattcccggcccatgca

SEQ ID NO: 108 - DNA, 71 bp, Homo sapiens
gcattggtggttcagtggtagaattctcgcctgccacgcgggaggcccgggttcgattcccggccaatgca

SEQ ID NO: 109 - DNA, 71 bp, Homo sapiens
gcattggtggttcagtggtagaattctcgcctgccacgcgggaggcccgggtttgattcccggccagtgca

SEQ ID NO: 110 - DNA, 71 bp, Homo sapiens
gcataggtggttcagtggtagaattcttgcctgccacgcaggaggcccaggtttgattcctggcccatgca

SEQ ID NO: 111 - DNA, 71 bp, Homo sapiens
gcattggtggttcagtggtagaattctcgcctgccatgcgggcggccgggcttcgattcctggccaatgca

SEQ ID NO: 112 - DNA, 72 bp, Homo sapiens
gcgttggtggtatagtggttagcatagctgccttccaagcagttgacccgggttcgattcccggccaacgca

SEQ ID NO: 113 - DNA, 72 bp, Homo sapiens
gcgttggtggtatagtggtgagcatagctgccttccaagcagttgacccgggttcgattcccggccaacgca

SEQ ID NO: 114 - DNA, 72 bp, Homo sapiens
gcgttggtggtatagtggtaagcatagctgccttccaagcagttgacccgggttcgattcccggccaacgca

SEQ ID NO: 115 - DNA, 72 bp, Homo sapiens
gcgttggtggtatagtggtgagcatagttgccttccaagcagttgacccgggctcgattcccgcccaacgca

SEQ ID NO: 116 - DNA, 72 bp, Homo sapiens
gccgtgatcgtatagtggttagtactctgcgttgtggccgcagcaacctcggttcgaatccgagtcacggca

SEQ ID NO: 117 - DNA, 72 bp, Homo sapiens
gccatgatcgtatagtggttagtactctgcgctgtggccgcagcaacctcggttcgaatccgagtcacggca

SEQ ID NO: 118 - DNA, 74 bp, Homo sapiens
ggccggttagctcagttggttagagcgtggcgctaataacgccaaggtcgcgggttcgatccccgtacgggcca

SEQ ID NO: 119 - DNA, 74 bp, Homo sapiens
ggccggttagctcagttggttagagcgtggtgctaataacgccaaggtcgcgggttcgatccccgtactggcca

SEQ ID NO: 120 - DNA, 74 bp, Homo sapiens
ggctggttagctcagttggttagagcgtggtgctaataacgccaaggtcgcgggttcgatccccgtactggcca

SEQ ID NO: 121 - DNA, 74 bp, Homo sapiens
ggccggttagctcagttggttagagcgtggtgctaataacgccaaggtcgcgggttcgaaccccgtacgggcca

SEQ ID NO: 122 - DNA, 74 bp, Homo sapiens
ggccggttagctcagttggttagagcgtggtgctaataacgccaaggtcgcgggttcgatccccgtacgggcca

SEQ ID NO: 123 - DNA, 74 bp, Homo sapiens
ggccggttagctcagttggttagagcgtggtgctaataacgctaaggtcgcgggttcgatccccgtactggcca

SEQ ID NO: 124 - DNA, 74 bp, Homo sapiens
ggccggttagctcagttggtcagagcgtggtgctaataacgccaaggtcgcgggttcgatccccgtacgggcca

SEQ ID NO: 125 - DNA, 74 bp, Homo sapiens
ggccggttagctcagtcggctagagcgtggtgctaataacgccaaggtcgcgggttcgatccccgtacgggcca

SEQ ID NO: 126 - DNA, 74 bp, Homo sapiens
ggctggttagttcagttggttagagcgtggtgctaataacgccaaggtcgtgggttcgatccccatatcggcca

SEQ ID NO: 127 - DNA, 74 bp, Homo sapiens
ggccggttagctcagttggtaagagcgtggtgctgataacaccaaggtcgcgggctcgactcccgcaccggcca

SEQ ID NO: 128 - DNA, 93 bp, Homo sapiens
gctccagtggcgcaatcggttagcgcgcggtacttatatgacagtgcgagcggagcaatgccgaggttgtgagttcgatcctcacctggagca

SEQ ID NO: 129 - DNA, 93 bp, Homo sapiens
gctccagtggcgcaatcggttagcgcgcggtacttatacagcagtacatgcagagcaatgccgaggttgtgagttcgagcctcacctggagca

SEQ ID NO: 130 - DNA, 94 bp, Homo sapiens
gctccagtggcgcaatcggttagcgcgcggtacttatatggcagtatgtgtgcgagtgatgccgaggttgtgagttcgagcctcacctggagca

SEQ ID NO: 131 - DNA, 94 bp, Homo sapiens
gctccagtggcgcaatcggttagcgcgcggtacttatacaacagtatatgtgcgggtgatgccgaggttgtgagttcgagcctcacctggagca

SEQ ID NO: 132 - DNA, 94 bp, Homo sapiens
gctccagtggcgcaatcggttagcgcgcggtacttataagacagtgcacctgtgagcaatgccgaggttgtgagttcaagcctcacctggagca

SEQ ID NO: 133 - DNA, 82 bp, Homo sapiens
ggtagcgtggccgagcggtctaaggcgctggattaaggctccagtctcttcggaggcgtgggttcgaatcccaccgctgcca

SEQ ID NO: 134 - DNA, 82 bp, Homo sapiens
ggtagcgtggccgagcggtctaaggcgctggattaaggctccagtctcttcgggggcgtgggttcgaatcccaccgctgcca

SEQ ID NO: 135 - DNA, 82 bp, Homo sapiens
ggtagcgtggccgagcggtctaaggcgctggattaaggctccagtctcttcgggggcgtgggttcaaatcccaccgctgcca

SEQ ID NO: 136 - DNA, 82 bp, Homo sapiens
ggtagcgtggccgagtggtctaagacgctggattaaggctccagtctcttcgggggcgtgggtttgaatcccaccgctgcca

SEQ ID NO: 137 - DNA, 106 bp, Homo sapiens
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, Homo sapiens
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, Homo sapiens
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, Homo sapiens
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, Homo sapiens
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, Homo sapiens
gcctccttagtgcagtaggtagcgcatcagtctcaaaatctgaatggtcctgagttcaagcctcagagggggca

SEQ ID NO: 143 - DNA, 84 bp, Homo sapiens
gtcaggatggccgagcagtcttaaggcgctgcgttcaaatcgcaccctccgctggaggcgtgggttcgaatcccacttttgaca

SEQ ID NO: 144 - DNA, 83 bp, Homo sapiens
gtcaggatggccgagcggtctaaggcgctgcgttcaggtcgcagtctcccctggaggcgtgggttcgaatcccactcctgaca

SEQ ID NO: 145 - DNA, 83 bp, Homo sapiens
gtcaggatggccgagcggtctaaggcgctgcgttcaggtcgcagtctcccctggaggcgtgggttcgaatcccacttctgaca

SEQ ID NO: 146 - DNA, 83 bp, Homo sapiens
accaggatggccgagtggttaaggcgttggacttaagatccaatggacatatgtccgcgtgggttcgaaccccactcctggta

SEQ ID NO: 147 - DNA, 83 bp, Homo sapiens
accgggatggccgagtggttaaggcgttggacttaagatccaatgggctggtgcccgcgtgggttcgaaccccactctcggta

SEQ ID NO: 148 - DNA, 83 bp, Homo sapiens
accagaatggccgagtggttaaggcgttggacttaagatccaatggattcatatccgcgtgggttcgaaccccacttctggta

SEQ ID NO: 149 - DNA, 83 bp, Homo sapiens
accgggatggctgagtggttaaggcgttggacttaagatccaatggacaggtgtccgcgtgggttcgagccccactcccggta

SEQ ID NO: 150 - DNA, 82 bp, Homo sapiens
ggtagcgtggccgagcggtctaaggcgctggatttaggctccagtctcttcggaggcgtgggttcgaatcccaccgctgcca

SEQ ID NO: 151 - DNA, 82 bp, Homo sapiens
ggtagtgtggccgagcggtctaaggcgctggatttaggctccagtctcttcgggggcgtgggttcgaatcccaccactgcca

SEQ ID NO: 152 - DNA, 82 bp, Homo sapiens
ggtagcgtggccgagtggtctaaggcgctggatttaggctccagtcatttcgatggcgtgggttcgaatcccaccgctgcca

SEQ ID NO: 153 - DNA, 73 bp, Homo sapiens
gcccggctagctcagtcggtagagcatgggactcttaatcccagggtcgtgggttcgagccccacgttgggcg

SEQ ID NO: 154 - DNA, 73 bp, Homo sapiens
gcccagctagctcagtcggtagagcataagactcttaatctcagggttgtggattcgtgccccatgctgggtg

SEQ ID NO: 155 - DNA, 74 bp, Homo sapiens
gcagctagctcagtcggtagagcatgagactcttaatctcagggtcatgggttcgtgccccatgttgggtgcca

SEQ ID NO: 156 - DNA, 73 bp, Homo sapiens
gcccggctagctcagtcggtagagcatgagactcttaatctcagggtcgtgggttcgagccccacgttgggcg

SEQ ID NO: 157 - DNA, 73 bp, Homo sapiens
gcccggctagctcagtcggtagagcatgagacccttaatctcagggtcgtgggttcgagccccacgttgggcg

SEQ ID NO: 158 - DNA, 73 bp, Homo sapiens
gcccggctagctcagtcggtagagcatgggactcttaatctcagggtcgtgggttcgagccccacgttgggcg

SEQ ID NO: 159 - DNA, 73 bp, Homo sapiens
gcccggctagctcagtcgatagagcatgagactcttaatctcagggtcgtgggttcgagccgcacgttgggcg

SEQ ID NO: 160 - DNA, 73 bp, Homo sapiens
gcccagctagctcagtcggtagagcatgagactcttaatctcagggtcatgggtttgagccccacgtttggtg

SEQ ID NO: 161 - DNA, 73 bp, Homo sapiens
gcctggctagctcagtcggcaaagcatgagactcttaatctcagggtcgtgggctcgagctccatgttgggcg

SEQ ID NO: 162 - DNA, 73 bp, Homo sapiens
gcccgactacctcagtcggtggagcatgggactcttcatcccagggttgtgggttcgagccccacattgggca

SEQ ID NO: 163 - DNA, 73 bp, Homo sapiens
gcctggatagctcagttggtagagcatcagacttttaatctgagggtccagggttcaagtccctgttcaggca

SEQ ID NO: 164 - DNA, 73 bp, Homo sapiens
acccagatagctcagtcagtagagcatcagacttttaatctgagggtccaaggttcatgtccctttttgggtg

SEQ ID NO: 165 - DNA, 73 bp, Homo sapiens
gcctggatagctcagttggtagagcatcagacttttaatctgagggtccagggttcaagtccctgttcaggcg

SEQ ID NO: 166 - DNA, 73 bp, Homo sapiens
gcccggatagctcagtcggtagagcatcagacttttaatctgagggtccagggttcaagtccctgttcgggcg

SEQ ID NO: 167 - DNA, 73 bp, Homo sapiens
gcctggatagctcagtcggtagagcatcagacttttaatctgagggtccagggttcaagtccctgttcaggcg

SEQ ID NO: 168 - DNA, 73 bp, Homo sapiens
gcccggatagctcagtcggtagagcatcagacttttaatctgagggtccggggttcaagtccctgttcgggcg

SEQ ID NO: 169 - DNA, 73 bp, Homo sapiens
gcctgggtagctcagtcggtagagcatcagacttttaatctgagggtccagggttcaagtccctgtccaggcg

SEQ ID NO: 170 - DNA, 73 bp, Homo sapiens
gcctggatagctcagttggtagaacatcagacttttaatctgacggtgcagggttcaagtccctgttcaggcg

SEQ ID NO: 171 - DNA, 73 bp, Homo sapiens
gcctcgttagcgcagtaggtagcgcgtcagtctcataatctgaaggtcgtgagttcgatcctcacacggggca

SEQ ID NO: 172 - DNA, 73 bp, Homo sapiens
gccctcttagcgcagtgggcagcgcgtcagtctcataatctgaaggtcctgagttcgagcctcagagagggca

SEQ ID NO: 173 - DNA, 73 bp, Homo sapiens
gcctccttagcgcagtaggcagcgcgtcagtctcataatctgaaggtcctgagttcgaacctcagagggggca

SEQ ID NO: 174 - DNA, 73 bp, Homo sapiens
gccctcttagcgcagcgggcagcgcgtcagtctcataatctgaaggtcctgagttcgagcctcagagagggca

SEQ ID NO: 175 - DNA, 73 bp, Homo sapiens
gccctcttagcgcagctggcagcgcgtcagtctcataatctgaaggtcctgagttcaagcctcagagagggca

SEQ ID NO: 176 - DNA, 73 bp, Homo sapiens
gcctcgttagcgcagtaggcagcgcgtcagtctcataatctgaaggtcgtgagttcgagcctcacacggggca

SEQ ID NO: 177 - DNA, 73 bp, Homo sapiens
gccctcttagtgcagctggcagcgcgtcagtttcataatctgaaagtcctgagttcaagcctcagagagggca

SEQ ID NO: 178 - DNA, 73 bp, Homo sapiens
gccgaaatagctcagttgggagagcgttagactgaagatctaaaggtccctggttcgatcccgggtttcggca

SEQ ID NO: 179 - DNA, 73 bp, Homo sapiens
gccgaaatagctcagttgggagagcgttagactgaagatctaaaggtccctggttcaatcccgggtttcggca

SEQ ID NO: 180 - DNA, 73 bp, Homo sapiens
gccgagatagctcagttgggagagcgttagactgaagatctaaaggtccctggttcaatcccgggtttcggca

SEQ ID NO: 181 - DNA, 74 bp, Homo sapiens
gccgaaatagctcagttgggagagcgttagaccgaagatcttaaaggtccctggttcaatcccgggtttcggca

SEQ ID NO: 182 - DNA, 74 bp, Homo sapiens
gctgaaatagctcagttgggagagcgttagactgaagatcttaaagttccctggttcaaccctgggtttcagcc

SEQ ID NO: 183 - DNA, 72 bp, Homo sapiens
ggctcgttggtctaggggtatgattctcgcttaggatgcgagaggtcccgggttcaaatcccggacgagccc

SEQ ID NO: 184 - DNA, 72 bp, Homo sapiens
ggctcgttggtctaggggtatgattctcgcttagggtgcgagaggtcccgggttcaaatcccggacgagccc

SEQ ID NO: 185 - DNA, 72 bp, Homo sapiens
ggctcgttggtctaggggtatgattctcgcttcgggtgcgagaggtcccgggttcaaatcccggacgagccc

SEQ ID NO: 186 - DNA, 72 bp, Homo sapiens
ggctcgttggtctaggggtatgattctcgcttcgggtgtgagaggtcccgggttcaaatcccggacgagccc

SEQ ID NO: 187 - DNA, 72 bp, Homo sapiens
ggctcgttggtctagtggtatgattctcgctttgggtgcgagaggtcccgggttcaaatcccggacgagccc

SEQ ID NO: 188 - DNA, 72 bp, Homo sapiens
ggctcgttggtctaggggtatgattctcggtttgggtccgagaggtcccgggttcaaatcccggacgagccc

SEQ ID NO: 189 - DNA, 72 bp, Homo sapiens
ggctcgttggtctaggggtatgattctcgctttgggtgcgagaggtcccgggttcaaatcccggacgagccc

SEQ ID NO: 190 - DNA, 87 bp, Homo sapiens
gcccggatgatcctcagtggtctggggtgcaggcttcaaacctgtagctgtctagcgacagagtggttcaattccacctttcgggcg

SEQ ID NO: 191 - DNA, 84 bp, Homo sapiens
gctcggatgatcctcagtggtctggggtgcaggcttcaaacctgtagctgtctagtgacagagtggttcaattccacctttgta

SEQ ID NO: 192 - DNA, 82 bp, Homo sapiens
gtagtcgtggccgagtggttaaggcgatggactagaaatccattggggtttccccgcgcaggttcgaatcctgccgactacg

SEQ ID NO: 193 - DNA, 82 bp, Homo sapiens
gtagtcgtggccgagtggttaaggcgatggactagaaatccattggggtctccccgcgcaggttcgaatcctgccgactacg

SEQ ID NO: 194 - DNA, 82 bp, Homo sapiens
gtagtcgtggccgagtggttaaggcgatggactagaaatccattggggtttccccacgcaggttcgaatcctgccgactacg

SEQ ID NO: 195 - DNA, 82 bp, Homo sapiens
gtagtcgtggccgagtggttaaggtgatggactagaaacccattggggtctccccgcgcaggttcgaatcctgccgactacg

SEQ ID NO: 196 - DNA, 82 bp, Homo sapiens
gctgtgatggccgagtggttaaggcgttggactcgaaatccaatggggtctccccgcgcaggttcgaatcctgctcacagcg

SEQ ID NO: 197 - DNA, 82 bp, Homo sapiens
gctgtgatggccgagtggttaaggcgttggactcgaaatccaatggggtctccccgcgcaggttcaaatcctgctcacagcg

SEQ ID NO: 198 - DNA, 82 bp, Homo sapiens
gctgtgatggccgagtggttaaggtgttggactcgaaatccaatgggggttccccgcgcaggttcaaatcctgctcacagcg

SEQ ID NO: 199 - DNA, 82 bp, Homo sapiens
gtcacggtggccgagtggttaaggcgttggactcgaaatccaatggggtttccccgcacaggttcgaatcctgttcgtgacg

SEQ ID NO: 200 - DNA, 82 bp, Homo sapiens
gacgaggtggccgagtggttaaggcgatggactgctaatccattgtgctctgcacgcgtgggttcgaatcccaccctcgtcg

SEQ ID NO: 201 - DNA, 82 bp, Homo sapiens
gacgaggtggccgagtggttaaggcgatggactgctaatccattgtgctctgcacgcgtgggttcgaatcccaccttcgtcg

SEQ ID NO: 202 - DNA, 82 bp, Homo sapiens
gacgaggtggccgagtggttaaggcgatggactgctaatccattgtgctttgcacgcgtgggttcgaatcccatcctcgtcg

SEQ ID NO: 203 - DNA, 82 bp, Homo sapiens
gacgaggtggccgagtggttaaggcgatggactgctaatccattgtgctctgcacgcgtgggttcgaatcccatcctcgtcg

SEQ ID NO: 204 - DNA, 82 bp, Homo sapiens
gacgaggtggccgagtggttaaggcgatggactgctaatccattgtgctctgcacacgtgggttcgaatcccatcctcgtcg

SEQ ID NO: 205 - DNA, 84 bp, Homo sapiens
ggagaggcctggccgagtggttaaggcgatggactgctaatccattgtgctctgcacgcgtgggttcgaatcccatcctcgtcg

SEQ ID NO: 206 - DNA, 82 bp, Homo sapiens
gcagcgatggccgagtggttaaggcgttggacttgaaatccaatggggtctccccgcgcaggttcgaaccctgctcgctgcg

SEQ ID NO: 207 - DNA, 82 bp, Homo sapiens
gtagtcgtggccgagtggttaaggcgatggacttgaaatccattggggtttccccgcgcaggttcgaatcctgccgactacg

SEQ ID NO: 208 - DNA, 82 bp, Homo sapiens
gtagtcgtggccgagtggttaaggcgatggacttgaaatccattggggtctccccgcgcaggttcgaatcctgccgactacg

SEQ ID NO: 209 - DNA, 82 bp, Homo sapiens
gtagtcgtggccgagtggttaaggcgatggacttgaaatccattggggtttccccgcgcaggttcgaatcctgtcggctacg

SEQ ID NO: 210 - DNA, 74 bp, Homo sapiens
ggcgccgtggcttagttggttaaagcgcctgtctagtaaacaggagatcctgggttcgaatcccagcggtgcct

SEQ ID NO: 211 - DNA, 74 bp, Homo sapiens
ggctccgtggcttagctggttaaagcgcctgtctagtaaacaggagatcctgggttcgaatcccagcggggcct

SEQ ID NO: 212 - DNA, 74 bp, Homo sapiens
ggctccgtagcttagttggttaaagcgcctgtctagtaaacaggagatcctgggttcgactcccagcggggcct

SEQ ID NO: 213 - DNA, 74 bp, Homo sapiens
ggcttcgtggcttagctggttaaagcgcctgtctagtaaacaggagatcctgggttcgaatcccagcgaggcct

SEQ ID NO: 214 - DNA, 74 bp, Homo sapiens
ggcgccgtggcttagctggttaaagcgcctgtctagtaaacaggagatcctgggttcgaatcccagcggtgcct

SEQ ID NO: 215 - DNA, 74 bp, Homo sapiens
ggccctgtggcttagctggtcaaagcgcctgtctagtaaacaggagatcctgggttcgaatcccagcggggcct

SEQ ID NO: 216 - DNA, 74 bp, Homo sapiens
ggctctatggcttagttggttaaagcgcctgtctcgtaaacaggagatcctgggttcgactcccagtggggcct

SEQ ID NO: 217 - DNA, 72 bp, Homo sapiens
ggcgcggtggccaagtggtaaggcgtcggtctcgtaaaccgaagatcacgggttcgaaccccgtccgtgcct

SEQ ID NO: 218 - DNA, 74 bp, Homo sapiens
ggctctgtggcttagttggctaaagcgcctgtctcgtaaacaggagatcctgggttcgaatcccagcggggcct

SEQ ID NO: 219 - DNA, 72 bp, Homo sapiens
ggcgcggtggccaagtggtaaggcgtcggtctcgtaaaccgaagatcgcgggttcgaaccccgtccgtgcct

SEQ ID NO: 220 - DNA, 74 bp, Homo sapiens
ggccctgtagctcagcggttggagcgctggtctcgtaaacctaggggtcgtgagttcaaatctcaccagggcct

SEQ ID NO: 221 - DNA, 74 bp, Homo sapiens
ggctctatggcttagttggttaaagcgcctgtcttgtaaacaggagatcctgggttcgaatcccagtagagcct

SEQ ID NO: 222 - DNA, 73 bp, Homo sapiens
ggctccatagctcagtggttagagcactggtcttgtaaaccaggggtcgcgagttcgatcctcgctggggcct

SEQ ID NO: 223 - DNA, 73 bp, Homo sapiens
ggctccatagctcaggggttagagcgctggtcttgtaaaccaggggtcgcgagttcaattctcgctggggcct

SEQ ID NO: 224 - DNA, 73 bp, Homo sapiens
ggctccatagctcaggggttagagcactggtcttgtaaaccaggggtcgcgagttcaaatctcgctggggcct

SEQ ID NO: 225 - DNA, 73 bp, Homo sapiens
ggccctatagctcaggggttagagcactggtcttgtaaaccaggggtcgcgagttcaaatctcgctggggcct

SEQ ID NO: 226 - DNA, 72 bp, Homo sapiens
ggctccatagctcaggggttagagcactggtcttgtaaaccagggtcgcgagttcaaatctcgctggggcct

SEQ ID NO: 227 - DNA, 72 bp, Homo sapiens
ggcctcgtggcgcaacggtagcgcgtctgactccagatcagaaggttgcgtgttcaaatcacgtcggggtca

SEQ ID NO: 228 - DNA, 72 bp, Homo sapiens
gacctcgtggcgcaatggtagcgcgtctgactccagatcagaaggttgcgtgttcaagtcacgtcggggtca

SEQ ID NO: 229 - DNA, 72 bp, Homo sapiens
gacctcgtggcgcaacggtagcgcgtctgactccagatcagaaggttgcgtgttcaaatcacgtcggggtca

SEQ ID NO: 230 - DNA, 72 bp, Homo sapiens
gacctcgtggcgcaacggtagcgcgtctgactccagatcagaaggctgcgtgttcgaatcacgtcggggtca

SEQ ID NO: 231 - DNA, 72 bp, Homo sapiens
gacctcgtggcgcaacggcagcgcgtctgactccagatcagaaggttgcgtgttcaaatcacgtcggggtca

SEQ ID NO: 232 - DNA, 93 bp, Homo sapiens
ccttcaatagttcagctggtagagcagaggactatagctacttcctcagtaggagacgtccttaggttgctggttcgattccagcttgaagga

SEQ ID NO: 233 - DNA, 91 bp, Homo sapiens
ccttcgatagctcagttggtagagcggaggactgtagttggctgtgtccttagacatccttaggtcgctggttcgaatccggctcgaagga

SEQ ID NO: 234 - DNA, 89 bp, Homo sapiens
ccttcgatagctcagttggtagagcggaggactgtagtggatagggcgtggcaatccttaggtcgctggttcgattccggctcgaagga

SEQ ID NO: 235 - DNA, 89 bp, Homo sapiens
ccttcgatagctcagttggtagagcggaggactgtaggctcattaagcaaggtatccttaggtcgctggttcgaatccggctcggagga

SEQ ID NO: 236 - DNA, 94 bp, Homo sapiens
ccttcgatagctcagctggtagagcggaggactgtagattgtatagacatttgcggacatccttaggtcgctggttcgattccagctcgaagga

SEQ ID NO: 237 - DNA, 93 bp, Homo sapiens
ccttcgatagctcagctggtagagcggaggactgtagctacttcctcagcaggagacatccttaggtcgctggttcgattccggctcgaagga

SEQ ID NO: 238 - DNA, 89 bp, Homo sapiens
ccttcgatagctcagctggtagagcggaggactgtaggcgcgcgcccgtggccatccttaggtcgctggttcgattccggctcgaagga

SEQ ID NO: 239 - DNA, 94 bp, Homo sapiens
ccttcgatagctcagctggtagagcggaggactgtagcctgtagaaacatttgtggacatccttaggtcgctggttcgattccggctcgaagga

SEQ ID NO: 240 - DNA, 94 bp, Homo sapiens
ccttcgatagctcagctggtagagcggaggactgtagattgtacagacatttgcggacatccttaggtcgctggttcgattccggctcgaagga

SEQ ID NO: 241 - DNA, 89 bp, Homo sapiens
ccttcgatagctcagctggtagagcggaggactgtagtacttaatgtgtggtcatccttaggtcgctggttcgattccggctcgaagga

SEQ ID NO: 242 - DNA, 89 bp, Homo sapiens
ccttcgatagctcagctggtagagcggaggactgtaggggtttgaatgtggtcatccttaggtcgctggttcgaatccggctcggagga

SEQ ID NO: 243 - DNA, 94 bp, Homo sapiens
ccttcgatagctcagctggtagagcggaggactgtagactgcggaaacgtttgtggacatccttaggtcgctggttcaattccggctcgaagga

SEQ ID NO: 244 - DNA, 90 bp, Homo sapiens
ctttcgatagctcagttggtagagcggaggactgtaggttcattaaactaaggcatccttaggtcgctggttcgaatccggctcgaagga

SEQ ID NO: 245 - DNA, 88 bp, Homo sapiens
tcttcaatagctcagctggtagagcggaggactgtaggtgcacgcccgtggccattcttaggtgctggtttgattccgacttggagag

SEQ ID NO: 246 - DNA, 73 bp, Homo sapiens
gtttccgtagtgtagtggttatcacgttcgcctaacacgcgaaaggtccccggttcgaaaccgggcggaaaca

SEQ ID NO: 247 - DNA, 73 bp, Homo sapiens
gtttccgtagtgtagtggtcatcacgttcgcctaacacgcgaaaggtccccggttcgaaaccgggcggaaaca

SEQ ID NO: 248 - DNA, 73 bp, Homo sapiens
gtttccgtagtgtagtggttatcacgttcgcctaacacgcgaaaggtccctggatcaaaaccaggcggaaaca

SEQ ID NO: 249 - DNA, 73 bp, Homo sapiens
gtttccgtagtgtagtggttatcacgttcgcctaacacgcgaaaggtccgcggttcgaaaccgggcggaaaca

SEQ ID NO: 250 - DNA, 73 bp, Homo sapiens
gtttccgtagtgtagtggttatcacgtttgcctaacacgcgaaaggtccccggttcgaaaccgggcagaaaca

SEQ ID NO: 251 - DNA, 72 bp, Homo sapiens
gggggtgtagctcagtggtagagcgtatgcttaacattcatgaggctctgggttcgatccccagcacttcca

SEQ ID NO: 252 - DNA, 73 bp, Homo sapiens
gtttccgtagtgtagtggttatcacgttcgcctcacacgcgaaaggtccccggttcgaaaccgggcggaaaca

SEQ ID NO: 253 - DNA, 73 bp, Homo sapiens
gcttctgtagtgtagtggttatcacgttcgcctcacacgcgaaaggtccccggttcgaaaccgggcagaagca

SEQ ID NO: 254 - DNA, 73 bp, Homo sapiens
gtttccgtagtgtagcggttatcacattcgcctcacacgcgaaaggtccccggttcgatcccgggcggaaaca

SEQ ID NO: 255 - DNA, 73 bp, Homo sapiens
gtttccgtagtgtagtggttatcacgttcgcctcacacgcgaaaggtccccggttcgaaactgggcggaaaca

SEQ ID NO: 256 - DNA, 74 bp, Homo sapiens
gtttccgtagtgtagtggttatcacgttcgcctcacacgcgtaaaggtccccggttcgaaaccgggcggaaaca

SEQ ID NO: 257 - DNA, 73 bp, Homo sapiens
gtttccgtagtggagtggttatcacgttcgcctcacacgcgaaaggtccccggtttgaaaccaggcggaaaca

SEQ ID NO: 258 - DNA, 73 bp, Homo sapiens
ggttccatagtgtagtggttatcacgtctgctttacacgcagaaggtcctgggttcgagccccagtggaacca

SEQ ID NO: 259 - DNA, 73 bp, Homo sapiens
ggttccatagtgtagcggttatcacgtctgctttacacgcagaaggtcctgggttcgagccccagtggaacca

SEQ ID NO: 260 - DNA, 73 bp, Homo sapiens
ggttccatagtgtagtggttatcacatctgctttacacgcagaaggtcctgggttcaagccccagtggaacca

SEQ ID NO: 261 - DNA, 73 bp, Homo sapiens
gtttccgtggtgtagtggttatcacattcgccttacacgcgaaaggtcctcgggtcgaaaccgagcggaaaca

SEQ ID NO: 262 - DNA, 72 bp, Homo sapiens
agcagagtggcgcagcggaagcgtgctgggcccataacccagaggtcgatggatcgaaaccatcctctgcta

SEQ ID NO: 263 - DNA, 72 bp, Homo sapiens
agcagagtggcgcagcggaagcgtgctgggcccataacccagaggtcgatggatctaaaccatcctctgcta

SEQ ID NO: 264 - DNA, 73 bp, Homo sapiens
tccctggtggtctagtggctaggattcggcgctttcaccgccgcggcccgggttcgattcccggtcagggaat

SEQ ID NO: 265 - DNA, 72 bp, Homo sapiens
gcgttggtggtttagtggtagaattctcgcctcccatgcgggagacccgggttcaattcccggccactgcac

SEQ ID NO: 266 - DNA, 72 bp, Homo sapiens
ggccttggtggtgcagtggtagaattctcgcctcccacgtgggagacccgggttcaattcccggccaatgca

SEQ ID NO: 267 - DNA, 73 bp, Homo sapiens
gtccctggtggtctagtggctaggattcggcgctttcaccgccgcggcccgggttcgattcccggccagggaa

SEQ ID NO: 268 - DNA, 75 bp, Homo sapiens
tgtctctgtggcgcaatcggttagcgcgttcggctgttaaccgaaagattggtggttcgagcccacccagggacg

SEQ ID NO: 269 - DNA, 86 bp, Homo sapiens
tggctccgtggcgcaatggatagcgcattggacttctagaggctgaaggcattcaaaggttccgggttcgagtcccggcggagtcg

SEQ ID NO: 270 - DNA, 74 bp, Homo sapiens
gcccggctagctcagtcggtagagcatgagactcttaatctcagggtcgtgggttcgagccccacgttgggcgc

SEQ ID NO: 271 - DNA, 73 bp, Homo sapiens
gccgtgatcgtatagtggttagtactctgcgttgtggccgcagcaacctcggttcgaatccgagtcacggcag

SEQ ID NO: 272 - DNA, 73 bp, Homo sapiens
gcgttggtggtatagtggtgagcatagctgccttccaagcagttgacccgggttcgattcccggccaacgcag

SEQ ID NO: 273 - DNA, 73 bp, Homo sapiens
tccctggtggtctagtggttaggattcggcgctctcaccgccgcggcccgggttcgattcccggtcagggaaa

SEQ ID NO: 274 - DNA, 73 bp, Homo sapiens
aggttccatggtgtaatggtgagcactctggactctgaatccagcgatccgagttcgagtctcggtggaacct

SEQ ID NO: 275 - DNA, 75 bp, Homo sapiens
tgtctctgtggcgtagtcggttagcgcgttcggctgttaaccgaaaagttggtggttcgagcccacccaggaacg

SEQ ID NO: 276 - DNA, 75 bp, Homo sapiens
tgtctctgtggcgcaatcggttagcgcgttcggctgttaaccgaaaggttggtggttcgagcccacccagggacg

SEQ ID NO: 277 - DNA, 75 bp, Homo sapiens
gtctctgtggcgcaatcggttagcgcattcggctgttaaccgaaaggttggtggttcgagcccacccagggacgc

SEQ ID NO: 278 - DNA, 75 bp, Homo sapiens
gtctctgtggcgcaatgggttagcgcgttcggctgttaaccgaaaggttggtggttcgagcccatccagggacgc

SEQ ID NO: 279 - DNA, 72 bp, Homo sapiens
gcactggtggttcagtggtagaattctcgcctcacacgcgggacacccgggttcaattcccggtcaaggcaa

SEQ ID NO: 280 - DNA, 74 bp, Homo sapiens
gtttccgtagtgtagtggttatcacgttcgcctcacacgcgaaaggtccccggttcgaaactgggcggaaacag

SEQ ID NO: 281 - DNA, 72 bp, Homo sapiens
gcactggtggttcagtggtagaattctcgcctcccacgcgggagacccgggtttaattcccggtcaagataa

SEQ ID NO: 282 - DNA, 75 bp, Homo sapiens
gtttccgtagtgtagtggttatcacgttcgcctcacacgcgtaaaggtccccggttcgaaaccgggcggaaacat

SEQ ID NO: 283 - DNA, 73 bp, Homo sapiens
tagcagagtggcgcagcggaagcgtgctgggcccataacccagaggtcgatggatcgaaaccatcctctgcta

SEQ ID NO: 284 - DNA, 74 bp, Homo sapiens
gtttccgtagtgtagtggttatcacgttcgcctcacacgcgaaaggtccccggttcgaaaccgggcggaaacaa

SEQ ID NO: 285 - DNA, 73 bp, Homo sapiens
tcctcgttagtatagtggtgagtatccccgcctgtcacgcgggagaccggggttcgattccccgacggggagg

SEQ ID NO: 286 - DNA, 72 bp, Homo sapiens
tgcatgggtggttcagtggtagaattctcgcctgccacgcgggaggcccgggttcgattcccggcccatgca

SEQ ID NO: 287 - DNA, 73 bp, Homo sapiens
tccctggtggtctagtggttaggattcggcgctctcaccgccgcggcccgggttcgattcccggtcagggaag

SEQ ID NO: 288 - DNA, 73 bp, Homo sapiens
atccttgttactatagtggtgagtatctctgcctgtcatgcgtgagagagggggtcgattccccgacggggag

SEQ ID NO: 289 - DNA, 72 bp, Homo sapiens
gcattggtggttcagtggtagaattctcgcctgccacgcgggaggcccgggttcgattcccggccaatgcac

SEQ ID NO: 290 - DNA, 84 bp, Homo sapiens
gtcaggatggccgagcggtctaaggcgctgcgttcaggtcgcagtctcccctggaggcgtgggttcgaatcccactcctgacaa

SEQ ID NO: 291 - DNA, 73 bp, Homo sapiens
cgcgttggtggtatagtggtgagcatagctgccttccaagcagttgacccgggttcgattcccggccaacgca

SEQ ID NO: 292 - DNA, 75 bp, Homo sapiens
cgtctctgtggcgcaatcggttagcgcgttcggctgttaaccgaaaggttggtggttcgatcccacccagggacg

SEQ ID NO: 293 - DNA, 73 bp, Homo sapiens
cgcgttggtggtgtagtggtgagcacagctgcctttcaagcagttaacgcgggttcgattcccgggtaacgaa

SEQ ID NO: 294 - DNA, 73 bp, Homo sapiens
cggctcgttggtctaggggtatgattctcgcttcgggtgcgagaggtcccgggttcaaatcccggacgagccc

SEQ ID NO: 295 - DNA, 73 bp, Homo sapiens
ggctcgttggtctaggggtatgattctcgcttagggtgcgagaggtcccgggttcaaatcccggacgagccct

SEQ ID NO: 296 - DNA, 74 bp, Homo sapiens
cgcccggatagctcagtcggtagagcatcagacttttaatctgagggtccagggttcaagtccctgttcgggcg

SEQ ID NO: 297 - DNA, 74 bp, Homo sapiens
gcccggatagctcagtcggtagagcatcagacttttaatctgagggtccagggttcaagtccctgttcgggcgt

SEQ ID NO: 298 - DNA, 107 bp, Homo sapiens
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, Homo sapiens
ttccctggtggtctagtggttaggattcggcgctctcaccgccgcggcccgggttcgattcccggtcaggaaa

SEQ ID NO: 300 - DNA, 90 bp, Homo sapiens
gccttcgatagctcagttggtagagcggaggactgtagtggatagggcgtggcaatccttaggtcgctggttcgattccggctcgaagga

SEQ ID NO: 301 - DNA, 74 bp, Homo sapiens
cgggggattagctcaaatggtagagcgctcgcttagcatgcgagaggtagcgggatcgatgcccgcatcctcca

SEQ ID NO: 302 - DNA, 94 bp, Homo sapiens
agctccagtggcgcaatcggttagcgcgcggtacttatacagcagtacatgcagagcaatgccgaggttgtgagttcgagcctcacctggagca

SEQ ID NO: 303 - DNA, 72 bp, Homo sapiens
gcgccgctggtgtagtggtatcatgcaagattcccattcttgcgacccgggttcgattcccgggcggcgcat

SEQ ID NO: 304 - DNA, 73 bp, Homo sapiens
tcccatatggtctagcggttaggattcctggttttcacccaggtggcccgggttcgactcccggtatgggaac

SEQ ID NO: 305 - DNA, 73 bp, Homo sapiens
gggggatgtagctcagtggtagagcgcgcgcttcgcatgtgtgaggtcccgggttcaatccccggcatctcca

SEQ ID NO: 306 - DNA, 72 bp, Homo sapiens
gcattggtggttcagtggtagaattctcgcctgccacgcgggaggcccgggttcgattcccggccaatgcaa

SEQ ID NO: 307 - DNA, 74 bp, Homo sapiens
gggccagtggcgcaatggataacgcgtctgactacggatcagaagattctaggttcgactcctggctggctcgc

SEQ ID NO: 308 - DNA, 74 bp, Homo sapiens
ggtttccgtagtgtagtggttatcacgttcgcctaacacgcgaaaggtccccggttcgaaaccgggcggaaaca

SEQ ID NO: 309 - DNA, 74 bp, Homo sapiens
agtttccgtagtgtagtggttatcacgttcgcctaacacgcgaaaggtccccggttcgaaaccgggcggaaaca

SEQ ID NO: 310 - DNA, 83 bp, Homo sapiens
aggtagcgtggccgagcggtctaaggcgctggattaaggctccagtctcttcgggggcgtgggttcgaatcccaccgctgcca

SEQ ID NO: 311 - DNA, 74 bp, Homo sapiens
gtttccgtagtgtagtggtcatcacgttcgcctaacacgcgaaaggtccccggttcgaaaccgggcggaaacat

SEQ ID NO: 312 - DNA, 73 bp, Homo sapiens
ggctcgttggtctaggggtatgattctcgctttgggtgcgagaggtcccgggttcaaatcccggacgagccca

SEQ ID NO: 313 - DNA, 73 bp, Homo sapiens
ggctccatagctcaggggttagagcactggtcttgtaaaccagggtcgcgagttcaaatctcgctggggcctg

SEQ ID NO: 314 - DNA, 73 bp, Homo sapiens
tggggatgtagctcagtggtagagcgcatgctttgcatgtatgaggccccgggttcgatccccggcatctcca

SEQ ID NO: 315 - DNA, 74 bp, Homo sapiens
cgcccggctagctcagtcggtagagcatgagactcttaatctcagggtcgtgggttcgagccccacgttgggcg

SEQ ID NO: 316 - DNA, 74 bp, Homo sapiens
gtttccgtagtgtagtggttatcacgttcgcctaacacgcgaaaggtccccggttcgaaaccgggcggaaacaa

SEQ ID NO: 317 - DNA, 74 bp, Homo sapiens
gcccggctagctcagtcggtagagcatgagactcttaatctcagggtcgtgggttcgagccccacgttgggcgt

SEQ ID NO: 318 - DNA, 74 bp, Homo sapiens
gtttccgtagtgtagtggttatcacgttcgcctcacacgcgaaaggtccccggttcgaaaccgggcggaaacac

SEQ ID NO: 319 - DNA, 73 bp, Homo sapiens
cagcagagtggcgcagcggaagcgtgctgggcccataacccagaggtcgatggatcgaaaccatcctctgcta

SEQ ID NO: 320 - DNA, 85 bp, Homo sapiens
ggagaggcctggccgagtggttaaggcgatggactgctaatccattgtgctctgcacgcgtgggttcgaatcccatcctcgtcgc

SEQ ID NO: 321 - DNA, 73 bp, Homo sapiens
ggccccatggtgtaatggttagcactctggactttgaatccagcgatccgagttcaaatctcggtgggacctg

SEQ ID NO: 322 - DNA, 73 bp, Homo sapiens
ggccccatggtgtaatggttagcactctggactttgaatccagcgatccgagttcaaatctcggtgggaccta

SEQ ID NO: 323 - DNA, 83 bp, Homo sapiens
gtagtcgtggccgagtggttaaggcgatggacttgaaatccattggggtctccccgcgcaggttcgaatcctgccgactacgg

SEQ ID NO: 324 - DNA, 73 bp, Homo sapiens
agcagagtggcgcagcggaagcgtgctgggcccataacccagaggtcgatggatcgaaaccatcctctgctat

SEQ ID NO: 325 - DNA, 74 bp, Homo sapiens
ggaccacgtggcctaatggataaggcgtctgacttcggatcagaagattgagggttcgaatccctccgtggtta

SEQ ID NO: 326 - DNA, 83 bp, Homo sapiens
tgtagtcgtggccgagtggttaaggcgatggactagaaatccattggggtctccccgcgcaggttcgaatcctgccgactacg

SEQ ID NO: 327 - DNA, 73 bp, Homo sapiens
agcagagtggcgcagcggaagcgtgctgggcccataacccagaggtcgatggatcgaaaccatcctctgctag

SEQ ID NO: 328 - DNA, 84 bp, Homo sapiens
cgtcaggatggccgagcggtctaaggcgctgcgttcaggtcgcagtctcccctggaggcgtgggttcgaatcccactcctgaca

SEQ ID NO: 329 - DNA, 75 bp, Homo sapiens
ggctccgtggcttagctggttaaagcgcctgtctagtaaacaggagatcctgggttcgaatcccagcggggcctg

SEQ ID NO: 330 - DNA, 74 bp, Homo sapiens
agggccagtggcgcaatggataacgcgtctgactacggatcagaagattccaggttcgactcctggctggctcg

SEQ ID NO: 331 - DNA, 74 bp, Homo sapiens
ggtttccgtagtgtagtggttatcacgttcgcctcacacgcgaaaggtccccggttcgaaaccgggcggaaaca

SEQ ID NO: 332 - DNA, 73 bp, Homo sapiens
aggggatgtagctcagtggtagagcgcatgcttcgcatgtatgaggtcccgggttcgatccccggcatctcca

SEQ ID NO: 333 - DNA, 75 bp, Homo sapiens
tggccggttagctcagttggttagagcgtggtgctaataacgccaaggtcgcgggttcgatccccgtacgggcca

SEQ ID NO: 334 - DNA, 73 bp, Homo sapiens
cggctcgttggtctaggggtatgattctcgcttagggtgcgagaggtcccgggttcaaatcccggacgagccc

SEQ ID NO: 335 - DNA, 74 bp, Homo sapiens
agcccggctagctcagtcggtagagcatgagactcttaatctcagggtcgtgggttcgagccccacgttgggcg

SEQ ID NO: 336 - DNA, 92 bp, Homo sapiens
tccttcgatagctcagttggtagagcggaggactgtagttggctgtgtccttagacatccttaggtcgctggttcgaatccggctcgaagga

SEQ ID NO: 337 - DNA, 74 bp, Homo sapiens
ggggaattagctcaaatggtagagcgctcgcttagcatgcgagaggtagcgggatcgatgcccgcattctccag

SEQ ID NO: 338 - DNA, 74 bp, Homo sapiens
cgccctcttagcgcagcgggcagcgcgtcagtctcataatctgaaggtcctgagttcgagcctcagagagggca

SEQ ID NO: 339 - DNA, 95 bp, Homo sapiens
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, Homo sapiens
tgccgtgatcgtatagtggttagtactctgcgttgtggccgcagcaacctcggttcgaatccgagtcacggca

SEQ ID NO: 341 - DNA, 75 bp, Homo sapiens
ggccggttagctcagttggttagagcgtggtgctaataacgccaaggtcgcgggttcgatccccgtacgggccac

SEQ ID NO: 342 - DNA, 74 bp, Homo sapiens
agtttccgtagtgtagtggttatcacgtttgcctaacacgcgaaaggtccccggttcgaaaccgggcagaaaca

SEQ ID NO: 343 - DNA, 74 bp, Homo sapiens
gcttctgtagtgtagtggttatcacgttcgcctcacacgcgaaaggtccccggttcgaaaccgggcagaagcaa

SEQ ID NO: 344 - DNA, 73 bp, Homo sapiens
ttcctcgttagtatagtggtgagtatccccgcctgtcacgcgggagaccggggttcgattccccgacggggag

SEQ ID NO: 345 - DNA, 83 bp, Homo sapiens
gtagtcgtggccgagtggttaaggcgatggacttgaaatccattggggtttccccgcgcaggttcgaatcctgtcggctacgg

SEQ ID NO: 346 - DNA, 73 bp, Homo sapiens
aggttccatggtgtaatggttagcactctggactctgaatccagcgatccgagttcaaatctcggtggaacct

SEQ ID NO: 347 - DNA, 73 bp, Homo sapiens
tcctcgttagtatagtggtgagtgtccccgtctgtcacgcgggagaccggggttcgattccccgacggggaga

SEQ ID NO: 348 - DNA, 74 bp, Homo sapiens
gtttccgtagtgtagtggttatcacgttcgcctaacacgcgaaaggtccctggatcaaaaccaggcggaaacaa

SEQ ID NO: 349 - DNA, 75 bp, Homo sapiens
cggccggttagctcagttggttagagcgtggtgctaataacgccaaggtcgcgggttcgatccccgtactggcca

SEQ ID NO: 350 - DNA, 73 bp, Homo sapiens
ggccccatggtgtaatggtcagcactctggactctgaatccagcgatccgagttcaaatctcggtgggaccca

SEQ ID NO: 351 - DNA, 73 bp, Homo sapiens
ggccccatggtgtaatggttagcactctggactttgaatccagcgatccgagttcaaatctcggtgggacctt

SEQ ID NO: 352 - DNA, 73 bp, Homo sapiens
tgggggtgtagctcagtggtagagcgcgtgcttagcatgtacgaggtcccgggttcaatccccggcacctcca

SEQ ID NO: 353 - DNA, 73 bp, Homo sapiens
ggggatgtagctcagtggtagagcgcatgcttagcatgcatgaggtcccgggttcgatccccagcatctccag

SEQ ID NO: 354 - DNA, 73 bp, Homo sapiens
agggggtgtagctcagtggtagagcgcgtgcttcgcatgtacgaggccccgggttcgacccccggctcctcca

SEQ ID NO: 355 - DNA, 73 bp, Homo sapiens
gggggtgtagctcagtggtagagcgcgtgcttagcatgcacgaggccccgggttcaatccccggcacctccat

SEQ ID NO: 356 - DNA, 73 bp, Homo sapiens
gggggtgtagctcagtggtagagcgcgtgcttagcatgcacgaggccccgggttcaatccccggcacctccag

SEQ ID NO: 357 - DNA, 107 bp, Homo sapiens
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, Homo sapiens
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, Homo sapiens
ggttccatggtgtaatggttagcactctggactctgaatccagcgatccgagttcaaatctcggtggaacctt

SEQ ID NO: 360 - DNA, 83 bp, Homo sapiens
ggtagcgtggccgagcggtctaaggcgctggattaaggctccagtctcttcgggggcgtgggttcgaatcccaccgctgccag

SEQ ID NO: 361 - DNA, 74 bp, Homo sapiens
tgcctccttagcgcagtaggcagcgcgtcagtctcataatctgaaggtcctgagttcgaacctcagagggggca

SEQ ID NO: 362 - DNA, 74 bp, Homo sapiens
agcccggatagctcagtcggtagagcatcagacttttaatctgagggtccagggttcaagtccctgttcgggcg

SEQ ID NO: 363 - DNA, 74 bp, Homo sapiens
gcctccttagcgcagtaggcagcgcgtcagtctcataatctgaaggtcctgagttcgaacctcagagggggcag

SEQ ID NO: 364 - DNA, 73 bp, Homo sapiens
ttccctggtggtctagtggttaggattcggcgctctcaccgccgcggcccgggttcgattcccggtcagggaa

SEQ ID NO: 365 - DNA, 84 bp, Homo sapiens
caccaggatggccgagtggttaaggcgttggacttaagatccaatggacatatgtccgcgtgggttcgaaccccactcctggta

SEQ ID NO: 366 - DNA, 73 bp, Homo sapiens
tggctcgttggtctaggggtatgattctcgcttagggtgcgagaggtcccgggttcaaatcccggacgagccc

SEQ ID NO: 367 - DNA, 74 bp, Homo sapiens
agccccagtggcctaatggataaggcattggcctcctaagccagggattgtgggttcgagtcccatctggggtg

SEQ ID NO: 368 - DNA, 73 bp, Homo sapiens
ggggatatagctcaggggtagagcatttgactgcagatcaagaggtccccggttcaaatccgggtgccccccc

SEQ ID NO: 369 - DNA, 94 bp, Homo sapiens
cccttcgatagctcagctggtagagcggaggactgtagctacttcctcagcaggagacatccttaggtcgctggttcgattccggctcgaagga

SEQ ID NO: 370 - DNA, 90 bp, Homo sapiens
cccttcgatagctcagctggtagagcggaggactgtaggcgcgcgcccgtggccatccttaggtcgctggttcgattccggctcgaagga

SEQ ID NO: 371 - DNA, 74 bp, Homo sapiens
tgggggattagctcaaatggtagagcgctcgcttagcatgcgagaggtagcgggatcgatgcccgcatcctcca

SEQ ID NO: 372 - DNA, 83 bp, Homo sapiens
gtagtcgtggccgagtggttaaggcgatggactagaaatccattggggtctccccgcgcaggttcgaatcctgccgactacgg

SEQ ID NO: 373 - DNA, 74 bp, Homo sapiens
gcctcgttagcgcagtaggtagcgcgtcagtctcataatctgaaggtcgtgagttcgatcctcacacggggcac

SEQ ID NO: 374 - DNA, 92 bp, Homo sapiens
ggctctgtggcgcaatggatagcgcattggacttctagctgagcctagtgtggtcattcaaaggttgtgggttcgagtcccaccagagtcga

SEQ ID NO: 375 - DNA, 75 bp, Homo sapiens
gtctctgtggcgcaatcggttagcgcgttcggctgttaaccgaaaggttggtggttcgagcccacccagggacgc

SEQ ID NO: 376 - DNA, 83 bp, Homo sapiens
ggcagcgatggccgagtggttaaggcgttggacttgaaatccaatggggtctccccgcgcaggttcgaaccctgctcgctgcg

SEQ ID NO: 377 - DNA, 74 bp, Homo sapiens
ggttccatagtgtagtggttatcacgtctgctttacacgcagaaggtcctgggttcgagccccagtggaaccat

SEQ ID NO: 378 - DNA, 74 bp, Homo sapiens
ggttccatagtgtagcggttatcacgtctgctttacacgcagaaggtcctgggttcgagccccagtggaaccac

SEQ ID NO: 379 - DNA, 87 bp, Homo sapiens
tggctctgtggcgcaatggatagcgcattggacttctagatagttagagaaattcaaaggttgtgggttcgagtcccaccagagtcg

SEQ ID NO: 380 - DNA, 84 bp, Homo sapiens
taccagaatggccgagtggttaaggcgttggacttaagatccaatggattcatatccgcgtgggttcgaaccccacttctggta

SEQ ID NO: 381 - DNA, 74 bp, Homo sapiens
ggcccggatagctcagtcggtagagcatcagacttttaatctgagggtccggggttcaagtccctgttcgggcg

SEQ ID NO: 382 - DNA, 74 bp, Homo sapiens
gccgaaatagctcagttgggagagcgttagactgaagatctaaaggtccctggttcgatcccgggtttcggcag

SEQ ID NO: 383 - DNA, 74 bp, Homo sapiens
gcccggatagctcagtcggtagagcatcagacttttaatctgagggtccagggttcaagtccctgttcgggcgg

SEQ ID NO: 384 - DNA, 74 bp, Homo sapiens
gccgaaatagctcagttgggagagcgttagactgaagatctaaaggtccctggttcaatcccgggtttcggcag

SEQ ID NO: 385 - DNA, 83 bp, Homo sapiens
ggacgaggtggccgagtggttaaggcgatggactgctaatccattgtgctttgcacgcgtgggttcgaatcccatcctcgtcg

SEQ ID NO: 386 - DNA, 73 bp, Homo sapiens
ggctcgttggtctaggggtatgattctcggtttgggtccgagaggtcccgggttcaaatcccggacgagcccc

SEQ ID NO: 387 - DNA, 83 bp, Homo sapiens
agtcacggtggccgagtggttaaggcgttggactcgaaatccaatggggtttccccgcacaggttcgaatcctgttcgtgacg

SEQ ID NO: 388 - DNA, 73 bp, Homo sapiens
ctcctcgttagtatagtggttagtatccccgcctgtcacgcgggagaccggggttcaattccccgacggggag

SEQ ID NO: 389 - DNA, 73 bp, Homo sapiens
ggacctcgtggcgcaacggtagcgcgtctgactccagatcagaaggctgcgtgttcgaatcacgtcggggtca

SEQ ID NO: 390 - DNA, 73 bp, Homo sapiens
ggggatgtagctcagtggtagagcgcatgctttgcatgtatgaggccccgggttcgatccccggcatctccat

SEQ ID NO: 391 - DNA, 74 bp, Homo sapiens
gccgaaatagctcagttgggagagcgttagactgaagatctaaaggtccctggttcgatcccgggtttcggcac

SEQ ID NO: 392 - DNA, 73 bp, Homo sapiens
aggggatgtagctcagtggtagagcgcatgctttgcacgtatgaggccccgggttcaatccccggcatctcca

SEQ ID NO: 393 - DNA, 75 bp, Homo sapiens
gtctctgtggcgcaatcggttagcgcgttcggctgttaaccgaaaggttggtggttcgagcccacccagggacgg

SEQ ID NO: 394 - DNA, 73 bp, Homo sapiens
tcccacatggtctagcggttaggattcctggttttcacccaggcggcccgggttcgactcccggtgtgggaac

SEQ ID NO: 395 - DNA, 74 bp, Homo sapiens
ggctccatagctcaggggttagagcgctggtcttgtaaaccaggggtcgcgagttcaattctcgctggggcctg

SEQ ID NO: 396 - DNA, 83 bp, Homo sapiens
tggtagtgtggccgagcggtctaaggcgctggatttaggctccagtctcttcgggggcgtgggttcgaatcccaccactgcca

SEQ ID NO: 397 - DNA, 74 bp, Homo sapiens
ggctccatagctcaggggttagagcactggtcttgtaaaccaggggtcgcgagttcaaatctcgctggggcctc

SEQ ID NO: 398 - DNA, 73 bp, Homo sapiens
tggctcgttggtctagtggtatgattctcgctttgggtgcgagaggtcccgggttcaaatcccggacgagccc

SEQ ID NO: 399 - DNA, 95 bp, Homo sapiens
ccttcgatagctcagctggtagagcggaggactgtagattgtacagacatttgcggacatccttaggtcgctggttcgattccggctcgaaggaa

SEQ ID NO: 400 - DNA, 74 bp, Homo sapiens
aggccctatagctcaggggttagagcactggtcttgtaaaccaggggtcgcgagttcaaatctcgctggggcct

SEQ ID NO: 401 - DNA, 90 bp, Homo sapiens
tccttcgatagctcagctggtagagcggaggactgtagtacttaatgtgtggtcatccttaggtcgctggttcgattccggctcgaagga

SEQ ID NO: 402 - DNA, 73 bp, Homo sapiens
tggctcgttggtctaggggtatgattctcgctttgggtgcgagaggtcccgggttcaaatcccggacgagccc

SEQ ID NO: 403 - DNA, 74 bp, Homo sapiens
gcccggctagctcagtcggtagagcatgggactcttaatcccagggtcgtgggttcgagccccacgttgggcgc

SEQ ID NO: 404 - DNA, 75 bp, Homo sapiens
cggccggttagctcagttggttagagcgtggtgctaataacgccaaggtcgcgggttcgatccccgtacgggcca

SEQ ID NO: 405 - DNA, 73 bp, Homo sapiens
tcccacatggtctagcggttaggattcctggttttcacccaggcggcccgggttcgactcccggtgtgggaat

SEQ ID NO: 406 - DNA, 83 bp, Homo sapiens
gacgaggtggccgagtggttaaggcgatggactgctaatccattgtgctctgcacgcgtgggttcgaatcccatcctcgtcga

SEQ ID NO: 407 - DNA, 73 bp, Homo sapiens
gccgtgatcgtatagtggttagtactctgcgttgtggccgcagcaacctcggttcgaatccgagtcacggcat

SEQ ID NO: 408 - DNA, 73 bp, Homo sapiens
cgccgtgatcgtatagtggttagtactctgcgttgtggccgcagcaacctcggttcgaatccgagtcacggca

SEQ ID NO: 409 - DNA, 73 bp, Homo sapiens
ggttccatggtgtaatggttagcactctggactctgaatccagcgatccgagttcaaatctcggtggaacctg

SEQ ID NO: 410 - DNA, 74 bp, Homo sapiens
tgcccggctagctcagtcggtagagcatgggactcttaatcccagggtcgtgggttcgagccccacgttgggcg

SEQ ID NO: 411 - DNA, 74 bp, Homo sapiens
gggccgcgtggcctaatggataaggcgtctgacttcggatcagaagattgcaggttcgagtcctgccgcggtcg

SEQ ID NO: 412 - DNA, 72 bp, Homo sapiens
gcgccgctggtgtagtggtatcatgcaagattcccattcttgcgacccgggttcgattcccgggcggcgcac

SEQ ID NO: 413 - DNA, 74 bp, Homo sapiens
gggccgcgtggcctaatggataaggcgtctgattccggatcagaagattgagggttcgagtcccttcgtggtcg

SEQ ID NO: 414 - DNA, 74 bp, Homo sapiens
cgccccggtggcctaatggataaggcattggcctcctaagccagggattgtgggttcgagtcccacccggggta

SEQ ID NO: 415 - DNA, 74 bp, Homo sapiens
gcccggctagctcagtcggtagagcatgagacccttaatctcagggtcgtgggttcgagccccacgttgggcgt

SEQ ID NO: 416 - DNA, 73 bp, Homo sapiens
aggcgcggtggccaagtggtaaggcgtcggtctcgtaaaccgaagatcacgggttcgaaccccgtccgtgcct

SEQ ID NO: 417 - DNA, 83 bp, Homo sapiens
ggtagcgtggccgagtggtctaaggcgctggatttaggctccagtcatttcgatggcgtgggttcgaatcccaccgctgccac

SEQ ID NO: 418 - DNA, 83 bp, Homo sapiens
gggtagcgtggccgagcggtctaaggcgctggattaaggctccagtctcttcgggggcgtgggttcgaatcccaccgctgcca

SEQ ID NO: 419 - DNA, 84 bp, Homo sapiens
agtcaggatggccgagcggtctaaggcgctgcgttcaggtcgcagtctcccctggaggcgtgggttcgaatcccacttctgaca

SEQ ID NO: 420 - DNA, 84 bp, Homo sapiens
gtcaggatggccgagcggtctaaggcgctgcgttcaggtcgcagtctcccctggaggcgtgggttcgaatcccacttctgacag

SEQ ID NO: 421 - DNA, 74 bp, Homo sapiens
gcctcgttagcgcagtaggcagcgcgtcagtctcataatctgaaggtcgtgagttcgagcctcacacggggcag

SEQ ID NO: 422 - DNA, 83 bp, Homo sapiens
ggtagcgtggccgagcggtctaaggcgctggatttaggctccagtctcttcggaggcgtgggttcgaatcccaccgctgccag

SEQ ID NO: 423 - DNA, 89 bp, Homo sapiens
tggctctgtggcgcaatggatagcgcattggacttctagtgacgaatagagcaattcaaaggttgtgggttcgaatcccaccagagtcg

SEQ ID NO: 424 - DNA, 72 bp, Homo sapiens
cgcattggtggttcagtggtagaattctcgcctgccacgcgggaggcccgggttcgattcccggccaatgca

SEQ ID NO: 425 - DNA, 83 bp, Homo sapiens
gctgtgatggccgagtggttaaggcgttggactcgaaatccaatggggtctccccgcgcaggttcgaatcctgctcacagcgt

SEQ ID NO: 426 - DNA, 75 bp, Homo sapiens
ggcgccgtggcttagctggttaaagcgcctgtctagtaaacaggagatcctgggttcgaatcccagcggtgcctg

SEQ ID NO: 427 - DNA, 73 bp, Homo sapiens
cgacctcgtggcgcaacggtagcgcgtctgactccagatcagaaggttgcgtgttcaaatcacgtcggggtca

SEQ ID NO: 428 - DNA, 83 bp, Homo sapiens
agacgaggtggccgagtggttaaggcgatggactgctaatccattgtgctctgcacgcgtgggttcgaatcccatcctcgtcg

SEQ ID NO: 429 - DNA, 75 bp, Homo sapiens
cggcgccgtggcttagttggttaaagcgcctgtctagtaaacaggagatcctgggttcgaatcccagcggtgcct

SEQ ID NO: 430 - DNA, 73 bp, Homo sapiens
ggcctcgtggcgcaacggtagcgcgtctgactccagatcagaaggttgcgtgttcaaatcacgtcggggtcaa

SEQ ID NO: 431 - DNA, 73 bp, Homo sapiens
agcgttggtggtatagtggtaagcatagctgccttccaagcagttgacccgggttcgattcccggccaacgca

SEQ ID NO: 432 - DNA, 73 bp, Homo sapiens
tcctcgttagtatagtggtgagtatccccgcctgtcacgcgggagaccggggttcgattccccgacggggaga

SEQ ID NO: 433 - DNA, 73 bp, Homo sapiens
ggctcgttggtctaggggtatgattctcgcttcgggtgcgagaggtcccgggttcaaatcccggacgagccct

SEQ ID NO: 434 - DNA, 75 bp, Homo sapiens
ggcgccgtggcttagttggttaaagcgcctgtctagtaaacaggagatcctgggttcgaatcccagcggtgcctt

SEQ ID NO: 435 - DNA, 83 bp, Homo sapiens
gtagtcgtggccgagtggttaaggcgatggactagaaatccattggggtctccccgcgcaggttcgaatcctgccgactacgt

SEQ ID NO: 436 - DNA, 73 bp, Homo sapiens
tgacctcgtggcgcaatggtagcgcgtctgactccagatcagaaggttgcgtgttcaagtcacgtcggggtca

SEQ ID NO: 437 - DNA, 73 bp, Homo sapiens
aggcgcggtggccaagtggtaaggcgtcggtctcgtaaaccgaagatcgcgggttcgaaccccgtccgtgcct

SEQ ID NO: 438 - DNA, 73 bp, Homo sapiens
agggggtatagctcagtggtagagcatttgactgcagatcaagaggtccccggttcaaatccgggtgccccct

SEQ ID NO: 439 - DNA, 73 bp, Homo sapiens
gggggtatagctcagtggtagagcatttgactgcagatcaagaggtccctggttcaaatccgggtgccccctc

SEQ ID NO: 440 - DNA, 73 bp, Homo sapiens
gggggtatagctcagtggtagagcatttgactgcagatcaagaggtccccggttcaaatccgggtgccccctc

SEQ ID NO: 441 - DNA, 73 bp, Homo sapiens
aggtcccatggtgtaatggttagcactctggactttgaatccagcgatccgagttcaaatctcggtgggacct

SEQ ID NO: 442 - DNA, 74 bp, Homo sapiens
gacccagtggcctaatggataaggcatcagcctccggagctggggattgtgggttcgagtcccatctgggtcgc

SEQ ID NO: 443 - DNA, 74 bp, Homo sapiens
agccccagtggcctaatggataaggcactggcctcctaagccagggattgtgggttcgagtcccacctggggta

SEQ ID NO: 444 - DNA, 74 bp, Homo sapiens
gccccagtggcctaatggataaggcactggcctcctaagccagggattgtgggttcgagtcccacctggggtgt

SEQ ID NO: 445 - DNA, 74 bp, Homo sapiens
agaccgcgtggcctaatggataaggcgtctgacttcggatcagaagattgagggttcgagtcccttcgtggtcg

SEQ ID NO: 446 - DNA, 75 bp, Homo sapiens
cgtctctgtggcgcaatcggttagcgcgttcggctgttaaccgaaaggttggtggttcgagcccacccagggacg

SEQ ID NO: 447 - DNA, 73 bp, Homo sapiens
ggcgttggtggtatagtggttagcatagctgccttccaagcagttgacccgggttcgattcccggccaacgca

SEQ ID NO: 448 - DNA, 74 bp, Homo sapiens
gtttccgtagtgtagcggttatcacattcgcctcacacgcgaaaggtccccggttcgatcccgggcggaaacag

SEQ ID NO: 449 - DNA, 75 bp, Homo sapiens
tggcgccgtggcttagttggttaaagcgcctgtctagtaaacaggagatcctgggttcgaatcccagcggtgcct

SEQ ID NO: 450 - DNA, 94 bp, Homo sapiens
gctccagtggcgcaatcggttagcgcgcggtacttatatgacagtgcgagcggagcaatgccgaggttgtgagttcgatcctcacctggagcac

SEQ ID NO: 451 - DNA, 72 bp, Homo sapiens
gcatgggtggttcagtggtagaattctcgcctgccacgcgggaggcccgggttcgattcccggcccatgcag

SEQ ID NO: 452 - DNA, 18 bp, Artificial Sequence
  source: /note=“Description of Artificial Sequence Synthetic oligonucleotide”
aaaatataaatatatttc

SEQ ID NO: 453 - DNA, 5 bp, Artificial Sequence
  source: /note=“Description of Artificial Sequence Synthetic oligonucleotide”
aagct

SEQ ID NO: 454 - DNA, 5 bp, Artificial Sequence
  source: /note=“Description of Artificial Sequence Synthetic oligonucleotide”
aagtt

SEQ ID NO: 455 - DNA, 15 bp, Artificial Sequence
  source: /note=“Description of Artificial Sequence Synthetic oligonucleotide”
aattcttcggaatgt

SEQ ID NO: 456 - DNA, 3 bp, Artificial Sequence
  source: /note=“Description of Artificial Sequence Synthetic oligonucleotide”
aga

SEQ ID NO: 457 - DNA, 5 bp, Artificial Sequence
  source: /note=“Description of Artificial Sequence Synthetic oligonucleotide”
agtcc

SEQ ID NO: 458 - DNA, 5 bp, Artificial Sequence
  source: /note=“Description of Artificial Sequence Synthetic oligonucleotide”
caacc

SEQ ID NO: 459 - DNA, 5 bp, Artificial Sequence
  source: /note=“Description of Artificial Sequence Synthetic oligonucleotide”
caatc

SEQ ID NO: 460 - DNA, 4 bp, Artificial Sequence
  source: /note=“Description of Artificial Sequence Synthetic oligonucleotide”
cagc

SEQ ID NO: 461 - DNA, 20 bp, Artificial Sequence
  source: /note=“Description of Artificial Sequence Synthetic oligonucleotide”
caggcgggttctgcccgcgc

SEQ ID NO: 462 - DNA, 18 bp, Artificial Sequence
  source: /note=“Description of Artificial Sequence Synthetic oligonucleotide”
catacctgcaagggtatc

SEQ ID NO: 463 - DNA, 15 bp, Artificial Sequence
  source: /note=“Description of Artificial Sequence Synthetic oligonucleotide”
cgaccgcaaggttgt

SEQ ID NO: 464 - DNA, 15 bp, Artificial Sequence
  source: /note=“Description of Artificial Sequence Synthetic oligonucleotide”
cgaccttgcggtcat

SEQ ID NO: 465 - DNA, 18 bp, Artificial Sequence
  source: /note=“Description of Artificial Sequence Synthetic oligonucleotide”
cgatgctaatcacatcgt

SEQ ID NO: 466 - DNA, 15 bp, Artificial Sequence
  source: /note=“Description of Artificial Sequence Synthetic oligonucleotide”
cgatggtgacatcat

SEQ ID NO: 467 - DNA, 16 bp, Artificial Sequence
  source: /note=“Description of Artificial Sequence Synthetic oligonucleotide”
cgatggtttacatcgt

SEQ ID NO: 468 - DNA, 13 bp, Artificial Sequence
  source: /note=“Description of Artificial Sequence Synthetic oligonucleotide”
cgccgtaaggtgt

SEQ ID NO: 469 - DNA, 12 bp, Artificial Sequence
  source: /note=“Description of Artificial Sequence Synthetic oligonucleotide”
cgccttaggtgt

SEQ ID NO: 470 - DNA, 15 bp, Artificial Sequence
  source: /note=“Description of Artificial Sequence Synthetic oligonucleotide”
cgcctttcgacgcgt

SEQ ID NO: 471 - DNA, 13 bp, Artificial Sequence
  source: /note=“Description of Artificial Sequence Synthetic oligonucleotide”
cgcttcacggcgt

SEQ ID NO: 472 - DNA, 15 bp, Artificial Sequence
  source: /note=“Description of Artificial Sequence Synthetic oligonucleotide”
cggcagcaatgctgt

SEQ ID NO: 473 - DNA, 13 bp, Artificial Sequence
  source: /note=“Description of Artificial Sequence Synthetic oligonucleotide”
cggctccgccttc

SEQ ID NO: 474 - DNA, 16 bp, Artificial Sequence
  source: /note=“Description of Artificial Sequence Synthetic oligonucleotide”
cgggtatcacagggtc

SEQ ID NO: 475 - DNA, 17 bp, Artificial Sequence
  source: /note=“Description of Artificial Sequence Synthetic oligonucleotide”
cggtgcgcaagcgctgt

SEQ ID NO: 476 - DNA, 18 bp, Artificial Sequence
  source: /note=“Description of Artificial Sequence Synthetic oligonucleotide”
cgtacgggtgaccgtacc

SEQ ID NO: 477 - DNA, 13 bp, Artificial Sequence
  source: /note=“Description of Artificial Sequence Synthetic oligonucleotide”
cgtcaaagacttc

SEQ ID NO: 478 - DNA, 13 bp, Artificial Sequence
  source: /note=“Description of Artificial Sequence Synthetic oligonucleotide”
cgtcgtaagactt

SEQ ID NO: 479 - DNA, 14 bp, Artificial Sequence
  source: /note=“Description of Artificial Sequence Synthetic oligonucleotide”
cgttgaataaacgt

SEQ ID NO: 480 - DNA, 5 bp, Artificial Sequence
  source: /note=“Description of Artificial Sequence Synthetic oligonucleotide”
ctgtc

SEQ ID NO: 481 - DNA, 4 bp, Artificial Sequence
  source: /note=“Description of Artificial Sequence Synthetic oligonucleotide”
ggcc

SEQ ID NO: 482 - DNA, 7 bp, Artificial Sequence
  source: /note=“Description of Artificial Sequence Synthetic oligonucleotide”
ggggatt

SEQ ID NO: 483 - DNA, 4 bp, Artificial Sequence
  source: /note=“Description of Artificial Sequence Synthetic oligonucleotide”
ggtc

SEQ ID NO: 484 - DNA, 5 bp, Artificial Sequence
  source: /note=“Description of Artificial Sequence Synthetic oligonucleotide”
ggttt

SEQ ID NO: 485 - DNA, 4 bp, Artificial Sequence
  source: /note=“Description of Artificial Sequence Synthetic oligonucleotide”
gtag

SEQ ID NO: 486 - DNA, 19 bp, Artificial Sequence
  source: /note=“Description of Artificial Sequence Synthetic oligonucleotide”
taactagatactttcagat

SEQ ID NO: 487 - DNA, 15 bp, Artificial Sequence
  source: /note=“Description of Artificial Sequence Synthetic oligonucleotide”
tactcgtatgggtgc

SEQ ID NO: 488 - DNA, 13 bp, Artificial Sequence
  source: /note=“Description of Artificial Sequence Synthetic oligonucleotide”
tactttgcggtgt

SEQ ID NO: 489 - DNA, 19 bp, Artificial Sequence
  source: /note=“Description of Artificial Sequence Synthetic oligonucleotide”
taggcgagtaacatcgtgc

SEQ ID NO: 490 - DNA, 19 bp, Artificial Sequence
  source: /note=“Description of Artificial Sequence Synthetic oligonucleotide”
taggcgtgaatagcgcctc

SEQ ID NO: 491 - DNA, 19 bp, Artificial Sequence
  source: /note=“Description of Artificial Sequence Synthetic oligonucleotide”
taggtcgcgagagcggcgc

SEQ ID NO: 492 - DNA, 19 bp, Artificial Sequence
  source: /note=“Description of Artificial Sequence Synthetic oligonucleotide”
taggtcgcgtaagcggcgc

SEQ ID NO: 493 - DNA, 17 bp, Artificial Sequence
  source: /note=“Description of Artificial Sequence Synthetic oligonucleotide”
taggtggttatccacgc

SEQ ID NO: 494 - DNA, 5 bp, Artificial Sequence
  source: /note=“Description of Artificial Sequence Synthetic oligonucleotide”
tagtc

SEQ ID NO: 495 - DNA, 5 bp, Artificial Sequence
  source: /note=“Description of Artificial Sequence Synthetic oligonucleotide”
tagtt

SEQ ID NO: 496 - DNA, 18 bp, Artificial Sequence
  source: /note=“Description of Artificial Sequence Synthetic oligonucleotide”
tatacgtgaaagcgtatc

SEQ ID NO: 497 - DNA, 21 bp, Artificial Sequence
  source: /note=“Description of Artificial Sequence Synthetic oligonucleotide”
tatagggtcaaaaactctatc

SEQ ID NO: 498 - DNA, 20 bp, Artificial Sequence
  source: /note=“Description of Artificial Sequence Synthetic oligonucleotide”
tatgcagaaatacctgcatc

SEQ ID NO: 499 - DNA, 15 bp, Artificial Sequence
  source: /note=“Description of Artificial Sequence Synthetic oligonucleotide”
tccccatacgggggc

SEQ ID NO: 500 - DNA, 14 bp, Artificial Sequence
  source: /note=“Description of Artificial Sequence Synthetic oligonucleotide”
tcccgaaggggttc

SEQ ID NO: 501 - DNA, 15 bp, Artificial Sequence
  source: /note=“Description of Artificial Sequence Synthetic oligonucleotide”
tctacgtatgtgggc

SEQ ID NO: 502 - DNA, 14 bp, Artificial Sequence
  source: /note=“Description of Artificial Sequence Synthetic oligonucleotide”
tctcataggagttc

SEQ ID NO: 503 - DNA, 14 bp, Artificial Sequence
  source: /note=“Description of Artificial Sequence Synthetic oligonucleotide”
tctcctctggaggc

SEQ ID NO: 504 - DNA, 15 bp, Artificial Sequence
  source: /note=“Description of Artificial Sequence Synthetic oligonucleotide”
tcttagcaataaggt

SEQ ID NO: 505 - DNA, 14 bp, Artificial Sequence
  source: /note=“Description of Artificial Sequence Synthetic oligonucleotide”
tcttgtaggagttc

SEQ ID NO: 506 - DNA, 15 bp, Artificial Sequence
  source: /note=“Description of Artificial Sequence Synthetic oligonucleotide”
tgaacgtaagttcgc

SEQ ID NO: 507 - DNA, 16 bp, Artificial Sequence
  source: /note=“Description of Artificial Sequence Synthetic oligonucleotide”
tgaactgcgaggttcc

SEQ ID NO: 508 - DNA, 4 bp, Artificial Sequence
  source: /note=“Description of Artificial Sequence Synthetic oligonucleotide”
tgac

SEQ ID NO: 509 - DNA, 15 bp, Artificial Sequence
  source: /note=“Description of Artificial Sequence Synthetic oligonucleotide”
tgaccgaaaggtcgt

SEQ ID NO: 510 - DNA, 15 bp, Artificial Sequence
  source: /note=“Description of Artificial Sequence Synthetic oligonucleotide”
tgaccgcaaggtcgt

SEQ ID NO: 511 - DNA, 14 bp, Artificial Sequence
  source: /note=“Description of Artificial Sequence Synthetic oligonucleotide”
tgagctctgctctc

SEQ ID NO: 512 - DNA, 18 bp, Artificial Sequence
  source: /note=“Description of Artificial Sequence Synthetic oligonucleotide”
tgaggcctcacggcctac

SEQ ID NO: 513 - DNA, 15 bp, Artificial Sequence
  source: /note=“Description of Artificial Sequence Synthetic oligonucleotide”
tgagggcaacttcgt

SEQ ID NO: 514 - DNA, 16 bp, Artificial Sequence
  source: /note=“Description of Artificial Sequence Synthetic oligonucleotide”
tgagggtcatacctcc

SEQ ID NO: 515 - DNA, 18 bp, Artificial Sequence
  source: /note=“Description of Artificial Sequence Synthetic oligonucleotide”
tgagggtgcaaatcctcc

SEQ ID NO: 516 - DNA, 13 bp, Artificial Sequence
  source: /note=“Description of Artificial Sequence Synthetic oligonucleotide”
tgccgaaaggcgt

SEQ ID NO: 517 - DNA, 13 bp, Artificial Sequence
  source: /note=“Description of Artificial Sequence Synthetic oligonucleotide”
tgccgtaaggcgt

SEQ ID NO: 518 - DNA, 14 bp, Artificial Sequence
  source: /note=“Description of Artificial Sequence Synthetic oligonucleotide”
tgcggtctccgcgc

SEQ ID NO: 519 - DNA, 11 bp, Artificial Sequence
  source: /note=“Description of Artificial Sequence Synthetic oligonucleotide”
tgctagagcat

SEQ ID NO: 520 - DNA, 16 bp, Artificial Sequence
  source: /note=“Description of Artificial Sequence Synthetic oligonucleotide”
tgctcgtatagagctc

SEQ ID NO: 521 - DNA, 15 bp, Artificial Sequence
  source: /note=“Description of Artificial Sequence Synthetic oligonucleotide”
tggacaattgtctgc

SEQ ID NO: 522 - DNA, 15 bp, Artificial Sequence
  source: /note=“Description of Artificial Sequence Synthetic oligonucleotide”
tggacagatgtccgt

SEQ ID NO: 523 - DNA, 15 bp, Artificial Sequence
  source: /note=“Description of Artificial Sequence Synthetic oligonucleotide”
tggacaggtgtccgc

SEQ ID NO: 524 - DNA, 15 bp, Artificial Sequence
  source: /note=“Description of Artificial Sequence Synthetic oligonucleotide”
tggacggttgtccgc

SEQ ID NO: 525 - DNA, 13 bp, Artificial Sequence
  source: /note=“Description of Artificial Sequence Synthetic oligonucleotide”
tggacttgtggtc

SEQ ID NO: 526 - DNA, 16 bp, Artificial Sequence
  source: /note=“Description of Artificial Sequence Synthetic oligonucleotide”
tggagattctctccgc

SEQ ID NO: 527 - DNA, 14 bp, Artificial Sequence
  source: /note=“Description of Artificial Sequence Synthetic oligonucleotide”
tggcataggcctgc

SEQ ID NO: 528 - DNA, 14 bp, Artificial Sequence
  source: /note=“Description of Artificial Sequence Synthetic oligonucleotide”
tggcttatgtctac

SEQ ID NO: 529 - DNA, 16 bp, Artificial Sequence
  source: /note=“Description of Artificial Sequence Synthetic oligonucleotide”
tgggagttaatcccgt

SEQ ID NO: 530 - DNA, 14 bp, Artificial Sequence
  source: /note=“Description of Artificial Sequence Synthetic oligonucleotide”
tgggatcttcccgc

SEQ ID NO: 531 - DNA, 16 bp, Artificial Sequence
  source: /note=“Description of Artificial Sequence Synthetic oligonucleotide”
tgggcagaaatgtctc

SEQ ID NO: 532 - DNA, 15 bp, Artificial Sequence
  source: /note=“Description of Artificial Sequence Synthetic oligonucleotide”
tgggcgttcgcccgc

SEQ ID NO: 533 - DNA, 14 bp, Artificial Sequence
  source: /note=“Description of Artificial Sequence Synthetic oligonucleotide”
tgggcttcgcccgc

SEQ ID NO: 534 - DNA, 16 bp, Artificial Sequence
  source: /note=“Description of Artificial Sequence Synthetic oligonucleotide”
tgggggataaccccgt

SEQ ID NO: 535 - DNA, 15 bp, Artificial Sequence
  source: /note=“Description of Artificial Sequence Synthetic oligonucleotide”
tgggggtttccccgt

SEQ ID NO: 536 - DNA, 4 bp, Artificial Sequence
  source: /note=“Description of Artificial Sequence Synthetic oligonucleotide”
tggt

SEQ ID NO: 537 - DNA, 15 bp, Artificial Sequence
  source: /note=“Description of Artificial Sequence Synthetic oligonucleotide”
tggtggcaacaccgt

SEQ ID NO: 538 - DNA, 14 bp, Artificial Sequence
  source: /note=“Description of Artificial Sequence Synthetic oligonucleotide”
tggtttatagccgt

SEQ ID NO: 539 - DNA, 19 bp, Artificial Sequence
  source: /note=“Description of Artificial Sequence Synthetic oligonucleotide”
tgtacggtaataccgtacc

SEQ ID NO: 540 - DNA, 15 bp, Artificial Sequence
  source: /note=“Description of Artificial Sequence Synthetic oligonucleotide”
tgtccgcaaggacgt

SEQ ID NO: 541 - DNA, 15 bp, Artificial Sequence
  source: /note=“Description of Artificial Sequence Synthetic oligonucleotide”
tgtcctaacggacgt

SEQ ID NO: 542 - DNA, 18 bp, Artificial Sequence
  source: /note=“Description of Artificial Sequence Synthetic oligonucleotide”
tgtcctattaacggacgt

SEQ ID NO: 543 - DNA, 16 bp, Artificial Sequence
  source: /note=“Description of Artificial Sequence Synthetic oligonucleotide”
tgtccttcacgggcgt

SEQ ID NO: 544 - DNA, 13 bp, Artificial Sequence
  source: /note=“Description of Artificial Sequence Synthetic oligonucleotide”
tgtcttaggacgt

SEQ ID NO: 545 - DNA, 18 bp, Artificial Sequence
  source: /note=“Description of Artificial Sequence Synthetic oligonucleotide”
tgtgcgttaacgcgtacc

SEQ ID NO: 546 - DNA, 16 bp, Artificial Sequence
  source: /note=“Description of Artificial Sequence Synthetic oligonucleotide”
tgtgtcgcaaggcacc

SEQ ID NO: 547 - DNA, 15 bp, Artificial Sequence
  source: /note=“Description of Artificial Sequence Synthetic oligonucleotide”
tgttcgtaaggactt

SEQ ID NO: 548 - DNA, 16 bp, Artificial Sequence
  source: /note=“Description of Artificial Sequence Synthetic oligonucleotide”
ttcacagaaatgtgtc

SEQ ID NO: 549 - DNA, 14 bp, Artificial Sequence
  source: /note=“Description of Artificial Sequence Synthetic oligonucleotide”
ttccctcgtggagt

SEQ ID NO: 550 - DNA, 14 bp, Artificial Sequence
  source: /note=“Description of Artificial Sequence Synthetic oligonucleotide”
ttccctctgggagc

SEQ ID NO: 551 - DNA, 14 bp, Artificial Sequence
  source: /note=“Description of Artificial Sequence Synthetic oligonucleotide”
ttcccttgtggatc

SEQ ID NO: 552 - DNA, 13 bp, Artificial Sequence
  source: /note=“Description of Artificial Sequence Synthetic oligonucleotide”
ttccttcgggagc

SEQ ID NO: 553 - DNA, 15 bp, Artificial Sequence
  source: /note=“Description of Artificial Sequence Synthetic oligonucleotide”
ttctagcaatagagt

SEQ ID NO: 554 - DNA, 16 bp, Artificial Sequence
  source: /note=“Description of Artificial Sequence Synthetic oligonucleotide”
ttctccactggggagc

SEQ ID NO: 555 - DNA, 15 bp, Artificial Sequence
  source: /note=“Description of Artificial Sequence Synthetic oligonucleotide”
ttctcgagagggagc

SEQ ID NO: 556 - DNA, 15 bp, Artificial Sequence
  source: /note=“Description of Artificial Sequence Synthetic oligonucleotide”
ttctcgtatgagagc

SEQ ID NO: 557 - DNA, 19 bp, Artificial Sequence
  source: /note=“Description of Artificial Sequence Synthetic oligonucleotide”
tttaaggttttcccttaac

SEQ ID NO: 558 - DNA, 14 bp, Artificial Sequence
  source: /note=“Description of Artificial Sequence Synthetic oligonucleotide”
tttcattgtggagt

SEQ ID NO: 559 - DNA, 14 bp, Artificial Sequence
  source: /note=“Description of Artificial Sequence Synthetic oligonucleotide”
tttcgaaggaatcc

SEQ ID NO: 560 - DNA, 13 bp, Artificial Sequence
  source: /note=“Description of Artificial Sequence Synthetic oligonucleotide”
tttcttcggaagc

SEQ ID NO: 561 - DNA, 16 bp, Artificial Sequence
  source: /note=“Description of Artificial Sequence Synthetic oligonucleotide”
tttggggcaactcaac

SEQ ID NO: 562 - RNA, 342 nt, Artificial Sequence
  source: /note=“Description of Artificial Sequence Synthetic polynucleotide”
  modified_base (5)..(6): a, c, u, or g
  modified_base (15)..(16): a, c, u, or g
  modified_base (21)..(21): a, c, u, or g
  modified_base (30)..(32): a, c, u, or g
  modified_base (34)..(34): a, c, u, or g
  modified_base (37)..(37): a, c, u, or g
  modified_base (41)..(45): a, c, u, or g
  misc_feature (47)..(317): /note=“This region may encompass 1-271 nucleotides”
  modified_base (47)..(320): a, c, u, or g
  modified_base (328)..(329): a, c, u, or g
  modified_base (333)..(334): a, c, u, or g
  modified_base (336)..(337): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rkssnndurghbyannyugrndgwdvdydnnnbnhbnryrnnnnndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrrduyrannyybnnhnnbwccd

SEQ ID NO: 563 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (328)..(328): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rksgrwdkrghbyavnyggudgarvrydynhkywbhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducrayncyydvhhyywccd

SEQ ID NO: 564 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ggggrwdurghbyavnyggudgarvrydynhkywkhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducraybccydvhhyyucca

SEQ ID NO: 565 - RNA, 343 nt, Artificial Sequence
  source: /note=“Description of Artificial Sequence Synthetic polynucleotide”
  modified_base (2)..(8): a, c, u, or g
  modified_base (12)..(13): a, c, u, or g
  modified_base (17)..(17): a, c, u, or g
  modified_base (22)..(24): a, c, u, or g
  modified_base (26)..(27): a, c, u, or g
  modified_base (30)..(35): a, c, u, or g
  modified_base (38)..(38): a, c, u, or g
  modified_base (43)..(43): a, c, u, or g
  modified_base (45)..(322): a, c, u, or g
  misc_feature (48)..(318): /note=“This region may encompass 1-271 nucleotides”
  modified_base (329)..(329): a, c, u, or g
  modified_base (333)..(342): a, c, u, or g
  variation (1)..(343): /replace=“ ”
  misc_feature (1)..(343): /note=“Variant nucleotides given in the sequence have no preference with respect to those in the annotations for variant positions”
  source: /note=“See specification as filed for detailed description of substitutions and preferred embodiments”
gnnnnnnnkvgnnhddnhwrrnnnrnnvynnnnnnbunckrhnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnguuyranyybnnnnnnnnnnd

SEQ ID NO: 566 - RNA, 342 nt, Artificial Sequence
  source: /note=“Description of Artificial Sequence Synthetic polynucleotide”
  modified_base (3)..(3): a, c, u, or g
  modified_base (6)..(7): a, c, u, or g
  modified_base (13)..(13): a, c, u, or g
  modified_base (26)..(26): a, c, u, or g
  modified_base (32)..(32): a, c, u, or g
  modified_base (37)..(37): a, c, u, or g
  modified_base (44)..(44): a, c, u, or g
  modified_base (47)..(317): a, c, u, or g
  misc_feature (47)..(317): /note=“This region may encompass 1-271 nucleotides”
  modified_base (328)..(328): a, c, u, or g
  modified_base (336)..(338): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvbnnvkbgbnydrwurgwygarnrydyynsvyunckrmkbnrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshvgguuyranuycbdbynnnbnhr

SEQ ID NO: 567 - RNA, 342 nt, Artificial Sequence
  source: /note=“Description of Artificial Sequence Synthetic polynucleotide”
  modified_base (3)..(3): a, c, u, or g
  modified_base (7)..(7): a, c, u, or g
  modified_base (47)..(317): a, c, u, or g
  misc_feature (47)..(317): /note=“This region may encompass 1-271 nucleotides”
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvyvnskkgssydawuggaygarsgyryykgmyuhckrakymrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshrgguuygavuycydbynbdbnyg

SEQ ID NO: 568 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(12): a, c, u, or g
  modified_base (17)..(17): a, c, u, or g
  modified_base (25)..(25): a, c, u, or g
  modified_base (28)..(30): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (334)..(337): a, c, u, or g
  modified_base (339)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnynnndurghnhvrynggbhurdnrynnnbsryyruuwahbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgukhvwnhcynnbnnnndnnr

SEQ ID NO: 569 - RNA, 341 nt, Artificial Sequence
  source: /note=“Description of Artificial Sequence Synthetic polynucleotide”
  modified_base (17)..(17): a, c, u, or g
  modified_base (28)..(28): a, c, u, or g
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrunggbyuarhgcnuuysryyruuwahbddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrrbr

SEQ ID NO: 570 - RNA, 341 nt, Artificial Sequence
  source: /note=“Description of Artificial Sequence Synthetic polynucleotide”
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrubggbyuaghgcbuuysryyruuaahyddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrryg

SEQ ID NO: 571 - RNA, 342 nt, Artificial Sequence
  source: /note=“Description of Artificial Sequence Synthetic polynucleotide”
  modified_base (2)..(6): a, c, u, or g
  modified_base (12)..(12): a, c, u, or g
  modified_base (16)..(16): a, c, u, or g
  modified_base (22)..(22): a, c, u, or g
  modified_base (26)..(26): a, c, u, or g
  modified_base (29)..(29): a, c, u, or g
  modified_base (31)..(32): a, c, u, or g
  modified_base (44)..(44): a, c, u, or g
  misc_feature (47)..(317): /note=“This region may encompass 1-271 nucleotides”
  modified_base (47)..(319): a, c, u, or g
  modified_base (328)..(328): a, c, u, or g
  modified_base (333)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
knnnnndyrrynhrrnyygrhnumdnrynbnnvhyurucrhdbnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsrgyubrhnyyysnnhnnnnbmr

SEQ ID NO: 572 - RNA, 339 nt, Artificial Sequence
  source: /note=“Description of Artificial Sequence Synthetic polynucleotide”
  modified_base (2)..(2): a, c, u, or g
  modified_base (6)..(6): a, c, u, or g
  modified_base (44)..(314): a, c, u, or g
  misc_feature (44)..(314): /note=“This region may encompass 1-271 nucleotides”
  modified_base (333)..(333): a, c, u, or g
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
knsbbnwyarywyrguggukmgwrubysyryyurucaygbrsrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnykgrgyubrhkyycchrwnvvksmr

SEQ ID NO: 573 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
umcuyruyaguaurguggukmguruyycygycugucaygyggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyggrguuyrmkyyccyrasrrggag

SEQ ID NO: 574 - RNA, 342 nt, Artificial Sequence
  source: /note=“Description of Artificial Sequence Synthetic polynucleotide”
  modified_base (3)..(6): a, c, u, or g
  modified_base (12)..(13): a, c, u, or g
  modified_base (16)..(16): a, c, u, or g
  modified_base (24)..(24): a, c, u, or g
  modified_base (26)..(26): a, c, u, or g
  modified_base (29)..(32): a, c, u, or g
  modified_base (34)..(34): a, c, u, or g
  modified_base (42)..(42): a, c, u, or g
  modified_base (44)..(319): a, c, u, or g
  misc_feature (47)..(317): /note=“This region may encompass 1-271 nucleotides”
  modified_base (328)..(328): a, c, u, or g
  modified_base (333)..(334): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
gsnnnndurvynnavnkcgdbyynrnvbnnnnvnyyrcarmnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdruucranyyyvnnhnnnnnyy

SEQ ID NO: 575 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsgsryrkrgyubasnkgdkuaravyahuugrcyrcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyydruucraruyyvgkuryychyy

SEQ ID NO: 576 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gggsryrkrgyuyasnugrkuagagcayuugacugcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyyrruucraauyyvgkurcycmyy

SEQ ID NO: 577 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(13): a, c, u, or g
  modified_base (16)..(16): a, c, u, or g
  modified_base (20)..(21): a, c, u, or g
  modified_base (24)..(24): a, c, u, or g
  modified_base (28)..(31): a, c, u, or g
  modified_base (33)..(33): a, c, u, or g
  modified_base (40)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnbnnnduvgbnnarnhggnnhanvvynnnnvnyubukrnnhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrruuyvanybynnbhnnnnnhd

SEQ ID NO: 578 - RNA, 340 nt, Artificial Sequence
  source: /note=“Description of Artificial Sequence Synthetic polynucleotide”
  modified_base (4)..(5): a, c, u, or g
  modified_base (12)..(12): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (335)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnnyduggynbarkmggdnavvrynnhbvryubukrrbhvdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadybyhnbhdnnvhmd

SEQ ID NO: 579 - RNA, 340 nt, Artificial Sequence
  source: /note=“Description of Artificial Sequence Synthetic polynucleotide”
  modified_base (4)..(4): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnvyduggydbarumggknavvrynnwsrryubukrabhsdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadysyhnbhdbnvhmk

SEQ ID NO: 580 - RNA, 342 nt, Artificial Sequence
  source: /note=“Description of Artificial Sequence Synthetic polynucleotide”
  modified_base (1)..(2): a, c, u, or g
  modified_base (5)..(6): a, c, u, or g
  modified_base (9)..(9): a, c, u, or g
  modified_base (12)..(12): a, c, u, or g
  modified_base (16)..(16): a, c, u, or g
  modified_base (20)..(21): a, c, u, or g
  modified_base (26)..(27): a, c, u, or g
  modified_base (29)..(33): a, c, u, or g
  modified_base (41)..(41): a, c, u, or g
  modified_base (44)..(318): a, c, u, or g
  misc_feature (47)..(317): /note=“This region may encompass 1-271 nucleotides”
  modified_base (320)..(321): a, c, u, or g
  modified_base (328)..(328): a, c, u, or g
  modified_base (333)..(336): a, c, u, or g
  modified_base (340)..(341): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnbbnndunrwnhrdnhygnnbhrdnnynnnnnvyuyusrnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnryubrwnyyhbnnnndvvnnd

SEQ ID NO: 581 - RNA, 340 nt, Artificial Sequence
  source: /note=“Description of Artificial Sequence Synthetic polynucleotide”
  modified_base (1)..(2): a, c, u, or g
  modified_base (5)..(5): a, c, u, or g
  modified_base (12)..(12): a, c, u, or g
  modified_base (20)..(20): a, c, u, or g
  modified_base (29)..(29): a, c, u, or g
  modified_base (31)..(31): a, c, u, or g
  modified_base (39)..(39): a, c, u, or g
  modified_base (43)..(43): a, c, u, or g
  misc_feature (45)..(315): /note=“This region may encompass 1-271 nucleotides”
  modified_base (45)..(316): a, c, u, or g
  modified_base (319)..(319): a, c, u, or g
  modified_base (326)..(326): a, c, u, or g
  modified_base (334)..(334): a, c, u, or g
  modified_base (338)..(338): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nnbynbdyrgunurdyagbnkrrvvywbnbnvyuyusanbvhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnydnryubrwnychbrkbndvvnvd

SEQ ID NO: 582 - RNA, 339 nt, Artificial Sequence
  source: /note=“Description of Artificial Sequence Synthetic polynucleotide”
  modified_base (1)..(1): a, c, u, or g
  modified_base (28)..(28): a, c, u, or g
  modified_base (30)..(30): a, c, u, or g
  modified_base (42)..(42): a, c, u, or g
  modified_base (44)..(314): a, c, u, or g
  misc_feature (44)..(314): /note=“This region may encompass 1-271 nucleotides”
  modified_base (318)..(318): a, c, u, or g
  modified_base (333)..(333): a, c, u, or g
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
ncsydbruggusuakygshkrrsaywbnknsyuyucahbvmndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbyrnryuyrwyucyyrgbnwrsraw

SEQ ID NO: 583 - RNA, 342 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (21)..(22): a, c, u, or g
  modified_base (26)..(26): a, c, u, or g
  modified_base (29)..(34): a, c, u, or g
  modified_base (41)..(319): a, c, u, or g
  misc_feature (47)..(317): /note=“This region may encompass 1-271 nucleotides”
  modified_base (328)..(328): a, c, u, or g
  modified_base (333)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbvnnnnuvrynyrrnyubdnnuadnrynnnnnnyybccvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvvsubbrwnhcbbnnnnnnbbyw

SEQ ID NO: 584 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(40): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gbvbnsvurrudyrrdcggkdadmaudnhhrhyubccannynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnrgsuubrwuuccyksbsnvygca

SEQ ID NO: 585 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(39): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gyvynsvurrudyrrwcggkkadmaudnhhrhyubccandynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbvrgsuuyrwuuccyksbsnvygca

SEQ ID NO: 586 - RNA, 342 nt, Artificial Sequence
  source: /note=“Description of Artificial Sequence Synthetic polynucleotide”
  modified_base (4)..(7): a, c, u, or g
  modified_base (13)..(13): a, c, u, or g
  modified_base (25)..(26): a, c, u, or g
  modified_base (29)..(31): a, c, u, or g
  modified_base (34)..(34): a, c, u, or g
  modified_base (42)..(42): a, c, u, or g
  modified_base (45)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
kgynnnnduvgbnhagbyugghyannvynnndbnyugugrhnvhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgyucranycynnbhnnnvrym

SEQ ID NO: 587 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gchrugaybguayagkgguuasyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwyggyucraaucygagucaygrca

SEQ ID NO: 588 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcmrugayyguauagugguuagyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwygguucraaucygagucaygrca

SEQ ID NO: 589 - RNA, 342 nt, Artificial Sequence
  source: /note=“Description of Artificial Sequence Synthetic polynucleotide”
  modified_base (1)..(1): a, c, u, or g
  modified_base (4)..(5): a, c, u, or g
  modified_base (7)..(7): a, c, u, or g
  modified_base (17)..(17): a, c, u, or g
  modified_base (31)..(32): a, c, u, or g
  modified_base (34)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (328)..(329): a, c, u, or g
  modified_base (332)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nvsnnbndurgybyrrnywggbhrdvrydbnnbnyunaurannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndsywydannchnnnhnnnnsba

SEQ ID NO: 590 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (326)..(327): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyybrkurgykyrruyggyhagmgcrygkurcudauaayryyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnryrrsywydanncycrymyngrsca

SEQ ID NO: 591 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyysrkurgykcaruyggyhagmgcgyggurcudauaayrcyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngygrgyucrabhcycrymybgrsca

SEQ ID NO: 592 - RNA, 341 nt, Artificial Sequence
  source: /note=“Description of Artificial Sequence Synthetic polynucleotide”
  modified_base (1)..(1): a, c, u, or g
  modified_base (3)..(6): a, c, u, or g
  modified_base (11)..(12): a, c, u, or g
  modified_base (16)..(17): a, c, u, or g
  modified_base (21)..(22): a, c, u, or g
  modified_base (25)..(26): a, c, u, or g
  modified_base (28)..(31): a, c, u, or g
  modified_base (41)..(41): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (335)..(338): a, c, u, or g
  modified_base (340)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
nbnnnndursnnbarnnhggnnddnnbnnnnvdhycauahnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgdusdanhmynnbhnnnnvnw

SEQ ID NO: 593 - RNA, 339 nt, Artificial Sequence
  source: /note=“Description of Artificial Sequence Synthetic polynucleotide”
  modified_base (4)..(4): a, c, u, or g
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rbcnbvkurgygcagydgghagyryryyrgkyycauaahyyvrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsdkrgdusdadmmymwsmbvbgsya

SEQ ID NO: 594 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rschbvkurgygcagydgghagcryryyrgkyycauaaycyrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnswkrgwusdadmcymwsmbvkgsya

SEQ ID NO: 595 - RNA, 342 nt, Artificial Sequence
  source: /note=“Description of Artificial Sequence Synthetic polynucleotide”
  modified_base (2)..(7): a, c, u, or g
  modified_base (9)..(12): a, c, u, or g
  modified_base (16)..(16): a, c, u, or g
  modified_base (23)..(23): a, c, u, or g
  modified_base (26)..(26): a, c, u, or g
  modified_base (28)..(34): a, c, u, or g
  modified_base (37)..(37): a, c, u, or g
  modified_base (41)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
vnnnnnnunnnnvrrnhhgkhhndhnvnnnnnnnhynardnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvbyuhvanymbnnbnnnnnnbr

SEQ ID NO: 596 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (41)..(41): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (328)..(328): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbbvvndurkhhsrrbhygkhhurwbrhdbhdnryuhardnynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvanymybnbbnbbvnya

SEQ ID NO: 597 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbbvvndurkhhsrrbhygkhhurwkrhdbhdnryuhardhynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvavymybnbynbbvnya

SEQ ID NO: 598 - RNA, 342 nt, Artificial Sequence
  source: /note=“Description of Artificial Sequence Synthetic polynucleotide”
  modified_base (1)..(7): a, c, u, or g
  modified_base (11)..(12): a, c, u, or g
  modified_base (16)..(16): a, c, u, or g
  modified_base (21)..(21): a, c, u, or g
  modified_base (26)..(26): a, c, u, or g
  modified_base (30)..(32): a, c, u, or g
  modified_base (44)..(320): a, c, u, or g
  misc_feature (47)..(317): /note=“This region may encompass 1-271 nucleotides”
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnnnnnnursnnharnhwrrnuudrnrydnnnsvyyyuummbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvryuyrwnhybnnbnnnnnnhd

SEQ ID NO: 599 - RNA, 340 nt, Artificial Sequence
  source: /note=“Description of Artificial Sequence Synthetic polynucleotide”
  modified_base (1)..(1): a, c, u, or g
  modified_base (6)..(6): a, c, u, or g
  modified_base (11)..(11): a, c, u, or g
  modified_base (29)..(29): a, c, u, or g
  modified_base (43)..(43): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (333)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nhvbdnvkrsnhharbhrrbudrdrydbndgryyyuummymhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgryuyrwbyysynbnnhndrhr

SEQ ID NO: 600 - RNA, 340 nt, Artificial Sequence
  source: /note=“Description of Artificial Sequence Synthetic polynucleotide”
  misc_feature (45)..(315): /note=“This region may encompass 1-271 nucleotides”
  modified_base (45)..(316): a, c, u, or g
  modified_base (333)..(333): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
vhvbrdvkascwharuhrrbudrwrcrkvdgacuyuumauchswnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrgryuyrwkyysyhbnhynkryr

SEQ ID NO: 601 - RNA, 341 nt, Artificial Sequence
  source: /note=“Description of Artificial Sequence Synthetic polynucleotide”
  modified_base (4)..(6): a, c, u, or g
  modified_base (29)..(31): a, c, u, or g
  modified_base (33)..(33): a, c, u, or g
  modified_base (41)..(41): a, c, u, or g
  modified_base (43)..(318): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(338): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
kbbnnnduagyyyarhyuggbwwgrryrnnnbnyygaarvnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdguucranychnnbhnnnnvma

SEQ ID NO: 602 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbyvwrruwgyyyaruuggsuagrrydykrbrcygaarrysyhmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyckgguucrayycysgguywbrvma

SEQ ID NO: 603 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcyrarauwgcucaruugggagagyguuasacygaagaucuwmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnycugguucrayycyggguuucrvca

SEQ ID NO: 604 - RNA, 342 nt, Artificial Sequence
  source: /note=“Description of Artificial Sequence Synthetic polynucleotide”
  modified_base (4)..(6): a, c, u, or g
  modified_base (16)..(16): a, c, u, or g
  modified_base (21)..(21): a, c, u, or g
  modified_base (29)..(34): a, c, u, or g
  modified_base (37)..(37): a, c, u, or g
  modified_base (41)..(320): a, c, u, or g
  misc_feature (47)..(317): /note=“This region may encompass 1-271 nucleotides”
  modified_base (328)..(328): a, c, u, or g
  modified_base (331)..(334): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
sksnnnduvgbbyagnyygbnyyrdvvynnnnnnhunggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndguucranycnnnnhnnnbmsm

SEQ ID NO: 605 - RNA, 338 nt, Artificial Sequence
  source: /note=“Description of Artificial Sequence Synthetic polynucleotide”
  modified_base (39)..(39): a, c, u, or g
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcusguuggkcuagkgburugruucucrsyuhggrysnragnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncyggguucaaaucvyrgasgagccc

SEQ ID NO: 606 - RNA, 338 nt, Artificial Sequence
  source: /note=“Description of Artificial Sequence Synthetic polynucleotide”
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcucguuggucuagkggurugruucucgsuuhggrysbgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggguucaaaucccggacgagccc

SEQ ID NO: 607 - RNA, 342 nt, Artificial Sequence
  source: /note=“Description of Artificial Sequence Synthetic polynucleotide”
  modified_base (2)..(7): a, c, u, or g
  modified_base (9)..(13): a, c, u, or g
  modified_base (16)..(16): a, c, u, or g
  modified_base (21)..(21): a, c, u, or g
  modified_base (25)..(28): a, c, u, or g
  modified_base (30)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (332)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
dnnnnnnynnnnnarnbhggnbbannnndnnnnnyynswrmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrkuuyradycynnnnnnnnnbd

SEQ ID NO: 608 - RNA, 341 nt, Artificial Sequence
  source: /note=“Description of Artificial Sequence Synthetic polynucleotide”
  modified_base (2)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (331)..(331): a, c, u, or g
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knnnnbdurgchsarkbugguuavdghrdbkdrcynswraybmvdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvyngkuuyradyccnrbhnnbnnyd

SEQ ID NO: 609 - RNA, 341 nt, Artificial Sequence
  source: /note=“Description of Artificial Sequence Synthetic polynucleotide”
  modified_base (2)..(3): a, c, u, or g
  modified_base (5)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knndnbrurgchsarkcugguuavdghrwykrrcunswrayymrdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvydgguuyraaycchrbynnynnyk

SEQ ID NO: 610 - RNA, 341 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (25)..(25): a, c, u, or g
  modified_base (29)..(31): a, c, u, or g
  modified_base (33)..(33): a, c, u, or g
  modified_base (36)..(36): a, c, u, or g
  modified_base (41)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(337): a, c, u, or g
  modified_base (341)..(341): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rbbnnnnurgynhadnhykghyrdnryrnnnsnhynguwansnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndgwyyvanbyhnnnnnnnrvyn

SEQ ID NO: 611 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (326)..(326): a, c, u, or g
  modified_base (331)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rbbnbnduvgynharbygghyrdnryryhbsbcyhguwavsvdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvndrgwuyvanbyyhnnbndnrvyb

SEQ ID NO: 612 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (326)..(326): a, c, u, or g
  modified_base (332)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rsynbnduvgynharbugghyrdnryryhksbcyhguaavsmdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvhdrgwuyvanbyyhdnbndnrsyu

SEQ ID NO: 613 - RNA, 342 nt, Artificial Sequence
  source: /note=“Description of Artificial Sequence Synthetic polynucleotide”
  modified_base (4)..(6): a, c, u, or g
  modified_base (29)..(32): a, c, u, or g
  modified_base (34)..(34): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(319): a, c, u, or g
  modified_base (321)..(321): a, c, u, or g
  modified_base (328)..(328): a, c, u, or g
  modified_base (333)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rvvnnndurrybhhrhyhgghyuadvvynnnnrnbuccavanbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnrnguucranychynbhnnnbbhd

SEQ ID NO: 614 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
grmmkvrkggysmaryuggharssykkybrrsuccasakbvrwnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsygkguucradycmcrkyksbgyma

SEQ ID NO: 615 - RNA, 339 nt, Artificial Sequence; identical to SEQ ID NO: 614
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”

SEQ ID NO: 616 - RNA, 342 nt, Artificial Sequence
  source: /note=“Description of Artificial Sequence Synthetic polynucleotide”
  modified_base (3)..(6): a, c, u, or g
  modified_base (9)..(10): a, c, u, or g
  modified_base (12)..(13): a, c, u, or g
  modified_base (16)..(16): a, c, u, or g
  modified_base (25)..(26): a, c, u, or g
  modified_base (30)..(32): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
bbnnnndunnynnarnyugsyhwrnnrbdnnnrdcuruarvnynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvgwuyranucbnnbhnnnnnvd

SEQ ID NO: 617 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkkbdauagyucagbugsyuagagydkwkracuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscuggwuyrahucyrrsubsnmsvd

SEQ ID NO: 618 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkksdauagyucagyuggyuagagcdkwkgacuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscugguuyrawuccrrsuysdmsva

SEQ ID NO: 619 - RNA, 341 nt, Artificial Sequence
  source: /note=“Description of Artificial Sequence Synthetic polynucleotide”
  modified_base (2)..(2): a, c, u, or g
  modified_base (4)..(7): a, c, u, or g
  modified_base (12)..(12): a, c, u, or g
  modified_base (15)..(16): a, c, u, or g
  modified_base (21)..(21): a, c, u, or g
  modified_base (24)..(25): a, c, u, or g
  modified_base (28)..(28): a, c, u, or g
  modified_base (30)..(33): a, c, u, or g
  modified_base (36)..(36): a, c, u, or g
  modified_base (40)..(319): a, c, u, or g
  misc_feature (46)..(316): /note=“This region may encompass 1-271 nucleotides”
  modified_base (327)..(327): a, c, u, or g
  modified_base (330)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dndnnnnuvrynbrnnhugknyannrynbnnnnhunacrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrdubranhynnnnnnnnnnyv

SEQ ID NO: 620 - RNA, 340 nt, Artificial Sequence
  source: /note=“Description of Artificial Sequence Synthetic polynucleotide”
  modified_base (15)..(16): a, c, u, or g
  modified_base (24)..(24): a, c, u, or g
  modified_base (27)..(27): a, c, u, or g
  modified_base (29)..(30): a, c, u, or g
  modified_base (41)..(43): a, c, u, or g
  modified_base (45)..(315): a, c, u, or g
  misc_feature (45)..(315): /note=“This region may encompass 1-271 nucleotides”
  modified_base (317)..(318): a, c, u, or g
  modified_base (326)..(326): a, c, u, or g
  modified_base (330)..(330): a, c, u, or g
  modified_base (333)..(333): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbshdurrydbrnnhgkuyadnrynynndhyuhacrhknnnrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnrrdubranhybndvndsdwvyv

SEQ ID NO: 621 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (317)..(318): a, c, u, or g
  modified_base (330)..(330): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbsydurgydbrbhugkuyadhryvyhhdyyuhacahkhddrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnngrdubradhyyndvhrsdwvya

SEQ ID NO: 622 - DNA, 72 bp, Artificial Sequence; identical to SEQ ID NO: 262
  source: /note=“Description of Artificial Sequence Synthetic oligonucleotide”

SEQ ID NO: 623 - DNA, 20 bp, Artificial Sequence
  source: /note=“Description of Artificial Sequence Synthetic primer”
tgagttggcaacctgtggta

SEQ ID NO: 624 - DNA, 20 bp, Artificial Sequence
  source: /note=“Description of Artificial Sequence Synthetic primer”
ttgggtgtccatgaaaatca

SEQ ID NO: 625 - RNA, 26 nt, Artificial Sequence
  source: /note=“Description of Artificial Sequence Synthetic probe”
uagcagaggaugguuucgauccauca

SEQ ID NO: 626 - DNA, 72 bp, Artificial Sequence; identical to SEQ ID NO: 262
  source: /note=“Description of Artificial Sequence Synthetic oligonucleotide”

SEQ ID NO: 627 - DNA, 26 bp, Artificial Sequence
  source: /note=“Description of Artificial Sequence Synthetic probe”
tagcagaggatggtttcgatccatca

SEQ ID NO: 628 - protein, 6 aa, Artificial Sequence
  source: /note=“Description of Artificial Sequence Synthetic 6xHis tag”
HHHHHH